(19) United States
(12) Patent Application Publication
(10) Pub. No.: US 2016/0083400 A1
(43) Pub. Date: Mar. 24, 2016

(54) TRICYCLIC GUANIDINE DERIVATIVE

(71) Applicants: Sunovion Pharmaceuticals Inc., Marlborough, MA (US); Sumitomo Dainippon Pharma Co., Ltd., Osaka (JP)

(72) Inventors: Douglas F. Burdi, Arlington, MA (US); Daisuke Tanaka, Osaka (JP); Hiroaki Fujiwara, Osaka (JP)

(21) Appl. No.: 14/857,682

(22) Filed: Sep. 17, 2015

Related U.S. Application Data
(60) Provisional application No. 62/052,359, filed on Sep. 18, 2014.

(51) Int. Cl. C07D 498/14; C07D 487/20; C07D 487/14

(52) U.S. Cl. CPC C07D 498/14; C07D 487/14; C07D 487/20

(57) ABSTRACT

Disclosed are compounds useful as inhibitors of Phosphodiesterase 1 (PDE1), compositions thereof, and methods of using the same.

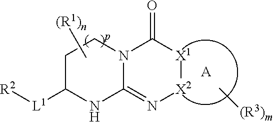

### BACKGROUND OF THE INVENTION

The prevalence of neurological and psychiatric disorders is increasing worldwide. Up to one billion people suffer from debilitating neurological conditions such as Alzheimer's disease and Parkinson's disease, with almost seven million people dying every year. “Neurological disorders: public health challenges” World Health Organization, 2006. Neurological and psychiatric disorders are prevalent in all countries, often without regard to age, sex, education or income. However, as many neurological disorders are correlated with increased age, as the global population ages, the impact of these disorders becomes more evident.

Despite the availability of treatments for some of these diseases, first line therapies (such as L-DOPA for Parkinson's) are often burdened by unfavorable side effects, or may lack efficacy. For instance, there is currently no approved treatment for the cognitive deficits in schizophrenia despite the high unmet medical need.

The continuing and increasing problem of neurological and psychiatric disorders, and the current lack of safe and effective drugs for treating them, highlight the overwhelming need for new drugs to treat these conditions and their underlying causes.

### SUMMARY OF THE INVENTION

It has now been found that compounds of this invention, and pharmaceutically acceptable compositions thereof, are effective as inhibitors of Phosphodiesterase 1 (PDE1) enzymes. Such compounds have the general formula I:

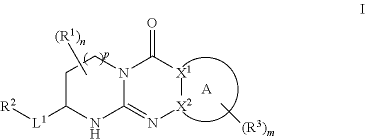

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with regulation of PDE1 enzymes. Such diseases, disorders, or conditions include those described herein.

Compounds provided by this invention are also useful for the study of PDE1 enzymes in biological and pathological phenomena; the study of intracellular signal transduction pathways occurring in PDE1-expressing tissues; and the comparative evaluation of new PDE1 inhibitors or other regulators neuronal activity in vitro or in vivo.

### DETAILED DESCRIPTION OF CERTAIN EMBODIMENTS

### 1. General Description of Compounds of the Invention

In certain embodiments, the present invention provides inhibitors of PDE1. In some embodiments, such compounds include those of formula I:

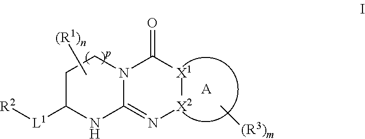

or a pharmaceutically acceptable salt thereof, wherein:
X¹ and X² are each independently C or N;
Ring A is a 5-6 membered heteroaryl ring having 1-4 heteroatoms independently selected from nitrogen, oxygen and sulfur;
L¹ is a covalent bond, or a C₁₋₄ bivalent straight or branched hydrocarbon chain, wherein one or more hydrogen atoms of the chain are optionally and independently replaced by halogen, and wherein one or two methylene units of the chain are optionally and independently repined by —N(R)—, —N(R)C(O)—, —C(O)N(R)—, —N(R)C(O)N(R)—, —N(R)C(S)N(R)—, —N(R)S(O)₂—, —S(O)₂N(R)—, —C(O)—, —OC(O)—, —C(O)O—, —O—, —S—, —S(O)— or —S(O)₂—;
each R¹ is independently halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)S(O)₂R, —S(O)₂N(R)₂, —OC(O)OR, —C(O)OR, —OC(O)R, —C(O)R; —S(O)R, or —S(O)₂R;
R² is halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)SO₂R, —S(O)₂N(R)₂, —C(O)R, —C(O)OR, —OC(O)R, —S(O)R, —S(O)₂R, or Cy;
Cy is a ring, substituted with q instances of R⁴; wherein said ring is a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; or an 8-10 membered bicyclic heteroaromatic ring having 1-5 heteroatoms independently selected from nitrogen, oxygen, and sulfur;
each R is independently

(i) a hydrogen,

(ii) a C₁₋₆ aliphatic (said group being optionally substituted with the same or different 1 to 4 group(s) selected from

(a) a halogen,

(b) a C₁₋₆ alkyl (said group being optionally substituted with the same or different 1 to 3 halogen),

(c) a C₁₋₆ alkoxy (said group being optionally substituted with the same or different 1 to 3 halogen),

(d) a hydroxy, and

(e) an oxo), or

(iii) a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring; a 5-6 membered monocyclic heteroaromatic ring; or an 8-10 membered bicyclic heteroaromatic ring, wherein each of said groups is optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) a cyano;
each R³ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, or —B(OR)₂;
each R⁴ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, or —B(OR)₂; or
two R⁴ are taken together with their intervening atoms to form a 5-6 membered saturated, partially unsaturated, or aromatic ring having 0-3 heteroatoms independently selected from nitrogen, oxygen, and sulfur;
wherein one or more of {two instances of R¹}, {R¹ and an R²}, and {two instances of R³} may be taken together with their intervening atoms to form a ring, substituted with r instances of R⁴; wherein said ring is a 3-8 membered saturated or partially unsaturated monocyclic carbocyclic ring; phenyl; a 4-8 membered saturated or partially unsaturated monocyclic heterocyclic ring having 1-2 heteroatoms independently selected from nitrogen, oxygen, and sulfur; or a 5-6 membered monocyclic heteroaromatic ring having 1-4 heteroatoms independently selected from nitrogen, oxygen, and sulfur;
m is 0-4;
n is 0-3;
p is 0-2;
q is 0-5; and
r is 0-5.

### 2. Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₇ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, boron, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆ or C₁₋₄) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₜ—, wherein t is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is a carbocyclic aromatic ring and wherein each ring in the system contains 3 to 7 ring members. The term “aryl” may be used interchangeably with the term “aryl ring.” In certain embodiments of the present invention, “aryl” refers to a carbocyclic aromatic ring system which includes, but not limited to, phenyl, naphthyl, anthracyl and the like, which may be optionally substituted. Also included within the scope of the term “aryl,” as it is used herein, is a group in which a carbocyclic aromatic ring is fused to one or more nonaromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, 9 or 10 ring atoms; having 6, 10, or 14 π electrons shared in a cyclic array; and having, in addition to carbon atoms, from one to five heteroatoms. Heteroaryl groups include, without limitation, thienyl, furanyl, pyrrolyl, imidazotyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3b]-1,4-oxazin-3(4H)-one. A heteroaryl group may be mono or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —NO₂; —CN; —N₃; —(CH₂)₀₋₄N(R^{∘})₂; —(CH₂)₀₋₄N(R^{∘})C(O)R^{∘}; —(CH₂)₀₋₄N(R^{∘})C(S)R^{∘}; —(CH₂)₀₋₄N(R^{∘})C(O)NR^{∘}₂; —(CH₂)₀₋₄N(R^{∘})C(S)NR^{∘}₂; —(CH₂)₀₋₄N(R^{∘})C(O)OR^{∘}; —(CH₂)₀₋₄N(R^{∘})N(R^{∘})C(O)R^{∘}; —(CH₂)₀₋₄N(R^{∘})N(R^{∘})C(O)NR^{∘}₂; —(CH₂)₀₋₄N(R^{∘})N(R^{∘})C(O)OR^{∘}; —(CH₂)₀₋₄C(O)R^{∘}; —(CH₂)₀₋₄C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}—, —(CH₂)₀₋₄SC(S)SR^{∘}; —(CH₂)₀₋₄SC(O)R^{∘}; —(CH₂)₀₋₄C(O)NR^{∘}₂; —(CH₂)₀₋₄C(S)NR^{∘}₂; —(CH₂)₀₋₄C(S)SR^{∘}; —(CH₂)₀₋₄SC(S)SR^{∘}, —(CH₂)₀₋₄OC(O)NR^{∘}₂; —(CH₂)₀₋₄C(O)N(OR^{∘})R^{∘}; —(CH₂)₀₋₄C(O)C(O)R^{∘}, —(CH₂)₀₋₄C(O)CH₂C(O)R^{∘}; —(CH₂)₀₋₄C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}: —(CH₂)₀₋₄OS(O)₂R^{∘}; —(CH₂)₀₋₄S(O)₂NR^{∘}₂; —(CH₂)₀₋₄S(O)R^{∘}; —(CH₂)₀₋₄N(R^{∘})S(O)₂NR^{∘}₂; —(CH₂)₀₋₄N(R^{∘})S(O)₂R^{∘}; —(CH₂)₀₋₄N(OR^{∘})R^{∘}; —(CH₂)₀₋₄C(NH)NR^{∘}₂; —(CH₂)₀₋₄P(O)₂R^{∘}; —(CH₂)₀₋₄P(O)R^{∘}₂; —(CH₂)₀₋₄OP(O)R^{∘}₂; —(CH₂)₀₋₄OP(O)(OR^{∘})₂; —(CH₂)₀₋₄SiR^{∘}₃; —(CH₂)₁₋₄O—N(R^{∘})₂; or —(CH₂)₁₋₄C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5- or 6-membered saturated, partially unsaturated, or aromatic ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aromatic mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{}, —(CH₂)₀₋₂OH, —(CH₂)₀₋₂OR^{}, —(CH₂)₀₋₂CH(OR^{})₂, —CN, —N₃, —(CH₂)₀₋₂C(O) R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or substituted with one or more halogens, and is independently selected from C₁₋₄ aliphatic, —CH₂Ph, —O(CH₂)₀₋₁Ph, or a 5- or 6-membered saturated, partially unsaturated, or aromatic ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5- or 6-membered saturated, partially unsaturated, or aromatic ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5- or 6-membered saturated, partially unsaturated, or aromatic ring having 0-4 heteroatoms independently selected from nitrogen, oxygen, or sulfur.

Suitable substituents on the aliphatic group of R* include halogen, —R^{}, —OH, —OR^{}, —CN, —C(O)OH, —C(O)OR^{}, —NH₂, —NHR^{}, —NR^{}₂, or —NO₂, wherein each R^{} is unsubstituted or substituted with one or more halogens, and is independently C₁₋₄ aliphatic, —CH₂Ph, —O(CH₂)₀₋₁Ph, or a 5- or 6-membered saturated, partially unsaturated, or aromatic ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5- or 6-membered saturated, partially unsaturated, or aromatic ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aromatic mono or bicyclic ring having 0-4 heteroatoms independently selected from nitrogen, oxygen, or sulfur.

Suitable substituents on the aliphatic group of R^{†} are independently halogen, —R^{†}, —OH, —OR^{†}, —CN, —C(O)OH, —C(O)OR^{†}, —NH₂, —NHR^{†}, —NR^{†}₂, or —NO₂, wherein each R^{†} is unsubstituted or substituted with one or more halogens, and is independently C₁₋₄ aliphatic, —CH₂Ph, —O(CH₂)₀₋₁Ph, or a 5- or 6-membered saturated, partially unsaturated, or aromatic ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

### 3. Description of Exemplary Embodiments

In certain embodiments, the present invention provides inhibitors of PDE1. In some embodiments, such compounds include those of formula I:

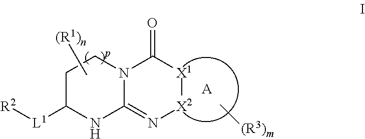

or a pharmaceutically acceptable salt thereof, wherein:
X¹ and X² are each independently C or N;
Ring A is a 5-6 membered heteroaryl ring having 1-4 heteroatoms independently selected from nitrogen, oxygen and sulfur;
L¹ is a covalent bond, or a C₁₋₄ bivalent straight or branched hydrocarbon chain, wherein one or more hydrogen atoms of the chain are optionally and independently replaced by halogen, and wherein one or two methylene units of the chain are optionally and independently replaced by —N(R)—, —N(R)C(O)—, —C(O)N(R)—, —N(R)C(O)N(R)—, —N(R)C(S)N(R)—, —N(R)S(O)₂—, —S(O)₂N(R)—, —C(O)—, —OC(O)—, —C(O)O—, —O—, —S—, —S(O)— or —S(O)₂—;
each R¹ is independently halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)S(O)₂R, —S(O)₂N(R)₂, —OC(O)OR, —C(O)OR, —OC(O)R, —C(O)R; —S(O)R, or —S(O)₂R;
R² is halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)SO₂R, —S(O)₂N(R)₂, —C(O)R, —C(O)OR, —OC(O)R, —S(O)R, —S(O)₂R, or Cy;
Cy is a ring, substituted with q instances of R⁴; wherein said ring is a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; or an 8-10 membered bicyclic heteroaromatic ring having 1-5 heteroatoms independently selected from nitrogen, oxygen, and sulfur;
each R is independently

(i) a hydrogen,

(ii) a C₁₋₆ aliphatic (said group being optionally substituted with the same or different 1 to 4 group(s) selected from

(a) a halogen,

(b) a C₁₋₆ alkyl (said group being optionally substituted with the same or different 1 to 3 halogen),

(c) a C₁₋₆ alkoxy (said group being optionally substituted with the same or different 1 to 3 halogen),

(d) a hydroxy, and

(e) an oxo), or

(iii) a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring; a 5-6 membered monocyclic heteroaromatic ring; or an 8-10 membered bicyclic heteroaromatic ring, wherein each of said groups is optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) a cyano;
each R³ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, or —B(OR)₂;
each R⁴ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, or —B(OR)₂; or
two R⁴ are taken together with their intervening atoms to form a 5-6 membered saturated, partially unsaturated, or aromatic ring having 0-3 heteroatoms independently selected from nitrogen, oxygen, and sulfur;
wherein one or more of {two instances of R¹}, {R¹ and an R²}, and {two instances of R³} may be taken together with their intervening atoms to form a ring, substituted with r instances of R⁴; wherein said ring is a 3-8 membered saturated or partially unsaturated monocyclic carbocyclic ring; phenyl; a 4-8 membered saturated or partially unsaturated monocyclic heterocyclic ring having 1-2 heteroatoms independently selected from nitrogen, oxygen, and sulfur; or a 5-6 membered monocyclic heteroaromatic ring having 1-4 heteroatoms independently selected from nitrogen, oxygen, and sulfur;
m is 0-4;
n is 0-3;
p is 0-2;
q is 0-5; and
r is 0-5.

As defined generally above, X¹ and X² are each independently C or N. In some embodiments, X¹ is C, and X² is N. In some embodiments, X¹ is N, and X² is C. In some embodiments both of X¹ and X² are C.

As defined generally above, Ring A is a 5-6 membered heteroaryl ring having 1-4 heteroatoms independently selected from nitrogen, oxygen and sulfur. In some embodiments, Ring A is pyrrolo. In some embodiments, Ring A is furano. In some embodiments, Ring A is thieno. In some embodiments, Ring A is pyrazolo. In some embodiments, Ring A is imidazolo. In some embodiments, Ring A is oxazolo. In some embodiments, Ring A is isoxazolo. In some embodiments, Ring A is thiazolo. In some embodiments, Ring A is isothiazolo. In some embodiments, Ring A is triazolo. In some embodiments, Ring A is tetrazolo. In some embodiments, Ring A is pyridino. In some embodiments, Ring A is pyrimidino. In some embodiments, Ring A is pyridizino. In some embodiments, Ring A is selected from pyrazolo and imidazolo. In some embodiments, Ring A is not pyrrolo, thieno, or furano.

As defined generally above, each R¹ is independently halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)S(O)₂R, —S(O)₂N(R)₂, —OC(O)OR, —C(O)OR, —OC(O)R, —C(O)R; —S(O)R, or —S(O)₂R. In certain embodiments, R¹ is hydrogen. In some embodiments, R¹ is C₁₋₄ aliphatic. In some embodiments, R¹ is methyl. In some embodiments, R¹ is trifluoromethyl. In some embodiments, R¹ is —R, where —R is not hydrogen. In some embodiments, R¹ is phenyl (said group being optionally substituted with the same or different 1 to 4 group(s) selected from

(a) a halogen,

(b) a C₁₋₆ alkyl (said group being optionally substituted with the same or different 1 to 3 halogen), and

(c) a C₁₋₆ alkoxy (said group being optionally substituted with the same or different 1 to 3 halogen)).

As defined generally above, L¹ is a covalent bond, or a C₁₋₄ bivalent straight or branched hydrocarbon chain, wherein one or more hydrogen atoms of the chain are optionally and independently replaced by halogen, and wherein one or two methylene units of the chain are optionally and independently replaced by —N(R)—, —N(R)C(O)—, —C(O)N(R)—, —N(R)C(O)N(R)—, —N(R)C(S)N(R)—, —N(R)S(O)₂—, —S(O)₂N(R)—, —C(O)—, —C(O)O—, —OC(O)—, —O—, —S—, —S(O)— or —S(O)₂—. In some embodiments, L¹ is a covalent bond. In some embodiments, L¹ is a C₁₋₄ bivalent straight or branched hydrocarbon chain, wherein one or more hydrogen atoms of the chain are optionally and independently replaced by halogen. In some embodiments, L¹ is a C₁₋₄ bivalent straight or branched hydrocarbon chain, wherein one or two methylene units of the chain are optionally and independently replaced by —N(R)—, —N(R)C(O)—, —C(O)N(R)—, —N(R)S(O)₂—, —S(O)₂N(R)—, —C(O)O—, —OC(O)—, —C(O)—, —O—, —S—, —S(O)— or —S(O)₂—. In some embodiments, L¹ is selected from methylene, ethylene, propylene and butylene. In some embodiments, L¹ is methylene. In some embodiments, L¹ is —O—. In some embodiments, L¹ is —S—, —S(O)—, or —S(O)₂—.

As defined generally above, R² is halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)SO₂R, —S(O)₂N(R)₂, —C(O)R, —C(O)OR, —OC(O)R, —S(O)R, —S(O)₂R, or Cy. In some embodiments, R² is hydrogen. In some embodiments, R² is not hydrogen. In some embodiments. R² is Cy. In some embodiments, R² is C₃₋₇ cycloaliphatic; a phenyl; a 5-6 membered monocyclic heteroaryl, or a 4-8 membered saturated or partially unsaturated monocyclic heterocyclyl, wherein each of said groups is optionally substituted with the same or different 1 to 4 group(s) selected from

(a) a halogen,

(b) a C₁₋₆ alkyl (said group being optionally substituted with the same or different 1 to 3 halogen),

(c) a C₁₋₆ alkoxy (said group being optionally substituted with the same or different 1 to 3 halogen),

(d) a hydroxy, and

(e) a cyano.

As defined generally above, Cy is a ring, substituted with q instances of R⁴; wherein said ring is a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; or an 8-10 membered bicyclic heteroaromatic ring having 1-5 heteroatoms independently selected from nitrogen, oxygen, and sulfur. In some embodiments, said ring of Cy is phenyl. In some embodiments, said ring of Cy is a 5-6 membered monocyclic heteroaromatic ring having 1-4 heteroatoms independently selected from nitrogen, oxygen, and sulfur. In some embodiments, said ring of Cy is a 6-membered monocyclic heteroaromatic ring having 1-2 nitrogens. In some embodiments, said ring of Cy is pyrimidinyl. In some embodiments, said ring of Cy is pyridyl.

As defined generally above, each R³ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)SO₂R, —S(O)₂N(R)₂, —C(O)R, —C(O)OR, —OC(O)R, —S(O)R, —S(O)₂R, or —B(OR)₂. In some embodiments, each R³ is —R. In some embodiments, an R³ is an optionally substituted C₁₋₆ aliphatic group. In some embodiments, an R³ is methyl, ethyl, propyl, isopropyl, butyl, sec-butyl, isobutyl, tert-butyl, pentyl, tert-pentyl, neopentyl, isopentyl, sec-pentyl (pentan-2-yl), or 3-pentyl (pentan-3-yl), In some embodiments, an R³ is isopropyl. In some embodiments, an R³ is methyl. In some embodiments, an R³ is propyl. In some embodiments, an R³ is neopentyl. In some embodiments, an R³ is an optionally substituted 3-8 membered saturated or partially unsaturated monocyclic carbocyclic ring. In some embodiments, an R³ is optionally substituted cyclopropyl, cyclobutyl, cyclopentyl or cyclohexyl. In some embodiments, an R³ is optionally substituted cyclobutyl or cyclopentyl. In some embodiments, an R³ is optionally substituted phenyl. In some embodiments, an R³ is an optionally substituted 4-8 membered saturated or partially unsaturated monocyclic heterocyclic ring having 1-2 heteroatoms independently selected from nitrogen, oxygen, and sulfur. In some embodiments, an R³ is optionally substituted tetrahydropyranyl or tetrahydrofuranyl. In some embodiments, each R³ is the same. In some embodiments, each R³ is different.

As defined generally above, each R⁴ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)SO₂R, —SO₂N(R)₂, —C(O)R, —C(O)OR, —OC(O)R, —S(O)R, —SO₂R, or —B(OR)₂; or two R⁴ are taken together with their intervening atoms to form a 5-6 membered saturated, partially unsaturated, or aromatic ring having 0-3 heteroatoms independently selected from nitrogen, oxygen, and sulfur. In some embodiments, each R⁴ is independently halogen, —R, —CN, or —OR. In some embodiments, each R⁴ is independently halogen, phenyl, methyl, ethyl, trifluoromethyl, —CN, methoxy, ethoxy, propoxy, or isopropoxy. In some embodiments, q is 1-2 and each R⁴ is independently halogen, phenyl, methyl, ethyl, trifluoromethyl, —CN, methoxy, ethoxy, propoxy, or isopropoxy. In some embodiments q is 2, and one R⁴ is —OR, and the other is halogen. In some embodiments, each R⁴ is the same. In some embodiments, each R⁴ is different. In some embodiments, two R⁴ are taken together with their intervening atoms to form a 5-6 membered saturated, partially unsaturated, or aromatic ring having 0-3 heteroatoms independently selected from nitrogen, oxygen, and sulfur. In some embodiments, two R⁴ are taken together with their intervening atoms to form a dioxolo ring.

As defined generally above one or more of {two instances of R¹}, {R¹ and an R²}, and {two instances of R³} may be taken together with their intervening atoms to form a ring, substituted with r instances of R⁴; wherein said ring is a 3-8 membered saturated or partially unsaturated monocyclic carbocyclic ring; phenyl; a 4-8 membered saturated or partially unsaturated monocyclic heterocyclic ring having 1-2 heteroatoms independently selected from nitrogen, oxygen, and sulfur; or a 5-6 membered monocyclic heteroaromatic ring having 1-4 heteroatoms independently selected from nitrogen, oxygen, and sulfur. In some embodiments, none of {two instances of R¹}, {R¹ and an R²}, and {two instances of R³} are taken together with their intervening atoms to form a ring. In some embodiments, one of {two instances of R¹}, {R¹ and an R²}, and {two instances of R³} is taken together with their intervening atoms to form a ring substituted with r instances of R⁴. In some embodiments, two of {two instances of R¹}, {R¹ and an R²}, and {two instances of R³} are taken together with their intervening atoms to form a ring substituted with r instances of R⁴. In some embodiments, two instances of R¹ are taken together with their intervening atoms to form a ring substituted with r instances of R⁴. In some embodiments, R¹ and an R² are taken together with their intervening atoms to form a ring substituted with r instances of R⁴. In some embodiments, two instances of R³ are taken together with their intervening atoms to form a ring substituted with r instances of R⁴.

As defined generally above, m is 0-4. In some embodiments, m is 0-1. In some embodiments, m is 1.

As defined generally above, n is 0-4. In some embodiments, n is 0-1. In some embodiments, n is 0.

As defined generally above, p is 0-2. In some embodiments, p is 0-1. In some embodiments, p is 0. In some embodiments, p is 1.

As defined generally above, q is 0-5. In some embodiments, q is 0. In some embodiments, q is 1-5. In some embodiments, q is 1-2. In some embodiments, q is 1. In some embodiments, q is 2. In some embodiments, q is 3.

As defined generally above, r is 0-5. In some embodiments, r is 0. In some embodiments, r is 1-5. In some embodiments, r is 1-2. In some embodiments, r is 1. In some embodiments, r is 2. In some embodiments, r is 3.

In some embodiments, the present invention provides a compound of formula I selected from formulas I-a, I-b, and I-c:

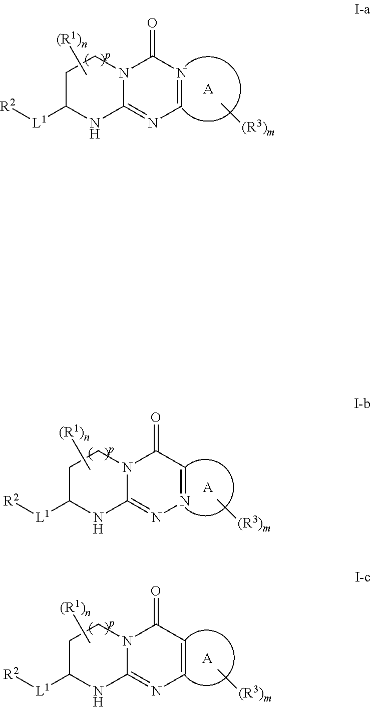

or a pharmaceutically acceptable salt thereof; wherein each of L¹, Ring A, R¹, R², R³, p, n, and m is as described in embodiments for formula I, supra, or described in embodiments herein, both singly and in combination.

In certain embodiments, the present invention provides a compound of formula I selected from formulas II-a, II-b, II-c, II-d, II-e, II-f, II-g, II-h, II-i, II-j, II-k, II-l, II-m, and II-n:

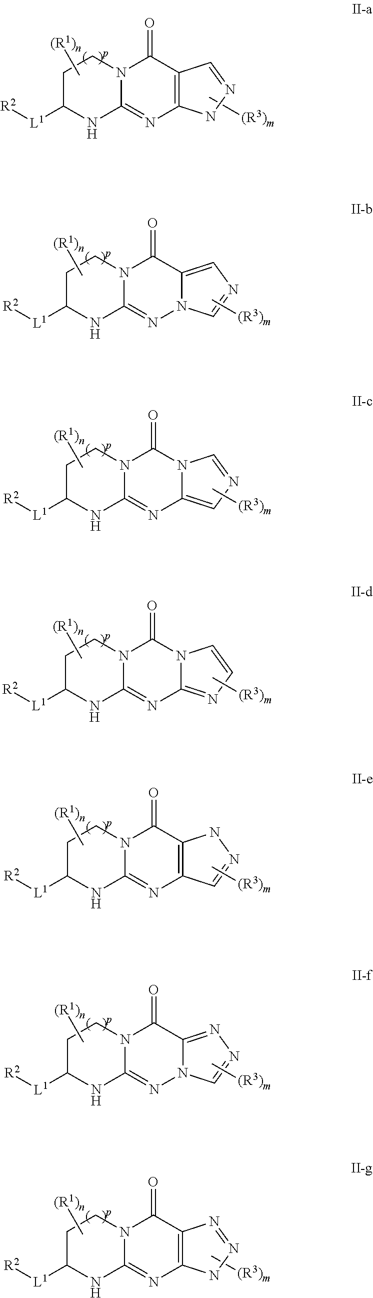

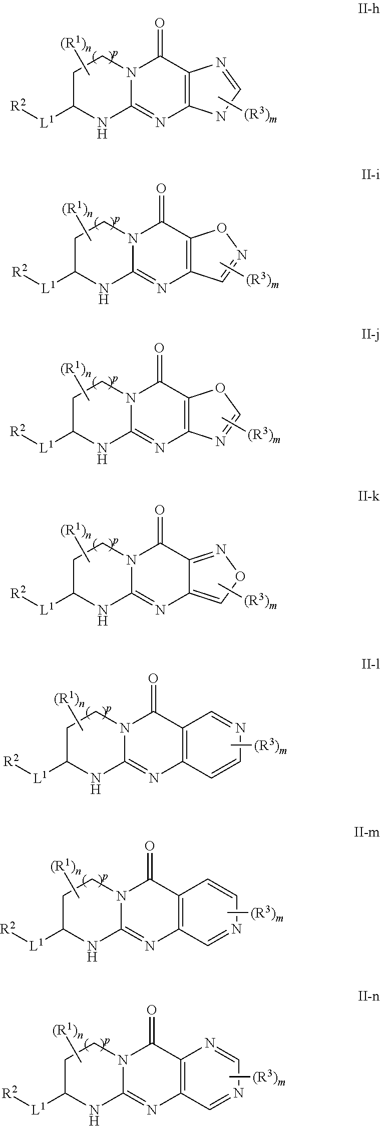

or a pharmaceutically acceptable salt thereof, wherein:
each of R¹, R², R³, p, n, and m is as described in embodiments for formula I, supra, or described in embodiments herein, both singly and in combination.

In certain embodiments, the present invention provides a compound of one of formulas II-a, II-b, II-c, II-d, II-e, II-f, II-g, II-h, II-i, II-j, II-k, II-l, II-m, and II-n, wherein p is 1, or a pharmaceutically acceptable salt thereof. In certain embodiments, the present invention provides a compound of one of formulas II-a, II-b, II-c, II-d, II-e, II-f, II-g, II-h, II-i, II-j, II-k, II-l, II-m, and II-n, wherein p is 0, or a pharmaceutically acceptable salt thereof. In certain embodiments, the present invention provides a compound of one of formulas II-a, II-b, II-c, II-d, II-e, II-f, II-g, II-h, II-i, II-j, II-k, II-l, II-m, and II-n, wherein L¹ is a covalent bond, or a pharmaceutically acceptable salt thereof. In certain embodiments, the present invention provides a compound of one of formulas II-a, II-b, II-c, II-d, II-e, II-f, II-g, II-h, II-i, II-j, II-k, II-l, II-m, and II-n, wherein L¹ is a covalent bond, and R² is not hydrogen, or a pharmaceutically acceptable salt thereof. In certain embodiments, the present invention provides a compound of one of formulas II-a, II-b, II-c, II-d, II-e, II-f, II-g, II-h, II-i, II-j, II-k, II-l, II-m, and II-n, wherein L¹ is methylene, or a pharmaceutically acceptable salt thereof. In certain embodiments, the present invention provides a compound of one of formulas II-a, II-b, II-c, II-d, II-e, II-f, II-g, II-h, II-i, II-j, II-k, II-l, II-m, and II-n, wherein L¹ is methylene, and R² is not hydrogen, or a pharmaceutically acceptable salt thereof.

In some embodiments, compounds of the invention are not selected from the following formulas:

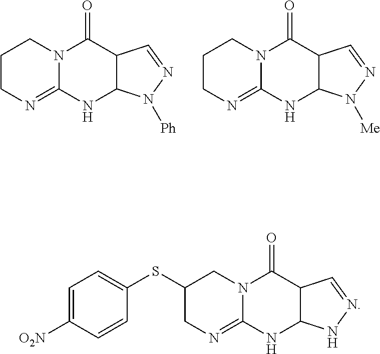

In certain embodiments, the present invention provides any compound selected from those depicted in the Examples disclosed herein, or a pharmaceutically acceptable salt thereof,

### 4. Uses, Formulation and Administration and Pharmaceutically Acceptable Compositions

According to another embodiment, the invention provides a composition comprising a compound of this invention or a pharmaceutically acceptable salt, ester, or salt of ester thereof and a pharmaceutically acceptable carrier, adjuvant, or vehicle. The amount of compound in compositions of this invention is such that is effective to measurably inhibit PDE1, in a biological sample or in a patient. In certain embodiments, the amount of compound in compositions of this invention is such that is effective to measurably inhibit PDE1, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

As used herein, the term “inhibitorily active metabolite or residue thereof” means that a metabolite or residue thereof is also an inhibitor of PDE1.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon a variety of factors, including the host treated and the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

Phosphodiesterases (PDE's) are enzymes that catalyze the hydrolysis of the cyclic phosphate bonds of cyclic guanosine monophosphate (cGMP) and/or cyclic adenosine monophosphate (cAMP). Lugnier, C., Pharmacology & Therapeutics (2006), 109, 366. The PDE superfamily can be grouped into 11 families (PDE1-11) based on their sequence, regulation and substrate specificity. Each family can contain multiple subtypes, each the product of individual genes. In particular, the PDE1 family, consisting of PDE1A, PDE1B and PDE1C, are so-called dual substrate enzymes that hydrolyze both cGMP and cAMP, and are regulated by Ca²⁺ and calmodulin. PDE1A is expressed throughout the brain, especially in the hippocampus and cerebellum, and at lower levels in the striatum, as well as in the peripheral vasculature. PDE1B, by contrast, is expressed primarily in the striatum and cerebellum, and is often found in regions of high dopaminergic tone and dopamine D1 receptor expression. PDE1C is primarily expressed in the heart, olfactory epithelium and striatum. Considering these expression patterns, a compound that is selective for PDE1B over PDE1A and/or PDE1C may have fewer effects on the cardiovascular system.

Due to the expression pattern of the PDE1 family, inhibition of PDE1 may be useful in the treatment of disorders involving learning and memory by enhancing neuronal plasticity. The increased levels of intracellular cAMP and cGMP caused by PDE1 inhibition trigger cascades that ultimately lead to the phosphorylation and activation of the transcription factors cAMP Responsive Element Binding Protein (CREB) and Serum Response Factor (SRF). Josselyn, S. A., Nguyen, P. V., Current Drug Targets—CNS & Neurological Disorders (2005) 4, 481. Activation of CREB and SRF can lead to the expression of plasticity-related genes which mediate the processes that are critical for neuronal plasticity such as the remodeling of dendritic spines. PDE1 inhibitors may therefore be useful in the treatment of cognitive symptoms of disorders such as Alzheimer's Disease, Parkinson's Disease, Stroke, Schizophrenia, Down Syndrome, Fetal Alcohol Syndrome and others.

Due to its location in the striatum and its role in modulating levels of secondary messengers such as cyclic nucleotides, PDE1 is also a regulator of locomotor activity. Reed, T. M. J., et al., Journal of Neuroscience (2002) 22, 5189). Due to their ability to increase levels of cyclic nucleotides in the striatum, PDE1 inhibitors are expected to potentiate the effects of D1 agonists by inhibiting the degradation of cAMP and cGMP. This potentiation of dopamine signaling may be useful in the treatment of diseases including, but not limited to Parkinson's Disease, depression and cognitive disorders including Cognitive Impairment Associated with Schizophrenia.

The activity of a compound utilized in this invention as an inhibitor of PDE1 or a treatment for a neurological or, psychiatric disorder, may be assayed in vitro or in vivo. An in vivo assessment of the efficacy of the compounds of the invention may be made using an animal model of a neurological or psychiatric disorder, e.g., a rodent or primate model. Cell-based assays may be performed using, e.g., a cell line isolated from a tissue that expresses PDE1, or a cell line that recombinantly expresses PDE1. Additionally, biochemical or mechanism-based assays, e.g., measuring cAMP or cGMP levels, Northern blot, RT-PCR, etc., may be performed. In vitro assays include assays that determine cell morphology, protein expression, and/or the cytotoxicity, enzyme inhibitory activity, and/or the subsequent functional consequences of treatment of cells with compounds of the invention. Alternate in vitro assays quantify the ability of the inhibitor to bind to protein or nucleic acid molecules within the cell. Inhibitor binding may be measured by radiolabelling the inhibitor prior to binding, isolating the inhibitor/target molecule complex and determining the amount of radiolabel bound. Alternatively, inhibitor binding may be determined by running a competition experiment where new inhibitors are incubated with purified proteins or nucleic acids bound to known radioligands. Detailed conditions for assaying a compound utilized in this invention as an inhibitor of PDE1 are set forth in the Examples below. The aforementioned assays are exemplary and not intended to limit the scope of the invention. The skilled practitioner can appreciate that modifications can be made to conventional assays to develop equivalent assays that obtain the same result.

As used herein, the terms “treatment,” “treat.” and “treating” refer to reversing, alleviating, delaying the onset of or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The compounds and compositions, according to the method of the present invention, may be administered using any amount and any route of administration effective for treating or lessening the severity of a neurological or psychiatric disorder.

In some embodiments, the compounds and compositions, according to the method of the present invention, may be administered using any amount and any route of administration effective for treating or lessening the severity of a disease associated with PDE1.

In some embodiments, the compounds and compositions, according to the method of the present invention, may be administered using any amount and any route of administration effective for treating or lessening the severity of a neurological or psychiatric disorder.

In some embodiments, the neurological or psychiatric disorder is selected from schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (phencyclidine, ketamine and other dissociative anesthetics, amphetamine and other psychostimulants and cocaine) psychosispsychotic disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both positive, negative, and cognitive symptoms of schizophrenia and other psychoses; cognitive disorders including dementia (associated with Alzheimer's disease, ischemia, multi-infarct dementia, trauma, vascular problems or stroke, HIV disease, Parkinson's disease, Huntington's disease, Down syndrome, Pick's disease, Creutzfeldt-Jacob disease, perinatal hypoxia, other general medical conditions or substance abuse); delirium, amnestic disorders or age related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders; depression including unipolar depression, seasonal depression and post-partum depression, premenstrual syndrome (PMS) and premenstrual dysphoric disorder (PDD), mood disorders due to a general medical condition, and substance-induced mood disorders; learning disorders, pervasive developmental disorder including autistic disorder, attention disorders including attention-deficit hyperactivity disorder (ADHD) and conduct disorder; disorders such as autism, depression, benign forgetfulness, childhood learning disorders and closed head injury; movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, Parkinsonism-ALS dementia complex and basal ganglia calcification), medication-induced Parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, muscular spasms and disorders associated with muscular spasticity or weakness including tremors; dyskinesias {including drug e.g. L-DOPA induced dyskinesia tremor (such as rest tremor, postural tremor, intention tremor), chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia)}; urinary incontinence, neuronal damage including ocular damage, retinopathy or macular degeneration of the eye, tinnitus, hearing impairment and loss, and brain edema; emesis; and sleep disorders including insomnia and narcolepsy

In some embodiments, the neurological or psychiatric disorder is selected from the group consisting of Alzheimer's Disease, Parkinson's Disease, depression, cognitive impairment, stroke, schizophrenia, Down Syndrome, and Fetal Alcohol Syndrome. In some embodiments, the neurological or psychiatric disorder is Alzheimer's Disease. In some embodiments, the neurological or psychiatric disorder is Parkinson's Disease. In some embodiments, the neurological or psychiatric disorder is depression. In some embodiments, the neurological or psychiatric disorder is cognitive impairment. In some embodiments, the neurological or psychiatric disorder is stroke. In some embodiments, the neurological or psychiatric disorder is schizophrenia. In some embodiments, the neurological or psychiatric disorder is Down Syndrome. In some embodiments, the neurological or psychiatric disorder is Fetal Alcohol Syndrome.

In some embodiments, the neurological or psychiatric disorder involves a deficit in cognition (cognitive domains as defined by the Diagnostic and Statistical Manual of Mental Disorders, 5th Ed., American Psychiatric Publishing (2013) (“DSM-5”) are: complex attention, executive function, learning and memory, language, perceptual-motor, social cognition). In some embodiments, the neurological or psychiatric disorder is associated with a deficit in dopamine signaling. In some embodiments, the neurological or psychiatric disorder is associated with basal ganglia dysfunction. In some embodiments, the neurological or psychiatric disorder is associated with dysregulated locomotor activity.

In some embodiments, the neurological or psychiatric disorder is associated with a deficit in cyclic nucleotide signaling molecules. In some embodiments, the neurological or psychiatric disorder is associated with a deficit in cAMP and/or cGMP. In some embodiments, the neurological or psychiatric disorder is associated with low activity of cAMP Responsive Element Binding Protein (CREB), Serum Response Factor (SRF), or both.

In some embodiments, the present invention provides a method of treating a neurological or psychiatric disorder described herein, comprising administering a compound of the invention in conjunction with one or more pharmaceutical agents. Suitable pharmaceutical agents that may be used in combination with the compounds of the present invention include anti-Parkinson's drugs, anti-Alzheimer's drugs, anti-depressants, anti-psychotics, anti-ischemics, CNS depressants, anti-cholinergics, and nootropics.

Suitable anti-Parkinson's drugs include, but are not limited to, dopamine replacement therapy (e.g. L-DOPA, carbidopa, COMT inhibitors such as entacapone), dopamine agonists (e.g. D1 agonists, D2 agonists, mixed D1/D2 agonists; bromocriptine, pergolide, cabergoline, ropinirole, pramipexole, or apomorphine in combination with domperidone), histamine H2 antagonists, and monoamine oxidase inhibitors such as selegiline and tranylcypromine.

In some embodiments, compounds of the invention may be used in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexyl)hydrochloride, COMT inhibitors such as entacapone, MAO A/B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexole are commonly used in a non-salt form.

Suitable anti-Alzheimer's drugs include, but are not limited to, beta-secretase inhibitors, gamma-secretase inhibitors, HMG-CoA reductase inhibitors, NSAIDs including ibuprofen, vitamin E, and anti-amyloid antibodies. In some embodiments, an anti-Alzheimer's drug is memantine.

Suitable anti-depressants and anti-anxiety agents include, but are not limited to norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists.

Specific suitable anti-depressant and anti-anxiety agents include, but are not limited to, amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

The exact amount required will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, our drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, the invention relates to a method of inhibiting PDE1 in a biological sample comprising the step of contacting said biological sample with a compound of this invention, or a composition comprising said compound. In some embodiments, the PDE1 is PDE1A. In some embodiments, the PDE1 is PDE1B. In some embodiments, the PDE1 is PDE1C. In some embodiments, the invention provides a method of inhibiting PDE1B selectively over PDE1A and/or PDE1C. In some embodiments, the invention provides a method of inhibiting PDE1B selectively over PDE1A. In some embodiments, the invention provides a method of inhibiting PDE1B selectively over PDE1C. In some embodiments, the invention provides a method of inhibiting PDE1B selectively over PDE1A and PDE1C. In some embodiments, the selectivity for PDE1B over PDE1A and/or PDE1C is up to and including five-fold. In some embodiments, the selectivity for PDE1B over PDE1A and/or PDE1C is up to and including ten-fold. In some embodiments, the selectivity for PDE1B over PDE1A and/or PDE1C is up to and including twenty-fold. In some embodiments, the selectivity for PDE1B over PDE1C is up to and including fifty-fold. In some embodiments, the selectivity for PDE1B over PDE1C is up to and including one hundred-fold. In some embodiments, the selectivity for PDE1B over PDE1C is up to and including two hundred-fold. Selectivity for one PDE1 isoform over another refers to the inverse ratio of IC₅₀ values against each respective isoform as determined using the HTRF PDE1 inhibition assay described in the Examples. For example, the selectivity of a compound of this invention for PDE1B over PDE1C refers to the ratio IC₅₀(PDE1C)/IC₅₀(PDE1B), wherein IC₅₀(PDE1C) is the IC₅₀ value of the compound against PDE1C as determined using the described HTRF PDE1 inhibition assay, and IC₅₀(PDE1B) is the IC₅₀ value of the compound against PDE1B as determined using the described HTRF PDE1 inhibition assay.

In certain embodiments, the invention relates to a method of modulating cyclic nucleotide levels in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of enzymes in a biological sample is useful for a variety of purposes that are known to one of skill in the art. Examples of such purposes include, but are not limited to biological assays, gene expression studies, and biological target identification.

Another embodiment of the present invention relates to a method of inhibiting PDE1 in a patient comprising the step of administering to said patient a compound of the present invention, or a composition comprising said compound. In some embodiments, the PDE1 is PDE1B. In some embodiments, the invention provides a method of inhibiting PDE1B in a patient selectively over PDE1A and/or PDE1C. In some embodiments, the invention provides a method of inhibiting PDE1B in a patient selectively over PDE1A. In some embodiments, the invention provides a method of inhibiting PDE1B in a patient selectively over PDE1C. In some embodiments, the invention provides a method of inhibiting PDE1B in a patient selectively over PDE1A and PDE1C. In some embodiments, the selectivity for PDE1B over PDE1A and/or PDE1C is up to and including five-fold. In some embodiments, the selectivity for PDE1B over PDE1A and/or PDE1C is up to and including ten-fold. In some embodiments, the selectivity for PDE1B over PDE1A and/or PDE1C is up to and including twenty-fold. In some embodiments, the selectivity for PDE1B over PDE1C is up to and including fifty-fold. In some embodiments, the selectivity for PDE1B over PDE1C is up to and including one hundred-fold. In some embodiments, the selectivity for PDE1B over PDE1C is up to and including two hundred-fold. Selectivity for one PDE1 isoform over another refers to the inverse ratio of IC₅₀ values against each respective isoform as determined using the HTRF PDE1 inhibition assay described in the Examples. For example, the selectivity of a compound of this invention for PDE1B over PDE1C refers to the ratio IC₅₀(PDE1C)/IC₅₀(PDE1B), wherein IC₅₀(PDE1C) is the IC₅₀ value of the compound against PDE1C as determined using the described HTRF PDE1 inhibition assay, and IC₅₀(PDE1B) is the IC₅₀ value of the compound against PDE1B as determined using the described HTRF PDE1 inhibition assay.

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated”.

In certain embodiments, a combination of 2 or more therapeutic agents may be administered together with compounds of the invention. In certain embodiments, a combination of 3 or more therapeutic agents may be administered with compounds of the invention.

Other examples of agents the inhibitors of this invention may also be combined with include, without limitation: vitamins and nutritional supplements, antiemetics (e.g. 5-HT/receptor antagonists, dopamine antagonists. NK1 receptor antagonists, histamine receptor antagonists, cannabinoids, benzodiazepines, or anticholinergics), agents for treating Multiple Sclerosis (MS) such as beta interferon (e.g., Avonex® and Rebif®), Copaxone®, and mitoxantrone; treatments for asthma such as albuterol and Singulair®; anti-inflammatory agents such as corticosteroids, TNF blockers, IL-1 RA, azathioprine, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, agents for treating cardiovascular disease such as beta-blockers, ACE inhibitors, diuretics, nitrates, calcium channel blockers, and statins, fibrates, cholesterol absorption inhibitors, bile acid sequestrants, and niacin; agents for treating liver disease such as corticosteroids, cholestyramine, interferons, and anti-viral agents; agents for treating blood disorders such as corticosteroids, anti-leukemic agents, and growth factors; agents for treating immunodeficiency disorders such as gamma globulin; and anti-diabetic agents such as biguanides (metformin, phenformin, buformin), thiazolidinediones (rosiglitazone, pioglitazone, troglitazone), sulfonylureas (tolbutamide, acetohexamide, tolazamide, chlorpropamide, glipizide, glyburide, glimepiride, gliclazide), meglitinides (repaglinide, nateglinide), alpha-glucosidase inhibitors (miglitol, acarbose), incretin mimetics (exenatide, liraglutide, taspoglutide), gastric inhibitory peptide analogs, DPP-4 inhibitors (vildagliptin, sitagliptin, saxagliptin, linagliptin, alogliptin), amylin analogs (pramlintide), and insulin and insulin analogs.

In certain embodiments, compounds of the present invention, or a pharmaceutically acceptable composition thereof are administered in combination with antisense agents, a monoclonal or polyclonal antibody or an siRNA therapeutic.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another, normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of formula I, an additional therapeutic agent, and a pharmaceutically acceptable carrier, adjuvant, or vehicle.

The amount of both, an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-100 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In some embodiments, the present invention provides a medicament comprising at least one compound of formula I or a pharmaceutically acceptable salt thereof and a pharmaceutically acceptable carrier.

In some embodiments, the present invention provides the use of a compound of formula I in the manufacture of a medicament for the treatment of a neurological or psychiatric disorder.

### EXEMPLIFICATION

As depicted in the Examples below, in certain exemplary embodiments, compounds are prepared according to the following procedures. It will be appreciated that, although the general methods depict the synthesis of certain compounds of the present invention, the following methods, and other methods known to one of ordinary skill in the art, can be applied to all compounds and subclasses and species of each of these compounds, as described herein.

In the examples below, unless otherwise indicated, all temperatures are set forth in degrees Celsius and all parts and percentages are by weight. Reagents were purchased from commercial suppliers, such as Sigma-Aldrich Chemical Company, and were used without further purification unless otherwise indicated. Reagents were prepared following standard literature procedures known to those skilled in the art. Solvents were purchased from Aldrich in Sure-Seal bottles and used as received. All solvents requiring purification or drying were treated using standard methods known to those skilled in the art, unless otherwise indicated.

The reactions set forth below were done generally at ambient temperature, unless otherwise indicated. The reaction flasks were fitted with rubber septa for introduction of substrates and reagents via syringe. Analytical thin layer chromatography (TLC) was performed using glass-backed silica gel pre-coated plates (Merck Art 5719) and eluted with appropriate solvent ratios (v/v). Reactions were assayed by TLC or LCMS, and terminated as judged by the consumption of starting material. Visualization of the TLC plates was done with UV light (254 wavelength) or with an appropriate TLC visualizing solvent, such as basic aq. KMnO₄ solution activated with heat. Flash column chromatography (See, e.g., Still et al., J. Org. Chem., 43: 2923 (1978)) was performed using silica gel 60 (Merck Art 9385) or various MPLC systems. Reactions under microwave irradiation conditions were performed using a Biotage initiator microwave system.

The compound structures in the examples below were confirmed by one or more of the following methods: proton magnetic resonance spectroscopy, mass spectroscopy, and melting point. Proton magnetic resonance (¹H NMR) spectra were determined using a JEOL or Bruker NMR spectrometer operating at 400 MHz field strength. Chemical shifts are reported in the form of delta (δ) values given in parts per million (ppm) relative to an internal standard, such as tetramethylsilane (TMS). Alternatively, ¹H NMR spectra were referenced to signals from residual protons in deuterated solvents as follows: CDCl₃=7.25 ppm; DMSO-d₆=2.49 ppm; C₆D₆=7.16 ppm; CD₃OD=3.30 ppm. Peak multiplicities are designated as follows: s, singlet; d, doublet; dd, doublet of doublets; t, triplet; dt, doublet of triplets; q, quartet; quint, quintet; sept, septet; br, broadened; and m, multiplet. Coupling constants are given in Hertz (Hz). Mass spectra (MS) data were obtained using Agilent Technologies 1200 Series/Agilent Technologies 6110 Quadrupole LCMS, Waters ACQUITY UPLC or Shimadzu LCMS-2020. Chiral analyzation was performed using a instrument of Chiral-analyzation SFC Method Station (Thar, Waters). Chiral preparation was performed using SFC-80, SFC-200, or SFC-350 (Thar, Waters). Chiral compounds were separated with the following columns (AD-H, OJ-H, AS-H, OZ-H, OD-H, AY-H, and IC: Daicel, 4.6×250 mm, 5 μm; or (R,R)-Whelk-Ol RegisCell: Regis, 4.6×250 mm, 5 μm) and with the following methods (Table 1).

TABLE 1
Separation methods

| Method | Column | Column temp. | Solvent | Flow rate |
|---|---|---|---|---|
| 1 | AD-H | 40° C. | CO₂:MeOH(0.1% DEA) = 70:30 | 3.0 mL/min |
| 2 | AD-H | 40° C. | CO₂:MeOH(0.1% DEA) = 65:35 | 3.0 mL/min |
| 3 | AD-H | 40° C. | CO₂:MeOH = 65:35 | 3.0 mL/min |
| 4 | AD-H | 40° C. | CO₂:MeOH(0.1% DEA) = 60:40 | 3.0 mL/min |
| 5 | AD-H | 40° C. | CO₂:MeOH = 55:45 | 3.0 mL/min |
| 6 | AD-H | 40° C. | n-Hexanes(0.1% DEA)/ | 1.0 mL/min |
| | | | EtOH(0.1% DEA) = 60:40 | |
| 7 | OJ-H | 40° C. | CO₂:EtOH/MeCN(1:1, 0.5% DEA) = | 3.0 mL/min |
| | | | 80:20 | |
| 8 | OJ-H | 40° C. | CO₂:MeOH = 65:35 | 3.0 mL/min |
| 9 | AS-H | 40° C. | CO₂:MeOH(0.1% DEA) = 80:20 | 3.0 mL/min |
| 10 | AS-H | 40° C. | CO₂:MeOH(0.1% DEA) = 75:25 | 3.0 mL/min |
| 11 | AS-H | 40° C. | CO₂:MeOH = 75:25 | 3.0 mL/min |
| 12 | AS-H | 40° C. | CO₂:MeOH = 70:30 | 3.0 mL/min |
| 13 | AS-H | 40° C. | CO₂:MeOH = 65:35 | 3.0 mL/min |
| 14 | AS-H | 40° C. | CO₂:MeOH(0.1% DEA) = 60:40 | 3.0 mL/min |
| 15 | OZ-H | 40° C. | CO₂:MeOH(0.1% DEA) = 75:25 | 3.0 mL/min |
| 16 | OZ-H | 40° C. | CO₂:MeOH = 65:35 | 3.0 mL/min |
| 17 | (R,R)- | 40° C. | CO₂:MeOH(0.1% DEA) = 65:35 | 3.0 mL/min |
| | Whelk-Ol | | | |
| 18 | (R,R)- | 40° C. | CO₂:MeOH = 60:40 | 3.0 mL/min |
| | Whelk-Ol | | | |
| 19 | OD-H | 40° C. | CO₂:MeOH(0.1% DEA) = 70:30 | 3.0 mL/min |
| 20 | OD-H | 40° C. | CO₂:MeOH(0.1% DEA) = 65:35 | 3.0 mL/min |
| 21 | IC | 40° C. | CO₂:MeOH(0.1% DEA) = 70:30 | 3.0 mL/min |
| 22 | IC | 40° C. | CO₂:EtOH(0.1% DEA) = 70:30 | 3.0 mL/min |
| 23 | IC | 40° C. | CO₂:IPA(0.1% DEA) = 60:40 | 3.0 mL/min |
| 24 | AY-H | 40° C. | n-Hexanes(0.1% DEA)/ | 1.0 mL/min |
| | | | EtOH(0.1% DEA) = 70:30 | |
| 25 | AY-H | 40° C. | n-Hexanes(0.1% DEA)/ | 1.0 mL/min |
| | | | EtOH(0.1% DEA) = 80:20 | |
| 26 | RegisCell | 40° C. | CO₂:MeOH(0.1% DEA) = 85:15 | 3.0 mL/min |
| 27 | RegisCell | 40° C. | CO₂:MeOH(0.1% DEA) = 75:25 | 3.0 mL/min |
| 28 | OJ-H | 40° C. | CO₂:MeOH(0.1% DEA) = 80:20 | 3.0 mL/min |
| 29 | AD-H | 40° C. | CO₂:MeOH(0.1% NH₄OH) = 80:20 | 3.0 mL/min |

As used herein, and unless otherwise specified, “Me” means methyl, “Et” means ethyl, “i-Pr” means isopropyl, “t-Bu” means tert-butyl, “Ac” means acetyl, “Boc” means tert-butoxycarbonyl, “DAST” means N,N-diethylaminosulfur trifluoride, “DCC” means dicyclohexylcarbodiimide, “Dess-Martin reagent” means 1,1,1-tris(acetyloxy)-1,1-dihydro-1,2-benziodoxol-3-(1H)-one, “DCM” means dichloromethane, “DEAD” means diethylazodicarboxylate, “DIAD” means diisopropylazodicarboxylate, “DIPEA” means N,N-diisopropylethylamine, “DME” means dimethoxyethane, “DMF” means dimethylformamide, “DMSO” means dimethylsulfoxide, “EDCI” means N-(3-dimethylaminopropyl)-N′-ethylcarbodiimide hydrochloride, “EtOAc” means ethyl acetate, “EtOH” means ethanol, “HATU” means O-(7-azabenzotriazol-1-yl)-N,N,N,N′-tetramethyluronium hexafluorophosphate, “HOBt” means hydroxybenzotriazole, “LDA” means lithium diisopropylamide, “LiHMDS” means lithium bis(trimethylsilyl)amide, “m-CPBA” means 3-chloro-perbenzoic acid, “MeCN” means acetonitrile, “MeOH” means methanol, “Ms” means methanesulfonyl, “NBS” means N-bromosuccinimide, “PE” means petroleum ether, “RT” or “rt” means room temperature, “TBAF” means tetrabutylammonium fluoride, “TBAI” means tetrabutylammonium iodide, “TBS” or “TBDMS” means tert-butyldimethylsilyl, “TBTU” means 2-(1H-benzotriazole-1-yl)-1,1,3,3-tetramethyluronium tetrafluoroborate, “TEA” means triethylamine, “TFA” means trifluoroacetic acid, “TFAA” means trifluoroaceti anhydride, “THF” means tetrahydrofuran, “TMS” means trimethylsilyl, “p-Ts” means para-tolenesulfonyl, “h” or “hr” means hour(s), “min” means minute(s), “cat.” means catalytic, “aq.” means aqueous, and “sat.” means saturated.

### Reference Examples

### Reference Example 1

### 6-Chloro-1-isopropyl-1,5-dihydro-4H-pyrazolo[3,4-d]pyrimidin-4-one

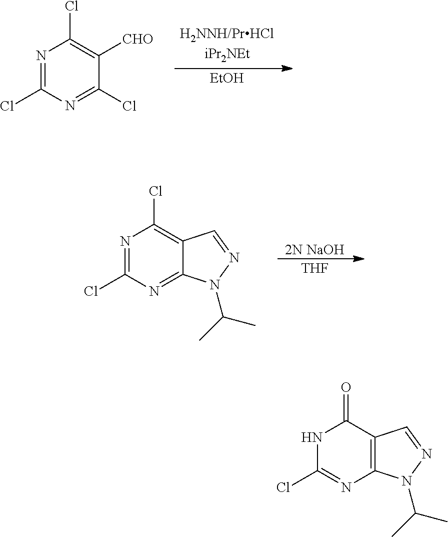

To a solution of 4,6-dichloro-1-isopropyl-1H-pyrazolo[3,4-d]pyrimidine (6.14 g, 26.57 mmol) in THF (80 mL) was added 2M sodium hydroxide (240 mL, 159.42 mmol) and the mixture was stirred at 50° C. for 12.5 h. After concentration under reduce pressure, the residue was added 5 M hydrochloric acid (26 mL) and filtrated to give title compound (5.53 g, 98%) as white solids. ¹H NMR (400 MHz, CDCl₃): δ 10.82 (br s, 1H), 8.10 (s, 1H), 5.01 (sept, J=6.6 Hz, 1H), 1.54 (d, J=6.6 Hz, 6H). LCMS: m/z=213 [M+H]⁺.

### 4,6-Dichloro-1-isopropyl-1H-pyrazolo[3,4-d]pyrimidine

To a solution of 7,4,6-trichloro-5-pyrimidinecarboxaldehyde (6.00 g, 28.38 mmol) in EtOH (120 mL) was cooled to −78° C. and added isopropylhydrazine hydrochloric (3.14 g, 28.38 mmol) under a N₂ atmosphere. To a solution was added DIPEA (14.83 mL, 85.14 mmol) dropwise. The mixture was stirred at −78° C. for 2 h and then warmed up to rt and stirred at this temperature for 1 h. Water (60 mL) was added to the reaction solution and concentrated under reduced pressure. The mixture was extracted with EtOAc, washed with brine and dried over sodium sulfate. After concentration under reduced pressure, the residue was purified by silica gel column chromatography to give title compound (6.15 g, 94%) as white solids. ¹H NMR (400 MHz, CDCl₃): δ 8.14 (s, 1H), 5.18 (sept, J=6.8 Hz, 1H), 1.58 (d, J=6.8 Hz, 6H). LCMS: m/z=231 [M+H]⁺.

### Reference Example 2

### 2-Chloro-7-(tetrahydro-2H-pyran-4-yl)imidazo[5,1-f][1,2,4]triazin-4(1H)-one

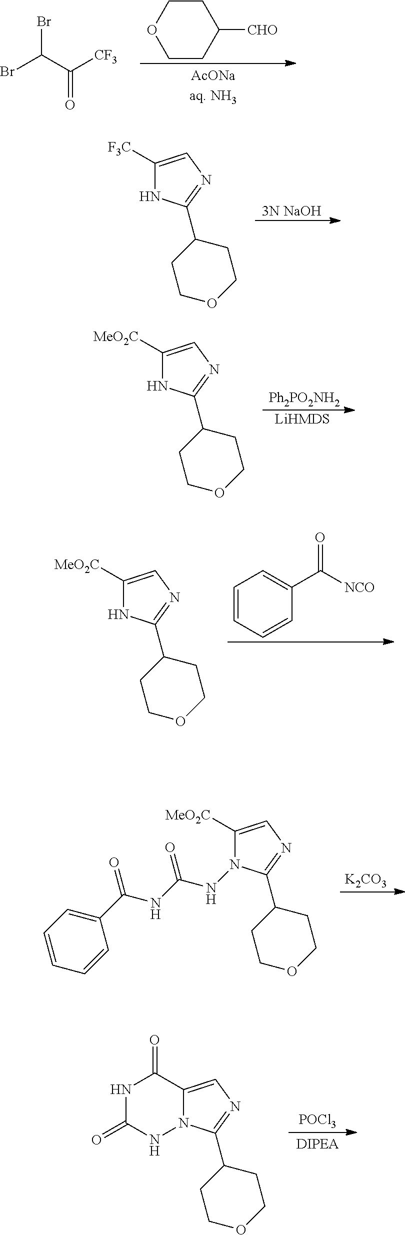

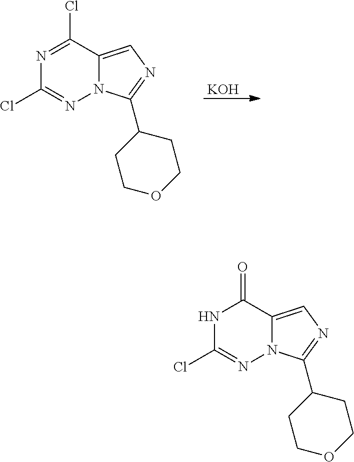 [SMILES: Clc1nc(Cl)c2cnc(C3CCOCC3)n2n1 ; "KOH" ; O=c1[nH]c(Cl)nn2c(C3CCOCC3)ncc12]

To the solution of 2,4-dichloro-7-(tetrahydro-2H-pyran-4-yl)imidazo[5,1-f][1,2,4]triazine (1.2 g) in tetrahydrofuran (20 mL) was added 2M potassium hydroxide aq. solution (20 mL). The mixture was stirred for 2 h at 50° C., and then was neutralized with 1M hydrochloric acid. The mixture was filtered to get the titled compound (0.78 g, 70%). ¹H NMR (400 MHz, DMSO-d₆) δ 1.79-1.88 (m, 4H), 3.34-3.38 (m, 1H), 3.46-3.53 (m, 2H), 3.91-3.95 (m, 2H), 7.76 (s, 1H), 13.01 (br s, 1H). LCMS: m/z=255 [M+H]⁺.

### 2-(Tetrahydro-2H-pyran-4-yl)-4-(trifluoromethyl)-1H-imidazole

A mixture of sodium acetate trihydrate (27.2 g, 200 mmol) and 3,3-dibromo-1,1,1-trifluoroacetone (26.98 g, 100 mmol) in water (75 ml) was heated under reflux for 1 h. The mixture was then cooled to rt and was slowly added to a solution of tetrahydro-2H-pyran-4-carbaldehyde (90 mmol, 10.27 g) and concentrated ammonium hydroxide solution (50 mL) in MeOH (150 mL). The mixture was stirred at rt for 18 hand was then evaporated under reduced pressure. The aqueous residue was extracted with EtOAc (150 mL×3) and the combined organic solution was dried over magnesium sulfate and concentrated in vacuo to give the crude as an oil.

The oil was then triturated in water with a trace of MeOH to afford the title compound as crystalline solids (19.8 g, 90%). LCMS: m/z=221 [M+H]⁺.

### Methyl 2-(tetrahydro-2H-pyran-4-yl)-1H-imidazole-4-carboxylate

To a solution of methyl 2-(tetrahydro-2H-pyran-4-yl)-4-(trifluoromethyl)-1H-imidazole (85 mmol) in MeOH (200 mL) was added sodium hydroxide aq. solution (2.7 M, 50 mL) and the mixture was stirred at 95° C. overnight. Then conc. hydrochloric acid (25 mL) was added. The mixture was stirred at that temperature for 4 h. EtOAc (250 mL) was added to the reaction vessel and the resulting biphasic mixture was transferred to a separatory funnel. The layers were separated and the water phase was extracted with EtOAc (150 mL×3). The combined organics were dried over anhydrous sodium sulfate, filtered and concentrated in vacuum to afford the title compound as solids (16.5 g, 80%). LCMS: m/z=210 [M+H]⁺.

### Methyl 1-amino-2-(tetrahydro-2H-pyran-4-yl)-1H-imidazole-5-carboxylate

To a solution of methyl 2-(tetrahydro-2H-pyran-4-yl)-1H-imidazole-4-carboxylate (70 g, 0.34 mol) in DCM (250 mL) was added O-(mesitylsulfonyl) hydroxylamine (110 g, 0.51 mol) and potassium carbonate (94 g, 0.64 mol). The reaction mixture was cooled to 0° C. and stirred at that temperature for 15 h. Water (50 mL) was added to the reaction vessel and the resulting biphasic mixture was transferred to a separatory funnel. The layers were separated. The combined organics were dried over anhydrous sodium sulfate, filtered and concentrated in vacuum. The resulting solid was purified by flash column chromatography to provide the titled compound (25 g, 35%) as white solids. LCMS: m/z=225 [M+H]⁺.

### Methyl 1-[(benzoylcarbamoyl)amino]-2-(tetrahydro-2H-pyran-4-yl)-1H-imidazole-5-carboxylate

To a solution of methyl methyl 1-amino-2-(tetrahydro-2H-pyran-4-yl)-1H-imidazole-5-carboxylate (5 g, 22.2 mmol) in THF (75 mL) was added benzoyl isocyanate (3.59 g, 24.4 mmol). The reaction mixture was heated to and stirred at that temperature for 12 h. The combined organics was concentrated in vacuo to get the titled compound (5 g, 80%). ¹H NMR (400 MHz, DMSO-d₆) δ 1.67-1.88 (m, 4H), 3.04-3.12 (m, 1H), 3.40-3.46 (m, 2H), 3.71 (s, 3H), 3.89-3.94 (m, 2H), 7.56-7.60 (m, 2H), 7.67-7.71 (m, 2H), 8.06-8.08 (m, 2H), 11.19 (s, 1H), 11.34 (s, 1H). LCMS: m/z=373 [M+H]⁺.

### 7-(Tetrahydro-2H-pyran-4-yl)imidazo[5,1-f][1,2,4]triazine-2,4(1H,3H)-dione

To a solution of methyl 1-[(benzoylcarbamoyl)amino]-2-(tetrahydro-2H-pyran-4-yl)-1H-imidazole-5-carboxylate (5 g, 13.43 mmol) in MeOH (45 mL) was added potassium carbonate (2.23 g, 16.11 mmol). The combined organics concentrated in vacuo. Water (20 mL) was added to the reaction. The mixture was neutralized with 1M hydrochloric acid, filtered and washed with MeOH to get the titled compound (1.8 g, 56%). NMR (400 MHz, DMSO-d₆): δ 1.75-1.85 (m, 4H), 3.32-3.35 (m, 1H), 3.38-3.49 (m, 2H), 3.92-3.95 (m, 2H), 7.50 (hr s, 1H), 7.74 (s, 1H), 11.15 (s, 1H). LCMS: m/z=237 [M±H]⁺.

### 2,4-Dichloro-7-(tetrahydro-2H-pyran-4-yl)imidazo[5,1-f][1,2,4]triazine

To the mixture of 7-(tetrahydro-2H-pyran-4-yl)imidazo[5,1-f][1,2,4]triazine-2,4(1H,3H)-dione (1.8 g) in phosphoryl trichloride (20 mL), DIPEA (1.48 g) was add. The mixture was stirred for 3 h at 120° C. The pH was adjusted to 7-8 and a white precipitate formed. After filtration, the solid was collected (1.2 g, 60%) as yellow solids. ¹H NMR (400 MHz, DMSO-d₆) 1.24-1.31 (m, 2H), 1.82-1.87 (m, 2H), 3.40-3.54 (m, 1H), 3.47-3.54 (m, 2H), 3.92-3.96 (m, 2H), 7.88 (s, 1H). LCMS: m/z=273 [M+H]⁺.

### Reference Example 3

### 4-Amino-5-(tetrahydro-2H-pyran-4-yl)nicotinic acid

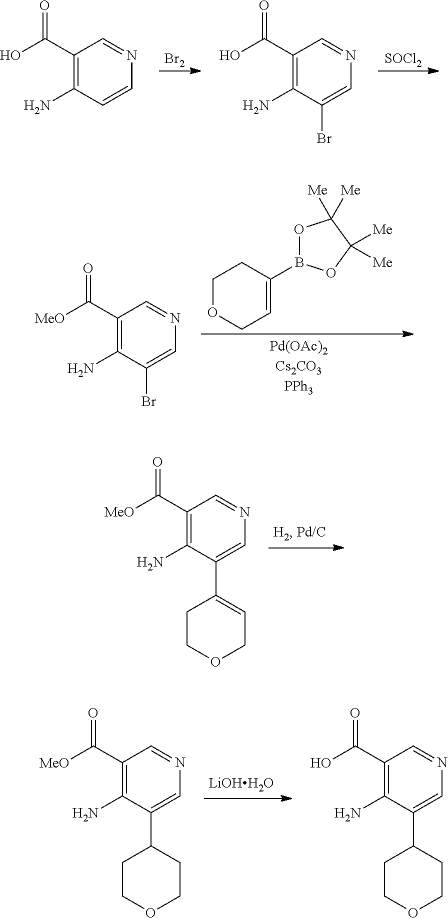

A solution of methyl 4-amino-5-(tetrahydro-2H-pyran-4-yl)nicotinate (650 mg, 2.75 mmol) and lithium hydroxide monohydrate (578 mg, 13.8 mmol) in THF (I mL) and H₂O (1 L) was stirred at 10° C. for 16 h. The solvent was removed under reduced pressure. The residue was acidified to pH=5-6 with 1M hydrochloric acid. The aqueous was concentrated to dryness. The residue was triturated with water (1 mL) to give the titled compound (590 mg, 97%) as white solids. ¹H NMR (400 MHz, DMSO-d₆): δ 9.17-8.85 (m, 2H), 8.67 (s, 1H), 8.11 (s, 1H), 7.63-7.55 (m, 1H), 3.97-3.93 (m, 2H), 3.46-3.41 (m, 1H), 3.15-3.10 (m, 2H), 1.74-1.58 (m, 4H).

### 4-Amino-5-bromonicotinic acid

A mixture of 4-aminonicotinic acid (20.0 g, 145 mmol) in acetic acid (150 mL) and water (150 mL) was heated to 70° C. and stirred for 1 h. After cooled to 50° C., bromine (25 mL) was added dropwise. Then the mixture was stirred at 50° C. for 16 h. The mixture was cooled to 0° C. and the precipitate was collected by filtration. The cake was washed with ice-water and dried to give the titled compound (29.0 g, crude) as yellow solids.

### Methyl 4-amino-5-bromonicotinate

To a solution of 4-amino-5-bromonicotinic acid (29.0 g, crude) in anhydrous MeOH (200 mL) was added dropwise thionyl chloride (50 mL) at 0° C. Then the mixture was heated to reflux for 16 h. The solvent was removed under reduced pressure. The residue was basified to pH=10 with potassium carbonate aq. solution and extracted with EtOAc (80 mL×4). The combined organic layers was dried over sodium sulfate and concentrated to dryness. The residue was triturated with methyl tert-butyl ether (50 mL) to give the titled compound (4.80 g, 14% for two steps) as brown solids.

### Methyl 4-amino-5-(3,6-dihydro-2H-pyran-4-yl)nicotinate

A mixture of methyl 4-amino-5-bromonicotinate (1.0 g, 4.35 mmol), 4-(4,4,5,5-tetramethyl-1,3,2-dioxaborolan-2-yl)-3,6-dihydro-2H-pyran (1.37 g, 6.52 mmol), cesium carbonate (3.12 g, 9.57 mmol), triphenylphosphine (150 mg) and palladium acetate (150 mg) in THF/H₂O (18 mL, v/v=6/1) was heated to reflux 16 h under N₂ atmosphere. The mixture was extracted with EtOAc (15 mL×3). The combined organic layers was dried over sodium sulfate and concentrated to dryness. The residue was purified by chromatography on silica gel (EtOAc:PE=1:3) to give the titled compound (750 mg, 75%) as brown solids.

### Methyl 4-amino-5-(tetrahydro-2H-pyran-4-yl)nicotinate

A mixture of methyl 4-amino-5-(3,6-dihydro-2H-pyran-4-yl)nicotinate (750 mg, 3.21 mmol) and palladium on carbon (wet, 10%, 50 mg) in EtOAc (9 mL) and MeOH (1 mL) was stirred under H₂ balloon at 10° C. for 16 h. The mixture was filtered through the celite and the filtrate was concentrated to dryness to give the titled compound (650 mg, 86%) as white solids.

### Reference Example 4

### 3-Amino-2-isopropylisonicotinic acid

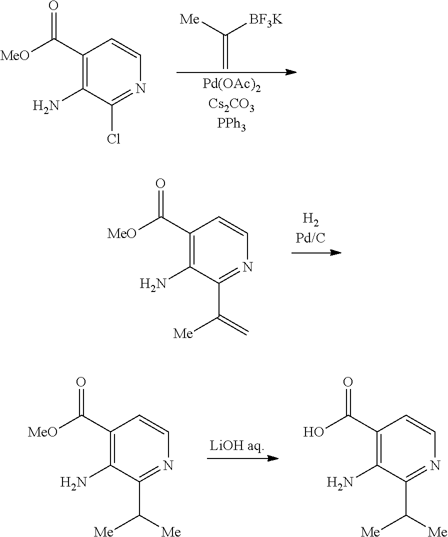

A solution of methyl 3-amino-2-isopropylisonicotinate (2.40 g, 12.4 mmol) and lithium hydroxide monohydrate (2.6 g, 61.9 mmol) in THF (10 mL) and H₂O (10 mL) was stirred at 10° C. for 16 h. The solvent was removed under reduced pressure. The residue was acidified to pH=5-6 with 1M hydrochloric acid. The solvent was removed under reduced pressure to about 5 mL solution. After cooled to rt, the precipitate was collected by filtration. The cake was washed with water and then dried to give the titled (2.20 g, 99%) as white solids. ¹H NMR (400 MHz, DMSO-d₆): δ 7.77-7.76 (m, 1H), 7.58-7.57 (m, 1H), 3.38-3.35 (m, 1H), 1.22 (d, J=6.8 Hz, 6H).

### Methyl 3-amino-2-isopropenylisonicotinate

A mixture of methyl 3-amino-2-chloroisonicotinate (3.0 g, 16.1 mmol), potassium isopropenyltrifluoroborate (2.35 g, 16.1 mmol), cesium carbonate (11.6 g, 35.5 mmol), triphenylphosphine (210 mg) and palladium acetate (150 mg) in THF/H₂O (50 mL, v/v=5/1) was heated to reflux 16 h under N₂ atmosphere. The mixture was extracted with EtOAc (30 mL×3). The combined organic layers were dried over sodium sulfate and concentrated to dryness. The residue was purified by chromatography on silica gel (EtOAc:PE=1:4) to give the titled compound (2.40 g, 77%) as yellow solids.

### Methyl 3-amino-2-isopropylisonicotinate

A mixture of methyl 3-amino-2-isopropenylisonicotinate (2.40 g, 12.5 mmol) and palladium on carbon (wet, 10%, 200 mg) in EtOAc (10 mL) and MeOH (10 mL) was stirred under H₂ balloon at 10° C. for 16 h. The mixture was filtered through the celite and the filtrate was concentrated to dryness to give the titled compound (2.40 g, 99%) as a yellow oil.

### Reference Example 5

### 5-Amino-6-isopropyl-4-pyrimidinecarboxyiic acid

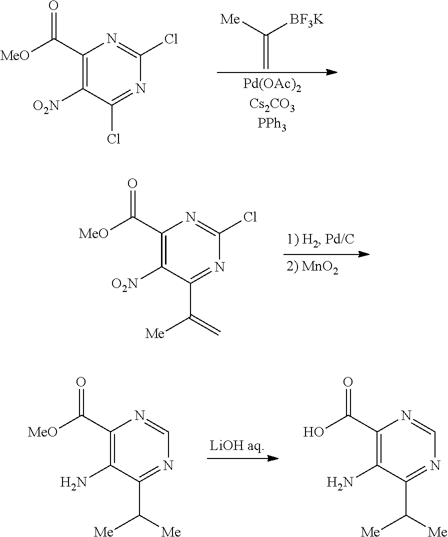

A solution of methyl 5-amino-6-isopropyl-4-pyrimidinecarboxylate (1.30 g, 6.22 mmol) and lithium hydroxide monohydrate (1.31 g, 31.1 mmol) in THF (10 mL) and H₂O (10 L) was stirred at 10° C. for 4 h. The solvent was removed under reduced pressure. The residue was acidified to pH=5-6 with 1M hydrochloric acid. The aqueous was extracted with DCM:MeOH (v/v=10:1) (20 mL×10). The combined organic layers were dried over sodium sulfate and concentrated to dryness to give the titled compound (1.05 g, 93%) as yellow solids. ¹H NMR (400 MHz, CDCl₃): δ 8.51 (s, 1H), 3.11 (br s s, 1H), 1.32-1.25 (m, 6H).

### Methyl 2-chloro-6-isopropenyl-5-nitro-4-pyrimidinecarboxylate

A mixture of methyl 2,6-dichloro-5-nitro-4-pyrimidinecarboxylate (3.40 g, 14.5 mmol), potassium isopropenyltrifluoroborate (2.14 g, 14.5 mmol), cesium carbonate (10.4 g, 31.8 mmol), triphenylphosphine (260 mg) and palladium acetate (280 mg) in THF/H₂O (50 mL, v/v=10/1) was heated to reflux 16 h under N₂ atmosphere. The solvent was removed under reduced pressure. The residue was diluted with water (50 mL) and filtered through the celite. The filtrate was extracted with EtOAc (40 mL×3). The combined organic layers was dried over sodium sulfate and concentrated to dryness. The residue was purified by chromatography on silica gel (EtOAc:PE=1:9) to give the titled compound (3.10 g, 89%) as a yellow oil.

### Methyl 5-amino-6-isopropyl-4-pyrimidinecarboxylate

A mixture of methyl 2-chloro-6-isopropenyl-5-nitro-4-pyrimidinecarboxylate (3.10 g, 12.9 mmol) and palladium on carbon (dry, 10%, 600 mg) in EtOH (50 mL) was stirred under H₂ (30 psi) at 10° C. for 16 h. The mixture was filtered through the celite and the filtrate was concentrated to dryness. The residue was dissolved in DCM (30 mL) and manganese dioxide (5.0 g) was added. The mixture was stirred at 10° C. for 20 h. The mixture was filtered through the celite and the filtrate was concentrated to dryness. The residue was purified by chromatography on silica gel (EtOAc:PE=1:9) to give the titled compound (1.20 g, 45%) as white solids.

### Reference Example 6

### 1-Amino-2-(tetrahydro-2H-pyran-4-yl)-1H-imidazole-5-carboxylic acid

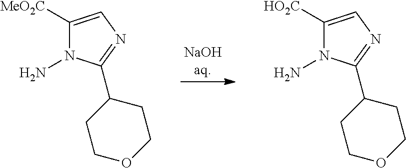 [SMILES: COC(=O)c1cnc(C2CCOCC2)n1N ; Nn1c(C(=O)O)cnc1C1CCOCC1]

To a solution of methyl 1-amino-2-(tetrahydro-2H-pyran-4-yl)-1H-imidazole-5-carboxylate (see Reference Example 2 intermediate, 5 g, 22 mmol) in THF (20 mL) was added 2 N sodium hydroxide aq. solution (45 mL, 44 mmol). Then the mixture was stirred at rt for 2 h. Adjusted the pH to 5-6. After concentration in vacuo and dissolved in MeOH, filtered and concentrated to give the titled compound (3.8 g, 82%) as yellow solids. ¹H NMR (400 MHz, CDCl₃) δ 7.46 (d, J=2.0 Hz, 1H), 3.93-3.90 (d, J=11.2 Hz, 2H), 3.45-3.39 (m, 2H), 3.26-3.22 (t, J=7.6 Hz, 1H), 1.75 (s, 4H). MS (ESI): m/e=212 [M+1]⁺.

### Reference Example 7

### (4R)-4-(4-Chlorophenyl)-2-(methylthio)-1,4,5,6-tetrahydropyrimidine

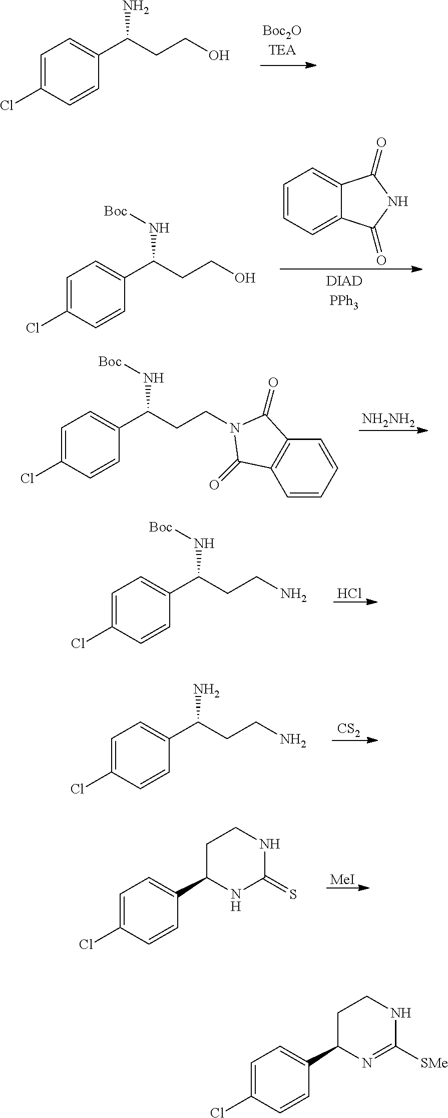

To a solution of (4R)-4-(4-chlorophenyl)tetrahydro-2(1H)-pyrimidinethione (510 mg, 2.26 mmol) in acetone (3 mL) was added methyl iodide (353 mg, 2.49 mmol). The mixture was heated to reflux in a sealed tube for 1 h. The solvent was removed to give the crude as yellow solids (500 mg, 92%) which was used in the next step without further purification. ¹H NMR (400 MHz, CDCl₃): δ 7.34-7.25 (m, 4H), 6.38 (br s, 1H), 4.59-4.56 (m, 1H), 3.46-3.37 (m, 2H), 2.44 (s, 3H), 2.14-2.07 (m, 1H), 1.78-1.75 (m, 1H). LCMS: m/z=241 [M+H]⁺.

### 2-Methyl-2-propanyl[(1R)-1-(4-chlorophenyl)-3-hydroxypropyl]carbamate

To a solution of (3R)-3-amino-3-(4-chlorophenyl)-1-propanol (1.85 g, 0.01 mol) in THF (20 mL) were added TEA (2.02 g, 0.02 mol) and di-tert-butyl dicarbonate (2.6 g, 0.012 mol). The mixture was stirred at rt for 2 h. The solvent was removed and the residue was purified by chromatography on silica gel (EtOAc:PE=1:10 to 1:6) to give the titled compound (2.75 g, 96%) as white solids. LCMS: m/z=286 [M+H]⁺.

### 2-Methyl-2-propanyl[(1R)-1-(4-chlorophenyl)-3-(1,3-dioxo-1,3-dihydro-2H-isoindol-2-yl)propyl]carbamate

To a solution of 2-methyl-2-propanyl[(1S)-1-(4-chlorophenyl)-3-hydroxypropyl]carbamate (2.75 g, 9.6 mmol) and phthalimide (1.4 g, 9.6 mmol) in THF (20 mL) was added triphenylphosphine (5 g, 19 mmol) under N₂. The mixture was cooled to 0° C. and DIAD (3.88 g, 19 mmol) was added. The reaction was stirred at rt for 15 h and quenched with water. The solvent was removed and the residue was purified by chromatography on silica gel (EtOAc:PE=1:10 to 1:8) to give the titled compound (3.0 g, 76%) as white solids. LCMS: m/z=415 [M+H]⁺.

### 2-Methyl-2-propanyl[(1R)-3-amino-1-(4-chlorophenyl)propyl]carbamate

To a solution of 2-methyl-2-propanyl[(1R)-1-(4-chlorophenyl)-3-(1,3-dioxo-1,3-dihydro-2H-isoindol-2-yl)propyl]carbamate (3.0 g, 7.2 mmol) in EtOH (10 mL) was added hydrazine (900 mg, 14.4 mmol). The mixture was heated to reflux for 2 h. The solvent was removed and the residue was purified by chromatography on silica gel (EtOAc:PE=1:8 to 1:5) to give the titled compound (1.4 g, 70%) as white solids. LCMS: m/z=285 [M+H]⁺.

### (1R)-1-(4-Chlorophenyl)-1,3-propanediamine

2-Methyl-2-propanyl[(1R)-3-amino-1-(4-chlorophenyl)propyl]carbamate (1.4 g, 5 mmol) was dissolved in hydrochloric acid in dioxane (4 mL). The mixture was stirred at rt for 2 h. The solvent was removed to give crude (0.9 g, 98%) as white solids, which was used in the next step without further purification. LCMS: m/z=185 [M+H]⁺.

### (4R)-4-(4-chlorophenyl)tetrahydro-2(1H)-pyrimidinethione

To a solution of (1R)-1-(4-chlorophenyl)-1,3-propanediamine (900 mg, 4.9 mmol) in water (10 mL) was added carbon disulfide (486 mg, 6.4 mmol), triethanolamine (1 mL) and Lac sulfur powder (20 mg). The mixture was heated to reflux for 4 h. The solvent was removed and the residue was purified by chromatography on silica gel (EtOAc:PE=1:10 to 1:3) to give the titled compound (500 mg, 45%) as white solids. LCMS: m/z=227 [M+H]⁺.

### Reference Example 8

### 2-(Methylthio)-1,2′,3′,5-tetrahydrospiro[imidazole-4,1′-indene]

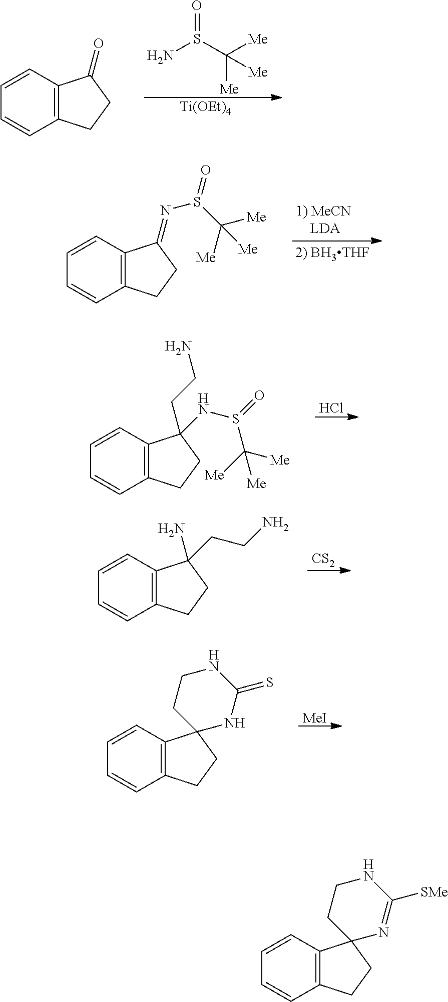

To a solution of 2,3,5′,6′-tetrahydro-1′H-spiro[indene-1,4′-pyrimidine]-2′(3′H)-thione (130 mg, 0.6 mmol) in MeOH (10 mL) was added methyl iodide (127 mg, 0.89 mmol). The solution was stirred at 80° C. for 16 h. The product was purified through flash column to get the titled compound (76 mg, 55%). ¹H NMR (400 MHz, CDCl₃): δ 2.16-2.29 (m, 2H), 2.44-2.55 (m, 1H), 2.80 (s, 3H), 2.88-2.96 (m, 1H), 3.01-3.08 (m, 1H), 3.58-3.67 (m, 2H), 3.76-3.83 (m, 1H), 7.16-7.17 (m, 1H), 7.21-7.28 (m, 3H), 7.85 (s, 1H). LCMS: m/z=233 [M+1]⁺.

### N-[(1E)-2,3-Dihydro-1H-inden-1-ylidene]-2-methyl-2-propanesulfinamide

A solution of 1-indanone (5 g, 37 mmol), 2-methylpropane-2-sulfinamide (6.9 g, 56.8 mmol) and titanium ethoxide (17.2 g, 75.8 mmol) in anhydrous THF (100 mL) was heated to 80° C. for 8 h under N₂ protection. The reaction was quenched by adding 10 mL water, then purified through gel column to give the titled compound (3 g, 33%). ¹H NMR (400 MHz, CDCl₃): δ 1.32 (s, 9H), 3.01-3.09 (m, 1H), 3.13-3.16 (m, 2H), 3.41-3.48 (m, 1H), 7.31-7.34 (m, 1H), 7.34-7.41 (m, 1H), 7.48-7.51 (m, 1H), 7.79-7.81 (m, 1H). LCMS: m/z=236 [M+1]⁺.

### N-[1-(2-Aminoethyl)-2,3-dihydro-1H-inden-1-yl]-2-methyl-2-propanesulfinamide

To a solution of anhydrous MeCN (174 mg, 4.25 mmol) in anhydrous THF (15 mL) was added 2M lithium diisopropylamide (1.5 mL, 3.19 mmol) at −78° C. under N₂ for 10 min. The mixture was stirred at −78° C. for another 0.5 h. (N-[(1E)-2,3-dihydro-1H-inden-1-ylidene]-2-methyl-2-propanesulfinamide (500 mg, 2.12 mmol) in anhydrous THF (20 mL) was dropped into upper solution at −78° C. The solution was slowly warmed to −50° C., and then stirred at −50° C. for another 5 h. The solution was quenched by adding 20 mL saturated ammonium chloride solution. The crude product was extracted from water by EtOAc (30 mL×3), and then the solvents were removed in vacuo. The crude product was dissolved in anhydrous THF (10 mL), then 1M borane-THF complex solution (5 mL) was added dropwise. The reaction was heated to 80° C. for 10 min under N₂ protection. The reaction was cooled to 25° C., and then quenched by adding 1 mL MeOH. The product was purified through flash column to give the titled compound (235 mg, 39%). LCMS: m/z=281 [M+1]⁺.

### 1-(2-Aminoethyl)-1-indanamine

A solution of N-[1-(2-Aminoethyl)-2,3-dihydro-1H-inden-1-yl]-2-methyl-2-propanesulfinamide (200 mg, 0.71 mmol) in THF (10 mL) was added 1M hydrochloric acid/THF (4 mL) at 0° C. The reaction was stirred at 25° C. for 2 h. The mixture was evaporated under vacuum to get the crude product. The crude product was purified through flash column to get the titled compound (110 mg, 88%). ¹H NMR (400 MHz, CDCl₃): δ 1.94-2.03 (m, 2H), 2.85-2.89 (m, 4H), 3.98-4.01 (m, 4H), 7.12-7.19 (m, 2H), 8.00-8.05 (m, 2H), 8.78-8.79 (m, 2H). LCMS: m/z=160 [M-NH₂]⁺.

### 2,3,5′,6′-Tetrahydro-1′H-spiro[indene-1,4′-pyrimidine]-2′(3′H)-thione

To a solution of 1-(2-aminoethyl)-1-indanamine (200 mg, 1.13 mmol) in water (10 mL) was added carbon disulfide (104 mg, 1.36 mmol), triethanolamine (486 mmol, 4.54 mmol) and S (350 mg, 11 mmol). The solution was stirred at 100° C. for 3 h. The crude product was extracted from water by EtOAc (30 mL×3), then purified through flash column to get the titled compound (130 mg, 52%). ¹HNMR (400 MHz, CDCl₃): δ 1.86-1.92 (m, 2H), 2.12-2.21 (m, 2H), 2.76-2.83 (m, 1H), 2.88-2.97 (m, 1H), 3.64-3.96 (m, 2H), 6.53 (s, 1H), 6.95 (s, 1H), 7.18-7.20 (m, 4H). LCMS: m/z=219 [M+H]⁺.

### Reference Example 9

### 2-(Methylthio)-1,2′,3′,5-tetrahydrospiro[imidazole-4,1′-indene]

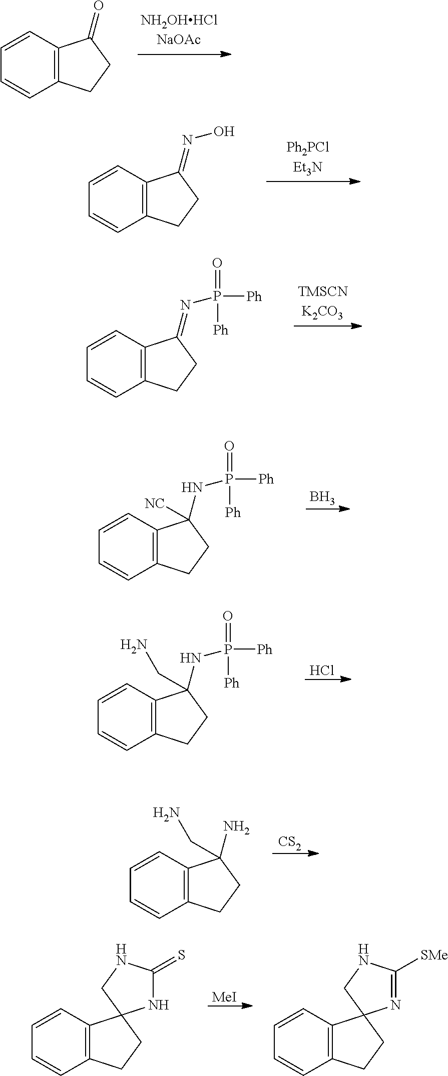

To a solution of 2′,3′-dihydro-2H-spiro[imidazolidine-4,1′-indene]-2-thione (20 mg, 0.097 mmol) in MeOH (3 mL) was added methyl iodide (20.8 mg, 0.15 mmol). The mixture was stirred at 70° C. for 3 h. The product was concentrated to get crude product which was used in the next step without further purification. ¹H NMR (400 MHz, CD₃OD): δ 2.31-2.44 (m, 2H), 2.60 (s, 3H), 2.83-3.03 (m, 2H), 3.98-4.08 (m, 2H), 7.22-7.34 (m, 4H). LCMS: m/z=219 [M+H]⁺.

### (1E)-1-Indanone oxime

To a solution of 1-indanone (2.64 g, 20 mmol) in EtOH (15 mL) was added hydroxylamine hydrochloride (1.66 g, 24 mmol) and sodium acetate (1.97 g, 24 mmol). The mixture was stirred at 70° C. for 3 h. Then the mixture was concentrated to get the crude product as white solids. LCMS: m/z=148 [M+H]⁺.

### N-[(1E)-2,3-Dihydro-1H-inden-1-ylidene]-P,P-diphenylphosphinic amide

To a bottle was charged (1E)-1-indanone oxime (735 mg, 5.0 mmol) in DCM (20 mL). The mixture was stirred at −40° C. for 20 mins. Then chlorodiphenylphosphine (1.1 g, 5.0 mmol) and TEA (1.01 g, 10.0 mmol) was added. The reaction mixture was stirred at −40° C. to rt overnight. The product was purified by column chromatography (EA/PE=1/2) as a yellow oil. ¹H NMR (400 MHz, CD₃OD): δ 3.17-3.20 (m, 4H), 7.44-7.67 (m, 10H), 7.88-7.99 (m, 4H). LCMS: m/z=332 [M+H]⁺.

### N-(1-Cyano-2,3-dihydro-1H-inden-1-yl)-P,P-diphenylphosphinic amide

To a solution of N-[(1E)-2,3-dihydro-1H-inden-1-ylidene]-P,P-diphenylphosphinic amide (500 mg, 1.5 mmol) in DMF (10 mL) was added trimethylsilylcyanide (179 mg, 1.8 mmol) and potassium carbonate (417 mg, 3.0 mmol). The mixture was stirred at rt overnight. The product was purified by column chromatography (EA/PE=1/1) as a oil. ¹H NMR (400 MHz, DMSO-d₆): δ 2.66 (s, 2H), 2.82 (s, 2H), 6.46 (d, J=9.1 Hz, 1H), 7.14-7.15 (m, 2H), 7.32-7.49 (m, 7H), 7.59-7.64 (m, 4H), 7.88 (s, 1H). LCMS: m/z=359 [M+H]⁺.

### N-[1-(Aminomethyl)-2,3-dihydro-1H-inden-1-yl]-P,P-diphenylphosphinic amide

To a bottom was charged N-(1-cyano-2,3-dihydro-1H-inden-1-yl)-P,P-diphenylphosphinic amide (500 mg, 1.4 mmol) in 1 M borane-THF complex solution (3 mL). The mixture was stirred at rt for 2 h. The product was purified by column chromatography (EA/PE=1/1) as yellow solids. ¹H NMR (400 MHz, DMSO-d₆): δ 2.06-2.10 (m, 1H), 2.21-2.28 (m, 1H), 2.58-2.72 (m, 3H), 3.15-3.16 (m, 1H), 5.54-5.64 (m, 1H), 7.01-7.21 (m, 4H), 7.35-7.52 (m, 8H), 7.61-7.73 (m, 4H). LCMS: m/z=363 [M+H]⁺.

### 1-(Aminomethyl)-1-indanamine

To a round bottom was charged N-(1-(aminomethyl)-2,3-dihydro-1H-inden-1-yl)-P,P-diphenylphosphinic amide (200 mg, 0.55 mmol) in hydrochloric acid/dioxane (5 mL). The mixture was stirred at 50° C. for 4 h. Then the mixture was concentrated to get crude product, which was used for next step directly. ¹H NMR (400 MHz, CD₃OD): δ 2.12-2.31 (m, 1H), 2.81-3.25 (m, 3H), 3.40 (s, 1H), 3.89 (s, 1H), 7.19-7.49 (m, 4H). LCMS: m/z=146 [M+H]⁺.

### 2′,3′-dihydro-2H-spiro[imidazolidine-4,1′-indene]-2-thione

To a solution of 1-(aminomethyl)-1-indanamine (120 mg, 0.74 mmol) in H₂O (3 mL) was added carbon disulfide (112.6 mg, 1.48 mmol), S (1.19 mg, 0.037 mmol) and 2,2′,2″-nitrilotriethanol (552 mg, 3.7 mmol). The mixture was stirred at 100° C. for 2 h. The product was purified by reversed phase (0.01% NH₃ in water and MeCN) as yellow solids. ¹H NMR (400 MHz, DMSO-d₆): δ 2.07-2.15 (m, 1H), 2.23-2.29 (m, 1H), 2.72 (s, 2H), 2.88 (s, 2H), 3.49 (d, J=10.0 Hz, 1H), 3.67 (d, J=10.0 Hz, 1H), 7.23-7.27 (no, 4H). LCMS: m/z=205 [M+H]⁺.

### EXAMPLES

### Example 1

### 1-Cyclopentyl-6-(3-hydroxy-1-(4-methoxyphenyl)propylamino)-1H-pyrazolo[3,4-d]pyrimidin-4(5H)-one

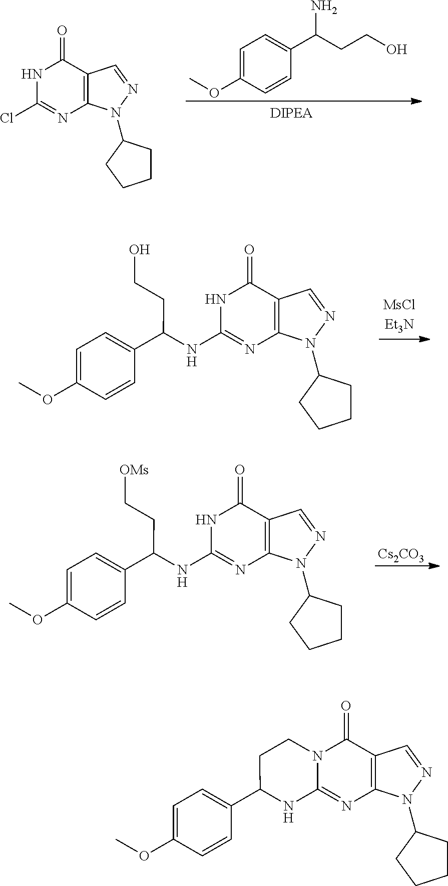

3-[(1-Cyclopentyl-4-oxo-4,5-dihydro-1H-pyrazolo[3,4-d]pyrimidin-6-yl)amino]-3-(4-methoxyphenyl)propyl methanesulfonate (133 mg, 0.29 mmol) and cesium carbonate (189 mg, 0.58 mmol) in dioxane (2 mL) was heated to 100° C. for 1 h. The solvent was removed. Water was added and the aqueous was extracted with EtOAc (20 mL×3). The combined organic phase was washed with brine, dried with sodium sulfate and concentrated. The crude product was purified by Pre-TLC (DCM:MeOH=20:1) to give the titled compound (20 mg, 19%) as yellow solids. ¹H NMR (400 MHz, CDCl₃): δ 7.93 (s, 1H), 7.25 (d, J=8.8 Hz, 2H), 6.91 (d, =8.4 Hz, 2H), 5.65 (s, 1H), 4.90 (quint, J=7.6 Hz, 1H), 4.66-4.63 (m, 1H), 4.30 (dt, J1=14.0 Hz, J2=4.8 Hz, 1H), 3.84-3.77 (m, 4H), 2.34-2.29 (m, 1H), 2.09-1.89 (m, 7H), 1.69-1.62 (m, 2H). LCMS: m/z=366 [M+H]⁺.

### 1-Cyclopentyl-6-{[3-hydroxy-1-(4-methoxyphenyl)propyl]amino}-1,5-dihydro-4H-pyrazolo[3,4-d]pyrimidin-4-one

A mixture of 6-chloro-1-cyclopentyl-1,5-dihydro-4H-pyrazolo[3,4-d]pyrimidin-4-one (synthesized in a similar manner to Reference Example 1, 107 mg, 0.45 mmol), 3-amino-3-(4-methoxyphenyl)-1-propanol (151 mg, 1.0 mmol) and DIPEA (174 mg, 1.35 mmol) in 1-butanol (2 mL) was heated to 120° C. and stirred for 3 h. The solvent was removed. Water was added and the aqueous was extracted with EtOAc (20 mL×3). The combined organic phase was washed with brine, dried with sodium sulfate and concentrated. The crude was purified by Pre-TLC (DCM:MeOH=20:1) to give the titled compound (146 mg, 85%) as yellow solids. LCMS: m/z=384 [M+1]⁺.

### 3-[(1-Cyclopentyl-4-oxo-4,5-dihydro-1H-pyrazolo[3,4-d]pyrimidin-6-yl)amino]-3-(4-methoxyphenyl)propyl methanesulfonate

A mixture of 1-cyclopentyl-6-{[3-hydroxy-1-(4-methoxyphenyl)propyl]amino}-1,5-dihydro-4H-pyrazolo[3,4-d]pyrimidin-4-one (110 mg, 0.29 mmol) and TEA (88 mg, 0.87 mmol) in DCM (5 mL) was stirred at 0° C. Methanesulfonyl chloride (66 mg, 0.58 mmol) was added. The resulting mixture was stirred at rt overnight. Water was added and the organic phase was separated. The separated organic phase was washed with brine, dried with sodium sulfate and concentrated to give crude (133 mg, 100%) which was used in the next step without further purification. LCMS: m/z=462 [M+1]⁺.

The compounds of Example 2 to 17 were synthesized in a similar manner to Example 1.

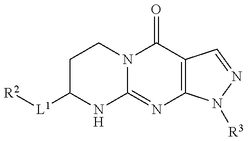

| Ex. | | | | |
|---|---|---|---|---|
| No. | R²— | L¹ | R³— | ¹H-NMR |
| 2 | 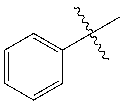 [SMILES: *c1ccccc1] | -bond- | 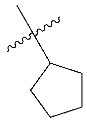 [SMILES: *C1CCCC1] | ¹H NMR (400 MHz, CDCl₃): δ 7.94 (s, 1H), 7.44-7.35 (m, 5H), 5.57 (s, 1H), 4.92 (quint, J = 7.6 Hz, 1H), 4.74-4.71 (m, 1H), 4.28-4.23 (m, 1H), 3.92-3.85 (m, 1H), 2.38-2.33 (m, 1H), 2.11-1.93 (m, 7H), 1.68 (br s, 2H) |
| 3 | 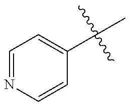 [SMILES: *c1ccncc1] | -bond- | 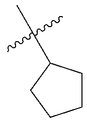 [SMILES: *C1CCCC1] | ¹H NMR (400 MHz, CDCl₃): δ 8.67-8.66 (m, 2H), 7.95 (s, 1H), 7.31-7.29 (m, 2H), 5.57 (s, 1H), 4.97-4.89 (m, 1H), 4.78-4.74 (m, 1H), 4.03 (t, J = 5.6 Hz, 2H), 2.43-2.36 (m, 1H), 2.12-2.00 (m, 5H), 1.99-1.93 (m, 2H), 1.71- 1.69 (m, 2H) |
| 4 | 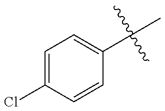 [SMILES: *c1ccc(Cl)cc1] | -bond- | 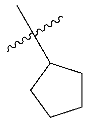 [SMILES: *C1CCCC1] | ¹H NMR (400 MHz, CDCl₃): δ 7.94 (s, 1H), 7.39 (d, J = 8.4 Hz, 2H), 7.30 (d, J = 8.8 Hz, 2H), 5.55 (s, 1H), 4.92 (quint, J = 7.6 Hz, 1H), 4.73-4.70 (m, 1H), 4.25-4.19 (m, 1H), 3.93- 3.86 (m, 1H), 2.36-2.33 (m, 1H), 2.11-1.90 (m, 7H), 1.72-1.65 (m, 2H) |
| 5 | 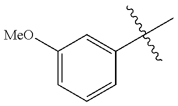 [SMILES: *c1cccc(OC)c1] | -bond- | 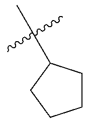 [SMILES: *C1CCCC1] | ¹H NMR (400 MHz, CDCl₃): 7.81 (s, 1H), 7.27 (t, J = 8 Hz, 1H), 6.92 (d, J = 8 Hz, 1H), 6.88 (s, 1H), 6.80 (dd, J = 7.6, 1.6 Hz, 1H), 5.32 (br s, 1H), 4.86 (dt, J = 13.6, 6.8 Hz, 1H), 4.22- 4.20 (m, 1H), 3.89 (dt, J = 11.2, 4.4 Hz, 1H), 3.78 (s, 3H), 3.45 (s, 1H), 2.49-2.42 (m, 1H), 2.34-2.30 (m, 1H), 2.22-2.02 (m, 3H), 1.92- 1.90 (m, 3H), 1.65-1.59 (m, 2H) |
| 6 | 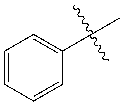 [SMILES: *c1ccccc1] | —CH₂— | 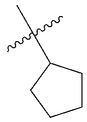 [SMILES: *C1CCCC1] | ¹H NMR (400 MHz, CDCl₃): δ 7.91 (s, 1H), 7.41-7.24 (m, 5H), 5.24 (s, 1H), 4.87 (quint, J = 7.6 Hz, 1H), 4.52 (dt, J1 = 14.0 Hz, J2 = 4.8 Hz, 1H), 3.81-3.77 (m, 1H), 3.67-3.60 (m, 1H), 2.97 (dd, J1 = 13.6 Hz, J2 = 4.0 Hz, 1H), 2.79 (dd, J1 = 13.6 Hz, J2 = 8.4 Hz, 1H), 2.23-2.18 (m, 1H), 2.08-1.55 (m, 9H) |
| 7 | 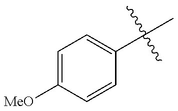 [SMILES: *c1ccc(OC)cc1] | —CH₂— | 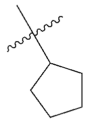 [SMILES: *C1CCCC1] | ¹H NMR (400 MHz, CD₃OD) δ: 7.81 (s, 1H), 7.21 (d, J = 8.4 Hz, 2H), 6.91 (d, J = 8.4 Hz, 2H), 4.95 (dd, J = 7.2 Hz, 14.8 Hz, 1 H), 4.29 (dd, J = 10.0 Hz, 14.0 Hz, 1 H), 3.80 (s, 3H), 3.67-3.78 (m, 2H), 2.97 (dd, J = 4.5 Hz, 13.6 Hz, 1 H), 2.78 (dd, J = 8.0 Hz, 13.6 Hz, 1 H), 1.92-2.08 (m, 7H), 1.69-1.77 (m, 3H) |
| 8 | 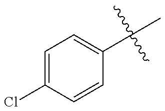 [SMILES: *c1ccc(Cl)cc1] | —CH₂— | 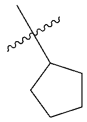 [SMILES: *C1CCCC1] | ¹HNMR (400 MHz, CDCl₃): δ 7.93 (s, 1H), 7.37-7.40 (m, 2H), 7.20-7.22 (m, 2H), 4.88- 4.91 (m, 1H), 4.46-4.50 (m, 1H), 3.78 (m, 1H), 3.65-3.71 (m, 1H), 2.93-2.98 (m, 1H), 2.80- 2.84 (m, 1H), 2.19-2.20 (m, 1H), 1.68-2.10 (m, 10H) |
| 9 | 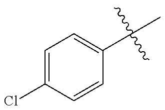 [SMILES: *c1ccc(Cl)cc1] | -bond- | 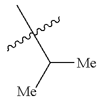 [SMILES: *C(C)C] | ¹H NMR (400 MHz, CDCl₃): δ 7.95 (s, 1H), 7.39 (d, J = 8.8 Hz, 2H), 7.30 (d, J = 8.4 Hz, 2H), 5.52 (s, 1H), 4.78 (sept, J = 6.8 Hz, 1H), 4.81-4.70 (m, 1H), 4.26-4.20 (m, 1H), 3.93- 3.86 (m, 1H), 2.38-2.33 (m, 1H), 2.06-2.02 (m, 1H), 1.51-1.48 (m, 6H) |
| 10 | 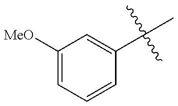 [SMILES: *c1cccc(OC)c1] | -bond- | 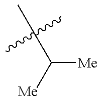 [SMILES: *C(C)C] | ¹H NMR (400 MHz, CDCl₃): δ 7.95 (s, 1H), 7.34 (t, J = 8.0 Hz, 1H), 6.94 (d, J = 7.6 Hz, 1H), 6.95-6.91 (m, 1H), 6.89 (s, 1H), 5.62 (br s, 1H), 4.79 (sept, J = 6.4 Hz, 1H), 4.72-4.69 (m, 1H), 4.29-4.23 (m, 1H), 3.93-3.87 (m, 1H), 3.84 (s, 3H), 2.39-2.33 (m, 1H), 2.12-2.02 (m, 1H), 1.52-1.49 (m, 6H) |
| 11 | 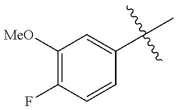 [SMILES: *c1ccc(F)c(OC)c1] | -bond- | 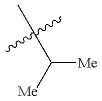 [SMILES: *C(C)C] | ¹H NMR (400 MHz, CDCl₃): δ 7.96 (s, 1H), 7.12 (dd, J1 = 10.8 Hz, J2 = 8.0 Hz, 1H), 6.96- 6.90 (m, 2H), 5.51 (br s, 1H), 4.79 (sept, J = 6.8 Hz, 1H), 4.71-4.68 (m, 1H), 4.36-4.31 (m, 1H), 3.94 (s, 3H), 3.90-3.84 (m, 1H), 2.38-2.32 (m, 1H), 2.09-2.02 (m, 1H), 1.53-1.49 (m, 6H) |
| 12 | 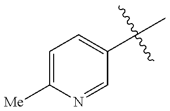 [SMILES: *c1ccc(C)nc1] | -bond- | 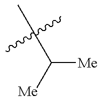 [SMILES: *C(C)C] | ¹H NMR (400 MHz, CDCl₃): δ 8.51 (s, 1H), 7.75 (br s, 1H), 7.63 (d, J = 6.4 Hz, 1H), 7.17 (d, J = 8.0 Hz, 1H), 5.21 (br s, 1H), 4.81 (sept, J = 6.4 Hz, 1H), 4.34-4.31 (m, 1H), 4.04 (br s, 1H), 2.54-2.49 (m, 4H), 2.36-2.32 (m, 1H), 2.03 (br s, 1H), 1.58 (d, J = 6.0 Hz, 3H), 1.48 (d, J = 6.8 Hz, 3H) |
| 13 | 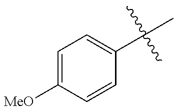 [SMILES: *c1ccc(OC)cc1] | —CH₂— | 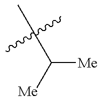 [SMILES: *C(C)C] | ¹H NMR (400 MHz, CD₃OD): δ 7.82 (s, 1H), 7.22 (d, J = 8.4 Hz, 2H), 6.92 (d, J = 8.4 Hz, 2H), 4.87-4.79 (m, 1H), 4.59 (br s, 1H), 4.32- 4.26 (m, 1H), 3.80 (s, 3H), 3.78-3.68 (m, 2H), 2.96 (dd, J1 = 13.2 Hz, J2 = 5.6 Hz, 1H), 2.77 (dd, J1 = 13.6 Hz, J2 = 8.4 Hz, 1H), 2.09-2.02 (m, 1H), 1.76-1.69 (m, 1H), 1.47-1.44 (m ,6H) |
| 14 | 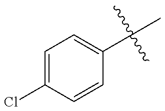 [SMILES: *c1ccc(Cl)cc1] | —CH₂— | 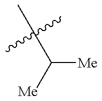 [SMILES: *C(C)C] | ¹H NMR (400 MHz, CDCl₃): δ 7.93 (s, 1H), 7.38-7.40 (m, 2H), 7.20-7.22 (m, 2H), 5.14 (s, 1H), 4.73-4.77 (m, 1H), 4.46-4.51 (m, 1H), 3.77-3.79 (m, 1H), 3.64-3.71 (m, 1H), 2.93- 2.98 (m, 1H), 2.77-2.82 (m, 1H), 2.19-2.24 (m, 1H), 1.79-1.82 (m, 1H), 1.45-1.50 (m ,6H) |
| 15 | 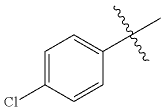 [SMILES: *c1ccc(Cl)cc1] | -bond- | 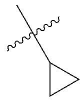 [SMILES: *C1CC1] | ¹H NMR (400 MHz, CDCl₃): δ 7.88 (s, 1H), 7.41 (d, J = 8.8 Hz, 2H), 7.32 (d, J = 8.4 Hz, 2H), 5.61 (s, 1H), 4.73-4.76 (m, 1H), 4.21-4.28 (m, 1H), 3.88-3.95 (m, 1H), 3.55 (sept, J = 6.8 Hz, 1H), 2.34-2.40 (m, 1H), 2.03-2.10 (m, 1H), 1.24-1.29 (m, 2H), 1.04-1.09 (m ,2H) |
| 16 | 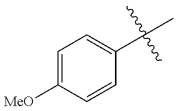 [SMILES: *c1ccc(OC)cc1] | —CH₂— | 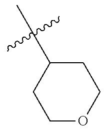 [SMILES: *C1CCOCC1] | ¹H NMR (400 MHz, CDCl₃): δ 7.91 (s, 1H), 7.17-7.19 (d, 2H), 6.93-6.95 (d, 2H), 5.20 (s, 1H), 4.49-4.55 (m, 2H), 4.08-4.13 (m, 2H), 3.85 (s, 3H), 3.61-3.62 (m, 1H), 3.55-3.56 (m, 3 H), 2.92-2.96 (m, 1 H), 2.69-2.75 (m, 1 H), 2.28-2.36 (m, 3 H), 1.90-1.87 (m, 3H) |
| 17 | 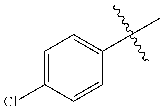 [SMILES: *c1ccc(Cl)cc1] | —CH_{2=l —} | 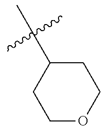 [SMILES: *C1CCOCC1] | ¹H NMR (400 MHz, CDCl₃): δ 7.93 (s, 1H), 7.39 (dd, J = 8.4 Hz, 2H), 7.21 (dd, J = 8.4 Hz, 2H), 5.16 (br s, 1H), 4.46-4.60 (m, 2H), 4.09- 4.15 (m, 2H), 3.54-3.82 (m, 4H), 2.79-2.97 (m, 2 H), 2.26-2.40 (m, 3 H), 1.76-1.88 (m, 3H) |

### Example 18

### (2R)-2-(4-Chlorophenyl)-9-isopropyl-1,2,3,4-tetrahydro-6H-imidazo[5,1-f]pyrimido[2,1-c][1,2,4]triazin-6-one

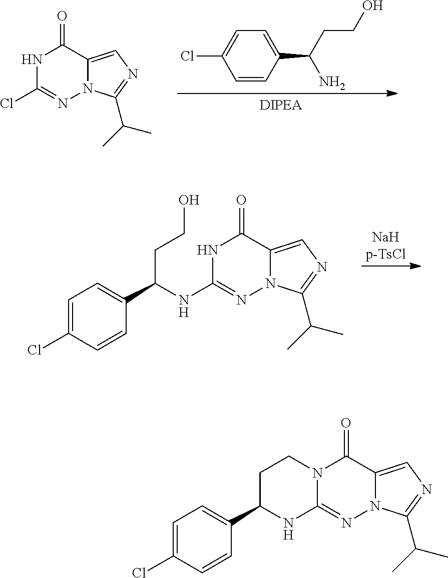 [SMILES: CC(C)c1ncc2c(=O)[nH]c(Cl)nn12 ; N[C@H](CCO)c1ccc(Cl)cc1 ; CC(C)c1ncc2c(=O)[nH]c(N[C@H](CCO)c3ccc(Cl)cc3)nn12 ; CC(C)c1ncc2c(=O)n3c(nn12)N[C@@H](c1ccc(Cl)cc1)CC3]

(2R)-2-(1-(4-Chlorophenyl)-3-hydroxypropylamino)-7-isopropylimidazo[1,5-f][1,2,4]triazin-4(3H)-one (100 mg, 0.28 mmol) and sodium hydride (0.03 g, 1.4 mmol) was dissolved in THF (1.5 mL) under N₂ protection. After 10 mins, p-tosyl chloride (0.06 g, 0.34 mmol) in THF (0.5 mL) was added slowly. The reaction mixture was stirred for 30 mins, and monitored by LCMS, and then purified by Prep-HPLC to give the titled compound (50 mg, 52.6%) as white solids. ¹H NMR (400 MHz, CD₃OD): δ 8.21 (s, 1H), 7.43-7.45 (m, 4H), 4.80 (dd, J1=7.6 Hz, J2=4.4 Hz, 1H), 3.95-4.11 (m, 2H), 3.71 (sept, J=6.8 Hz, |H), 2.38-2.43 (m, 1H), 2.09-2.13 (m, 1H), 1.47-1.50 (m, 6H). LCMS: m/z=344 [M+H]⁺.

### (2R)-2-(1-(4-Chlorophenyl)-3-hydroxypropylamino)-7-isopropylimidazo[1,5-f][1,2,4]triazin-4(3H)-one

(3R)-3-Amino-3-(4-chlorophenyl)-1-propanol (240 mg, 1.13 mmol) and 2-chloro-7-isopropylimidazo[5,1-f][1,2,4]triazin-4(1H)-one (synthesized in a similar manner to Reference Example 2, 270 mg, 1.46 mmol) in 1-butanol (6 mL), then DIPEA (0.49 g, 3.8 mmol) was added. The mixture was heated under microwave for 18 h at 170° C. The progress of the reaction mixture was monitored by LCMS and then purified by column chromatography (DCM:MeOH=20:1) to give the titled compound (0.09 g, 22.5%) as a colorless oil. LCMS: m/z=362 [M+H]⁺.

The compounds of Example 19 to 68 were synthesized in a similar manner to Example 18 and Reference Example 1.

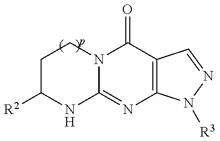

| Ex. | | | | | |
|---|---|---|---|---|---|
| No. | p | R²— | L¹ | R³— | ¹H-NMR |
| 19 | 0 | 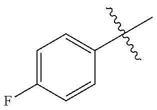 [SMILES: *c1ccc(F)cc1] | -bond- | 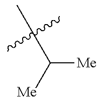 [SMILES: *C(C)C] | ¹H NMR (400 MHz, CDCl₃): δ 7.93 (s, 1 H), 7.34-7.39 (m, 2 H), 7.09-7.14 (m, 2 H), 5.70 (s, 1 H), 5.13-5.17 (m, 1 H), 4.74-4.81 (m, 1 H), 4.55-4.61 (m, 1 H), 3.93-3.97 (m, 1 H), 1.47-1.50 (m, 6 H) |
| 20 | 0 | 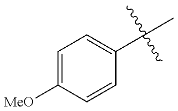 [SMILES: *c1ccc(OC)cc1] | -bond- | 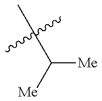 [SMILES: *C(C)C] | ¹H NMR (400 MHz, CDCl₃): δ 7.93 (s, 1 H), 7.27-7.30 (d, J = 12 Hz, 2 H), 6.91- 6.93 (d, J = 8 Hz, 2 H), 5.65 (s, 1 H), 5.10 (t, J = 10 Hz, 1 H), 4.75-4.78 (m, 1 H), 4.52-4.57 (m, 1 H), 3.94-3.98 (m, 1 H), 3.81 (s, 3 H), 1.47-1.49 (m, 6 H) |
| 21 | 0 | 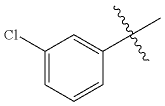 [SMILES: *c1cccc(Cl)c1] | -bond- | 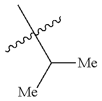 [SMILES: *C(C)C] | ¹H NMR (400 MHz, CDCl₃): δ 7.94 (s, 1 H), 7.35-7.39 (m, 3 H), 7.24-7.26 (m ,1 H), 5.67 (s, 1 H), 5.11-5.16 (m, 1 H), 4.76-4.80 (m, 1 H), 4.57-4.63 (m, 1 H), 3.95-3.40 (m, 1 H), 1.48-1.51 (m, 6 H) |
| 22 | 0 | 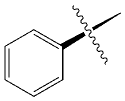 | -bond- | 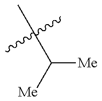 [SMILES: *C(C)C] | ¹H NMR (400 MHz, CDCl₃): δ 7.93 (s, 1H), 7.44-7.36 (m, 5 H), 5.66 (br s, 1H), 51.7-5.13 (m, 1H), 4.79-4.74 (m, 1H), 4.62-4.56 (m, 1H), 4.05-3.98 (m, 1H), 1.49-1.47 (m, 6H) |
| 23 | 0 | 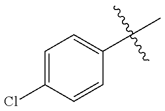 [SMILES: *c1ccc(Cl)cc1] | -bond- | 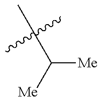 [SMILES: *C(C)C] | ¹H NMR (400 MHz, CDCl₃): δ 7.95 (s, 1 H), 7.39-7.42 (m, 2 H), 7.32-7.34 (m, 2 H), 5.49 (s, 1 H), 5.14 (m, 1 H), 4.79-4.82 (m, 1 H), 4.57-4.63 (m, 1 H), 3.94-3.98 (m, 1 H), 1.50-1.52 (m, 6 H). |
| 24 | 0 | 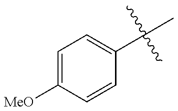 [SMILES: *c1ccc(OC)cc1] | -bond- | 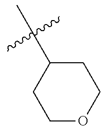 [SMILES: *C1CCOCC1] | ¹H NMR (400 MHz, CDCl₃): δ 7.96 (s, 1 H), 7.31 (d, J = 8.8 Hz, 2 H), 6.95 (d, J = 8.8 Hz, 2 H), 5.37 (s, 1 H), 5.12 (t, J = 8.8 Hz, 1 H), 4.64-4.55 (m, 2 H), 4.17-4.13 (m, 2 H), 4.00 (dd, J1 = 7.6 Hz, J2 = 11.6 Hz, 1 H), 3.84 (s, 3 H), 3.62-3.56 (m, 2 H), 2.39-2.35 (m, 2 H), 1.93-1.89 (m, 2 H) |
| 25 | 1 | 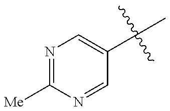 [SMILES: *c1cnc(C)nc1] | -bond- | 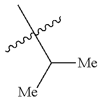 [SMILES: *C(C)C] | ¹H NMR (400 MHz, CDCl₃): 8.70 (s, 2 H), 7.97 (s, 1 H), 5.59 (br s, 1 H), 4.83-4.76 (m, 2 H), 4.34-4.28 (m, 1 H), 3.99-3.92 (m, 1 H), 2.80 (s, 3 H), 2.46-2.40 (m, 1 H), 2.19-2.10 (m, 1 H), 1.51 (t, J = 6.8 Hz, 6 H) |
| 26 | 1 | 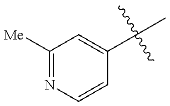 [SMILES: *c1ccnc(C)c1] | -bond- | 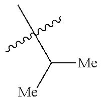 [SMILES: *C(C)C] | ¹H NMR (400 MHz, CDCl₃): δ 8.53 (d, J = 5.2 Hz, 1H), 7.95 (s, 1H), 7.15 (s, 1H), 7.09 (d, J = 4.8 Hz, 1H), 5.55 (s, 1H), 4.78 (sept, J = 6.8 Hz, 1H), 4.71 (s, 1H), 3.99- 4.08 (m, 2H), 2.59 (s, 3H), 2.34-2.40 (m, 1H), 2.05-2.10 (m, 1H), 1.48-1.52 (m, 6H) |
| 27 | 1 | 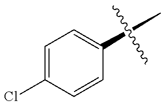 | -bond- | 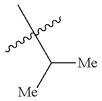 [SMILES: *C(C)C] | ¹H NMR (400 MHz, CDCl₃): δ 7.95 (s, 1H), 7.39 (d, J = 8.8 Hz, 2H), 7.30 (d, J = 8.4 Hz, 2H), 5.52 (s, 1H), 4.78 (sept, J = 6.8 Hz, 1H), 4.74-4.71 (m, 1H), 4.26-4.22 (m, 1H), 3.93-3.87 (m, 1H), 2.36-2.31 (m, 1H), 2.07-2.00 (m, 1H), 1.55-147 (m, 6H) |
| 28 | 1 | 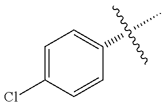 | -bond- | 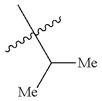 [SMILES: *C(C)C] | ¹H NMR (400 MHz, CDCl₃): δ 7.95 (s, 1H), 7.39 (d, J = 8.8 Hz, 2H), 7.30 (d, J = 8.4 Hz, 2H), 5.52 (s, 1H), 4.78 (sept, J = 6.8 Hz, 1H), 4.74-4.71 (m, 1H), 4.26-4.22 (m, 1H), 3.93-3.87 (m, 1H), 2.36-2.31 (m, 1H), 2.07-2.00 (m, 1H), 1.55-1.47 (m, 6H) |
| 29 | 1 | 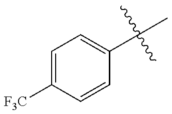 [SMILES: *c1ccc(C(F)(F)F)cc1] | -bond- | 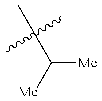 [SMILES: *C(C)C] | ¹H NMR (400 MHz, DMSO-d₆): δ 8.42 (s, 1H), 7.81 (s, 1H), 7.75 (d, J = 8.0 Hz, 2H), 7.59 (d, J = 8.0 Hz, 2H), 4.85 (br s, 1H), 4.75 (sept, J = 6.8 Hz, 1H), 3.97-4.03 (m, 1H), 3.54-3.60 (m, 1H), 2.24-2.29 (m, 1H), 1.97-2.06 (m, 1H), 1.41 (d, J = 6.8 Hz, 3H), 1.38 (d, J = 6.8 Hz, 3H) |
| 30 | 1 | 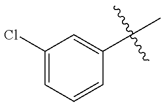 [SMILES: *c1cccc(Cl)c1] | -bond- | 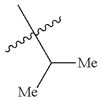 [SMILES: *C(C)C] | ¹H NMR (400 MHz, CDCl₃): δ 7.96 (s, 1H), 7.37 (t, J = 0.8 Hz, 3H), 7.25-7.28 (m, 1H), 5.54-5.57 (m, 1H), 4.79 (sept, J = 6.8 Hz, 1H), 4.72-4.75 (m, 1H), 4.19-4.24 (m, 1H), 3.90-3.97 (m, 1H), 2.36-2.41 (m, 1H), 2.02-2.10 (m, 1H), 1.52 (d, J = 6.8 Hz, 3H), 1.50 (d, J = 6.8 Hz, 3H) |
| 31 | 1 | 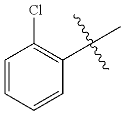 [SMILES: *c1ccccc1Cl] | -bond- | 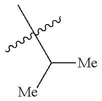 [SMILES: *C(C)C] | ¹H NMR (400 MHz, CDCl₃): δ 7.97 (s, 1H), 7.46-7.43 (m, 2H), 7.37-7.28 (m, 2H), 5.52 (s, 1H), 5.23-5.19 (m, 1H), 4.81 (sept, J = 6.8 Hz, 1H), 4.19-4.13 (m, 1H), 3.96- 3.90 (m, 1H), 2.44-2.39 (m, 1H), 2.17-2.14 (m, 1H), 1.53 (d, J = 6.8 Hz, 3H), 1.51 (d, J = 6.8 Hz, 3H) |
| 32 | 1 | 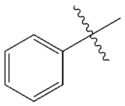 [SMILES: *c1ccccc1] | -bond- | 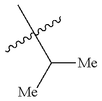 [SMILES: *C(C)C] | ¹H NMR (400 MHz, CDCl₃): δ 7.95 (s, 1H), 7.45-7.36 (m, 5H), 5.57 (s, 1H), 4.83- 4.72 (m, 2H), 4.31-4.25 (m, 1H), 3.93-3.86 (m, 1H), 2.40-2.36 (m, 1H), 2.12-2.06 (m, 1H), 1.52 (d, J = 6.8 Hz, 3H), 1.50 (d, J = 6.8 Hz, 3H) |
| 33 | 1 | 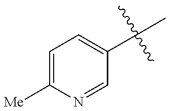 [SMILES: *c1ccc(C)nc1] | —CH₂— | 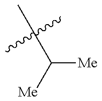 [SMILES: *C(C)C] | ¹H NMR (400 MHz, CDCl₃): δ 8.43 (s, 1H), 7.92 (s, 1H), 7.48 (dd, J1 = 8.0 Hz, J2 = 2.0 Hz, 1H), 7.19 (d, J = 8.0 Hz, 1H), 5.16 (br s, 1H), 4.73 (sept, J = 6.4 Hz, 1H), 4.48-4.45 (m, 1H), 3.78-3.69 (m, 2H), 2.93-2.78 (m, 2H), 2.59 (s, 3H), 2.18 (d, J = 4.4 Hz, 1H), 1.83-1.79 (m, 1H), 1.49- 1.44 (m, 6H) |
| 34 | 1 | 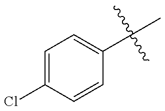 [SMILES: *c1ccc(Cl)cc1] | -bond- | 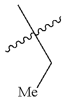 [SMILES: *CC] | ¹H NMR (400 MHz, CDCl₃): δ 7.96 (s, 1 H), 7.37-7.40 (m, 2 H), 7.28-7.31 (m, 2 H), 5.60 (s, 1 H), 4.71-4.74 (m, 1 H), 4.18-4.25 (m, 3 H), 3.85-3.92 (m, 1 H), 2.33-2.35 (m, 1 H), 2.03-2.05 (m, 1 H), 1.44-1.47 (m, 3 H) |
| 35 | 1 | 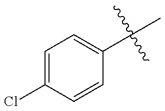 [SMILES: *c1ccc(Cl)cc1] | -bond- | 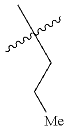 [SMILES: *CCC] | ¹H NMR (400 MHz, CDCl₃): δ 7.93 (s, 1 H), 7.37-7.40 (m, 2 H), 7.29-7.32 (m, 2 H), 5.52 (s, 1 H), 4.70-4.73 (m, 1 H), 4.22-4.27 (m, 1 H), 4.07-4.11 (m, 2 H), 3.85-3.91 (m, 1 H), 2.33-2.35 (m, 1 H), 2.03-2.05 (m, 1 H), 1.86-1.92 (m, 2 H), 0.93 (t, J = 6.0 Hz, 3H) |
| 36 | 1 | 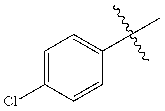 [SMILES: *c1ccc(Cl)cc1] | -bond- | 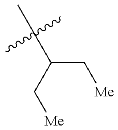 [SMILES: *C(CC)CC] | ¹H NMR (400 MHz, CDCl₃): δ 7.98 (s, 1 H), 7.38-7.40 (m, 2 H), 7.30-7.32 (m, 2 H), 5.56 (s, 1 H), 4.68-4.71 (m, 1 H), 4.24-4.28 (m, 2 H), 3.85-3.92 (m, 1 H), 2.33-2.36 (m, 1 H), 1.95-1.99 (m, 3 H), 1.79-1.83 (m, 2 H), 0.72-0.80 (m, 6 H) |
| 37 | 1 | 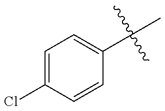 [SMILES: *c1ccc(Cl)cc1] | -bond- | 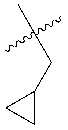 [SMILES: *CC1CC1] | ¹H NMR (400 MHz, CDCl₃): δ 7.94 (s, 1 H), 7.38-7.40 (d, J = 8.0 Hz, 2 H), 7.28- 7.31 (d, J = 12 Hz, 2 H), 5.55 (s, 1 H), 4.70-4.73 (m, 1 H), 4.22-4.26 (m, 1 H), 3.88-4.01 (m, 3 H), 2.24-2.39 (m, 1 H), 2.01-2.04 (m, 1 H), 1.33 (m, 1 H), 0.53- 0.58 (m, 2 H), 0.40-0.43 (m, 2 H) |
| 38 | 1 | 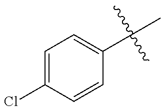 [SMILES: *c1ccc(Cl)cc1] | -bond- | 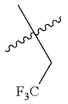 [SMILES: *CC(F)(F)F] | ¹H NMR (400 MHz, CDCl₃): δ 7.99 (s, 1 H), 7.40 (d, J = 8.0 Hz, 2 H), 7.29 (d, J = 8.0 Hz, 2 H), 5.65 (s, 1 H), 4.68-4.74 (m, 3 H), 4.21-4.27 (m, 1 H), 3.85-3.92 (m, 1 H), 2.35-2.39 (m, 1 H), 2.03-2.07 (m, 1 H) |
| 39 | 1 | 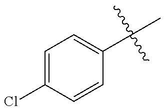 [SMILES: *c1ccc(Cl)cc1] | -bond- | 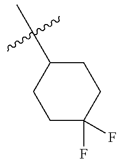 [SMILES: *C1CCC(F)(F)CC1] | ¹H NMR (400 MHz, CDCl₃): δ 7.93 (s, 1 H), 7.38-7.40 (m, 2 H), 7.28-7.31 (m, 2 H), 5.51 (s, 1H), 4.71-4.72 (m, 1 H), 4.48 (m, 1 H), 4.20-4.21 (m, 1 H), 3.89-3.91 (m, 1 H), 2.29-2.38 (m, 5 H), 1.98-2.05 (m, 5 H) |
| 40 | 1 | 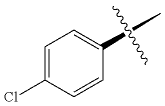 | -bond- | 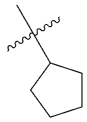 [SMILES: *C1CCCC1] | ¹H NMR (400 MHz, CDCl₃): δ 7.96 (s, 1H), 7.40 (d, J = 8.8 Hz, 2H), 7.31 (d, J = 8.4 Hz, 2H), 5.48 (s, 1H), 4.93 (quint, J = 7.6 Hz, 1H), 4.76-4.70 (m, 1H), 4.25-4.21 (m, 1H), 3.93-3.90 (m, 1H), 2.39-2.35 (m, 1H), 2.13-1.93 (m, 7H), 1.74-1.68 (m, 2H) |
| 41 | 1 | 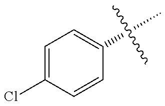 | -bond- | 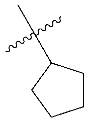 [SMILES: *C1CCCC1] | ¹H NMR (400 MHz, CDCl₃): δ 7.96 (s, 1H), 7.40 (d, J = 8.8 Hz, 2H), 7.31 (d, J = 8.4 Hz, 2H), 5.48 (s, 1H), 4.93 (quint, J = 7.6 Hz, 1H), 4.76-4.70 (m, 1H), 4.25-4.21 (m, 1H), 3.93-3.90 (m, 1H), 2.39-2.35 (m, 1H), 2.13-1.93 (m, 7H), 1.74-1.68 (m, 2H) |
| 42 | 1 | 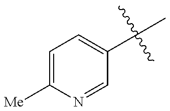 [SMILES: *c1ccc(C)nc1] | —CH₂— | 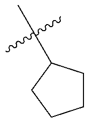 [SMILES: *C1CCCC1] | ¹H NMR (400 MHz, CDCl₃): δ 8.43 (s, 1H), 7.91 (s, 1H), 7.50 (d, J = 6.8 Hz, 1H), 7.21 (d, J = 7.6 Hz, 1H), 5.18 (br s, 1H), 4.87 (quint, J = 7.6 Hz, 1H), 4.47-4.43 (m, 1H), 3.79-3.68 (m, 2H), 2.91-2.80 (m, 1H), 2.60 (s, 3H), 2.21-1.66 (m, 10H) |
| 43 | 1 | 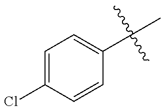 [SMILES: *c1ccc(Cl)cc1] | -bond- | 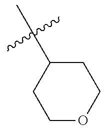 [SMILES: *C1CCOCC1] | ¹H NMR (400 MHz, CDCl₃): δ 7.96 (s, 1 H), 7.42-7.28 (m, 4 H), 5.55 (s, 1H), 4.75- 4.57 (m, 2 H), 4.21-4.12 (m, 3 H), 3.94- 3.87 (m, 1 H), 3.57 (t, J = 24.4 Hz, 2 H), 2.40-2.33 (m, 3 H), 2.07-1.86 (m, 3 H) |
| 44 | 1 | 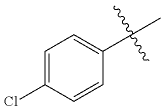 [SMILES: *c1ccc(Cl)cc1] | -bond- | 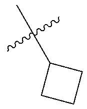 [SMILES: *C1CCC1] | ¹H NMR (400 MHz, CDCl₃): δ 7.96 (s, 1H), 7.39-7.27 (m, 4H), 5.62 (s, 1H), 5.03 (quint, J = 8.4 Hz, 1H), 4.71 (br s, 1H), 4.20-4.16 (m, 1H), 3.91-3.86 (m, 1H), 2.75-2.72 (m, 2H), 2.40-2.35 (m, 3H), 2.03-1.77 (m, 3H) |
| 45 | 1 | 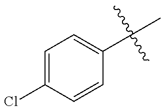 [SMILES: *c1ccc(Cl)cc1] | -bond- | 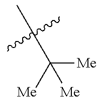 [SMILES: *C(C)(C)C] | ¹H NMR (400 MHz, CDCl₃): δ 7.73 (s, 1H), 7.35 (d, J = 8.8 Hz, 2H), 7.11 (d, J = 8.4 Hz, 2H), 6.26 (br s, 1H), 5.40 (br s, 1H), 4.45 (d, J = 4.8 Hz, 1H), 4.20-4.17 (m, 1H), 2.58-2.55 (m, 1H), 2.27-2.23 (m, 1H), 1.75 (s, 9H) |

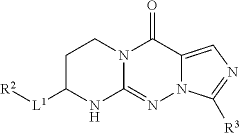

| Ex. | | | | |
|---|---|---|---|---|
| No. | R²— | L¹ | R³— | ¹H NMR |
| 46 | 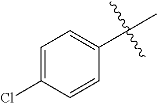 [SMILES: *c1ccc(Cl)cc1] | -bond- | 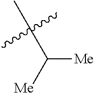 [SMILES: *C(C)C] | ¹H NMR (400 MHz, CDCl₃): δ 7.77 (s, 1H), 7.39 (d, J = 8.4 Hz, 2H), 7.31 (d. J = 8.4 Hz, 2H), 5.15 (br s, 1H), 4.68-4.65 (m, 1H), 4.22 (dt, J1 = 13.6 Hz, J2 = 6.0 Hz, 1H), 3.86-3.80 (m, 1H), 3.36 (sept, J = 7.2 Hz, 1H), 2.38- 2.33 (m, 1H), 2.06-2.02 (m, 1H), 1.38-1.34 (m, 6H) |
| 47 | 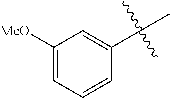 [SMILES: *c1cccc(OC)c1] | -bond- | 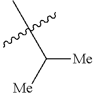 [SMILES: *C(C)C] | ¹H NMR (400 MHz, CDCl₃): δ 7.78 (s, 1 H), 7.33 (t, J = 8.4 Hz, 1 H), 6.95 (d, J = 7.6 Hz, 1 H), 6.90-6.88 (m, 2 H), 5.11 (br s, 1 H), 4.67- 4.64 (m, 1 H), 4.28-4.25 (m, 1 H), 3.86-3.81 (m, 4 H), 3.37 (sept, J = 6.8 Hz, 1 H), 2.36- 2.33 (m, 1 H), 2.08-1.98 (m, 1 H), 1.39-1.31 |
| | | | | (m, 6 H) |
| 48 | 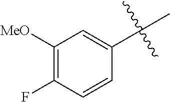 [SMILES: *c1ccc(F)c(OC)c1] | -bond- | 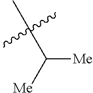 [SMILES: *C(C)C] | ¹H NMR (400 MHz, CDCl₃): δ 7.78 (s, 1H), 7.11 (dd, J1 = 10.8 Hz, J2 = 8.8 Hz, 1H), 6.96-6.90 (m, 2H), 5.03 (br s, 1H), 4.65-4.62 (m, 1H), 4.35-4.31 (m, 1H), 3.92 (s, 3H), 3.82-3.76 (m, 1H), 3.37 (sept, J = 6.8 Hz, 1H), 2.36-2.32 (m, 1H), 2.10-1.98 (m, 1H), 1.39-1.35 (m, 6H) |
| 49 | 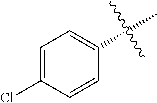 | -bond- | 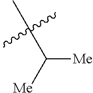 [SMILES: *C(C)C] | ¹H NMR (400 MHz, CDCl₃): δ 7.80 (s, 1H), 7.42 (d, J = 7.6 Hz, 2H), 7.34 (d, J = 8.0 Hz, 2H), 5.07 (s, 1H), 4.70-4.67 (m, 1H), 4.29- 4.25 (m, 1H). 3.87-3.83 (m, 1H), 3.38 (sept, J = 6.8 Hz, 1H), 2.39-2.35 (m, 1H), 2.09-1.99 (m, 1H), 1.40 (d, J = 7.2 Hz, 3H), 1.37 (d, J = 6.8 Hz, 3H) |
| 50 | 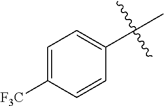 [SMILES: *c1ccc(C(F)(F)F)cc1] | -bond- | 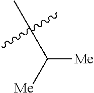 [SMILES: *C(C)C] | ¹H NMR (400 MHz, CDCl₃): δ 7.83 (s, 1H), 7.72 (d, J = 8.0 Hz, 2H), 7.54 (d, J = 8.0 Hz, 2H), 5.11 (br s, 1H), 4.80 (br s, 1H), 4.26- 4.23 (m, 1H), 3.94-3.90 (m, 1H), 3.47-3.38 (m, 1H), 2.45-2.42 (m, 1H), 2.12-2.10 (m, 1H), 1.42 (d, J = 7.6 Hz, 3H), 1.40 (d, J = 6.8 Hz, 3H) |
| 51 | 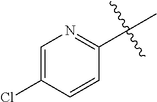 [SMILES: *c1ccc(Cl)cn1] | -bond- | 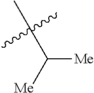 [SMILES: *C(C)C] | ¹H NMR (400 MHz, CD₃OD): δ 8.58 (d, J = 2.0 Hz, 1H), 7.90 (dd, J1 = 8.4 Hz, J2 = 2.4 Hz, 1H), 7.65 (s, 1H), 7.56 (d, J = 8.4 Hz, 1H), 4.82 (t, J = 5.6 Hz, 1H), 4.11-4.06 (m, 1H), 3.89-3.82 (m, 1H), 3.50 (sept, J = 6.8 Hz, 1H), 2.45-2.41 (m, 1H), 2.28-2.23 (m, 1H), 1.38-1.33 (m, 6H) |
| 52 | —H | -bond- | 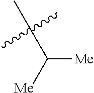 [SMILES: *C(C)C] | ¹H NMR (400 MHz, CDCl₃): δ 7.60 (s, 1H), 4.91 (br s, 1H), 4.00 (t, J = 6.0 Hz, 2H), 3.48- 3.32 (m, 3H), 2.13-2.07 (m, 2H), 1.33 (d, J = 6.8 Hz, 6H) |
| 53 | 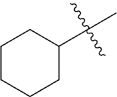 [SMILES: *C1CCCCC1] | -bond- | 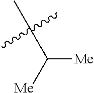 [SMILES: *C(C)C] | 1H NMR (400 MHz, CD₃OD): δ 7.60 (s, 1H), 4.36-4.30 (m, 1H), 3.49-3.44 (m, 1H), 3.33- 3.29 (m, 1H), 2.11-2.05 (m, 1H), 1.90-1.82 (m, 5H), 1.75-1.72 (d, J = 5.8 Hz, 2H), 1.61 (s, 1H), 1.35-1.18 (m, 7H), 1.18-1.10 (m, 4H) |
| 54 | 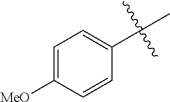 [SMILES: *c1ccc(OC)cc1] | -bond- | 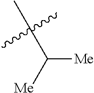 [SMILES: *C(C)C] | 1H NMR (400 MHz, CD₃OD): δ 7.64 (s, 1 H), 7.35-7.32 (m, 2 H), 6.98-6.94 (m, 2 H), 4.69-4.66 (m, 1 H), 4.14-4.08 (m, 1 H), 3.91- 3.85 (m, 1 H), 3.81 (s, 3 H), 3.54-3.33 (m, 1 H), 2.34-2.29 (m, 1 H), 2.10-2.06 (m, 1 H), 1.36 (t, J = 7.2 Hz, 6 H) |
| 55 | 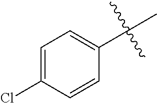 [SMILES: *c1ccc(Cl)cc1] | -bond- | 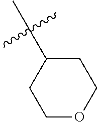 [SMILES: *C1CCOCC1] | ¹H NMR (400 MHz, CDCl₃): δ 7.82 (s, 1H), 7.42 (d, J = 8.0 Hz, 2H), 7.33 (d, J = 8.4 Hz, 2H), 5.07 (br s, 1H), 4.71-4.68 (m, 1H), 4.32- 4.26 (m, 1H), 4.11-4.09 (m, 2H), 3.88-3.82 (m, 1H), 3.61-3.55 (m, 2H), 3.37-3.32 (m, 1H), 2.41-2.37 (m, 1H), 2.15-2.03 (m, 3H), 1.92 (br s, 2H) |
| 56 | 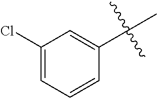 [SMILES: *c1cccc(Cl)c1] | -bond- | 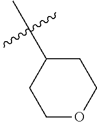 [SMILES: *C1CCOCC1] | ¹H NMR (400 MHz, DMSO-d₆): δ 7.99 (s, 1H), 7.59 (s, 1H), 7.48 (s, 1H), 7.43-7.34 (m, 3H), 4.70 (br s, 1H), 3.97-3.88 (m, 3H), 3.71- 3.68 (m, 1H), 3.49-3.43 (m, 2H), 3.33-3.30 (m, 2H), 2.27-2.25 (m, 1H), 2.02-1.82 (m, 4H) |
| 57 | 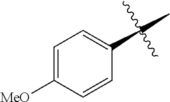 | -bond- | 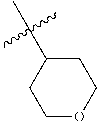 [SMILES: *C1CCOCC1] | ¹H NMR (400 MHz, CDCl₃): δ 7.79 (s, 1 H), 7.29-7.31 (m, 2 H), 6.94-6.96 (m, 2 H), 5.04 (s, 1 H), 4.62-4.65 (m, 1 H), 4.34-4.37 (m, 1 H), 4.06-4.10 (m, 2 H), 3.84 (s, 3 H), 3.78- 3.80 (m, 1 H), 3.54-3.61 (m, 2 H), 3.32 (m, 1 H), 2.10-2.12 (m, 1 H), 2.05-2.09 (m, 3 H), 1.89-1.94 (m, 2 H) |
| 58 | 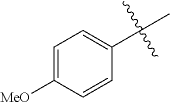 [SMILES: *c1ccc(OC)cc1] | —CH₂— | 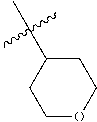 [SMILES: *C1CCOCC1] | ¹H NMR (400 MHz, CDCl₃): δ 7.77 (s, 1H), 7.18 (d, J = 8.4 Hz, 2H), 6.96 (d, J = 8.4 Hz, 2H), 4.76 (br s, 1H), 4.43 (dt, J1 = 18.4 Hz, J2 = 4.8 Hz, 1H), 4.10-4.08 (m, 2H), 3.86 (s, 3H), 3.73-3.54 (m, 4H), 3.33-3.29 (m, 1H), 2.94 (dd, J1 = 12.8 Hz, J2 = 5.6 Hz, 1H), 2.73 (dd, J1 = 14.0 Hz, J2 = 8.8 Hz, 1H), 2.25-2.05 (m, 3H), 1.91-1.77 (m, 3H) |
| 59 | 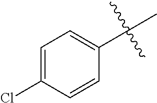 [SMILES: *c1ccc(Cl)cc1] | —CH₂— | 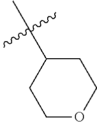 [SMILES: *C1CCOCC1] | ¹H NMR (400 MHz, CDCl₃): δ 7.78 (s, 1H), 7.40 (d, J = 8.4 Hz, 2H), 7.21 (d, J = 8.4 Hz, 2H), 4.71 (br s, 1H), 4.42 (dt, J1 = 8.0 Hz, J2 = 4.0 Hz, 1H), 4.12-4.08 (m, 2H), 3.76-3.54 (m, 4H), 3.33-3.29 (m, 1H), 2.94 (dd, J1 = 13.6 Hz, J2 = 5.6 Hz, 1H), 2.80 (dd, J1 = 13.2 Hz, J2 = 8.4 Hz, 1H), 2.25-2.06 (m, 3H), 1.89-1.81 (m, 3H) |
| 60 | 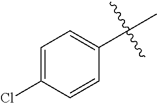 [SMILES: *c1ccc(Cl)cc1] | -bond- | 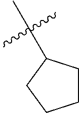 [SMILES: *C1CCCC1] | ¹H NMR (400 MHz, CDCl₃): δ 7.78 (s, 1H), 7.39 (d, J = 8.4 Hz, 2H), 7.31 (d, J = 8.4 Hz, 2H), 5.01 (s, 1H), 4.67 (br s, 1H), 4.25-4.21 (m, 1H) 3.86-3.82 (m, 1H) 3.46-3.42 ( m, 1H), 2.38-2.33 (m, 1H), 2.10-1.75 (m, 7H), 1.68 (br s, 2H) |
| 61 | 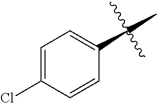 | -bond- | 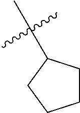 [SMILES: *C1CCCC1] | ¹H NMR (400 MHz, CDCl₃): δ 7.78 (s, 1H), 7.42-7.39 (m, 2H), 7.35-7.32 (m, 2H), 5.01 (s, 1H), 4.70-4.67 (m, 1H), 4.27-4.22 (m, 1H), 3.87-3.80 (m, 1H), 3.48-3.43 (m, 1H), 2.36 (d, 1H), 2.12-1.80 (m, 7H), 1.74-1.62 (m, 2H) |
| 62 | 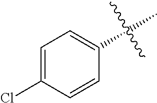 | -bond- | 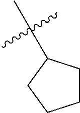 [SMILES: *C1CCCC1] | ¹H NMR, (400 MHz, CD₃OD): δ 7.63 (s, 1H), 7.44-7.40 (m, 4H), 4.75-4.72 (m, 1H), 4.05- 3.91 (m, 2H), 3.60-3.56 (m, 1H), 2.37-2.33 (m, 1H), 2.10-2.03 (m, 3H), 1.88 (br s, 4H), 1.7) (br s, 2H) |
| 63 | 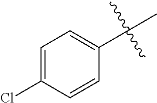 [SMILES: *c1ccc(Cl)cc1] | -bond- | 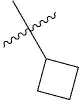 [SMILES: *C1CCC1] | ¹H NMR (400 MHz, CDCl₃): δ 7.83 (s, 1H), 7.43-7.40 (m, 2H), 7.34-7.32 (m, 2H), 5.05 (s, 1H), 4.70-4.67 (m, 1H), 4.28-4.22 (m, 1H), 3.93-3.81 (m, 2H), 2.60-2.50 (m, 2H), 2.41- 2.34 (m, 3H), 2.13-2.00 (m, 3H) |
| 64 | 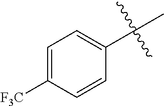 [SMILES: *c1ccc(C(F)(F)F)cc1] | -bond- | 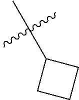 [SMILES: *C1CCC1] | ¹H NMR (400 MHz, CDCl₃): δ 7.85 (s, 1H), 7.71 (d, J = 8.0 Hz, 2H), 7.53 (d, J = 8.0 Hz, 2H), 5.10 (s, 1H). 4.81-4.78 (m, 1H), 4.24- 4.21 (m, 1H), 3.94-3.87 (m, 2H), 2.59-2.52 (m, 2H), 2.43-2.37 (m, 3H), 2.14-2.03 (m, 3H) |
| 65 | 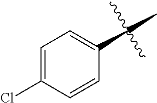 | -bond- | 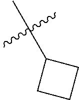 [SMILES: *C1CCC1] | ¹H NMR (400 MHz, CD₃OD): δ 7.67 (s, 1H), 7.42 (s, 4H), 4.75-4.71 (m, 1H), 4.05-3.91 (m, 3H), 2.52-2.47 (m, 2H), 2.40-2.33 (m, 3H), 2.13-2.01 (m, 3H) |
| 66 | 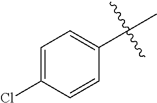 [SMILES: *c1ccc(Cl)cc1] | —CH₂— | 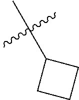 [SMILES: *C1CCC1] | ¹H NMR (400 MHz, CDCl₃): δ 7.79 (s, 1 H), 7.39 (d, J = 4.2 Hz, 2 H), 7.20 (d, J = 4.2 Hz, 2 H), 4.71 (s, 1 H), 4.43-4.37 (m, 1 H), 3.88- 3.69 (m, 1 H), 3.68-3.62 (m, 2 H), 2.95-2.90 (m, 1 H), 2.81-2.756 (m, 1 H), 2.55-2.46 (m, 2 H), 2.39-2.32 (m, 2 H), 2.22-2.11 (m, 1 H), 2.09-1.98 (m, 2 H), 1.97-1.78 (m, 1 H) |
| 67 | 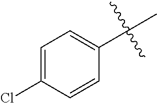 [SMILES: *c1ccc(Cl)cc1] | -bond- | 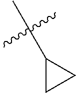 [SMILES: *C1CC1] | ¹H NMR (400 MHz, CDCl₃): δ 7.74 (s, 1H), 7.43-7.40 (m, 2H), 7.36-7.33 (m, 2H), 5.08 (s, 1H), 4.72-4.69 (m, 1H), 4.29-4.23 (m, 1H), 3.89-3.82 (m, 1H), 2.38-2.28 (m, 2H), 2.07- 2.05 (m, 1H), 1.20 (br s, 2H), 1.10-1.07 (m, 2H) |
| 68 | 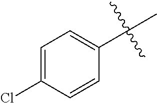 [SMILES: *c1ccc(Cl)cc1] | -bond- | 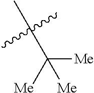 [SMILES: *C(C)(C)C] | ¹H NMR (400 MHz, CDCl₃): δ 7.79 (s, 1H), 7.44-7.42 (m, 2H), 7.36-7.34 (m, 2H), 5.01 (s, 1H), 4.70-4.67 (m, 1H), 4.32-4.26 (m, 1H), 3.88-3.81 (m, 1H), 2.41-2.36 (m, 1H), 2.10- 2.06 (m, 1H), 1.55 (s, 9H) |

### Example 69

### (2R)-2-(4-Chlorophenyl)-9-(tetrahydro-2H-pyran-4-yl)-1,2,3,4-tetrahydro-6H-imidazo[5,1-f]pyrimido[2,1-c][1,2,4]triazin-6-one

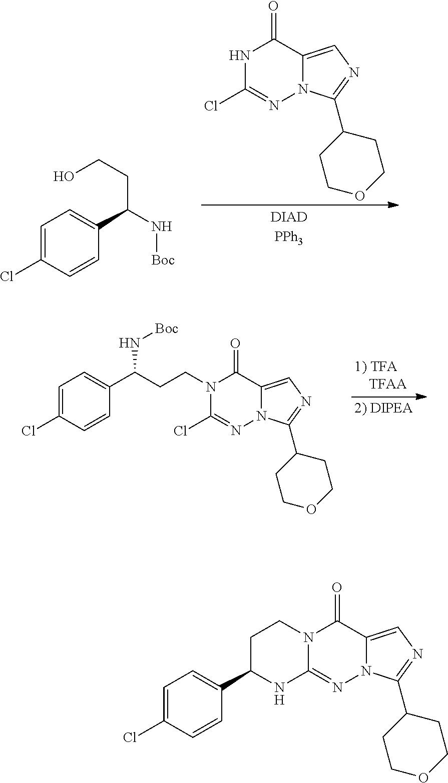 [SMILES: CC(C)(C)OC(=O)N[C@H](CCO)c1ccc(Cl)cc1 ; O=c1[nH]c(Cl)nn2c(C3CCOCC3)ncc12 ; CC(C)(C)OC(=O)N[C@H](CCn1c(Cl)nn2c(C3CCOCC3)ncc2c1=O)c1ccc(Cl)cc1 ; O=c1c2cnc(C3CCOCC3)n2nc2n1CC[C@H](c1ccc(Cl)cc1)N2]

A mixture of 2-methyl-2-propanyl[(1R)-3-[2-chloro-4-oxo-7-(tetrahydro-2H-pyran-4-yl)imidazo[5,1-1][1,2,4]triazin-3(4H)-yl]-1-(4-chlorophenyl)propyl]carbamate (3.98 g, 7.62 mmol), TFA (5.9 mL, 76.2 mmol), and TFAA (51 μL, 0.381 mL) in DCM (15 mL) was stirred at rt overnight. The resulting mixture was concentrated in vacuo. To an ice-cooled mixture of the above residue in THF (25 mL) was added TEA (10.6 mL, 76.2 mmol), and the reaction mixture was stirred at rt for 3 h. The resulting mixture was diluted with water, and extracted with EtOAc. The combined organic extracts were washed with brine, dried over sodium sulfate, filtered, and concentrated in vacuo. The solid was washed with EtOAc to give the titled compound (2.58 g, 88%) as white solids. ¹H NMR (CDCl₃) δ: 7.79 (s, 1H), 7.40 (d, J=8.5 Hz, 2H), 7.31 (d, J=8.5 Hz, 2H), 5.07 (s, 1H), 4.69-4.66 (m, 1H), 4.29-4.23 (m, 1H), 4.11-4.05 (m, 2H), 3.86-3.79 (m, 1H), 3.59-3.52 (m, 2H), 3.34-3.26 (m, 1H), 2.39-2.35 (m, 1H), 2.16-2.00 (m, 3H), 1.93-1.86 (2H).

### 2-Methyl-2-propanyl[(1R)-3-[2-chloro-4-oxo-7-(tetrahydro-2H-pyran-4-yl)imidazo[5,1-f][1,2,4]triazin-3(4H)-yl]-1-(4-chlorophenyl)propyl]carbamate

To an ice-cooled mixture of (1R)-tert-butyl 1-(4-chlorophenyl)-3-hydroxypropylcarbamate (see Reference Example 7 intermediate, 7.72 g, 27.0 mmol), 2-chloro-7-(tetrahydro-2H-pyran-4-yl)imidazo[5,1-f][1,2,4]triazin-4(3H)-one (Reference Example 2, 6.43 g, 22.5 mmol), and triphenylphosphine (8.87 g, 33.8 mmol) in DCM (75 mL) was added dropwise DIAD (6.7 mL, 33.8 mmol), and the reaction mixture was stirred at rt for 24 h. The resulting mixture was concentrated in vacuo. Dichloro magnesium (6.43 g, 67.5 mmol) and toluene (45 mL) were added to the residue, and the mixture was stirred at 60° C. for 1 h. The precipitates were filtered, and washed with toluene. The filtrate was concentrated in vacuo. The residue was purified by silica gel chromatography (hexane:EtOAc) to give the titled compound (3.98 g, 34%).

### Example 70

### 1-Cyclopentyl-8-[(5-methyl-2-pyrimidinyl)methyl]-6,7,8,10-tetrahydropyrazolo[3,4-d]pyrimido[1,2-a]pyrimidin-4(1H)-one

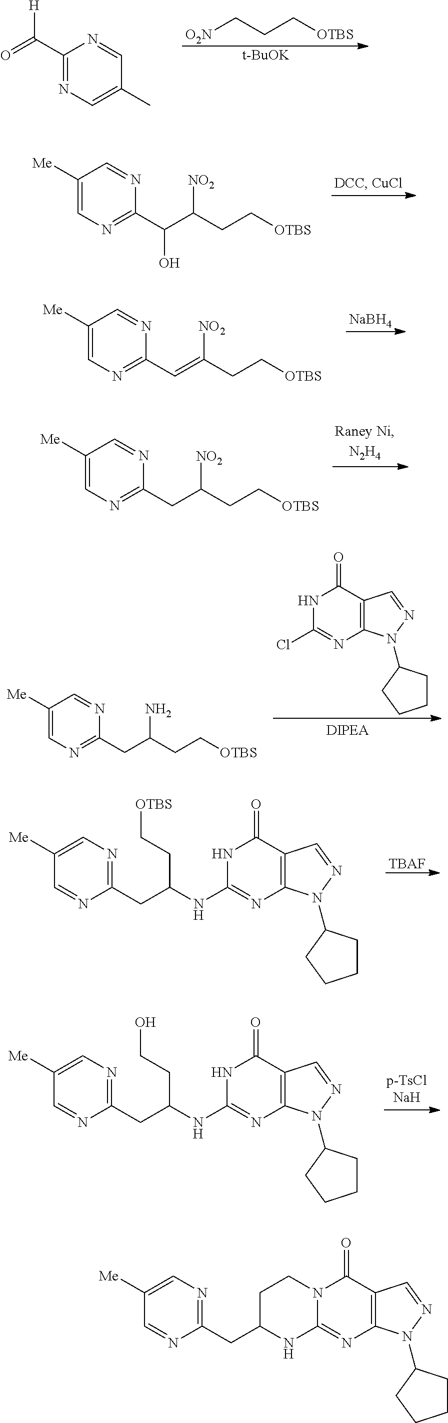

To a solution of 1-cyclopentyl-6-{[4-hydroxy-1-(5-methyl-2-pyrimidinyl)-2-butanyl]amino}-1,5-dihydro-4H-pyrazolo[3,4-d]pyrimidin-4-one (73 mg, 0.19 mmol) in THF (4 mL) was added sodium hydride (10 mg, 0.42 mmol) and the mixture was stirred for 10 mins. p-Tosyl chloride (44 mg, 0.23 mmol) was added and the mixture was stirred at rt for 3 h. 5 mL of water was added and the solution was extracted with ethyl acetate and the combined organic layer was washed with brine and dried over sodium sulfate. After filtration and evaporation of solvent, the residue was purified by HPLC to give the titled compound (30 mg, 43%) as white solids. ¹H NMR (400 MHz, CDCl₃): δ 8.60 (s, 2H), 7.93 (s, 1H), 6.73 (br s, 1H), 4.98-4.95 (m, 1H), 4.55-4.51 (m, 1H), 4.12 (br s, 1H), 3.74-3.68 (m, 1H), 3.29-3.14 (m, 2H), 2.36 (s, 3H), 2.31-2.27 (m, 1H), 2.07-1.90 (m, 7H), 1.68 (br s, 2H).

### 4-{[Dimethyl(2-methyl-2-propanyl)silyl]oxy}-1-(5-methyl-2-pyrimidinyl)-2-nitro-1-butanol

To the solution of tert-butyldimethyl(3-nitropropoxy)silane (1.29 g, 5.90 mmol) in THF (10 mL) was added potassium tert-buthoxide (660 mg, 5.90 mmol) at 0° C. The solution was stirred at 0° C. for 30 mins and 5-methyl-2-pyrimidinecarbaldehyde (600 mg, 4.92 mmol) was added. The mixture was stirred at rt for 2 h. 2 mL of water was added to quench the reaction. The solution was extracted with EtOAc (3×30 mL) and the combined organic layer was washed with brine and dried over sodium sulfate. After evaporation of solvent, the crude was purified with column chromatography (DCM/MeOH=100/1-20/1) to give the crude (1.35 g, 80%) as white solids. MS (ESI): m/e=342 [M+1]⁺.

### 2-[(1Z)-4-{[Dimethyl(2-methyl-2-propanyl)silyl]oxy}-2-nitro-1-buten-1-yl]-5-methylpyrimidine

To a solution of 4-{[dimethyl(2-methyl-2-propanyl)silyl]oxy}-1-(5-methyl-2-pyrimidinyl)-2-nitro-1-butanol (1.35 g, 3.96 mmol) in MeCN (20 mL) was added copper(I) chloride (1.57 g, 15.84 mmol) and DCC (4.9 g, 23.76 mmol) at 0° C. The solution was stirred at 60° C. for 15 h. 2 mL of acetic acid was added to quench the reaction. The solution was extracted with EtOAc (3×30 mL) and the combined organic layer was washed with brine and dried over sodium sulfate. After evaporation of solvent, the crude was purified with column chromatography (DCM/MeOH=100/1-50/1) to give the crude (690 mg, 70%) as white solids. MS (ESI): m/e=[M+1]⁺.

### 2-(4-{[Dimethyl(2-methyl-2-propanyl)silyl]oxy}-2-nitrobutyl)-5-methylpyrimidine

To a solution of 2-[(1Z)-4-{[dimethyl(2-methyl-2-propanyl)silyl]oxy}-2-nitro-1-buten-1-yl]-5-methylpyrimidine (300 mg, 1.44 mmol) in MeOH (15 mL) was added sodium borohydride (55 mg, 1.44 mmol) at 0° C. The solution was stirred at 0° C. for 1 h. Water was added to quench the reaction. The solution was extracted with EtOAc (3×50 mL) and the combined organic layer was washed with brine and dried over sodium sulfate. After evaporation of solvent, the crude was purified with column chromatography (DCM/MeOH=100/1-50/1) to give the crude (320 mg, 44%) as white solids. MS (ESI): m/e=326 [M+1]⁺.

### 4-{[Dimethyl(2-methyl-2-propanyl)silyl]oxy}-1-(5-methyl-2-pyrimidinyl)-2-butanamine

To a solution of 2-(4-{[dimethyl(2-methyl-2-propanyl)silyl]oxy}-2-nitrobutyl)-5-methylpyrimidine (320 mg, 0.98 mmol) in MeOH (10 mL) was added Raney nickel (50 mg) and hydrazine (47 mg, 1.47 mmol) at 0° C. The system was evacuated and then refilled dry nitrogen. The solution was stirred at rt for 1 h. Filtration and evaporation of solvent gave the crude (160 mg, 56%) as a oil. MS (ESI): m/e=296 [M+1]⁺.

### 1-Cyclopentyl-6-{[4-{[dimethyl(2-methyl-2-propanyl)silyl]oxy}-1-(5-methyl-2-pyrimidinyl)-2-butanyl]amino}-1,5-dihydro-4H-pyrazolo[3,4-d]pyrimidin-4-one

To a solution of 6-chloro-1-cyclopentyl-1,5-dihydro-4H-pyrazolo[3,4-d]pyrimidin-4-one (synthesized in a similar manner to Reference Example 1, 71 mg, 0.30 mmol) in 1-butanol (4 mL) was added 4-{[dimethyl(2-methyl-2-propanyl)silyl]oxy}-1-(5-methyl-2-pyrimidinyl)-2-butanamine (80 mg, 0.27 mmol), DIPEA (105 mg, 0.81 mmol). The mixture was stirred at 120° C. in microwave for 12 h. After evaporation of solvent, the residue was purified by chromatography (DCM/MeOH=100/1-20/1) to give the titled compound (125 mg, 92%) as yellow solids. MS (ESI): m/e=498 [M+1]⁺.

### 1-Cyclopentyl-6-{[4-hydroxy-1-(5-methyl-2-pyrimidinyl)-2-butanyl]amino}-1,5-dihydro-4H-pyrazolo[3,4-d]pyrimidin-4-one

To a solution of 1-cyclopentyl-6-{[4-{[dimethyl(2-methyl-2-propanypsilyl]oxy}-1-(5-methyl-2-pyrimidinyl)-2-butanyl]amino}-1,5-dihydro-4H-pyrazolo[3,4-d]pyrimidin-4-one (125 mg, 0.25 mmol) in THF (4 mL) was added tetrabuthylammonium fluoride (197 mg, 0.76 mmol) and the mixture was stirred at 25° C. for 8 h. After evaporation of solvent, the crude was purified with chromatography (DCM/MeOH=100/1-20/1) to give the titled compound (76 mg, 79%) as yellow solids. MS (ESI): m/e=384 [M+1]⁺.

The compounds of Example 71 to 73 were synthesized in a similar manner to Example 70.

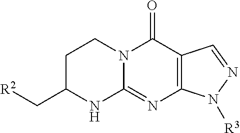

| Ex. | | | |
|---|---|---|---|
| No. | R²— | R³— | ¹H NMR |
| 71 | 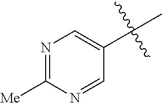 [SMILES: *c1cnc(C)nc1] | 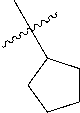 [SMILES: *C1CCCC1] | ¹H NMR (400 MHz, CDCl₃): δ 8.59 (s, 2H), 7.94 (s, 1H), 5.33 (br s, 1H), 4.87 (quint, J = 7.6 Hz, 1H), 4.46-4.40 (m, 1H), 3.84-3.74 (m, 2H), 2.95-2.82 (m, 2H), 2.79 (s, 3H), 2.24-2.20 (m, 1H), 2.10-1.68 (m, 9H) |
| 72 | 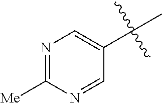 [SMILES: *c1cnc(C)nc1] | 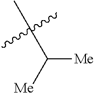 [SMILES: *C(C)C] | ¹H NMR (400 MHz, CDCl₃): δ 8.60 (s, 2H), 7.94 (s, 1H), 5.21 (br s, 1H), 4.75 (sept, J = 6.8 Hz, 1H), 4.42 (dd, J = 4.0 Hz, 18.8 Hz, 1H), 3.83-3.76 (m, 2H), 2.91-2.86 (m, 2H), 2.79 (s, 3H), 2.25-2.19 (m, 1H), 1.88-1.80 (m, 1H), 1.49 (d, J = 6.8 Hz, 3H), 1.47 (d, J = 6.8 Hz, 3H) |
| 73 | 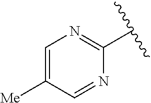 [SMILES: *c1ncc(C)cn1] | 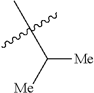 [SMILES: *C(C)C] | ¹H NMR (400 MHz, CDCl₃): δ 8.60 (s, 2H), 7.93 (s, 1H), 6.73 (br s, 1H), 4.83 (sept, J = 5.6 Hz, 1H), 4.55 (dt, J1 = 11.2 Hz, J2 = 3.6 Hz, 1H), 4.13-4.09 (m, 1H), 3.72-3.67 (m, 1H), 3.29-.3.15 (m, 2H), 2..36 (s, 3H), 2.31-2.27 (m, 1H), 1.95-1.87 (m, 1H), 1.49 (d, J = 5.2 Hz, 6H) |

### Example 74

### 6-(3-Chlorophenyl)-3-cyclopentyl-4,6,7,8-tetrahydro-10H-[1,2]oxazolo[4,3-d]pyrimido[1,2-a]pyrimidin-10-one

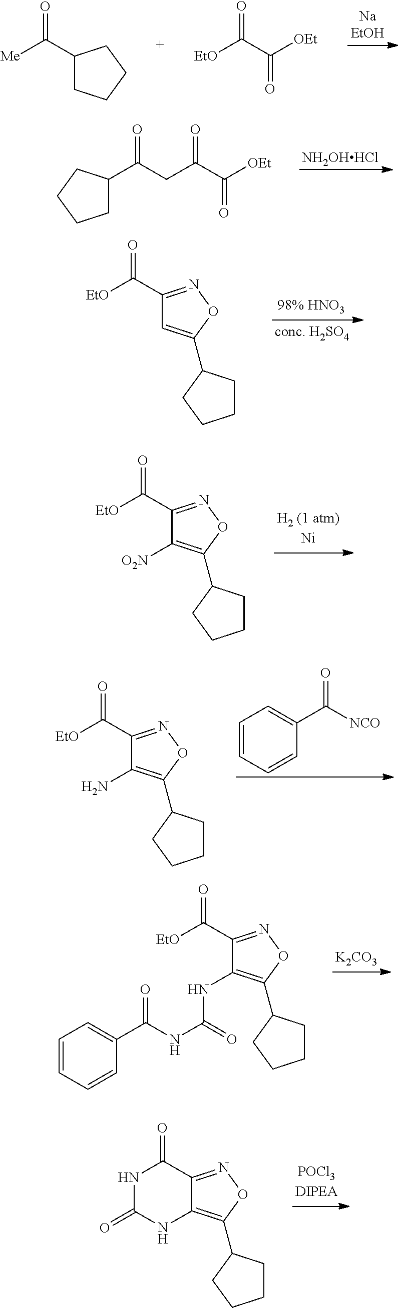

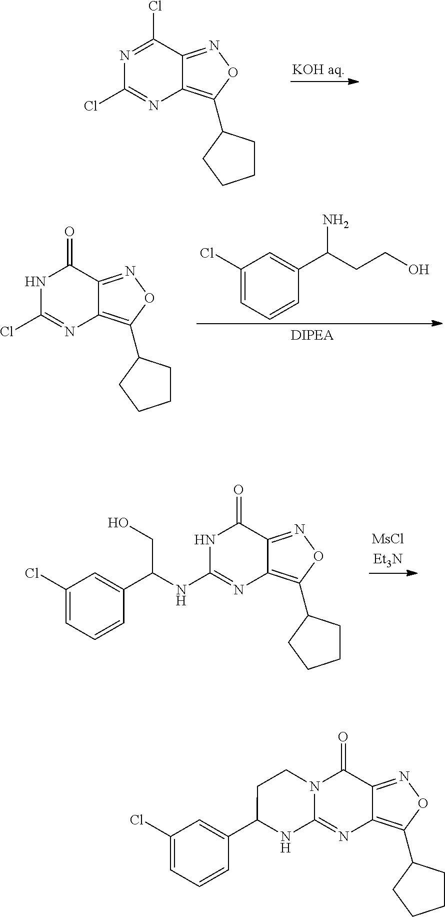 [SMILES: Clc1nc(Cl)c2noc(C3CCCC3)c2n1 ; O=c1[nH]c(Cl)nc2c(C3CCCC3)onc12 ; NC(CCO)c1cccc(Cl)c1 ; O=c1[nH]c(NC(CO)c2cccc(Cl)c2)nc2c(C3CCCC3)onc12 ; O=c1c2noc(C3CCCC3)c2nc2n1CCC(c1cccc(Cl)c1)N2]

To a solution of 5-(1-(3-chlorophenyl)-3-hydroxypropylamino)-3-cyclopentylisoxazolo[4,3-d]pyrimidin-7(6H)-one (55 mg, 0.15 mmol) in DCM (5 mL) was added TEA (0.05 g, 0.45 mmol) and methanesulfuryl chloride (0.02 g, 0.18 mmol) at 60° C. The mixture was stirred at 60° C. for 2 h. Concentrated, the resulting oil was purified by reverse phase HPLC with MeCN (50%) and water (50%) to provide the titled compound (4.22 mg, 10%) as white solids. ¹H NMR (400 MHz, CDCl₃): δ 7.37-7.32 (m, 3H), 7.26-7.23 (m, 1H), 5.44 (s, 1 H), 4.70-4.67 (m, 1H), 4.30-4.25 (m, 1H), 3.97-3.90 (m, 1H), 3.51-3.43 (m, 1H), 2.40-2.34 (m, 1H), 2.13-1.95 (m, 5H), 1.90-1.86 (m, 2H), 1.76-1.70 (m, 2H). MS (ESI): m/e=371 [M+H]⁺.

### Ethyl 4-cyclopentyl-2,4-dioxobutanoate

To a solution of EtOH (20 mL) was added sodium (0.82 g, 35.7 mmol). The reaction mixture was cooled to and stirred at rt for 1 h. A solution of 1-cyclopentylethanone (4 g, 35.7 mmol) and diethyl oxalate (15.63 g, 107 mmol) was added. The reaction was stirred at rt for 1 h. Then the reaction mixture was heated to 80° C. and stirred at that temperature for 1 h, then neutralized with 1M sulfuric acid aq. solution. EtOAc (150 mL) was added to the reaction vessel and the resulting biphasic mixture was transferred to a separatory funnel. The layers were separated and the organic phase was washed with brine (100 mL×2). The combined organics were dried over anhydrous sodium sulfate, filtered and concentrated in vacuo. The resulting oil was purified by flash column chromatography to give the titled compound (4 g, 53%). MS (ESI): m/e=213 [M+H]⁺.

### Ethyl 5-cyclopentyl-1,2-oxazole-3-carboxylate

To a solution of ethyl 4-cyclopentyl-2,4-dioxobutanoate (9.8 g, 46.2 mmol) in EtOH (100 mL), was added hydroxylammonium chloride (9.63 g, 139 mmol). The reaction mixture was heated to 80° C. and stirred at that temperature for 1 h. The solvents were concentrated in vacuo. Water (50 L) was added to the reaction vessel and extracted with EtOAc. The combined organics were dried over anhydrous sodium sulfate, filtered and concentrated in vacuo. The resulting oil was purified by flash column chromatography to give the titled compound (8.6 g, 89%). MS (ESI): m/e=210 [M+H]⁺.

### Ethyl 5-cyclopentyl-4-nitro-1,2-oxazole-3-carboxylate

To a solution of ethyl 5-cyclopentyl-1,2-oxazole-3-carboxylate (8.6 g, 41.1 mmol) in sulfuric acid (80 mL), was added nitric acid (12.95 g, 206 mmol). The reaction mixture was cooled to 0° C. and stirred at that temperature for 5 h. Icewater (200 mL) was added to the reaction vessel and the resulting biphasic mixture was transferred to a separatory funnel. The layers were separated and the organic phase was washed with sat. sodium bicarbonate aq. solution (300 mL×2). The combined organics were dried over anhydrous sodium sulfate, filtered and concentrated in vacuo. The resulting oil was purified by flash column chromatography to give the titled compound (5.2 g, 55%). MS (ESI): m/e=255 [M+H]⁺.

### Ethyl 4-amino-5-cyclopentyl-1,2-oxazole-3-carboxylate

To a solution of ethyl 5-cyclopentyl-4-nitro-1,2-oxazole-3-carboxylate (8.0 g, 35.1 mmol) in MeOH (30 mL), was added Raney nickel (800 mg). The mixture was stirred at rt under H₂ atmosphere for 2 h. Filtered, the filtrate was concentrated and residue was purified by chromatography on silica gel (EtOAc:PE=10:1 to 3:1) to give the titled compound (6.0 g, 86%) as an orange oil. MS (ESI): m/e=199 [M+1]⁺.

### Ethyl 5-cyclopentyl-4-nitroisoxazole-3-carboxylate

To a solution of ethyl 4-amino-5-cyclopentyl-1,2-oxazole-3-carboxylate (5.2 g, 20.5 mmol) in EtOH (55 mL) was added Raney nickel (1.2 g, 20.5 mmol). The reaction was stirred at rt under H₂ atmosphere for 16 h. The mixture was filtered and concentrated in vacuo. The resulting oil was purified by flash column chromatography to give the titled compound (2.98 g, 65%) as yellow solids. MS (ESI): m/e=225 [M+H]⁺.

### Ethyl 4-[(benzoylcarbamoyl)amino]-5-cyclopentyl-1,2-oxazole-3-carboxylate

To a solution of ethyl 5-cyclopentyl-4-nitroisoxazole-3-carboxylate (3 g, 13.4 mmol) in THF (30 mL) was added benzoyl isocyanate (1.97 g, 13.4 mmol). The reaction mixture was heated to 50° C. and stirred at that temperature for 16 h. The reaction mixture was monitored by LCMS. The combined organics concentrated in vacuo to provide the titled compound (4.22 g, 85%) as white solids. MS (ESI): m/e=372 [M+H]⁺.

### 3-Cyclopentyl[1,2]oxazolo[4,3-d]pyrimidine-5,7(4H,6H)-dione

To a solution of ethyl 4-[(benzoylcarbamoyl)amino]-5-cyclopentyl-1,2-oxazole-3-carboxylate (4.22 g, 11.4 mmol) in MeOH (45 mL) was added potassium carbonate (4.71 g, 34.1 mmol). The reaction mixture was heated to 50° C. and stirred at that temperature for 2 h. Concentrated, water (20 mL) was added to the reaction, then neutralized with 2M hydrochloric acid, filtered and washed with MeOH to get the titled compound (1.76 g, 70%) as white solids. MS (ESI): m/e=222 [M+H]⁺.

### 5,7-Dichloro-3-cyclopentyl[1,2]oxazolo[4,3-d]pyrimidine

To a solution of 3-cyclopentyl[1,2]oxazolo[4,3-d]pyrimidine-5,7(4H,6H)-dione (0.66 g, 3 mmol) in 1,2-dichloroethane (7 mL), was added phosphorus oxychloride (2.3 g, 15 mmol) and DIPEA (1.16 g, 9 mmol). The reaction mixture was heated to reflux for 6 h. Concentrated in vacuo, water (3 mL) was added to the reaction, then neutralized with sodium bicarbonate aq. solution, filtered and washed with water to get the titled compound (0.58 g, 75%) as white solids. MS (ESI): m/e=258 [M+H]⁺.

### 5-Chloro-3-cyclopentyl[1,2]oxazolo[4,3-d]pyrimidin-7(4H)-one

To a solution of 5,7-dichloro-3-cyclopentyl[1,2]oxazolo[4,3-d]pyrimidine (0.1 g, 0.39 mmol) in THF (1 mL), was added 2 M potassium hydroxide aq. solution (1 mL). The mixture was stirred for 2 h at 50° C. Cooled, neutralized with 1M hydrochloric acid, filtered to get the titled compound (0.08 g, 85%) as white solids. MS (ESI): m/e=240 [M+H]⁺.

### 5-(1-(3-Chlorophenyl)-3-hydroxypropylamino)-3-cyclopentylisoxazolo[4,3-d]pyrimidin-7(6H)-one

To a solution of 5-chloro-3-cyclopentyl[1,2]oxazolo[4,3-d]pyrimidin-7(4H)-one (0.08 g, 0.33 mmol) in 1-butanol (0.8 mL), was added 3-amino-3-(3-chlorophenyl)-1-propanol (0.12 g, 0.66 mmol) and DIPEA (0.13 g, 0.99 mmol). The reaction mixture was heated to 100° C. and stirred at that temperature for 12 h. The progress of the reaction mixture was monitored by LCMS and then purified by column chromatography to get the titled compound (0.09 g, 69%) as white solids. MS (ESI): m/e=389 [M+H]⁺.

The compounds of Example 75 to 85 were synthesized in a similar manner to Example 74, Reference Example 1, and 2.

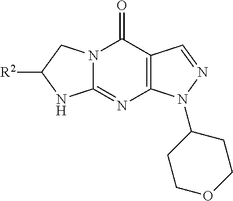

| Ex. | | |
|---|---|---|
| No. | R²— | ¹H-NMR |
| 75 | 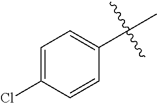 [SMILES: *c1ccc(Cl)cc1] | ¹H NMR (400 MHz, CDCl₃): δ 7.97 (d, J = 2.0 Hz, 1H), 7.42 (d, J = 10.4 Hz, 2 H) 7.34 (d, J = 10.4 Hz, 2 H), 5.49 (s, 1 H), 5.16 (t, J = 8.0 Hz, 1 H), 4.65-4.58 (m, 2 H), 4.15 (d, J = 9.2 Hz, 2 H), 4.00-3.96 (m, 1 H), 3.59 (t, J = 12.4 Hz, 2 H), 2.39-2.36 (m, 2 H), 1.93-1.89 (m, 2 H) |
| 76 | 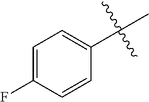 [SMILES: *c1ccc(F)cc1] | ¹H NMR (400 MHz, CDCl₃): δ 7.96 (s, 1 H), 7.40-7.36 (m, 2 H) 7.16- 7.11 (m, 2 H), 5.47 (s, 1 H), 5.17 (t, J = 8.4 Hz, 1 H), 4.67-4.58 (m, 2 H), 4.17-4.13 (m, 2 H), 4.01-3.96 (m, 1 H), 3.62-3.56 (m, 2 H), 2.42- 2.32 (m, 2 H), 1.93-1.89 (m, 2 H) |

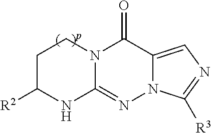

| Ex. | | | | |
|---|---|---|---|---|
| No. | P | R²— | R³— | ¹H-NMR |
| 77 | 0 | 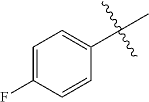 [SMILES: *c1ccc(F)cc1] | 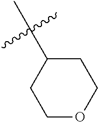 [SMILES: *C1CCOCC1] | ¹H NMR (400 MHz, CDCl₃): δ 7.80 (s, 1 H), 7.38- 7.41 (m, 2 H), 7.12-7.16 (m, 2 H), 5.11-5.15 (m, 2 H), 4.54-4.59 (m, 1 H), 4.08-4.13 (m, 2 H), 3.89- 3.94 (m, 1 H), 3.56-3.63 (m, 2 H), 3.34 (m, 1 H), 2.06-2.13 (m, 2 H), 1.91-1.94 (m, 2 H) |
| 78 | 0 | 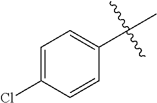 [SMILES: *c1ccc(Cl)cc1] | 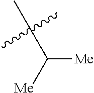 [SMILES: *C(C)C] | ¹H NMR: (400 MHz, CDCl₃): δ 7.79 (s, 1 H), 7.45- 7.42 (m, 2 H), 7.37 (t, J = 3.6 Hz, 2 H), 5.12 (t, J = 7.8 Hz, 1 H), 5.06 (s, 1 H), 4.60-4.55 (m, 1 H), 3.40 (t, J = 6.8 Hz, 1 H), 3.93-3.88 (m, 1 H), 1.42-1.39 (m, 6 H) |
| 79 | 0 | 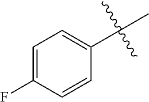 [SMILES: *c1ccc(F)cc1] | 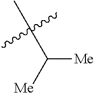 [SMILES: *C(C)C] | ¹H NMR (400 MHz, CDCl₃): δ 7.78 (s, 1 H), 7.38- 7.41(m, 2 H), 7.12-7.16 (m, 2 H), 5.20 (s, 1 H), 5.12 (t, J = 8.6 Hz, 1 H), 4.53-4.58 (m, 1 H), 3.87- 3.92 (m, 1 H), 3.37-3.41 (m, 1 H), 1.37-1.41 (m, 6 H) |
| 80 | 0 | 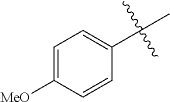 [SMILES: *c1ccc(OC)cc1] | 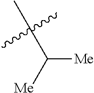 [SMILES: *C(C)C] | ¹H NMR (400 MHz, CDCl₃) δ 7.78 (s, 1 H), 7.28- 7.34 (m, 2 H), 6.95-6.98 (m, 2 H), 5.07 (t, J = 7.6 Hz, 2 H), 4.53 (dd, J1 = 11.6 Hz, J2 = 8.8 Hz, 1 H), 3.92 (dd, J1 = 13.2 Hz, J2 = 7.6 Hz, 1 H), 3.85 (s, 3 H), 3.36-3.43 (m, 1 H), 1.39 (dd, J = 4.2 Hz, 6.8 Hz, 6 H) |
| 81 | 0 | 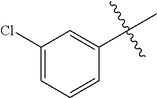 [SMILES: *c1cccc(Cl)c1] | 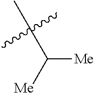 [SMILES: *C(C)C] | ¹H NMR (400 MHz, CDCl₃): δ 7.79 (s, 1 H), 7.39- 7.43 (m, 3 H), 7.28-7.31 (m, 1 H), 5.11-5.14 (m, 2 H), 4.56-4.61 (m, 1 H), 3.92-3.94 (m, 1 H), 3.38- 3.42 (m, 1 H), 1.38-1.41 (m, 6 H) |
| 82 | 0 | 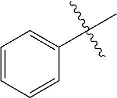 [SMILES: *c1ccccc1] | 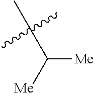 [SMILES: *C(C)C] | ¹H NMR (400 MHz, CDCl₃): δ 7.79 (s, 1 H), 7.47- 7.39 (m, 5 H), 5.15-5.11 (m, 2 H), 4.59-4.55 (m, 1 H), 3.98-3.94 (t, J = 9.6 Hz, 1 H), 3.42-3.39 (t, J = 7.0 Hz, 1 H), 1.42-1.39 (m, 6 H) |
| 83 | 0 | 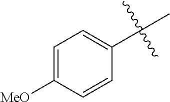 [SMILES: *c1ccc(OC)cc1] | 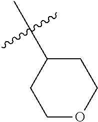 [SMILES: *C1CCOCC1] | ¹H NMR (400 MHz, CDCl₃): δ 7.79 (s, 1 H), 7.28- 7.33 (m, 2 H), 6.95-6.97 (m, 2 H), 5.02-5.08 (m, 2 H), 4.51-4.56 (m, 1 H), 4.09-4.12 (m, 2 H), 3.90- 3.95 (m, 1 H), 3.83 (s, 3 H), 3.56-3.63 (m, 2 H), 3.34 (m, 1 H), 2.07-2.13 (m, 2 H), 1.91-1.94 (m, 2 H) |
| 84 | 0 | 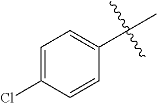 [SMILES: *c1ccc(Cl)cc1] | 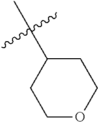 [SMILES: *C1CCOCC1] | ¹H NMR (400 MHz, CDCl₃): δ 7.80 (s, 1 H), 7.45- 7.43 (m, 2 H), 7.39 (t, J = 11.2 Hz, 2 H), 5.12 (t, J = 8.2 Hz, 1 H), 4.59 (t, J = 10.0 Hz, 1 H), 4.13-4.09 (m, 2 H), 3.93-3.89 (m, 1 H), 3.63-3.57 (m, 1 H), 3.37-3.33 (m, 2 H), 3.31 (s, 1 H), 2.13-2.07 (m, 2 H), 1.95-1.91 (m, 2 H) |
| 85 | 1 | 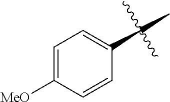 | 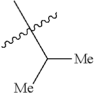 [SMILES: *C(C)C] | ¹H NMR (400 MHz, CDCl₃): δ 7.77 (s, 1 H), 7.26- 7.29 (m, 2 H), 6.92-6.95 (m, 2 H), 5.01 (s, 1 H), 4.59-4.63 (m, 1 H), 4.30-4.34 (m, 1 H), 3.82 (s, 3 H), 3.73-3.79 (m, 1 H), 3.36 (t, J = 6 Hz, 1 H), 2.30- 2.32 (m, 1 H), 2.02-2.05 (m, 1 H), 1.34-1.38 (m, 6 H) |

### Example 86

### 4-[4-Oxo-1-(tetrahydro-2H-pyran-4-yl)-4,6,7,8-tetrahydro-1H-imidazo[1,2-a]pyrazolo[3,4-d]pyrimidin-7-yl]benzonitrile

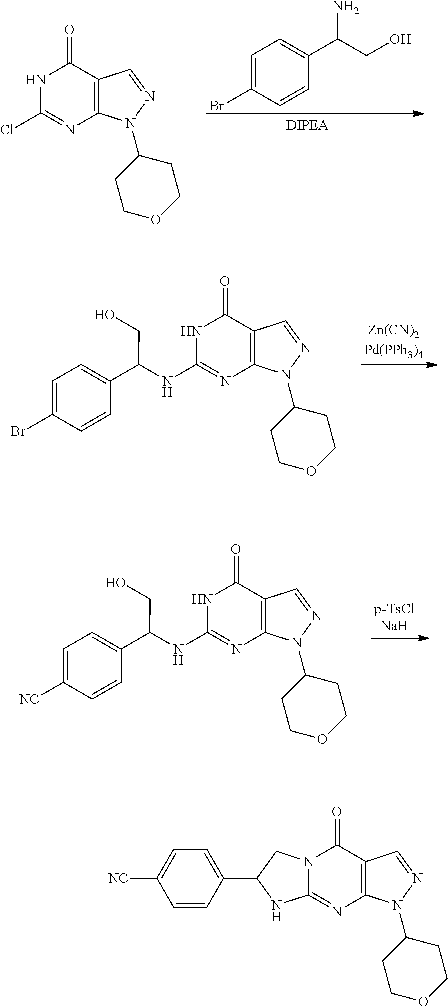 [SMILES: O=c1[nH]c(Cl)nc2c1cnn2C1CCOCC1 ; NC(CO)c1ccc(Br)cc1 ; O=c1[nH]c(NC(CO)c2ccc(Br)cc2)nc2c1cnn2C1CCOCC1 ; N#Cc1ccc(C(CO)Nc2nc3c(cnn3C3CCOCC3)c(=O)[nH]2)cc1 ; N#Cc1ccc(C2Cn3c(nc4c(cnn4C4CCOCC4)c3=O)N2)cc1]

To a solution of 4-(2-hydroxy-1-{[4-oxo-1-(tetrahydro-2H-pyran-4-yl)-4,5-dihydro-1H-pyrazolo[3,4-d]pyrimidin-6-yl]amino}ethyl)benzonitrile (0.02 g, 0.05 mmol) in THF (0.2 mL) was added sodium hydride (0.01 g, 0.25 mmol) and p-tosyl chloride (0.01 g, 0.06 mmol). The reaction mixture was stirred at rt for 10 min. The progress of the reaction mixture was monitored by LCMS and then purified by prep-HPLC to give the titled compound (5 mg, 40%) as white solids. ¹H NMR (400 MHz, DMSO-d₆): δ 8.79 (s, 1H), 7.90 (d, J=8.4 Hz, 2H), 7.83 (s, 1H), 7.64 (d, J=8.4 Hz, 2H), 5.31-5.26 (m, 1H), 4.59-4.52 (m, 2H), 4.00-3.98 (m, 2H), 3.78-3.74 (m, 1H), 3.52-3.33 (m, 2H), 2.14-2.05 (m, 2H), 1.80-1.78 (m, 2H). LCMS: m/z=363 [M+H]⁺.

### 6-{[1-(4-Bromophenyl)-2-hydroxyethyl]amino}-1-(tetrahydro-2H-pyran-4-yl)-1,5-dihydro-4H-pyrazolo[3,4-d]pyrimidin-4-one

To a solution of 2-amino-2-(4-bromophenyl)ethanol (0.11 g, 0.5 mmol) in 1-butanol (2 mL) was added 6-chloro-1-(tetrahydro-2H-pyran-4-yl)-1,5-dihydro-4H-pyrazolo[3,4-d]pyrimidin-4-one (synthesized in a similar manner to Reference Example 1, 0.13 g, 0.5 mmol) and DIPEA (0.19 g, 1.5 mmol). The reaction mixture was heated to 120° C. and stirred at that temperature for 12 h. The progress of the reaction mixture was monitored by LCMS and then purified by column chromatography (DCM:MeOH=20:1) to give the titled compound (200 mg, 90%) as a colorless oil. LCMS: m/z=435 [M+H]⁺.

### 4-(2-Hydroxy-1-{[4-oxo-1-(tetrahydro-2H-pyran-4-yl)-4,5-dihydro-1H-pyrazolo[3,4-d]pyrimidin-6-yl]amino}ethyl)benzonitrile

To a solution of 6-{[1-(4-bromophenyl)-2-hydroxyethyl]amino}-1-(tetrahydro-2H-pyran-4-yl)-1,5-dihydro-4H-pyrazolo[3,4-d]pyrimidin-4-one (0.2 g, 0.45 mmol) in DMF (2 mL) was added zinc cyanide (0.11 g, 0.9 mmol) and tetrakis(triphenylphosphine) palladium (0.06 g, 0.05 mmol). The reaction mixture was heated to 100° C. and stirred at that temperature for 16 h. The progress of the reaction mixture was monitored by LCMS and then purified by HPLC to give the titled compound (160 mg, 6096) as white solids. LCMS: m/z=381 [M+H]⁺.

The compounds of Example 87 to 95 were synthesized in a similar manner to Example 86 and Reference Example 2.

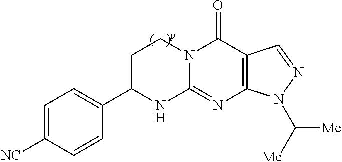

| Ex. | | |
|---|---|---|
| No. | P | ¹H NMR |
| 87 | 0 | ¹H NMR (400 MHz, CDCl₃): δ 7.95 (s, 1 H), 7.74 (d, J = 8 Hz, 2 |
| | | H), 7.54 (d, J = 12 Hz, 2 H), 5.54 (s, 1 H), 5.24 (t, J = 10 Hz, 1 H), |
| | | 4.80-4.83 (m, 1 H), 4.63-4.68 (m, 1 H), 3.94-3.99 (m, 1 H), 1.50- |
| | | 1.53 (m, 6 H) |
| 88 | 1 | ¹H NMR (400 MHz, CDCl₃): δ 7.97 (s, 1H), 7.74 (d, J = 8.0 Hz, |
| | | 2H), 7.52 (d, J = 8.0 Hz, 2H), 5.60 (br s, 1H), 4.86-4.77 (m, 2H), |
| | | 4.18-3.97 (m, 2H), 2.44-2.38 (m, 1H), 2.13-2.05 (m, 1H), 1.54- |
| | | 1.50 (m, 6H) |

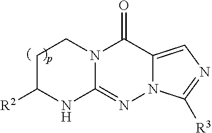

| Ex. | | | | |
|---|---|---|---|---|
| No. | P | R²— | R³— | ¹H-NMR |
| 89 | 0 | 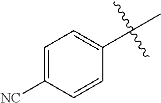 [SMILES: *c1ccc(C#N)cc1] | 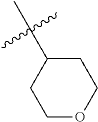 [SMILES: *C1CCOCC1] | ¹H NMR (400 MHz, CDCl₃): δ 7.81 (s, 1 H), 7.77 (d, J= 8 Hz, 2 H), 7.56 (d, J = 8.4 Hz, 2 H), 5.29 (s, 1 H), 5.21 (t, J = 8 Hz, 1 H), 4.61-4.66 (m, 1 H), 4.09-4.12 (m, 2 H), 3.89-3.94 (m, 1 H), 3.59 (t, J = 10 Hz, 2 H), 3.34-3.35 (m, 1 H), 2.07-2.13 (m, 2 H), 1.90-1.94 (m, 2 H) |
| 90 | 0 | 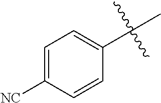 [SMILES: *c1ccc(C#N)cc1] | 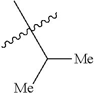 [SMILES: *C(C)C] | ¹H NMR (400 MHz, DMSO-d₆) δ 8.45 (s, 1 H), 7.90 (d, J = 8.0 Hz, 2 H), 7.69 (d, J = 8.4 Hz, 2 H), 7.57 (s, 1 H), 5.21-5.25 (m, 1 H), 4.52-4.57 (m, 1 H), 3.73 (dd, J1 = 10.8 Hz, J2 = 7.2 Hz, 1 H), 3.29-3.34 (m, 1 H), 1.28-1.31 (m, 6 H) |
| 91 | 1 | 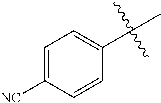 [SMILES: *c1ccc(C#N)cc1] | 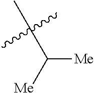 [SMILES: *C(C)C] | ¹H NMR (400 MHz, CDCl₃): δ 7.81 (s, 1H), 7.75 (d, J = 8.0 Hz, 2H), 7.54 (d, J = 8.4 Hz, 2H), 5.16 (s, 1H), 4.81-4.79 (m, 1H), 4.21-4.15 (m, 1H), 3.96-3.89 (m, 1H), 3.39 (sept, J = 6.8 Hz, IH), 2.46-2.41 (m, 1H), 2.13-2.04 (m, 1H), 1.40 (d, J = 7.2 Hz, 3H), 1.37 (d, J = 8.0 Hz, 3H) |
| 92 | 1 | 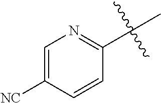 [SMILES: *c1ccc(C#N)cn1] | 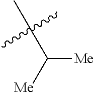 [SMILES: *C(C)C] | ¹H NMR (400 MHz, CDCl₃): δ 8.93 (m, 1 H), 8.09- 8.06 (m, 1 H), 7.80 (s, 1 H), 7.59 (d, J = 5.8 Hz, 1 H), 5.72 (s, 1 H), 4.91-4.87 (m 1 H), 4.17-4.11 (m, 1 H), 4.04-3.97 (m, 1 H), 3.46-3.39 (m, 1 H), 2.59-2.54 (m, 1 H), 2.26-2.21 (m, 1 H), 1.41-1.28 (m, 6 H) |
| 93 | 1 | 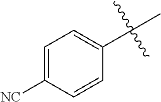 [SMILES: *c1ccc(C#N)cc1] | 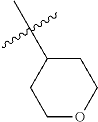 [SMILES: *C1CCOCC1] | ¹H NMR (400 MHz, CDCl₃): δ 7.81 (s, 1H), 7.75 (d, J = 8.4 Hz, 2H), 7.54 (d, J = 8.0 Hz, 2H), 5.20 (br s, 1H), 4.82-4.79 (m, 1H), 4.21-4.12 (m, 3H), 3.95-3.91 (m, 1H), 3.61-3.54 (m, 2H), 3.34-3.32 (m, 1H), 2.43- 2.41 (m, 1H), 2.15-2.07 (m, 3H), 1.93-1.89 (m, 2H) |
| 94 | 1 | 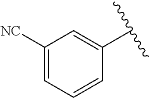 [SMILES: *c1cccc(C#N)c1] | 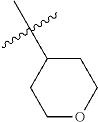 [SMILES: *C1CCOCC1] | ¹H NMR (400 MHz, CDCl₃): δ 7.82 (s, 1H), 7.72- 7.57 (m, 4H), 5.17 (s, 1H), 4.79-4.76 (m, 1H), 4.24- 4.21 (m, 1H), 4.10 (br s, 2H), 3.95-3.90 (m, 1H), 3.61-3.55 (m, 2H), 3.36-3.31 (m, 1H), 2.46-2.42 (m, 1H), 2.15-2.03 (m, 3H), 1.95-1.89 (m, 2H) |
| 95 | 1 | 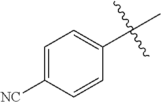 [SMILES: *c1ccc(C#N)cc1] | 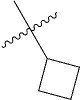 [SMILES: *C1CCC1] | ¹H NMR (400 MHz, CDCl₃) δ 7.84 (s, 1 H), 7.75 (dd, J1 = 6.4 Hz, J2 = 2.0 Hz, 2 H), 7.53 (d, J = 8.4 Hz, 2 H), 5.09 (s, 1 H), 4.78-4.80 (m, 1 H), 4.15-4.19 (m, 1 H), 3.88-3.95 (m, 2 H), 2.50-2.62 (m, 2 H), 2.36-2.42 (m, 3 H), 1.61-2.11 (m, 3 H) |

### Example 96

### (2S)-8-Isopropyl-2-(4-methylphenyl)-2,10-dihydro-3H,5H-diimidazo[2,1-c:5′,1′-f][1,2,4]triazin-5-one

### Example 97

### (2R)-8-Isopropyl-2-(4-methylphenyl)-2,10-dihydro-3H,5H-diimidazo[2,1-c:5′,1′-f][1,2,4]triazin-5-one

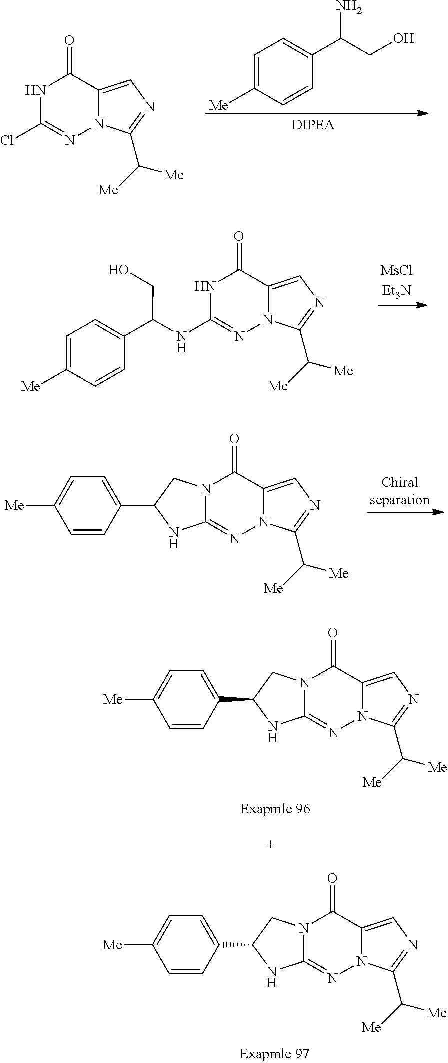 [SMILES: CC(C)c1ncc2c(=O)[nH]c(Cl)nn12 ; Cc1ccc(C(N)CO)cc1 ; Cc1ccc(C(CO)Nc2nn3c(C(C)C)ncc3c(=O)[nH]2)cc1 ; Cc1ccc(C2Cn3c(nn4c(C(C)C)ncc4c3=O)N2)cc1 ; Cc1ccc([C@H]2Cn3c(nn4c(C(C)C)ncc4c3=O)N2)cc1 ; Cc1ccc([C@@H]2Cn3c(nn4c(C(C)C)ncc4c3=O)N2)cc1]

Chiral separation of 8-isopropyl-2-(4-methylphenyl)-2,10-dihydro-3H,5H-diimidazo[2,1-c:5′,1′-f][1,2,4]triazin-5-one (600 mg, 1.9 mmol) gave the titled compounds (Example 96: 133 mg, 22%; Example 97: 144 mg, 24%).

Example 96: ¹H NMR (400 MHz, CDCl₃): δ 7.77 (s, 1H), 7.27-7.29 (m, 3H), 7.23-7.25 (m, 1H), 5.06-5.10 (m, 2H), 4.51-4.56 (m, 1H), 3.90-3.95 (m, 1H), 3.35-3.42 (m, 1H), 2.39 (s, 3H), 1.37-1.40 (m, 6H). Chiral separation: retention time 3.18 min (Method 13).

Example 97: ¹H NMR (400 MHz, CDCl₃): δ 7.77 (s, 1H), 7.27-7.29 (m, 3H), 7.22-7.24 (m, 1H), 5.06-5.09 (m, 2H), 4.50-4.55 (m, 1H), 3.89-3.94 (m, 1H), 3.36-3.40 (m, 1H), 2.38 (s, 3H), 1.37-1.40 (m, 6H). Chiral separation: retention time 5.51 min (Method 13).

### 2-{[2-Hydroxy-1-(4-methylphenyl)ethyl]amino}-7-isopropylimidazo[5,1-f][1,2,4]triazin-4(1H)-one

To solution of 2-chloro-7-isopropylimidazo[5,1-f][1,2,4]triazin-4(1H)-one (synthesized in a similar manner to Reference Example 2, 1.6 g, 7.55 mmol) in 1-butanol (8 mL) was added DIPEA (2.92 g, 22.65 mmol), sodium iodide (1.33 g, 7.55 mmol) and 2-amino-2-p-tolylethanol (1.14 g, 7.55 mmol). The mixture was stirred at 170° C. for 17 h in microwave. Then it was filtered and the filtrate was concentrated. The crude product was purified by silica gel chromatography (eluted with DCM:MeOH=10:1) to give the titled compound as white solids (960 mg, 38.9%). LCMS: m/z=328 [M+H]⁺.

### 8-Isopropyl-2-(4-methylphenyl)-2,10-dihydro-3H,5H-diimidazo[2,1-c:5′,1′-f][1,2,4]triazin-5-one

To a solution of 2-{[2-hydroxy-1-(4-methylphenyl)ethyl]amino}-7-isopropylimidazo[5,1-f][1,2,4]triazin-4(1H)-one (960 mg, 2.94 mmol) in DCM (10 mL) was added TEA (2.08 g, 20.55 mmol). The mixture was stirred at 0° C. for 20 mins then mesyl chloride (670 mg, 5.88 mmol) was added. The mixture was stirred at rt for 2 h. The crude product was purified by Prep-TLC (eluted with DCM:MeOH=20:1) to give the titled compound as white solids (600 mg, 66%). LCMS: m/z=310 [M+H]⁺.

The compounds of Example 98 to 135 were synthesized in a similar manner to Example 96 and 97 and Reference Example 2.

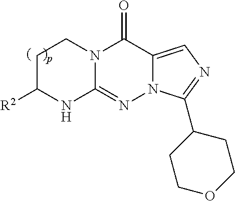

| | | | | Separation: |
|---|---|---|---|---|
| Ex. | | | | retention time |
| No. | p | R²— | ¹H NMR | method |
| 98 | 0 | 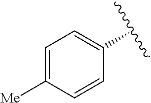 | ¹H NMR (400 MHz, DMSO-d₆) δ 8.35 (s, 1 H), 7.58 (s, 1 H), 7.32 (d, J = 8.0 Hz, 2 H), 7.20 (d, J = 8.0 Hz, 2 H), 5.07 (t, J = 8.0 Hz, 1 H), 4.44- 4.49 (m, 1 H), 3.94-3.96 (m, 2 H), 3.66-3.71 (m, 1 H), 3.43-3.49 (m, 2 H), 3.24-3.30 (m, 1 H), 2.30 (s, 3 H), 1.80-1.87 (m, 4 H) | 2.50 min Method 10 |
| 99 | 0 | 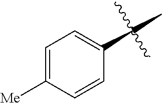 | ¹H NMR (400 MHz, DMSO-d₆) δ 8.35 (s, 1 H), 7.58 (s, 1 H), 7.32 (d, J = 8.0 Hz, 2 H), 7.20 (d, J = 8.0 Hz, 2 H), 5.07 (t, J = 8.0 Hz, 1 H), 4.44- 4.49 (m, 1 H), 3.94-3.96 (m, 2F), 3.66-3.71 (dd, J1 = 10.8 Hz, J2 = 3.2 Hz, 1 H), 3.43-3.49 (m, 2 H), 3.27-3.30 (m, 1 H), 2.30 (s, 3 H), 1.80-1.87 (m, 4 H) | 4.47 min Method 10 |
| 100 | 0 | 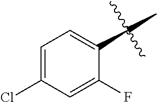 | ¹H NMR (400 MHz, CDCl₃): δ 8.42 (s, 1 H), 7.59-7.50 (m, 3 H), 7.38-7.35 (m, 1 H), 5.34- 5.30 (m, 1 H), 4.52-4.46 (m, 1 H), 3.97-3.93 (m, 2 H), 3.85-3.81 (m, 1 H), 3.49-3.43 (m, 2 H), 3.31-3.25 (m, 1 H), 1.87-1.79 (m, 4H) | 5.63 min Method 17 |
| 101 | 0 | 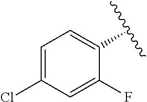 | ¹H NMR (400 MHz, CDCl₃): 8.42 (s, 1H), 7.59- 7.50 (m, 3H ), 7.38-7.35 (m, 1H), 5.34-5.30 (m, 1H), 4.52-4.46 (m, 1H), 3.97-3.93 (m, 2H), 3.85- 3.81 (m, 1H), 3.49-3.43 (m, 2H), 3.30-3.26 (m, 1H), 1.88-1.79 (m, 4H) | 8.69 min Method 17 |
| 102 | 0 | 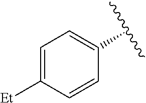 | ¹H NMR (400 MHz, CDCl₃): δ 7.78 (s, 1 H), 7.30 (d, J = 4.2 Hz, 2 H), 7.26 (d, J = 4.2 Hz, 2 H), 5.11-5.07 (m, 2 H), 4.56-4.51 (m, 1 H), 4.11- 4.08 (m, 2 H), 3.97-3.92 (m, 1 H), 3.62-3.56 (m, 2 H), 3.36-3.30 (m, 1 H), 2.71-2.65 (m, 2 H), 2.12-2.05 (m, 2 H), 1.94-1.89 (m, 2 H), 1.27- 1.24 (m, 3 H) | 2.38 min Method 10 |
| 103 | 0 | 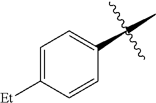 | ¹H NMR (400 MHz, CDCl₃): δ 7.78 (s, 1 H), 7.30 (d, J = 4.2 Hz, 2 H), 7.26 (d, J = 4.2 Hz, 2 H), 5.11-5.07 (m, 2 H), 4.56-4.51 (m, 1 H), 4.11- 4.08 (m, 2 H), 3.97-3.92 (m, 1 H), 3.62-3.56 (m, 2 H), 3.36-3.30 (m, 1 H), 2.71-2.65 (m, 2 H), 2.12-2.05 (m, 2 H), 1.94-1.89 (m, 2 H), 1.27- 1.24 (m, 3 H) | 4.26 min Method 10 |
| 104 | 0 | 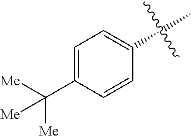 | ¹H NMR (400 MHz, CDCl₃): δ 7.79 (s, 1 H), 7.45-7.47 (m, 2 H), 7.28-7.33 (m, 2 H), 5.06- 5.10 (m, 2 H), 4.51-4.56 (m, 1 H), 4.09-4.11 (m, 2 H), 3.94-3.99 (m, 1 H), 3.59-3.63 (m, 2 H), 3.56-3.57 (m, 1 H), 2.10 (m, 2 H), 1.95 (m, 2 H), 1.35 (s, 9 H) | 1.96 min Method 10 |
| 105 | 0 | 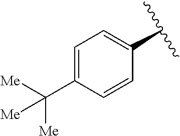 | ¹H NMR (400 MHz, CDCl₃): δ 7.79 (s, 1 H), 7.45-7.47 (m, 2 H), 7.28-7.33 (m, 2 H), 5.08- 5.10 (m, 2 H), 4.51-4.56 (m, 1 H), 4.09-4.11 (m, 2 H), 3.94-3.99 (m, 1 H), 3.56-3.63 (m, 2 H), 3.31-3.37 (m, 1 H), 2.06-2.13 (m, 2 H), 1.91- 1.94 (m, 2R), 1.35 (s, 9 H) | 3.27 min Method 10 |
| 106 | 0 | 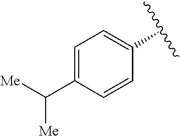 | ¹H NMR (400 MHz, DMSO-d₆): δ 8.32 (br s, 1 H), 7.58 (s, 1 H), 7.30 (dd, J1 = 34.0 Hz, J2 = 8.0 Hz, 4 H), 5.05-5.09 (m, 1 H), 4.44-4.46 (m, 1 H), 3.95 (dd, J1 = 10.0 Hz, J2 = 2.8 Hz, 2 H), 3.68- 3.73 (m, 1 H), 3.43-3.49 (m, 2 H), 3.25-3.32 (m, 1 H), 2.86-2.93 (m, I H), 1.79-1.87 (m, 4 H), 1.20 (d, J = 6.8 Hz, 6 H) | 2.04 min Method 10 |
| 107 | 0 | 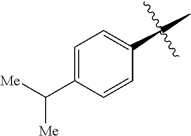 | ¹H NMR (400 MHz, DMSO-d₆): δ 7.58 (s, 1 H), 8.34 (br s, 1 H), 7.30 (dd, J1 = 34.0 Hz, J2 = 8.0 Hz, 4 H), 5.07 (t, J = 8.0 Hz, 1 H), 4.44-4.46 (m, 1 H), 3.93 (dd, J 1 = 10.8 Hz, J2 = 2.8 Hz, 2 H), 3.68-3.73 (m, 1 H), 3.43-3.49 (m, 2 H), 3.24- 3.32 (m, 1 H), 2.86-2.93 (m, 1 H), 1.79-1.85 (m, 4H), 1.20 (d, J = 6.8 Hz, 6 H) | 3.23 min Method 10 |
| 108 | 0 | 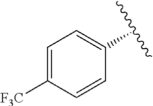 | ¹H NMR (400 MHz, CDCl₃): δ 7.81 (s, 1 H), 7.72-7.74 (m, 2 H), 7.55-7.57 (m, 2 H), 5.21- 5.23 (m, 2 H), 4.60-4.65 (m, 1 H), 4.09-4.12 (m, 2 H), 3.91-3.95 (m, 1 H), 3.56-3.63 (m, 2 H), 3.32-3.36 (m, 1 H), 2.06-2.13 (m, 2 H), 1.92- 1.95 (m, 2 H). | 1.71 min Method 10 |
| 109 | 0 | 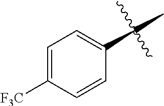 | ¹H NMR (400 MHz, CDCl₃): δ7.81 (s, 1 H), 7.72-7.74 (m, 2 H), 7.55-7.57 (m, 2 H), 5.21- 5.25 (m, 2 H), 4.60-4.65 (m, 1 H), 4.09-4.12 (m, 2 H), 3.91-3.95 (m, 1 H), 3.57-3.63 (m, 2 H), 3.32-3.36 (m, 1 H), 2.06-2.09 (m, 2 H), 1.93 (br s, 2 H) | 2.31 min Method 10 |
| 110 | 0 | 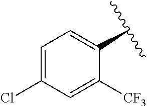 | ¹H NMR (400 MHz, CDCl3): δ 8.49 (s, 1 H), 7.91-7.85 (m, 3 H), 7.60 (s, 1 H), 5.39 (s, 1 H), 4.48-4.43 (m, 1 H), 3.97-3.94 (m, 2 H), 3.75- 3.71 (m, 1 H), 3.49-3.44 (m, 2 H), 3.34 (s, 1 H), 1.85-1.80 (m, 4 H) | 7.27 min Method 17 |
| 111 | 0 | 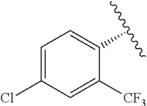 | ¹H NMR (400 MHz, CDCl₃): δ 8.49 (s, 1 H), 7.91-7.85 (m, 3 H), 7.60 (s, 1 H), 5.39 (s, 1 H), 4.48-4.43 (m, 1 H), 3.97-3.94 (m, 2 H), 3.75- 3.71 (m, 1 H), 3.49-3.44 (m, 2 H), 3.34 (s, 1 H), 1.85-1.80 (m, 4 H) | 4.24 min Method 17 |
| 112 | 0 | 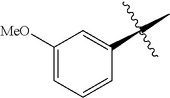 | ¹H NMR (400 MHz, CDCl₃): δ 7.80 (s, 1 H), 7.38-7.34 (m, 1 H), 6.98-6.93 (m, 3 H), 5.13- 5.06 (m, 2 H), 4.59-4.54 (m, 1 H), 4.12-4.09 (m, 2 H), 3.98-3.93 (m, 1 H), 3.85 (s, 3 H), 3.63-3.57 (m, 2 H), 3.36-3.32 (m, 1 H), 2.10-2.07 (m, 2 H), 1.96-1.91 (m, 2 H) | 3.92 min Method 4 |
| 113 | 0 | 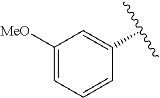 | ¹H NMR (400 MHz, CDCl₃): δ 7.80 (s, 1 H), 7.38-7.34 (m, 1 H), 6.98-6.93 (m, 3 H), 5.13- 5.06 (m, 2 H), 4.59-4.54 (m, 1 H), 4.12-4.09 (m, 2 H), 3.98-3.93 (m, 1 H), 3.85 (s, 3 H), 3.63-3.57 (m, 2 H), 3.36-3.32 (m, 1 H), 2.10-2.07 (m, 2 H), 1.96-1.91 (m, 2 H) | 6.72 min Method 4 |
| 114 | 0 | 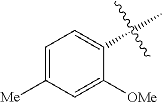 | ¹H NMR (400 MHz, CDCl₃): δ 7.81-7.77 (m, 1 H), 7.25-7.20 (m, 1 H), 6.86-6.77 (m, 2 H), 5.34- 5.32 (m, 1 H), 5.10-5.07 (m, 1 H), 4.58-4.53 (m, 1 H), 4.12-4.10 (m, 2 H), 4.02-3.98 (m, 1 H), 3.89-3.85 (m, 3 H), 3.66-3.57 (m, 2 H), 3.37- 3.33 (m, 1 H), 2.42-2.39 (m, 3 H), 2.16-2.07 (m, 2H), 1.95-1.92 (m, 2 H) | 2.70 min Method 10 |
| 115 | 0 | 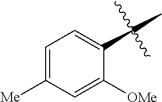 | ¹H NMR (400 MHz, CDCl₃): δ0 7.81-7.77 (m, 1 H), 7.25-7.20 (m, 1 H), 6.84-6.82 (m, 1H), 6.77 (s, 1 H), 5.33-5.32 (m, 1 H), 5.08 (s, 1 H), 4.58- 4.53 (m, 1 H), 4.12-4.09 (m, 2 H), 4.02-3.98 (m, 1 H), 3.85 (s, 3 H), 3.63-3.58 (m, 2 H), 3.37-3.33 (m, 1 H), 2.40 (s, 3 H), 2.14-2.07 (m, 2 H), 1.95- 1.92 (m, 2 H) | 3.48 min Method 10 |
| 116 | 0 | 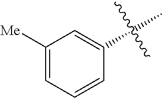 | ¹H NMR (400 MHz, CDCl₃): δ 7.79 (s, 1 H), 7.34-7.31 (m, 1 H), 7.22-7.17 (m, 3 H), 5.32 (s, 1 H), 5.11-5.07 (m, 1 H), 4.57-4.52 (m, 1 H), 4.11- 4.08 (m, 2 H), 3.97-3.92 (m, 1 H), 3.63-3.56 (m, 2 H), 3.37-3.31 (m, 1 H), 2.40 (s, 3 H), 2.12-2.02 (m, 2H), 1.95-1.90 (m, 2 H) | 2.33 min Method 10 |
| 117 | 0 | 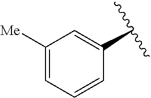 | ¹H NMR (400 MHz, CDCl₃): δ 7.80 (s, 1 H), 7.33-7.28 (m, 1 H), 7.23-7.18 (m, 3 H), 5.13- 5.09 (m, 2 H), 4.58-4.53 (m, 1 H), 4.12-4.09 (m, 2 H), 3.98-3.93 (m, 1 H), 3.64-3.57 (m, 2 H), 3.37-3.31 (m, 1 H), 2.40 (s, 3 H), 2.12-2.02 (m, 2 H), 1.95-1.90 (m, 2 H) | 3.92 min Method 10 |
| 118 | 0 | 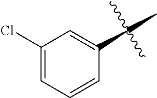 | ¹H NMR (400 MHz, CDCl₃): δ 7.80 (s, 1 H), 7.42-7.39 (m, 3 H), 7.30-7.28 (m, 1 H), 5.23 (s, 1 H), 5.12 (s, 1 H), 4.61-4.56 (m, 1 H), 4.12-4.09 (m, 2H), 3.96-3.91 (m, 1 H), 3.63-3.57 (m, 2H), 3.34 (m, 1 H), 2.13-2.07 (m, 2H), 1.96-1.93 (m, 2 H) | 3.46 min Method 2 |
| 119 | 0 | 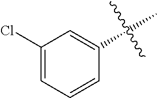 | ¹H NMR (400 MHz, CDCl₃): δ 7.80 (s, 1 H), 7.42-7.39 (m, 3 H), 7.30-7.28 (m, 1 H), 5.23 (s, 1 H), 5.12 (s, 1 H), 4.61-4.56 (m, 1 H), 4.12-4.09 (m, 2 H), 3.96-3.91 (m, 1 H), 3.63-3.57 (m, 2H), 3.34 (m, 1 H), 2.13-2.07 (m, 2 H), 1.96-1.93 (m, 2 H) | 4.87 min Method 2 |
| 120 | 0 | 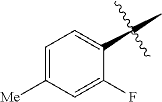 | ¹H NMR (400 MHz, CDCl₃): δ 7.78 (s, 1 H), 7.30-7.26 (m, 1 H), 7.01 (d, J = 8.4 Hz, 1 H), 6.95 (d, J = 11.2 Hz, 1 H), 5.35 (t, J = 8.0 Hz, 1 H), 5.06 (s, 1H), 4.57 (dd, J1 = 8.8 Hz, J2 = 11.2 Hz, 1 H), 4.11-4.07 (m, 3 H), 4.01 (dd, J1 = 6.4 Hz, J2 = 11.2 Hz, 1 H), 3.61-3.54 (m, 1 H), 3.36- 3.29 (m, 1 H), 2.37 (s, 3 H), 2.14-2.01 (m, 2 H), | 3.09 min Method 2 |
| | | | 1.93-1.88 (m, 2 H) | |
| 121 | 0 | 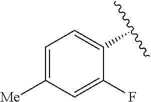 | ¹H NMR (400 MHz, CDCl₃): δ 7.78 (s, 1 H), 7.30-7.26 (m, 1 H), 7.01 (d, J = 8.0 Hz, 1 H), 6.95 (d, J= 12.0 Hz, 1 H), 5.37-5.33 (m, 1 H), 5.02 (s, 1H), 4.57 (dd,J1 = 8.8 Hz, J2 = 11.6 Hz, 1 H), 4.11-4.07 (m, 3 H), 4.01 (dd, J1 = 6.4 Hz, J2 = 11.2 Hz, 1 H), 3.61-3.55 (m, 1 H), 3.36-3.28 (m, 1 H), 2.38 (s, 3 H), 2.15-2.02 (m, 2 H), 1.93- | 4.98 min Method 2 |
| | | | 1.89 (m, 2H) | |
| 122 | 1 | 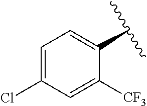 | ¹H NMR (400 MHz, CDCl₃): δ 7.83 (s, 1 H), 7.82-7.29 (m, 3 H), 5.08 (s, 1 H), 5.04 (s, 1 H), 4.44-4.41 (m, 1 H), 4.09 (s, 2 H), 3.84-3.78 (m, 1 H), 3.60-3.53 (m, 2 H), 3.34-3.29 (m, 1 H), 2.47- 2.45 (m, 1 H), 2.14-2.00 (m, 3 H), 1.93-1.89 (m, 2 H) | 7.00 min Method 15 |
| 123 | 1 | 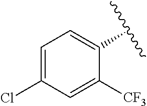 | ¹H NMR (400 MHz, CDCl₃): δ 7.83 (s, 1 H), 7.82-7.29 (m, 3 H), 5.08 (s, 1 H), 5.04 (s, 1 H), 4.44-4.41 (m, 1 H), 4.09 (s, 2 H), 3.84-3.78 (m, 1 H), 160-3.53 (m, 2 H), 3.34-3.29 (m, 1 H), 2.47- 2.45 (m, 1 H), 2.14-2.00 (m, 3 H), 1.93-1.89 (m, 2 H) | 6.07 min Method 15 |
| 124 | 1 | 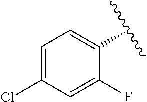 | ¹H NMR (400 MHz, CDCl₃): δ 7.81 (s, 1 H), 7.36-7.40 (m, 1 H), 7.17-7.24 (m, 2 H), 5.03- 5.08 (m, 2 H), 4.00-4.11 (m, 4 H), 3.55-3.61 (m, 2 H), 3.32-3.34 (m, 1 H), 2.39-2.42 (m, 1 H), 2.05-2.16 (m, 3 H), 1.89-1.93 (m, 2 H) | 2.04 min Method 11 |
| 125 | 1 | 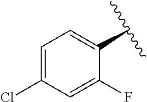 | ¹H NMR (400 MHz, CDCl₃): δ 7.81 (s, 1 H), 7.36-7.40 (m, 1 H), 7.17-7.24 (m, 2 H), 5.03- 5.07 (m, 2 H), 4.01-4.11 (m, 4 H), 3.55-3.61 (m, 2 H), 3.32-3.34 (m, 1 H), 2.40 (m, 1 H), 2.05- 2.16 (m, 3 H), 1.89-1.93 (m, 2 H) | 2.83 min Method 11 |
| 126 | 1 | 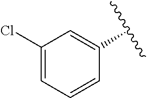 | ¹H NMR (400 MHz, CDCl₃): δ 7.81 (s, 1 H), 7.39-7.37 (m, 3 H), 7.29-7.26 (m, 1 H), 5.13 (s, 1 H), 4.69-4.28 (m, 1 H), 4.27-4.22 (m, 1 H), 4.12- 4.07 (m, 2 H), 3.90-3.83 (m, 1 H), 3.62-3.55 (m, 2 H), 3.36-3.30 (m, 1 H), 2.43-2.38 (m, 1 H), 2.15-2.06 (m, 3 H), 1.95-1.89 (m, 2 H) | 2.49 min Method 10 |
| 127 | 1 | 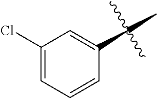 | ¹H NMR (400 MHz, CDCl₃): δ 7.81 (s, 1 H), 7.39-7.37 (m, 3 H), 7.29-7.26 (m, 1 H), 5.13 (s, 1 H), 4.69-4.28 (m, 1 H), 4.27-4.22 (m, 1 H), 4.12- 4.07 (m, 2 H), 3.90-3.83 (m, 1 H), 3.62-3.55 (m, 2 H), 3.36-3.30 (m, 1 H), 2.43-2.38 (m, 1 H), 2.15-2.06 (m, 3 H), 1.95-1.89 (m, 2 H) | 4.54 min Method 10 |
| 128 | 1 | 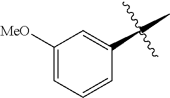 | ¹H NMR (400 MHz, CDCl₃): δ 7.81 (s, 1 H), 7.38-7.34 (m, 1 H), 6.98-6.96 (m, 1 H), 6.93- 6.91 (m, 2 H), 5.08 (s, 1 H), 4.69-4.66 (m, 1 H), 4.31-4.26 (m, 1 H), 4.13-4.09 (m, 2 H), 3.88- 3.82 (m, 4 H), 3.62-3.55 (m, 2 H), 3.34-3.30 (m, 1 H), 2.38-2.37 (m, 1 H), 2.18-2.06 (m, 3 H), 1.95-1.89 (m, 2 H) | 4.84 min Method 10 |
| 129 | 1 | 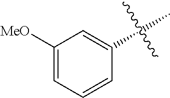 | ¹H NMR (400 MHz, CDCl₃): δ 7.81 (s, 1 H), 7.38-7.34 (m, 1 H), 6.98-6.96 (m, 1 H), 6.93- 6.91 (m, 2 H), 5.08 (s, 1 H), 4.69-4.66 (m, 1 H), 4.31-4.26 (m, 1 H), 4.13-4.09 (m, 2 H), 3.88- 3.82 (m, 4 H), 3.62-3.55 (m, 2 H), 3.34-3.30 (m, 1 H), 2.38-2.37 (m, 1 H), 2.18-2.06 (m, 3 H), 1.95-1.89 (m, 2 H) | 2.51 min Method 10 |

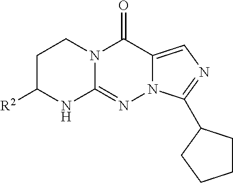

| | | | Separation: |
|---|---|---|---|
| Ex. | | | retention time |
| No. | R²— | ¹H NMR | method |
| 130 | 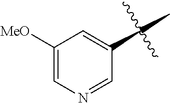 | ¹H NMR (400 MHz, CDCl₃): δ 8.26-8.33 (m, 2 H), 7.80 (s, 1 H), 7.21-7.22 (m, 1 H), 5.04 (s, 1 H), 4.72- 4.75 (m, 1 H), 4.28-4.32 (m, 1 H), 3.82-3.91 (m, 4 H), 3.42-3.46 (m, 1 H), 2.39-2.40 (m, 1 H), 2.06-2.18 (m, 3 H), 1.84-1.96 (m, 4 H), 1.67-1.70 (m, 2 H) | 2.45 min Method 14 |
| 131 | 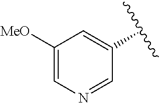 | ¹H NMR (400 MHz, CDCl₃): δ 8.26-8.33 (m, 2 H), 7.79 (s, 1 H), 7.21-7.22 (m, 1 H), 5.05 (s, 1 H), 4.73 4.74 (m, 1 H), 4.28 (m, 1 H), 3.85-3.91 (m, 4 H), 3.42-3.46 (m, 1 H), 2.39-2.40 (m, 1 H), 2.06-2.12 (m, 3 H), 1.84-1.93 (m, 4 H), 1.67-1.70 (m, 2 H) | 4.81 min Method 14 |
| 132 | 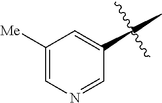 | ¹H NMR (400 MHz, CDCL₃): δ 8.44-8.46 (m, 2 H), 7.79 (s, 1 H), 7.53 (s, 1 H), 5.06 (s, 1 H), 4.70-4.71 (m, 1 H), 4.28 (d, J = 0.8 Hz, 1 H), 3.82-3.86 (m, 1 H), 3.42-3.46 (m, 1 H), 2.37-2.41 (m, 4 H), 2.05-2.10 (m, 3 H), 1.84-1.88 (m, 4H), 1.67-1.69 (m, 2 H) | 5.27 min Method 2 |
| 133 | 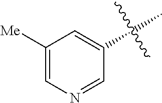 | ¹H NMR (400 MHz, CDCl₃): δ 8.44-8.46 (m, 2 H), 7.79 (s, 1 H), 7.53 (s, 1 H), 5.10 (s, 1 H), 4.69-4.72 (m, 1 H), 4.27-4.31 (m, 1 H), 3.84-3.86 (m, 1 H), 3.42-3.46 (m, 1 H), 2.37-2.39 (m, 4 H), 2.04-2.11 (m, 3 H), 1.84-1.96 (m, 4 H), 1.66-1.73 (m, 2 H | 4.00 min Method 2 |
| 134 | 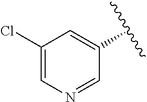 | ¹H NMR (400 MHz, CDCl₃): δ 1.69-1.73 (m, 2 H), 1.84-2.00 (m, 4 H), 2.05-2.17 (m, 3 H), 2.42-2.47 (m, 1 H), 3.42-3.50 (m, 1 H), 3.87-3.94 (m, 1 H), 4.23- 4.29 (m, 1 H). 4.76-4.79 (m, 1 H), 5.13 (s, 1 H), 7.77 (t, J = 2.0 Hz, 1 H), 7.81 (s, 1 H), 8.55 (t, J = 1.6 Hz, 1 H), 8.62 (t, J = 2.4 Hz, 1 H) | 2.56 min Method 10 |
| 135 | 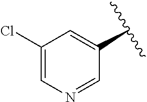 | ¹H NMR (400 MHz, CDCl3): δ 1.69-1.75 (m, 2 H), 1.87-2.02 (m, 4 H), 2.08-2.17 (m, 3 H), 2.41-2.48 (m, 1 H), 3.42-3.50 (m, 1 H), 3.87-3.94 (m, 1 H), 4.23- 4.29 (m, 1 H), 4.76-4.79 (m, 1 H), 5.12 (s, 1 H), 7.77 (t, J = 2.0 Hz, 1 H), 7.81 (s, 1 H), 8.55 (t, J = 1.6 Hz, 1 H), 8.62 (t, J = 2.4 Hz, 1 H) | 5.56 min Method 10 |

### Example 136

### (2R)-2-(4-Chlorophenyl)-8-isopropyl-2,10-dihydro-3H,5H-diimidazo[2,1-c:5′,1′-f][1,2,4]triazin-5-one

### Example 137

### (2S)-2-(4-Chlorophenyl)-8-isopropyl-2,10-dihydro-3H,5H-diimidazo[2,1-c:5′,1′-f][1,2,4]triazin-5-one

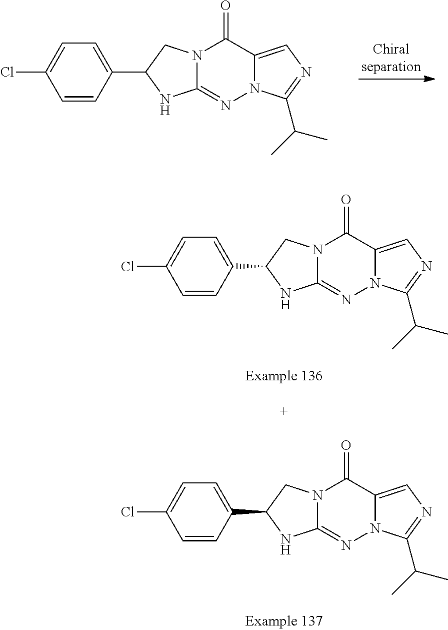 [SMILES: CC(C)c1ncc2c(=O)n3c(nn12)NC(c1ccc(Cl)cc1)C3 ; CC(C)c1ncc2c(=O)n3c(nn12)N[C@H](c1ccc(Cl)cc1)C3 ; CC(C)c1ncc2c(=O)n3c(nn12)N[C@@H](c1ccc(Cl)cc1)C3]

2-(4-chlorophenyl)-8-isopropyl-2,10-dihydro-3H,5H-diimidazo[2,1-c:5′,1′-f][1,2,4]triazin-5-one ((Example 78, 50 mg) was separated to afford titled compounds (Example 136: 10 mg, 20%; Example 137: 10 mg, 20%) as white solids.

Example 136: ¹H NMR (400 MHz, CDCl₃): δ 7.79 (s, 1H), 7.43 (m, 2H), 7.35 (d, J=8.8 Hz, 2H), 5.11-5.16 (m, 2H), 4.54-4.59 (m, 1H), 3.87-3.92 (m, 1H), 3.36-3.42 (m, 1H), 1.38-1.41 (m, 6H). Chiral separation: retention time 2.98 min (Method 24)

Example 137: ¹H NMR (400 MHz, CDCl₃): δ 7.79 (s, 1H), 7.43 (d, J=8.4 Hz, 2H), 7.35-7.37 (d, J=8.4, 2H), 5.11-5.15 (m, 2H), 4.54-4.59 (m, 1H), 3.87-3.92 (m, 1H), 3.39-3.41 (m, 1H), 1.38-1.41 (m, 6H), Chiral separation: retention time 4.31 min (Method 24)

The compounds of Example 138 to 150 were synthesized in a similar manner to Example 136 and 137.

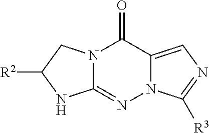

| | | | | Separation: |
|---|---|---|---|---|
| Ex. | | | | retention time |
| No. | R²— | R³— | ¹H NMR | method |
| 138 | 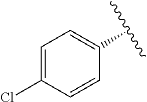 | 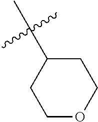 [SMILES: *C1CCOCC1] | ¹H NMR (400 MHz, CDCl₃): δ 7.80 (s, 1 H), 7.45-7.43 (m, 2 H), 7.39 (t, J = 11.2 Hz, 2 H), 5.13 (t, J = 8.2 Hz, 1 H), 4.58 (t, J = 10 Hz, 1 H), 4.13-4.09 (m, 2 H), 3.93-3.89 (m, 1 H), 3.63-3.57 (m, 1 H), 3.37-3.33 (m, 2 H), 3.31 (s, 1 H), 2.13- 2.07 (m, 2 H), 1.95-1.91 (m, 2 H) | 3.01 min Method 11 |
| 139 | 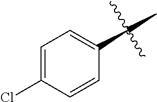 | 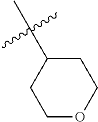 [SMILES: *C1CCOCC1] | ¹H NMR (400 MHz, CDCl₃): δ 7.80 (s, 1 H), 7.45-7.43 (m, 2 H), 7.39 (t, J = 11.2 Hz, 2 H), 5.12 (t, J = 8.2 Hz, 1 H), 4.57 (t, J = 10 Hz, 1 H), 4.13-4.09 (m, 2 H), 3.93-3.89 (m, 1 H), 3.63-3.57 (m, 1 H), 3.37-3.33 (m, 2 H), 3.31 (s, 1 H), 2.13- 2.07 (m, 2 H), 1.95-1.91 (m, 2 H) | 4.62 min Method 11 |
| 140 | 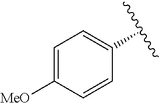 | 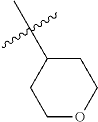 [SMILES: *C1CCOCC1] | ¹H NMR (400 MHz, CDCl₃): δ 7.79 (s, 1 H), 7.30-7.33 (m, 2 H), 6.94-6.69 (m, 2 H), 5.17 (s, 1 H), 5.08 (t, J = 8.42 Hz, 1H), 4.50-4.55 (m, 1 H), 4.08-4.11 (m, 2 H), 3.91-3.94 (m, 1 H), 3.84 (s, 3 H), 3.56- 3.62 (m, 2 H), 3.33 (m, 1 H), 2.06-2.12 (m, 2 H), 1.90-1.95 (m, 2 H) | 2.87 min Method 10 |
| 141 | 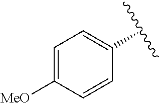 | 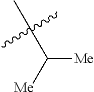 [SMILES: *C(C)C] | ¹H NMR (400 MHz, CDCl₃): δ 7.78 (s, 1 H), 7.31-7.33 (m, 2 H), 6.94-6.96 (m, 2 H), 5.05-5.09 (m, 2 H), 4.50-4.55 (m, 1 H), 3.89-3.93 (m, 1 H), 3.84 (s, 3 H), 3.37-3.41 (m, 1 H), 1.37-1.41 (m, 6 H) | 2.20 min Method 10 |
| 142 | 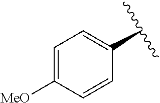 | 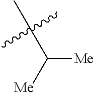 [SMILES: *C(C)C] | ¹H NMR (400 MHz, CDCl₃): δ 7.78 (s, 1 H), 7.31-7.33 (m, 2 H), 6.94-6.96 (m, 2 H), 5.05-5.09 (m, 2 H), 4.50-4.55 (m, 1 H), 3.89-3.93 (m, 1 H), 3.84 (s, 3 H), 3.37-3.41 (m, 1 H), 1.37-1.41 (m, 6 H) | 3.59 min Method 10 |

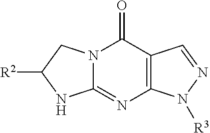

| | | | | Separation: |
|---|---|---|---|---|
| Ex. | | | | retention time |
| No. | R²— | R³— | ¹H NMR | method |
| 143 | 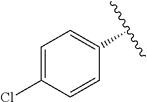 | 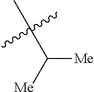 [SMILES: *C(C)C] | ¹H NMR (400 MHz, CDCl₃): δ 7.93 (s, 1 H), 7.38-7.41 (m, 2 H), 7.31-7.34 (m, 2 H), 5.62 (s, 1 H), 5.11-5.16 (m, 1 H), 4.74-4.81 (m, 1 H), 4.56-4.61 (m, 1 H), 3.92-3.97 (m, 1 H), 1.48-1.50 (m, 6 H) | 2.23 min Method 10 |
| 144 | 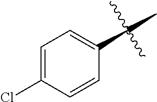 | 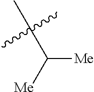 [SMILES: *C(C)C] | ¹H NMR (400 MHz, CDCl₃): δ 7.93 (s, 1 H), 7.38-7.41 (m, 2 H), 7.31-7.34 (m, 2 H), 5.62 (s, 1 H), 5.11-5.16 (m, 1 H), 4.74-4.81 (m, 1 H), 4.56-4.61 (m, 1 H), 3.92-3.97 (m, 1 H), 1.48-1.50 (m, 6 H) | 3.42 min Method 10 |
| 145 | 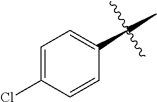 | 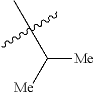 [SMILES: *C(C)C] | ¹H NMR (400 MHz, CDCl₃): δ 7.95 (s, 1 H), 7.32-7.31 (m ,2 H), 6.95-6.93 (m, 2 H), 5.60 (s, 1 H), 5.12 (t, J = 8.4 Hz, 1 H), 4.82-4.77 (m, 2 H), 4.59-4.54 (m, 2 H), 4.01-3.96 (m, 1 H), 3.84 (s, 3 H), 1.51-1.49 (m, 6 H) | 2.11 min Method 16 |
| 146 | 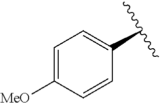 | 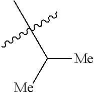 [SMILES: *C(C)C] | ¹H NMR (400 MHz, CDCl₃): δ 7.95 (s, 1 H), 7.32-7.31 (m, 2 H) 6.95-6.93 (m, 2 H), 5.60 (s, 1H), 5.12 (t, J = 8.4 Hz, 1 H), 4.82-4.77 (m, 2 H), 4.59-4.54 (m, 2 H), 4.01-3.96 (m, 1 H), 3.84 (s, 3 H), 1.51- 1.49 (m, 6 H) | 3.97 min Method 16 |
| 147 | 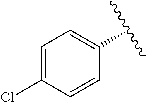 | 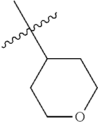 [SMILES: *C1CCOCC1] | ¹H NMR (400 MHz, CDCl₃): δ 7.96 (s, 1 H), 7.42 (d, J = 10.4 Hz, 2 H) 7.34 (d, J = 10.4 Hz, 2 H), 5.49 (s, 1 H), 5.16 (t, J = 8.0 Hz, 1 H), 4.65-4.58 (m, 2 H), 4.15 (d, J = 9.2 Hz, 2 H), 4.00-3.96 (m, 1 H), 3.59 (t, J = 12.4 Hz, 2 H), 2.39-2.36 (m, 2 H), 1.93-1.89 (m, 2 H) | 2.75 min Method 12 |
| 148 | 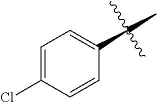 | 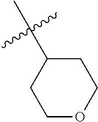 [SMILES: *C1CCOCC1] | ¹H NMR (400 MHz, CDCl₃): δ 7.96 (s, 1 H), 7.43-7.41 (d, J = 10.4 Hz, 2 H) 7.34 (d, J = 10.4 Hz, 2 H), 5.49 (s, 1 H), 5.16 (t, J = 8.0 Hz, 1 H), 4.65-4 58 (m, 2 H), 4.15 (d, J = 9.2 Hz, 2 H), 4.00-3.96 (m, 1 H), 3.59 (t, J = 12.4 Hz, 2 H), 2.39-2.36 (m, 2 H), 1.93-1.89 (m, 2 H) | 4.47 min Method 12 |
| 149 | 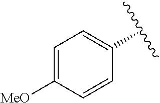 | 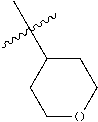 [SMILES: *C1CCOCC1] | ¹H NMR (400 MHz, CDCl₃): δ 7.95 (s, 1 H), 7.32-7.31 (m, 2 H), 6.95-6.93 (m, 2 H), 5.60 (s, 1 H), 5.12 (t, J = 8.4 Hz, 1 H), 4.82-4.77 (m, 1 H), 4.59-4.54 (m, 1 H), 4.01-3.96 (m, 1 H), 3.84 (s, 3 H), 1.51-1.49 (m, 6 H) | 4.83 min Method 10 |
| 150 | 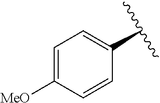 | 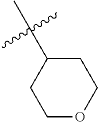 [SMILES: *C1CCOCC1] | ¹H NMR (400 MHz, CDCl₃): δ 7.95 (s, 1 H), 7.32-7.31 (m, 2 H) 6.95-6.93 (m, 2 H), 5.60 (s, 1H), 5.12 (t, J = 8.4 (Hz, 1 H), 4.82-4.77 (m, 1 H), 4.59-4.54 (m, 1 H), 4.01-3.96 (m, 1 H), 3.84 (s, 3 H), 1.51- 1.49 (m, 6 H) | 6.31 min Method 10 |

### Example 151

### (2S)-2-(4-Methoxyphenyl)-8-(tetrahydro-2H-pyran-4-yl)-2,10-dihydro-3H,5H-diimidazo[2,1-c:5′,1′-f][1,2,4]triazin-5-one

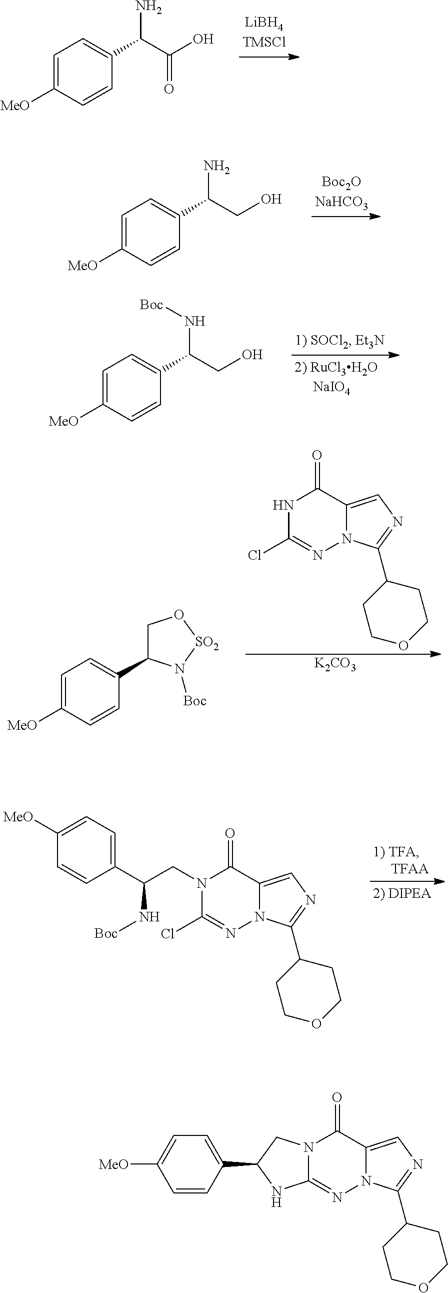

A mixture of 2-methyl-2-propanyl[(1S)-2-[2-chloro-4-oxo-7-(tetrahydro-2H-pyran-4-yl)imidazo[5,1-f][1,2,4]triazin-3(4H)-yl]-1-(4-methoxyphenyl)ethyl]carbamate (1.26 g, 2.50 mmol), TFA (1.90 mL, 25.0 mmol), and TFAA (17 pt, 0.125 mL) in DCM (8.30 mL) was stirred at rt for 4 h. The resulting mixture was concentrated in vacuo. To an ice-cooled mixture of the above residue in THF (8.30 mL) was added TEA (3.50 mL, 25.0 mmol), and the reaction mixture was stirred at rt overnight. The resulting mixture was diluted with water, and extracted with EtOAc. The combined organic extracts were dried over sodium sulfate, filtered, and concentrated in vacuo. The solid was washed with EtOAc to give the titled compound (666 mg) as white solids. ¹H NMR (CDCl₃) δ: 7.74 (s, 1H), 7.29-7.26 (m, 2H), 6.93-6.90 (m, 2H), 5.17 (s, 1H), 5.04 (t, J=8.0 Hz, 1H), 4.48 (dd, J=11.3, 8.7 Hz, 1H), 4.08-4.02 (m, 2H), 3.90-3.85 (1H), 3.80 (s, 3H), 3.58-3.52 (m, 2H), 3.33-3.25 (m, 1H), 2.11-1.98 (m, 2H), 1.91-1.85 (m, 2H).

### (2S)-2-Amino-2-(4-methoxyphenyl)ethanol

To an ice-cooled mixture of lithium borohydride (16.5 g, 759 mmol) in THF (270 mL) was added trimethylsilyl chloride (194 mL, 1.52 mol). After stirring for 30 minutes, a mixture of (2S)-2-amino-2-(4-methoxyphenyl)acetic acid (45.9 g, 253 mmol) in THF (1.00 L) was added dropwise to the reaction. Then the reaction mixture was stirred at rt overnight. The reaction was quenched with MeOH, and the resulting mixture was concentrated in vacuo. The residue was diluted with 1M sodium hydroxide aq. solution and chloroform, and filtered through a pad of Celite. The filtrate was diluted with brine, and organic layer was separated. The aqueous layer was extracted with chloroform. The combined organic extracts were dried over sodium sulfate, filtered, and concentrated in vacuo to give the titled compound (37.1 g) as red solids. NMR (CDCl₃) δ: 7.26-7.24 (m, 3H), 6.89 (d, J=8.3 Hz, 2H), 4.02-3.99 (m, 1H), 3.80 (s, 3H), 3.73-3.69 (m, 1H), 3.55-3.50 (m, 1H), 1.72 (br s, 3H).

### 2-Methyl-2-propanyl[(1S)-2-hydroxy-1-(4-methoxyphenyl)ethyl]carbamate

To a mixture of (2S)-2-amino-2-(4-methoxyphenyl)ethanol (37.1 g, 222 mmol) and sodium carbonate (24.7 g, 233 mmmol) in THF-water (750 mL, 2:1) was added Boc₂O, and the reaction mixture was stirred at rt for 12 h. The resulting mixture was filtered through a pad of Celite, and washed with EtOAc. The filtrate was extracted with EtOAc. The combined organic extracts were dried over sodium sulfate, filtered, and concentrated in vacuo to give the crude, which was used directly for next step without further purification.

### 2-Methyl-2-propanyl(4S)-4-(4-methoxyphenyl)-1,2,3-oxathiazolidine-3-carboxylate 2,2-dioxide

To a mixture of 2-methyl-2-propanyl[(1S)-2-hydroxy-1-(4-methoxyphenyl)ethyl]carbamate (60.9 g, 228 mmol) and TEA (95 mL, 684 mmol) in DCM (1.5 L) was added the mixture of thionyl chloride (20 mL, 274 mmol) in DCM (100 mL) at −40° C., and the reaction mixture was stirred at −40° C. for 1 h. The reaction was quenched with water, and extracted with chloroform. The combined organic extracts were dried over sodium sulfate, filtered, and concentrated in vacuo. To an ice-cooled mixture of the above residue and ruthenium chloride n-hydrate (473 mg, 2.28 mmol) in MeCN-water (1.14 L, 2:1) was added sodium periodate (73.2 g, 342 mmol) stirred at rt for 3 h. The resulting mixture was concentrated in vacuo. The residue was diluted with water and extracted with EtOAc. The combined organic extracts were washed with brine, dried over sodium sulfate, filtered, and concentrated in vacuo. The solid was washed with chloroform, diisopropylether, and MeOH to give the titled compound (24.4 g) as white solids. ¹H NMR (400 MHz, CDCl₃) δ: 7.36-7.33 (m, 2H), 6.95-6.91 (m, 2H), 5.25-5.23 (m, 1H), 4.86-4.82 (m, 1H), 4.41-4.38 (m, 1H), 3.82 (s, 3H), 1.44 (s, 9H).

### 2-Methyl-2-propanyl[(1S)-2-[2-chloro-4-oxo-7-(tetrahydro-2H-pyran-4-yl)imidazo[5,1-f][1,2,4]triazin-3(4H)-yl]-1-(4-methoxyphenyl)ethyl]carbamate

A mixture of 2-methyl-2-propanyl(4S)-4-(4-methoxyphenyl)-1,2,3-oxathiazolidine-3-carboxylate 2,2-dioxide (24.3 g, 73.6 mmol), 2-chloro-7-(tetrahydro-2H-pyran-4-yl)imidazo[5,1-f][1,2,4]triazin-4(1H)-one (Reference Example 2, 17.0 g, 66.9 mmol), and potassium carbonate (10.2 g, 73.6 mmol) in MeCN (335 mL) was stirred at 50° C. overnight. The reaction was quenched with 1M hydrochloric acid. After stirring for 1 h at rt, saturated sodium bicarbonate aq. solution was added to the ice-cooled mixture. The precipitate was collected. The solid was purified by silica gel chromatography (hexane:EtOAc) to give the compound (13.1 g) as white solids. ¹H NMR (400 MHz, CDCl₃) δ: 7.87 (s, 1H), 7.32-7.30 (m, 2H), 6.95-6.92 (m, 2H), 5.20-5.14 (m, 1H), 5.03 (d, =8.5 Hz, 1H), 4.81-4.74 (m, 1H), 4.13-4.08 (m, 3H), 3.82 (s, 3H), 3.64-3.57 (m, 2H), 3.48-3.40 (m, 1H), 2.15-2.00 (m, 2H), 1.93-1.87 (m, 2H), 1.16 (s, 9H).

### Example 152

### (6R)-6-(4-Chlorophenyl)-3-isopropyl-4,6,7,8-tetrahydro-10H-[1,2]oxazolo[4,3-d]pyrimido[1,2-a]pyrimidin-10-one

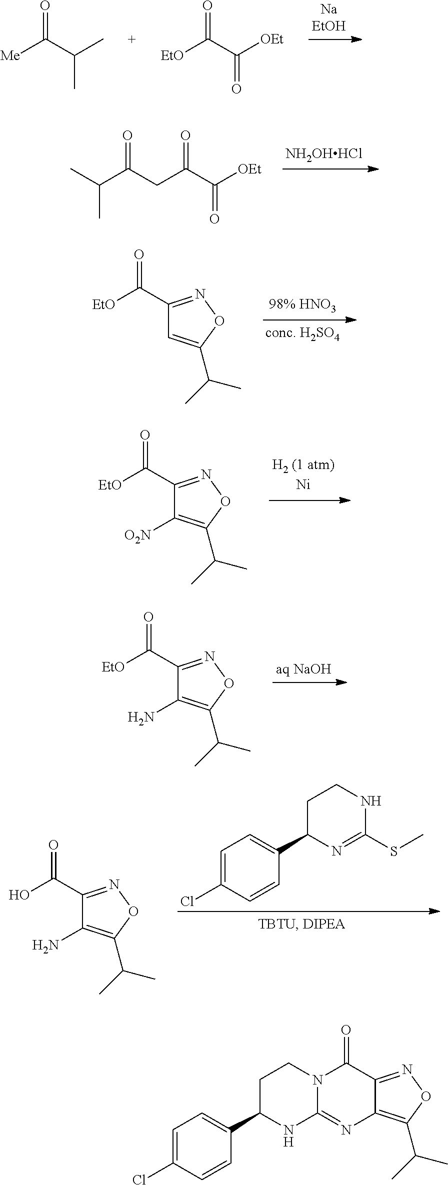

To a solution of 4-amino-5-isopropyl-1,2-oxazole-3-carboxylic acid (154 mg, 0.90 mmol) and (4R)-4-(4-chlorophenyl)-2-(methylthio)-1,4,5,6-tetrahydropyrimidine (Reference Example 7, 144 mg, 0.60 mmol) in DMF (2 mL), were added TBTU (289 mg, 0.9 mmol) and DIPEA (233 mg, 1.8 mmol). The mixture was stirred at rt for 15 h. The crude product was purified by reverse phase column chromatography eluting with MeCN:water=45:55 to give the titled compound (40 mg, 20%) as yellow solids. ¹H NMR (400 MHz, CDCl₃): 7.40-7.37 (m, 2H), 7.33-7.30 (m, 2H), 5.34 (br s, 1H), 4.69 (dd, J|=8.4 Hz, J2=4.4 Hz, 1H), 4.34-4.28 (m, 1H), 3.95-3.88 (m, 1H), 3.44-3.37 (m, 1H), 2.40-2.34 (m, 1H), 2.07-2.02 (m, 1H), 1.46-1.42 (m, 6H).

### Ethyl 5-methyl-2,4-dioxohexanoate

Sodium (6.7 g, 291 mmol) was added to EtOH (250 mL) at 0° C., and 3-methylbutan-2-one (25.0 g, 290 mmol) and diethyl oxalate (42.5 g, 291 mmol) were added. The resulting solid was allowed to stand for 1 h and then heated to 80° C. for 1 h. The reaction was cooled to rt and acidified to pH 2 with dilute sulfuric acid. Water (250 mL) was added and the product extracted with EtOAc (2×250 mL), dried over sodium sulfate, and the solvent was removed to give the titled compound (55 g, 100%) as an orange oil. LCMS: m/z=187 [M+H]⁺.

### Ethyl 5-isopropyl-1,2-oxazole-3-carboxylate

To a solution of ethyl 5-methyl-2,4-dioxohexanoate (55 g, 295 mmol) in EtOH (500 mL), was added hydroxylamine hydrochloride (61.1 g, 885 mmol). The reaction was stirred at rt for 1 h and then heated to reflux for 1 h. The mixture was cooled and concentrated. Water (250 mL) was added to the reaction vessel, extracted with EtOAc (3×250 mL) and dried over sodium sulfate. The solvent was removed to give the titled compound (50 g, 93%) as an orange oil. LCMS: m/z=184 [M+H]⁺.

### Ethyl 5-isopropyl-4-nitro-1,2-oxazole-3-carboxylate

To a solution of ethyl 5-isopropyl-1,2-oxazole-3-carboxylate (10 g, 0.055 mol) in concentrated sulfuric acid (50 mL), was added 98% nitric acid (17.3 g, 0.275 mol) dropwise at 0° C. After stirred at rt for 8 h, the mixture was poured over ice and extracted with chloroform (2×100 mL). The combined organic phases were dried over sodium sulfate, and concentrated to dryness. The residue was purified by chromatography on silica gel (EtOAc:PE=1:10 to 1:6) to give the titled compound (8.0 g, 64%) as an orange oil. LCMS: m/z=229 [M+H]⁺.

### Ethyl 4-amino-5-isopropyl-1,2-oxazole-3-carboxylate

To a solution of ethyl 5-isopropyl-4-nitro-1,2-oxazole-3-carboxylate (8.0 g, 35.1 mmol) in MeOH (30 mL), was added Raney nickel (800 mg). The mixture was stirred at rt under H₂ atmosphere for 2 h. The reaction was filtered, and the filtrate was concentrated and purified by chromatography on silica gel (EtOAc:petroleum ether=10:1 to 3:1) to give the titled compound (6.0 g, 86%) as an orange oil. LCMS: m/z=199 [M+H]⁺.

### 4-Amino-5-isopropyl-1,2-oxazole-3-carboxylic acid

To a solution of ethyl 4-amino-5-isopropyl-1,2-oxazole-3-carboxylate (4.0 g, 20 mmol) in THF (20 mL), was added 3M sodium hydroxide aq. solution (20 mL). The mixture was stirred at rt for 3 h. The reaction mixture was acidified to pH 6 with 3M hydrochloric acid, extracted with EtOAc (3×30 mL) and dried over sodium sulfate. The solvent was removed and the residue was purified by chromatography on silica gel (EtOAc:petroleum ether=1:3 to 1:1) to give the titled compound (1.47 g, 43%) as yellow solids. LCMS: m/z=171 [M+H]⁺.

The compounds of Example 153 to 167 were synthesized in a similar manner to Example 152.

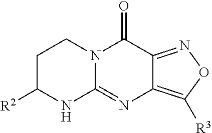

| Ex. | | | |
|---|---|---|---|
| No. | R²— | R³— | ¹H NMR |
| 153 | 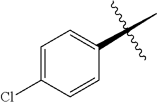 | 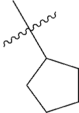 [SMILES: *C1CCCC1] | ¹H NMR (400 MHz, CDCl₃): δ 7.40-7.36 (m, 2H), 7.32- 7.29 (m, 2H), 5.35 (br s, 1H), 4.68 (dd, J1 = 8.4 Hz, J2 = 4.4 Hz, 1H), 4.33-4.27 (m, 1H), 3.95-3.88 (m, 1H), 3.51- 3.43 (m, 1H), 2.40-2.33 (m, 1H), 2.12-1.95 (m, 5H), 1.91- 1.82 (m, 2H), 1.75-1.69 (m, 2H) |
| 154 | 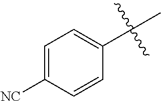 [SMILES: *c1ccc(C#N)cc1] | 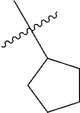 [SMILES: *C1CCCC1] | ¹H NMR (400 MHz, CDCl₃): δ 7.74 (d, J = 8.4 Hz, 2H), 7.53 (d, J = 8.4 Hz, 2H), 5.40 (s, 1H), 4.82 (dd, J1 = 4.8 Hz,. J2 = 7.6 Hz 1H), 4.26-4.20 (m, 1H), 4.05-3.98 (m, 1H) 3.52-3.48 (m, 1H), 2.46-2.41 (m, 1H), 2.12-1.99 (m, 5H), 1.91-1.86 (m, 2H), 1.76-1.70 (m, 2H) |
| 155 | 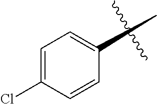 | 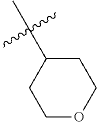 [SMILES: *C1CCOCC1] | ¹H NMR (400 MHz, CDCl₃): δ 7.38-7.41 (m, 2H), 7.31 (d, J = 8 Hz, 2H), 5.33 (s, 1H), 4.68-4.71 (m, 1H), 4.30-4.31 (m, 1H), 4.06 (d, J = 8 Hz, 2H), 3.92-3.94 (m, 1H), 3.53- 3.59 (m, 2H), 3.33-3.38 (m, 1H), 2.35-2.39 (m, 1H), 2.03- 2.16 (m, 3H), 1.93-1.97 (m, 2H) |
| 156 | 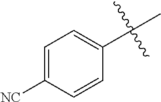 [SMILES: *c1ccc(C#N)cc1] | 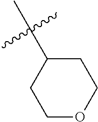 [SMILES: *C1CCOCC1] | ¹H NMR (400 MHz, CDCl₃): δ 7.73-7.74 (m, 2H), 7.52 (d, J = 8 Hz, 2H), 5.41 (s, 1H), 4.80-4.81 (m, 1H), 4.20-4.26 (m, 1H), 4.00-4.08 (m, 3H), 3.54-3.60 (m, 2H), 3.33-3.38 (m, 1H), 2.39-2.45 (m, 1H), 1.94-2.17 (m, 5 H) |
| 157 | 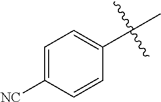 [SMILES: *c1ccc(C#N)cc1] | 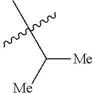 [SMILES: *C(C)C] | ¹H NMR (400 MHz, CDCl₃): δ 7.73-7.71 (m, 2H), 7.52- 7.50 (m, 2H), 5.38 (br s, 1H), 4.78 (dd, J1 = 8.0 Hz, J2 = 4.8 Hz, 1H), 4.25-4.19 (m, 1H), 4.03-3.96 (m, 1H), 3.44- 3.37 (m, 1H), 2.44-2.39 (m, 1H), 2.09-2.05 (m, 1H), 1.44- 1.42 (m, 6H) |
| 158 | 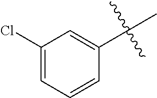 [SMILES: *c1cccc(Cl)c1] | 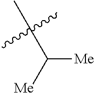 [SMILES: *C(C)C] | ¹H NMR (400 MHz, CDCl₃): δ 7.37 (s, 1H), 7.35-7.32 (m, 2H), 7.26-7.23 (m, 1H), 5.44 (br s, 1H), 4.69 (dd, J1 = 8.0 Hz, J2 = 4.0 Hz, 1H), 4.31-4.25 (m, 1H), 3.97-3.91 (m, 1H), 3.43-3.36 (m, 1H), 2.41-2.34 (m, 1H), 2.11-2.02 (m, 1H), 1.44-1.42 (m, 6H) |
| 159 | 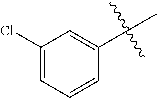 [SMILES: *c1cccc(Cl)c1] | 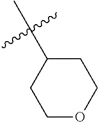 [SMILES: *C1CCOCC1] | ¹H NMR (400 MHz, CDCl₃): δ 7.34-7.36 (m, 3H), 7.24- 7.25 (m, 1H), 5.36 (s, 1H), 4.68-4.70 (m, 1H), 4.25-4.31 (m, 1H), 4.06 (d, J = 12 Hz, 2H), 3.94 (m, 1H), 3.53-3.59 (m, 2H), 3.36 (m, 1H), 2.35-2.37 (m, 1H), 2.08-2.16 (m, 3H), 1.94-1.97 (m, 2H) |

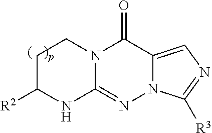

| Ex. | | | | |
|---|---|---|---|---|
| No. | p | R²— | R³— | ¹H NMR |
| 160 | 0 | 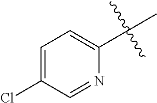 [SMILES: *c1ccc(Cl)cn1] | 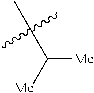 [SMILES: *C(C)C] | ¹H NMR (400 MHz, CDCl₃): δ 8.59 (d, J = 4 Hz, 1H), 7.75-7.78 (m, 2H), 7.37 (d, J = 8 Hz, 1H), 5.54 (s, 1H), 5.19 (t, J = 6.8 Hz, 1H), 4.54-4.59 (m, 1H), 4.19-4.23 (m, 1H), 3.38-3.41 (m, 1H), 1.37-1.40 (m, 6H) |
| 161 | 0 | 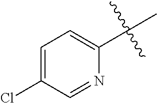 [SMILES: *c1ccc(Cl)cn1] | 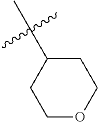 [SMILES: *C1CCOCC1] | ¹H NMR (400 MHz, CDCl₃): δ 8.31 (d, J = 2 Hz, 1H), 7.78-7.76 (m, 2H), 7.38-7.36 (m, 1H), 5.69 (s, 1H), 5.20 (dd, J1 = 5.2 Hz, J2 = 8.4 Hz, 1H), 4.57 (dd, J1 = 9.2 Hz, J2 = 11.6 Hz 1H), 4.22 (dd, J1 = 5.6 Hz, J2 = 11.6 Hz, 1H), 4.01-4.08 (m, 2H), 3.62-3.56 (m, 2H), 3.35-3.33 (m, 1H), 2.11-2.04 (m, 2H), 1.93-1.90 (m, 2H) |
| 162 | 0 | 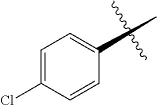 | 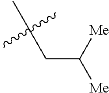 [SMILES: *CC(C)C] | ¹H NMR (400 MHz, CDCl₃): δ 7.76 (s, 1H), 7.40 (d, J = 8.0 Hz, 2H), 7.34 (d, J = 8.0 Hz, 2H), 5.56 (s, 1H), 5.10 (t, J = 10.0 Hz, 1H), 4.52-4.57 (m, 1H), 3.84-3.88 (m, 1H), 2.76 (d, J = 8.0 Hz, 2H), 2.20-2.26 (m, 1H), 0.97 (d, J = 4.0 Hz, 6H) |
| 163 | 0 | 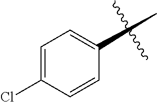 | 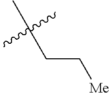 [SMILES: *CCC] | ¹H NMR (400 MHz, CDCl₃): δ 7.78 (s, 1H), 7.44-7.35 (m, 4H), 5.25 (s, 1H), 5.12 (t, J = 8.0 Hz 1H), 4.58 (dd, J1 = 9.2 Hz, J2 = 11.6 Hz 1H), 3.90 (dd, J1 = 7.2 Hz, J2 = 11.2 Hz 1H), 2.88 (t, J = 7.8 Hz, 2H), 1.87-1.82 (m, 2H), 1.02 (t, J = 7.4 Hz, 3H) |
| 164 | 0 | H | 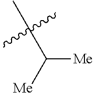 [SMILES: *C(C)C] | ¹H NMR (400 MHz, CDCl₃): δ 1.35-1.37 (d, J = 7.2 Hz, 6H), 3.31-3.38 (m, 1H) 3.78-3.82 (t, J = 8.0 Hz, 2H), 4.17-4.21 (t, J = 8.0 Hz, 2H), 4.64 (s, 1H), 7.75 (s, 1H). |
| 165 | 1 | 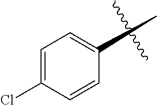 | 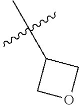 [SMILES: *C1COC1] | ¹H NMR (400 MHz, CDCl₃): δ 7.85 (s, 1H), 7.39-7.43 (m, 2H), 7.30-7.33 (m, 2H), 5.23 (s, 1H), 5.11-5.18 (m, 2H), 4.99-5.03 (m, 2H), 4.65-4.72 (m, 2H), 4.23-4.29 (m, 1H), 3.81-3.88 (m, 1H), 2.36-2.40 (m, 1H), 2.04- 2.08 (m, 1H) |
| 166 | 1 | 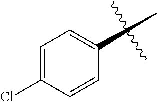 | 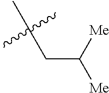 [SMILES: *CC(C)C] | ¹H NMR (400 MHz, DMSO-d₆) δ 7.93 (d, J = 1.0 Hz, 1H), 7.59 (s, 1H), 7.40-7.45 (m, 4H), 4.68-4.71 (m, 1H), 3.88-3.94 (m, 1H), 3.63-3.87 (m, 1H), 2.65-2.75 (m, 2H), 1.98-2.29 (m, 2H), 1.92-1.97 (m, 1H), 0.92 (d, J = 0.8 Hz, 6H) |
| 167 | 1 | 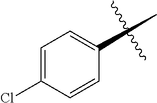 | 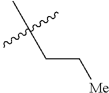 [SMILES: *CCC] | ¹H NMR (400 MHz, CDCl₃): 7.797 (s, 1H), 7.427-7.405 (m, 2H), 7.346-7.284 (m, 2H), 5.076 (s, 1H), 4.702-4.672 (m, 1H), 4.297-4.237 (m, 1H), 3.878-3.809 (m, 1H), 2.86 (t, J = 7.6 Hz. 2H), 2.405-2.359 (m, 1H), 2.104-2.024 (m, 1H), 1.883-1.790 (m, 2H), 1.02 (t, J = 7.4 Hz, 3H) |

The compounds of Example 168 to 171 were synthesized in a similar manner to the Examples herein and Reference Examples 3.

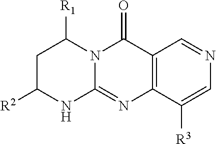

| Ex. | | | | |
|---|---|---|---|---|
| No. | R¹— | R²— | R³— | ¹H NMR |
| 168 | H | 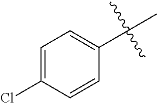 [SMILES: *c1ccc(Cl)cc1] | 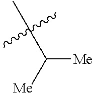 [SMILES: *C(C)C] | ¹H NMR (400 MHz, CDCl₃): δ 9.11 (s, 1H), 8.46 (s, 1H), 7.40 (d, J = 8.4 Hz , 2H), 7.32 (d, J = 8.4 Hz, 2H), 6.24 (br s, 1H), 4.77-4.74 (m, 1H), 4.27-4.22 (m, 1H), 3.98-3.94 (m, 1H), 3.60-3.52 (m, 1H), 2.42-2.38 (m, 1H), 2.12- 2.07 (m, 1H), 1.33-1.29 (m, 6H) |
| 169 | H | 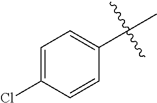 [SMILES: *c1ccc(Cl)cc1] | 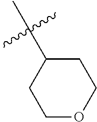 [SMILES: *C1CCOCC1] | ¹H NMR (400 MHz, CDCl₃): δ 9.18 (s, 1H), 8.48 (s, 1H), 7.43 (d, J = 8.8 Hz, 2H), 7.34 (d, J = 8.8 Hz, 2H), 5.84 (s, 1H), 4.79-4.75 (m, 1H), 4.34-4.26 (m, 1H), 4.14-4.06 (m, 2H), 4.00-3.92 (m, 1H), 3.63-3.56 (m, 2H), 3.51- 3.46 (m, 1H), 2.45-2.39 (m, 1H), 2.19-2.07 (m, 1H), 2.04-1.86 (m, 2H), 1.84-1.80 (m, 2H) |
| 170 | 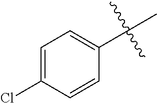 [SMILES: *c1ccc(Cl)cc1] | H | 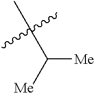 [SMILES: *C(C)C] | ¹H NMR (400 MHz, CDCl₃): δ 9.07 (s, 1H), 8.49 (s, 1H), 7.32 (d, J = 8.4 Hz, 2H), 7.08 (d, J = 8.4 Hz, 2H), 6.11 (br s, 1H), 5.71 (br s, 1H), 3.66-3.59 (m, 1H), 3.46-3.43 (m, 1H), 3.25-3.18 (m, 1H), 2.38-2.23 (m, 2H), 1.35- 1.31 (m, 6H) |
| 171 | 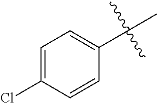 [SMILES: *c1ccc(Cl)cc1] | H | 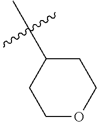 [SMILES: *C1CCOCC1] | ¹H NMR (400 MHz, CDCl₃): δ 9.11 (s, 1H), 8.49 (s, 1H), 7.34 (d, J = 8.4 Hz, 2H), 7.09 (d, J = 8.4 Hz, 2H), 6.14 (s, 1H), 5.76 (br s, 1H), 4.16-4.11 (m, 2H), 3.66-3.60 (m, 2H), 3.55- 3.45 (m, 2H), 3.28-3.21 (m, 1H), 2.41-2.26 (m, 2H), 2.05-1.84 (m, 4H) |

The compounds of Example 172 to 175 were synthesized in a similar manner to the Examples herein and Reference Examples 4.

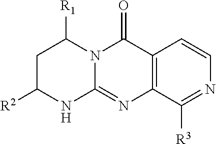

| Ex. | | | | |
|---|---|---|---|---|
| No. | R¹— | R²— | R³— | ¹H NMR |
| 172 | H | 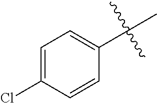 [SMILES: *c1ccc(Cl)cc1] | 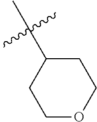 [SMILES: *C1CCOCC1] | ¹H NMR (400 MHz, CDCl₃): δ 8.35 (d, J = 5.2 Hz, 1H), 7.77 (d, J = 5.2 Hz, 1H), 7.43 (d, J = 8.4 Hz, 2H), 7.35 (d, J = 8.4 Hz, 2H), 5.61 (s, 1H), 4.77-4.74 (m, 1H), 4.34-4.27 (m, 1H), 4.15-4.07 (m, 2H), 4.01-3.93 (m, 1H), 3.97-3.88 (m, 1H), 3.68-3.59 (m, 2H), 2.45-2.39 (m, 1H), 2.10-2.00 (m, 3H), 1.82- 1.78 (m, 2H) |
| 173 | H | 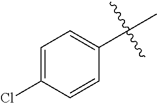 [SMILES: *c1ccc(Cl)cc1] | 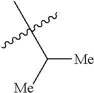 [SMILES: *C(C)C] | ¹H NMR (400 MHz, CDCl₃): δ 8.33 (d, J = 5.2 Hz, 1H), 7.73 (d, J = 5.2 Hz, 1H), 7.40 (d, J = 8.4 Hz, 2H), 7.33 (d, J = 8.4 Hz, 2H), 5.59 (br s, 1H), 4.76-4.72 (m, 1H), 4.32-4,24 (m, 1H), 3.97-3.93 (m, 2H), 2.42-2.37 (m, 1H), 2.13-2.09 (m, 1H), 1.33-1.28 (m, 6H) |
| 174 | 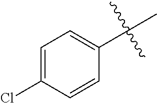 [SMILES: *c1ccc(Cl)cc1] | H | 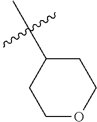 [SMILES: *C1CCOCC1] | ¹H NMR (400 MHz, CDCl₃) δ: 8.30-8.29 (m, 1H), 7.67-7.66 (m, 1H), 7.31-7.29 (m, 2H), 7.06-7.04 (m, 2H), 6.08 (s, 1H), 5.59 (br s, 1H), 4.13-4.08 (m, 2H), 3.88-3.81 (m, 1H), 3.64 (t, J = 11.6 Hz, 2H), 3.45-3.43 (m, 1H), 3.24-3.17 (m, 1H), 2.36-2.22 (m, 2H), 2.16- 1.98 (m, 2H), 1.81-1.78 (m, 2H) |
| 175 | 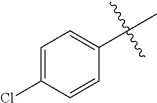 [SMILES: *c1ccc(Cl)cc1] | H | 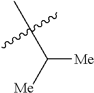 [SMILES: *C(C)C] | ¹H NMR (400 MHz, CDCl₃): δ 8.29 (d, J = 5.6 Hz, 1H), 7.66 (d, J = 5.6 Hz, 1H), 7.32 (d, J = 8.4 Hz, 2H), 7.08 (d, J = 8.4 Hz, 2H), 6.01 (s, 1H), 5.52 (br s, 1H), 4.03-3.96 (m, 1H), 3.45-3.42 (m, 1H), 3.27-3.18 (m, 1H), 2.38-2.23 (m, 2H), 1.36-1.31 (m, 6H) |

The compound of Example 176 was synthesized in a similar manner to the Examples herein and Reference Example 5.

| Ex. | | |
|---|---|---|
| No. | Structure | ¹H NMR |
| 176 | 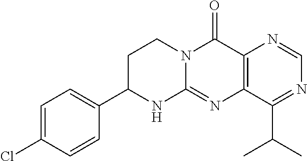 [SMILES: CC(C)c1ncnc2c(=O)n3c(nc12)NC(c1ccc(Cl)cc1)CC3] | ¹H NMR (400 MHz, CDCl₃) δ: 9.00 (s, 1H), 7.41- 7.27 (m, 4H), 5.73 (br s, 1H), 4.77 (br s, 1H), 4.37-4.34 (m, 1H), 4.00-3.90 (m, 2H), 2.40 (br s, 1H), 2.11-2.05 (m, 1H), 1.33-1.91 (m, 6H) |

### Example 177

### 9-(Tetrahydro-2H-pyran-4-yl)-2′,3,3′,4-tetrahydro-1H,6H-spiro[imidazo[5,1-f]pyrimido[2,1-c][1,2,4]triazine-2,1′-inden]-6-one

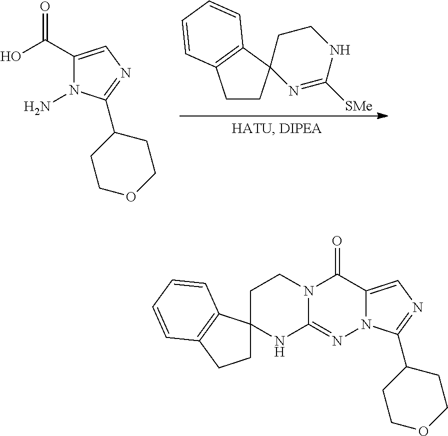 [SMILES: Nn1c(C(=O)O)cnc1C1CCOCC1 ; CSC1=NC2(CCN1)CCc1ccccc12 ; O=c1c2cnc(C3CCOCC3)n2nc2n1CCC1(CCc3ccccc31)N2]

A solution of 2′-(methylthio)-2,3,5′,6′-tetrahydro-1′H-spiro[indene-1,4′-pyrimidine] (Reference Example 8, 76 mg, 0.33 mmol) and 1-amino-2-(tetrahydro-2H-pyran-4-yl)-1H-imidazole-5-carboxylic acid (Reference Example 6, 104 mg, 0.49 mmol) in anhydrous DMF (10 mL) was added HATU (248 mg, 0.65 mmol) and DIPEA (422 mg, 3.3 mmol). The solution was stirred at 25° C. for 16 h. The product was purified through flash column to get the titled compound (4.9 mg, 4%). ¹H NMR (400 MHz, CD₃OD): 1.87-2.44 (m, 8H), 2.97-3.01 (m, 2H), 3.42-3.58 (m, 3H), 3.88-4.05 (m, 3H), 4.25-4.32 (m, 1H), 7.28-7.31 (m, 4H), 7.65 (s, 1H). LCMS: m/z=378 [M+1]⁺.

The compounds of Example 178 to 180 were synthesized in a similar manner to Example 177 and Reference Example 9.

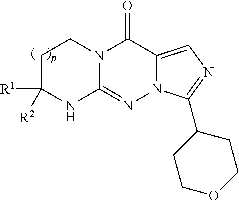

| Ex. | | | |
|---|---|---|---|
| No. | p | R¹ and R²— | ¹H NMR |
| 178 | 0 | 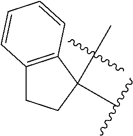 [SMILES: *C1(*)CCc2ccccc21] | ¹H NMR (400 MHz, DMSO-d₆): 1.90-1.93 (m, 2H), 2.03-2.14 (m, 2H), 2.28-2.36 (m, 1H), 2.51-2.57 (m, 1H), 2.98-3.09 (m, 2H), 3.27-3.35 (m, 1H), 3.54-3.60 (m, 2H), 4.06-4.11 (m, 3H), 4.28- 4.31 (m, 1H), 4.92 (s, 1H), 7.30-7.38 (m, 4H), 7.80 (s, 1H) |
| 179 | 0 | 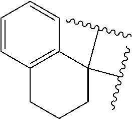 [SMILES: *C1(*)CCCc2ccccc21] | ¹H NMR (400 MHz, CDCl₃) δ 1.90-2.14 (m, 7H), 2.19-2.25 (m, 1H), 2.87-2.90 (m, 2H), 3.28-3.36 (m, 1H), 3.58 (dt, J = 2.2, 11.7 Hz, 2H), 4.05-4.11 (m, 3H), 4.28 (d, J = 11.4 Hz, 1H), 4.90 (s, 1H), 7.15-7.17 (m, 1H), 7.25-7.28 (m, 2H), 7.48-7.53 (m, 1H), 7.79 (s, 1H) |
| 180 | 1 | 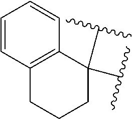 [SMILES: *C1(*)CCCc2ccccc21] | ¹H NMR (400 MHz, DMSO-d₆): δ1.78-1.86 (m, 6H), 1.97-2.16 (m, 4H), 2.75-2.78 (m, 2H), 3.24-3.30 (m, 1H), 3.38-3.44 (m, 2H), 3.77-3.94 (m, 4H), 7.08-7.10 (m, 1H), 7.14-7.17 (m, 2H), 7.34- 7.36 (m, 1H), 7.58 (s, 1H), 7.78 (s, 1H) |

### Example 181

### 2-(6-Chloro-3-pyridinyl)-8-isopropyl-2,10-dihydro-3H,5H-diimidazo[2,1-c:5′,1′-f][1,2,4]triazin-5-one

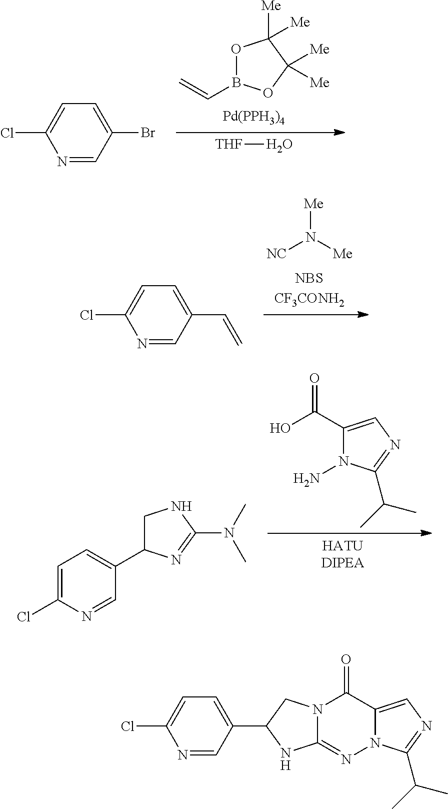

To a solution of 4-(6-chloro-3-pyridinyl)-N,N-dimethyl-4,5-dihydro-1H-imidazol-2-amine (0.11 g, 0.5 mmol) in DMF (1 mL) was added 1-amino-2-isopropyl-1H-imidazole-5-carboxylic acid (synthesized in a similar manner to Reference Example 2 and 6, 0.085 g, 0.5 mmol), HATU (0.38 g, 1.0 mmol) and DIPEA (0.19 g, 1.5 mmol). The reaction was stirred at rt for 16 h. The resulting oil was purified by prep-TLC eluting with PE:EtOAc=20:1 to provide the titled compound (4 mg, 2%) as white solids. ¹H NMR (400 MHz, CDCl₃): δ 8.45 (d, J=2.4 Hz, 1H), 7.78 (s, 1H), 7.76 (dd, J1=8.4, J2=2.4 Hz, 1H), 7.43 (d, J=8.4 Hz, 1H), 5.30 (s, 1H), 5.19-5.15 (m, 1H), 4.62 (dd, J1=11.6 Hz, J2=8.4 Hz, 1H), 3.88 (dd, J1=11.6 Hz, J2=7.6 Hz, 1H), 3.39-3.35 (m, 1H), 1.39-1.36 (m, 6H). LCMS: m/z=435 [M+H]⁺.

### 2-Chloro-5-vinylpyridine

To a solution of 5-bromo-2-chloropyridine (1.92 g, 10 mmol) in THF:H₂O=4:1 (100 mL) were added 4,4,5,5-tetramethyl-2-vinyl-1,3,2-dioxaborolane (2.31 g, 15 mmol), tetrakis(triphenylphosphine)palladium (231 mg, 0.2 mmol), and sodium carbonate (6.9 g, 50 mmol). The mixture was stirred at 75° C. under Ar for overnight. The crude product was purified by silica gel chromatography (eluted with petroleum:DCM=10:1) to give the titled compound (0.87 g, 62%) as an oil. MS: m/e=140 [M+H]⁺.

### 4-(6-Chloro-3-pyridinyl)-N,N-dimethyl-4,5-dihydro-1H-imidazol-2-amine

To a solution of 2-chloro-5-vinylpyridine (0.56 g, 4 mmol) in dimethylcyanamide (8 mL) were added 2,2,2-trifluoroacetamide (0.38 g, 3.4 mmol) and 1-bromo-2,5-pyrrolidinedione (0.71 g, 4 mmol). The reaction was stirred at rt in dark for 16 h. Sat. sodium bicarbonate aq. solution (20 mL) was added to the reaction vessel and the resulting biphasic mixture was transferred to a separatory funnel. The layers were separated and the aqueous phase was washed with DCM (100 mL×2). The combined organics were concentrated in vacuo. The resulting oil was purified by flash column chromatography to provide the titled compound (0.6 g, 67%) as a yellow oil. MS: m/e=225 [M+H]⁺.

The compounds of Example 182 to 184 were synthesized in a similar manner to Example 181.

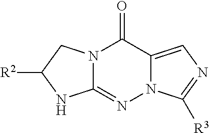

| Ex. | | | |
|---|---|---|---|
| No. | R²— | R³— | ¹H NMR |
| 182 | 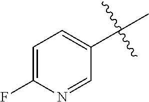 [SMILES: *c1ccc(F)nc1] | 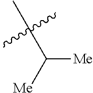 [SMILES: *C(C)C] | ¹H NMR (400 MHz, CDCl₃): δ 8.31 (d, J = 4 Hz, 1H), 7.916-7.920 (m, 1H), 7.80 (s, 1H), 7.05-7.08 (m, 1H), 5.20-5.23 (m, 2H), 4.61-4.66 (m, 1H), 3.89-3.93 (m, 1H), 3.37-3.41 (m, 1H), 1.38-1.40 (m, 6H) |
| 183 | 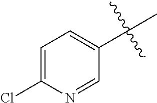 [SMILES: *c1ccc(Cl)nc1] | 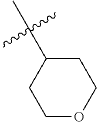 [SMILES: *C1CCOCC1] | ¹H NMR (400 MHz, DMSO-d₆: δ 8.49 (br s, 1H), 8.40 (br s, 1H), 8.00-7.98 (m, 1H), 7.59-7.56 (m, 2H), 5.20 (t, J = 8.0 Hz, 1H), 4.51 (t, J = 10.0 Hz, 1H), 3.94 (d, J = 11.6 Hz, 2H), 3.81-3.77 (m, 1H), 3.49-3.43 (m, 2H), 3.30-3.25 (m, 1H), 1.86-1.81 (m, 4H) |
| 184 | 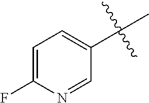 [SMILES: *c1ccc(F)nc1] | 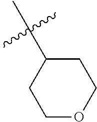 [SMILES: *C1CCOCC1] | ¹H NMR (400 MHz, DMSO-d₆): δ 8.40 (s, 1H), 8.32 (s, 1H), 8.13 (td, J1 = 2.4 Hz, J2 = 8.0 Hz, 1H) 7.59 (s, 1H), 7.25 (dd, J1 = 2.8 Hz, J2 = 8.4 Hz, 1H), 5.21 (t, J = 8.2 Hz, 1H), 4.52 (dd, J1 = 9.2 Hz, J2 = 11.2 Hz, 1H), 3.95 (d, J = 11.6 Hz, 2H), 3.79 (dd, J1 = 7.2 Hz, J2 = 11.2 Hz, 1H), 3.45 (td, J1 = 4.0 Hz, J2 = 11.2 Hz, 2H), 3.30-3.25 (m, 1H), 1.87-1.80 (m, 4H) |

### Example 185

### (2S)-2-(4-Chlorophenyl)-8-[(3S)-tetrahydro-3-furanyl]-2,10-dihydro-3H,5H-diimidazo[2,1-c:5′,1′-f][1,2,4]triazin-5-one

### Example 186

### (2S)-2-(4-Chlorophenyl)-8-[(3R)-tetrahydro-3-furanyl]-2,10-dihydro-3H,5H-diimidazo[2,1-c:5′,1′-f][1,2,4]triazin-5-one

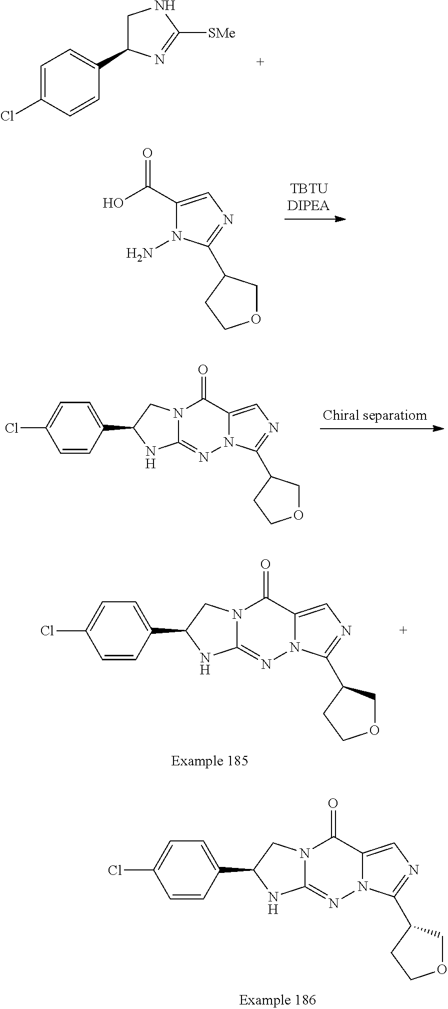

The (2S)-2-(4-chlorophenyl)-8-(tetrahydro-3-furanyl)-2,10-dihydro-3H,5H-diimidazo[2,1-c:5′,1′-f][1,2,4]triazin-5-one (250 mg) was separated by Chiral-HPLC to afford titled compounds (Example 185: 91 mg; Example 186: 106 mg).

Example 185: ¹H NMR (400 MHz, CDCl₃): δ 7.77 (s, 1H), 7.42 (d, J=8.4 Hz, 2H), 7.35 (d, J=8.8 Hz, 2H), 5.67 (s, 1H), 5.12 (t, J=8.2 Hz, 1H), 4.55 (dd, J1=9.2 Hz, J2=11.6 Hz, 1H), 4.24 (t, J=7.4 Hz, 1H), 4.08-3.81 (m, 5H), 2.51-2.31 (m, 2H), Chiral separation: retention time 3.65 min (Method 8).

Example 186: ¹H NMR (400 MHz, CDCl₃): δ 7.77 (s, 1H), 7.42 (d, J=8.4 Hz, 2H), 7.35 (d, J=8.8 Hz, 2H), 5.67 (s, 1H), 5.12 (t, J=8.2 Hz 1H), 4.55 (dd, J1=9.2 Hz, J2=11.6 Hz, 1H), 4.24 (t, J=7.4 Hz, 1H), 4.08-3.81 (m, 5H), 2.51-2.31 (m, 2H), Chiral separation: retention time 4.60 min (Method 8).

### (2S)-2-(4-chlorophenyl)-8-(tetrahydro-3-furanyl)-2,10-dihydro-3H,5H-diimidazo[2,1-c:5′,1′-f][1,2,4]triazin-5-one

To a solution of 1-amino-2-(tetrahydro-3-furanyl)-1H-imidazole-5-carboxylic acid (synthesized in a similar manner to Reference Example 2 and 6, 0.02 g, 0.1 mmol) in DMF (0.2 mL) was added (4S)-4-(4-chlorophenyl)-2-(methylthio)-4,5-dihydro-1H-imidazole (synthesized in a similar manner to Reference Example 7, 0.02 g, 0.1 mmol), TBTU (0.06 g, 0.2 mmol) and DIPEA (0.03 g, 0.2 mmol). The reaction was stirred at rt for 16 h. The resulting mixture was purified by reverse phase HPLC to provide titled compound (0.02 g, 50%) as a white states. MS (ESI): m/e=358 [M+H]⁺.

The compounds of Example 187 to 227 were synthesized in a similar manner to Example 185 and 186.

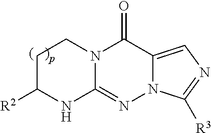

| | | | | | Separation: |
|---|---|---|---|---|---|
| Ex. | | | | | retention time |
| No. | p | R²— | R³— | ¹H NMR | method |
| 187 | 0 | 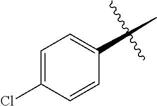 | 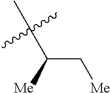 [SMILES: *[C@H](C)CC] | ¹H NMR (400 MHz, CDCl₃): δ 7.80 (s, 1H), 7.41 (d, J = 8.8 Hz, 2H), 7.34 (d, J = 8.4 Hz, 2H), 5.12- 5.06 (m, 2H), 4.55 (dd, J1 = 8.8 Hz, J2 = 11.6 Hz, 1H), 3.87 (dd, J1 = 7.2 Hz, J2 = 11.2 Hz, 1H), 3.24- 3.18 (m, 1H), 1.93-1.86 (m, 1H), 1.73-1.67 (m, 1H), 1.58 (d, J = 11.6 Hz, 3H), 0.88 (t, J = 7.4 Hz, 3H) | 3.02 min Method 21 |
| 188 | 0 | 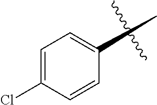 | 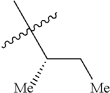 [SMILES: *[C@@H](C)CC] | ¹H NMR (400 MHz, CDCl₃): δ 7.80 (s, 1H), 7.41 (d, J = 8.8 Hz, 2H), 7.34 (d, J = 8.4 Hz, 2H), 5.12- 5.06 (m, 2H), 4.55 (dd, J1 = 8.8 Hz, J2 = 11.6 Hz, 1H), 3.87 (dd, J1 = 7.2 Hz, J2 = 11.2 Hz, 1H), 3.24- 3.18 (m, 1H), 1.93-1.86 (m, 1H), 1.73-1.67 (m, 1H), 1.58 (d, J = 11.6 Hz, 3H), 0.88 (t, J = 7.4 Hz, 3H) | 3.50 min Method 21 |
| 189 | 0 | 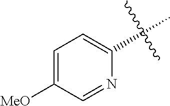 | 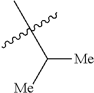 [SMILES: *C(C)C] | ¹H NMR (400 MHz, CDCl₃): δ 8.31 (d, J = 8.4 Hz, 1H), 7.76 (s, 1H) 7.33-7.29 (m, 1H), 7.26-7.23 (m, 1H), 5.51 (s, 1H), 5.16 (dd, J1 = 5.6 Hz, J2 = 8.8 Hz, 1H), 4.55- 4.50 (m, 1H), 4.24-4.20 (m, 1H), 3.89 (s, 3H), 3.41-3.38 (m, 1H), 1.40-1.37 (m, 6H) | 3.61 min Method 22 |
| 190 | 0 | 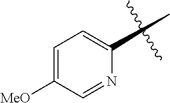 | 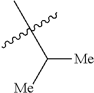 [SMILES: *C(C)C] | ¹H NMR (400 MHz, CDCl₃): δ 8.31 (d, J = 8.4 Hz, 1H), 7.76 (s, 1H) 7.33-7.29 (m, 1H), 7.26-7.23 (m, 1H), 5.51 (s, 1H), 5.16 (dd, J1 = 5.6 Hz, J2 = 8.4 Hz, 1H), 4.55- 4.50 (m, 1H), 4.24-4.20 (m, 1H), 3.89 (s, 3H), 3.41-3.38 (m, 1H), 1.40-1.37 (m, 6H) | 4.53 min Method 22 |
| 191 | 0 | 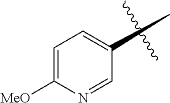 | 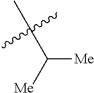 [SMILES: *C(C)C] | ¹H NMR (400 MHz, CDCl₃): δ 8.17 (d, J = 2.0 Hz, 1H), 7.77 (s, 1H), 7.66 (dd, J1 = 9.0 Hz, J2 = 2.6 Hz, 1H), 6.83 (d, J = 8.4 Hz, 1H), 5.43 (br s, 1H), 5.10 (t. J = 8.0 Hz, 1H), 4.58-4.53 (m, 1H), 3.97 (s, 3H), 3.92-3.87 (m, 1H), 3.39-3.36 (m, 1H), 1.39-1.36 (m, 6H) | 3.12 min Method 5 |
| 192 | 0 | 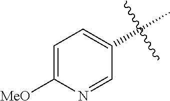 | 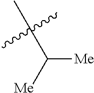 [SMILES: *C(C)C] | ¹H NMR (400 MHz, CDCl₃): δ 8.15 (d, J = 2.4 Hz, 1H), 7.75 (s, 1H), 7.63 (dd, J1 = 8.4 Hz, J2 = 2.4 Hz, 1H), 6.81 (d, J = 8.4 Hz, 1H), 5.45 (br s, 1H), 5.08 (t, J = 8.0 Hz, 1H), 4.55-4.50 (m, 1H), 3.94 (s, 3H), 3.90-3.85 (m, 1 H), 3.37-3.34 (m, 1H), 1.37-1.34 (m, 6H) | 5.91 min Method 5 |
| 193 | 0 | 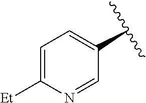 | 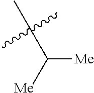 [SMILES: *C(C)C] | ¹H NMR (400 MHz. CDCl₃): δ 8.55 (d, J = 4.0 Hz, 1H), 7.76 (s, 1H), 7.70 (d, J = 8.0 Hz, 1H), 7.28 (t, J = 6.0 Hz, 1H), 5.63 (s, 1H), 5.15 (t, J = 8.0 Hz, 1H), 4.60 (t, J = 10.0 Hz, 1H), 3.90-3.95 (m, 1H), 3.37-3.40 (m, 1H), 2.84-2.90 (m, 2H), 1.28-1.39 (m, 9H) | 3.47 min Method 18 |
| 194 | 0 | 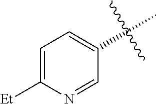 | 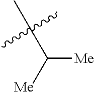 [SMILES: *C(C)C] | ¹H NMR (400 MHz, CDCl₃): δ 8.55 (s, 1H), 7.77 (s, 1H), 7.68- 7.70 (m, 1H), 7.26 (t, J = 6.0 Hz, 1H), 5.75 (s, 1H), 5.14 (t, J = 8.0 Hz, 1H), 4.56-4.61 (m, 1H), 3.89- 3.94 (m, 1H), 3.36-3.39 (m, 1H), 2.83-2.89 (m, 2H), 1.30-1.38 (m, 9H) | 5.12 min Method 18 |
| 195 | 0 | 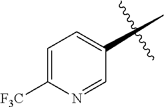 | 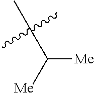 [SMILES: *C(C)C] | ¹H NMR (400 MHz, CDCl₃): δ 8.80 (s, 1H), 7.99 (d, J = 7.6 Hz, 1H), 7.80-7.71 (m, 2H), 5.44 (s, 1H), 5.27 (t, J = 7.6 Hz, 1H), 4.68 (t, J = 10.0 Hz, 1H), 3.92 (dd, J1 = 8 Hz, J2 = 11.2 Hz, 1H), 3.39-3.36 (m, 1H), 1.39-1.36 (m, 6H) | 5.04 min Method 1 |
| 196 | 0 | 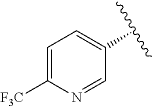 | 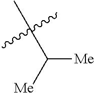 [SMILES: *C(C)C] | ¹H NMR (400 MHz, CDCl₃): δ 8.80 (s, 1H), 7.99 (d, J =7.6 Hz, 1H), 7.80-7.71 (m, 2H), 5.44 (s, 1H), 5.27 (t, J = 7.6 Hz, 1H), 4.68 (t, J = 10.0 Hz, 1H), 3.92 (dd, J1 = 8 Hz, J2 = 11.2 Hz, 1H), 3.39-3.36 (m, 1H), 1.39-1.36 (m, 6H) | 6.91 min Method 1 |
| 197 | 0 | 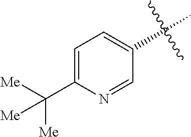 | 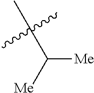 [SMILES: *C(C)C] | ¹H NMR (400 MHz, CDCl₃): δ 8.58 (d, J = 2.0 Hz, 1H), 7.78 (s, 1H), 7.68 (dd, J1 = 2.4 Hz, J2 = 8.4 Hz, 1H), 7.43 (d, J = 8.4 Hz, 1H), 5.15-5.11 (m, 2H), 4.57 (dd, J1 = 8.8 Hz, J2 = 11.2 Hz, 1H), 3.94 (dd, J1 = 7.2 Hz, J2 = 11.6 Hz, 1H), 3.41-3.39 (m, 1H), 1.39-1.36 (m, 15H) | 1.86 min Method 9 |
| 198 | 0 | 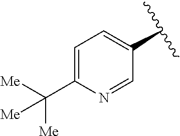 | 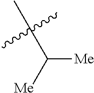 [SMILES: *C(C)C] | ¹H NMR (400 MHz, CDCl₃): δ 8.58 (d, J = 2.8 Hz, 1H), 7.78 (s, 1H), 7.68 (dd, J1 = 2.4 Hz, J2 = 8.4 Hz, 1H), 7.43 (d, J = 8.0 Hz, 1H), 5.15-5.11 (m, 2H), 4.57 (dd, J1 = 8.8 Hz, J2 = 11.2 Hz, 1H), 3.94 (dd, J1 = 7.6 Hz, J2 = 12.0 Hz, 1H), 3.41-3.34 (m, 1H), 1.39-1.36 (m, 15 H) | 2.38 min Method 9 |
| 199 | 0 | 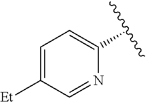 | 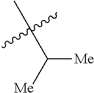 [SMILES: *C(C)C] | ¹H NMR (400 MHz, CDCl₃) δ 1.28 (t, J = 7.6 Hz, 3H), 1.39 (dd, J1 = 3.2 Hz, J2 = 6.8 Hz, 6H), 2.70 (dd, J1 = 7.6 Hz, J2 = 15.2 Hz, 2H), 3.36-3.43 (m, 1H), 4.24 (dd, J1 = 5.2 Hz, J2 = 11.6 Hz, 1H), 4.54 (dd, J1 = 9.2 Hz, J2 = 11.2 Hz, 1H), 5.17 (dd, J1 = 5.2 Hz, J2 = 6.0 Hz, 1H), 5.55 (s, 1H), 7.30 (d, J = 8.0 Hz, 1H), 7.59 (dd, J1 = 2.4 Hz, J2 = 4.0 Hz, 1H), 7.76 (s, 1H), 8.46 (d, J = 2.0 Hz, 1H) | 6.53 min Method 24 |
| 200 | 0 | 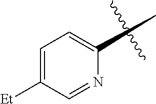 | 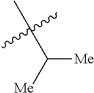 [SMILES: *C(C)C] | ¹H NMR (400 MHz, CDCl₃): δ 8.45 (d, J = 1.2 Hz, 1H), 7.75 (s, 1H), 7.59 (dd, J = 2.0 Hz, 8.0 Hz, 1H), 7.29 (t, J = 8.0 Hz, 1H), 5.70 (s, 1H), 5.17 (dd, J = 5.6 Hz, 8.4 Hz, 1H), 4.54 (dd, J = 9.2 Hz. 9.6 Hz, 1H), 4.23 (dd, J = 5.6 Hz, 9.6 Hz, 1H), 3.36-3.43 (m, 1H), 2.69 (dd, J = 7.2 Hz, 14.8 Hz, 2H), 1.37 (dd, J = 3.2 Hz, 6.4 Hz, 6H), 1.27 (t, J = 7.2 Hz, 3H) | 7.44 min Method 24 |
| 201 | 0 | 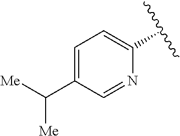 | 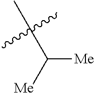 [SMILES: *C(C)C] | ¹H NMR (400 MHz, CDCl₃) δ 1.29-1.38 (m, 6H), 1.38-1.40 (m, 6H), 2.95-3.02 (m, 1H), 3.37-3.44 (m, 1H), 4.25 (dd, J1 = 5.2 Hz, J2 = 11.2 Hz, 1H), 4.55 (dd, J1 = 8.8 Hz, J2 = 11.6 Hz, 1H), 5.17 (dd, J1 = 5.2 Hz, J2 = 8.8 Hz, 1H), 5.50 (s, 1H), 7.31 (d, J = 8.0 Hz, 1H), 7.62 (dd, J1 = 2.0 Hz, J2 = 8.0 Hz, 1H), 7.77 (s, 1H), 8.49 (d, J = 2.4 Hz, 1H) | 4.50 min Method 23 |
| 202 | 0 | 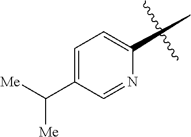 | 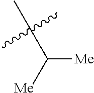 [SMILES: *C(C)C] | ¹H NMR (400 MHz, CDCl₃): δ 8.49 (d, J = 2.0 Hz, 1H), 7.77 (s, 1H), 7.62 (dd, J = 2.0 Hz, 8.0 Hz, 1H), 7.30 (t, J = 8.0 Hz, 1H), 5.45 (s, 1H), 5.17 (dd, J = 4.8 Hz. 8.0 Hz, 1H), 4.55 (dd, J = 8.8 Hz, 11.6 Hz, 1H), 4.26 (dd, J = 5.2 Hz, 11.2 Hz, 1H), 337-3.44 (m, 1H), 2.96- 3.02 (m, 1H), 1.39 (dd, J = 2.8 Hz, 7.2 Hz, 6H), 1.29 (t, J = 6.8 Hz, 6H) | 5.81 min Method 23 |
| 203 | 0 | 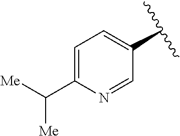 | 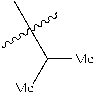 [SMILES: *C(C)C] | ¹H NMR (400 MHz, CDCl₃): δ 8.56 (d, J = 2.0 Hz, 1H), 7.78 (s, 1H), 7.68 (dd, J1 = 2.4 Hz, J2 = 8.4 Hz, 1H), 7.27-7.25 (m, 1H), 5.15- 5.08 (m, 2H), 4.58 (dd, J1 = 8.4 Hz, J2 = 11.2 Hz, 1H), 3.92 (dd, J1 = 7.2 Hz, J2 = 11.2 Hz, 1H), 3.41- 3.34 (m, 1H), 3.14-3.07 (m, 1H), 1.39-1.36 (m, 6H), 1.31 (d, J = 6.8 Hz, 6H) | 3.87 min Method 17 |
| 204 | 0 | 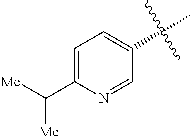 | 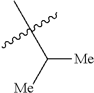 [SMILES: *C(C)C] | ¹H NMR (400 MHz, CDCl₃): δ 8.55 (d, J = 2.4 Hz, 1H), 7.77 (s, 1H), 7.68 (dd, J1 = 2.4 Hz, J2 = 8.0 Hz, 1H), 7.27-7.25 (m, 1H), 5.23 (s, 1H), 5.15-5.11 (m, 1H), 4.57 (dd, J1 = 8.8 Hz, J2 = 11.6 Hz, 1H), 3.93 (dd, J1 = 7.2 Hz, J2 = 11.6 Hz, 1H), 3.41-3.34 (m, 1H), 3.13- 3.07 (m, 1H), 1.39-1.36 (m, 6H), 1.31 (d, J = 7.2 Hz, 6H) | 6.02 min Method 17 |
| 205 | 0 | 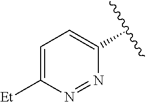 | 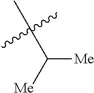 [SMILES: *C(C)C] | ¹H NMR (400 MHz, CD₃OD) δ: 7.84 (d, J = 8.8 Hz, 1H), 7.73 (d, J = 8.4 Hz, 1H), 7.64 (s, 1H), 5.44 (dd, J1 = 5.6 Hz, J2 = 8.8 Hz, 1H), 4.67-4.62 (m, 1H), 4.24 (dd, J1 = 6.0 Hz, J2 = 11.6 Hz, 1H), 3.49- 3.45 (m, 1H), 3.06-3.00 (m, 2H), 1.39-1.31 (m, 9H) | 3.92 min Method 28 |
| 206 | 0 | 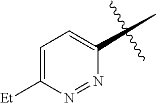 | 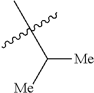 [SMILES: *C(C)C] | ¹H NMR (400 MHz. CD₃OD) δ: 7.84 (d, J = 8.4 Hz, 1H), 7.73 (d, J = 9.2 Hz, 1H), 7.65 (s, 1H), 5.44 (dd, J1 = 6.0 Hz, J2 = 9.2 Hz, 1H), 4.67-4.62 (m, 1H), 4.24 (dd, J1 = 5.6 Hz, J2 = 11.6 Hz, 1H), 3.51- 3.44 (m, 1H), 3.06-3.00 (m, 2H), 1.39-1.31 (m, 9H) | 5.22 min Method 28 |
| 207 | 0 | 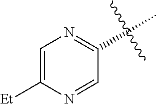 | 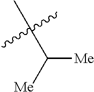 [SMILES: *C(C)C] | ¹H NMR (400 MHz, CDCl₃) δ 8.57 (d, J = 1.2 Hz, 1H), 8.47 (d, J = 1.2 Hz, 1H), 7.75 (s, 1H), 5.49 (s, 1H), 5.23-5.20 (m, 1H), 5.58-5.53 (m, 1H), 4.29-4.25 (m, 1H), 3.41-3.34 (m, 1H), 2.88 (q, J = 7.6 Hz, 2H), 1.37-1.25 (m, 9H) | 5.45 min Method 26 |
| 208 | 0 | 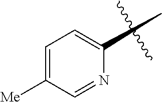 | 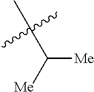 [SMILES: *C(C)C] | ¹H NMR (400 MHz, CDCl₃) δ 1.37 (dd, J1 = 4.4 Hz. J2 = 7.2 Hz, 6H), 2.36 (s, 3H), 3.34-3.41 (m, 1H), 4.18-4.23 (m, 1H), 4.54 (dd, J1 = 8.8 Hz, J2 = 11.2 Hz, 1H), 5.17 (dd, J1 = 5.6 Hz, J2 = 8.8 Hz, 1H), 5.94 (s, 1H), 7.27 (d, J = 7.2 Hz, 1H), 7.55 (dd, J1 = 1.6 Hz, J2 = 8.0 Hz, 1H), 7.73 (s, 1H), 8.42 (s, 1H) | 6.03 min Method 6 |
| 209 | 0 | 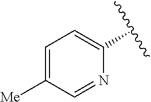 | 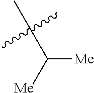 [SMILES: *C(C)C] | ¹H NMR (400 MHz, CDCl₃) δ 1.36 (dd, J1 = 4.4 Hz, J2 = 7.2 Hz, 6H), 2.36 (s, 3H), 3.33-3.40 (m, 1H), 4.18-4.21 (m, 1H), 4.54 (dd, J1 = 8.8 Hz, J2 = 11.6 Hz, 1H), 5.17 (dd, J1 = 5.6 Hz, J2 = 8.8 Hz, 1H), 6.04 (s, 1H), 7.27 (d, J = 7.6 Hz, 1H), 7.55 (dd, J1 = 1.6 Hz, J2 = 8.0 Hz, 1H), 7.73 (s, 1H), 8.42 (s, 1H) | 10.22 min Method 6 |
| 210 | 0 | 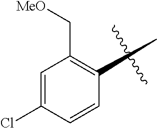 | 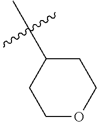 [SMILES: *C1CCOCC1] | ¹H NMR (400 MHz, CDCl₃): δ 7.78 (s, 1H), 7.50 (d, J = 8.0 Hz, 1H), 7.40 (dd, J1 = 2.0 Hz, J2 = 8.4 Hz, 1H), 7.34 (d, J = 1.6 Hz, 1H), 5.35 (t, J = 7.2 Hz, 1H), 5.12 (s, 1H), 4.57-4.51 (m, 2H), 4.43 (d, J = 12.0 Hz, 1H), 4.11-4.07 (m, 2H), 3.95 (dd, J1 = 6.4 Hz, J2 = 11.6 Hz, 1H), 3.61-3.55 (m, 2H), 3.41 (s, 3H), 3.35-3.28 (m, 1H), 2.14-2.02 (m, 2H), 1.93-1.88 (m, 2H) | 3.85 min Method 19 |
| 211 | 0 | 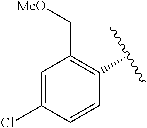 | 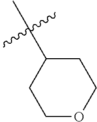 [SMILES: *C1CCOCC1] | ¹H NMR (400 MHz, CDCl₃): δ 7.78 (s, 1H), 7.50 (d, J = 8.4 Hz, 1H), 7.40 (dd, J1 = 2.0 Hz, J2 = 8.4 Hz, 1H), 7.34 (d, J = 2.0 Hz, 1H), 5.37-5.32 (m, 1H), 5.12 (s, 1H), 4.57-4.51 (m, 2H), 4.43 (d, J = 11.6 Hz, 1H), 4.11-4.07 (m, 2H), 3.95 (dd, J1 = 6.4 Hz, J2 = 11.6 Hz, 1H), 3.61-3.55 (m, 2H), 3.41 (s, 3H), 3.36-3.28 (m, 1H), 2.15-2.02 (m, 2H), 1.94-1.88 (m, 2H) | 4.75 min Method 19 |
| 212 | 0 | 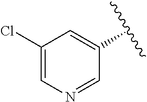 | 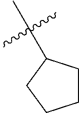 [SMILES: *C1CCCC1] | ¹H NMR (400 MHz, CDCl₃): δ 8.64 (d, J = 4, 1H), 8.56 (d, J = 4, 1H), 7.79-7.80 (m, 2H), 5.25 (s, 1H), 5.18 (m, 1H), 4.61-4.66 (m, 1H), 3.90-3.95 (m, 1H), 3.44-3.48 (m, 1H), 2.08-2.10 (m, 2H), 1.86- 1.96 (m, 4H), 1.63-1.72 (m, 2H) | 4.99 min Method 19 |
| 213 | 0 | 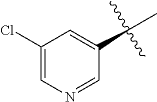 | 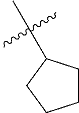 [SMILES: *C1CCCC1] | ¹H NMR (400 MHz, CDCl₃): δ 8.63 (d, J = 4, 1H), 8.55 (d, J = 4, 1H), 7.78-7.80 (m, 2H), 5.57 (s, 1H), 5.17 (m, 1H), 4.60-4.65 (m, 1H), 3.88-3.93 (m, 1H), 3.43-3.45 (m, 1H), 2.06-2.08 (m, 2H), 1.84- 1.93 (m, 4H), 1.68-1.72 (m, 2H) | 6.12 min Method 19 |
| 214 | 0 | 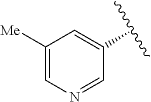 | 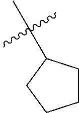 [SMILES: *C1CCCC1] | ¹H NMR (400 MHz, CDCl₃): δ 8.48 (d, J = 12, 2H), 7.78 (s, 1H), 7.57 (s, 1H), 5.42 (s, 1H), 5.13 (m, 1H), 4.57-4.62 (m, 1H), 3.90-3.95 (m, 1H), 3.44-3.48 (m, 1H), 2.39 (s, 3H), 2.08-2.11 (m, 2H), 1.86- 1.95 (m, 4H), 1.69-1.72 (m, 2H) | 3.23 min Method 27 |
| 215 | 0 | 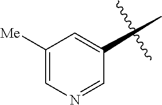 | 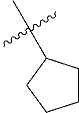 [SMILES: *C1CCCC1] | ¹H NMR (400 MHz, CDCl₃): δ 8.45-8.49 (m, 2H), 7.78 (s, 1H), 7.57 (s, 1H), 5.49 (s, 1H), 5.13 (m, 1H), 4.56-4.61 (m, 1H), 3.89-3.94 (m, 1H), 3.44-3.48 (m, 1H), 2.39 (s, 3H), 2.07-2.10 (m, 2H), 1.85- 1.92 (m, 4H), 1.68-1.71 (m, 2H) | 3.81 min Method 27 |
| 216 | 0 | 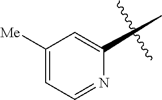 | 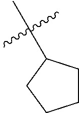 [SMILES: *C1CCCC1] | ¹H NMR (400 MHz, CDCl₃): δ 8.44 (d, J = 4 Hz, 1H), 7.74 (s, 1H), 7.17 (s, 1H), 7.10 (d, J = 4 Hz, 1H), 5.64 (s, 1H), 5.14-5.16 (m, 1H), 4.50-4.55 (m, 1H), 4.18-4.23 (m, 1H), 3.43-3.45 (m, 1H), 2.38 (s, 3H), 2.06-2.07 (m, 2H), 1.84- 1.86 (m, 4H), 1.66-1.69 (m, 2H) | 13.55 min Method 25 |
| 217 | 0 | 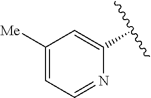 | 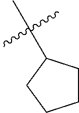 [SMILES: *C1CCCC1] | ¹H NMR (400 MHz, CDCl₃): δ 8.44 (d, J = 4 Hz, 1H), 7.74 (s, 1H), 7.18 (s, 1H), 7.11 (d, J = 4 Hz, 1H), 5.70 (s, 1H), 5.14-5.16 (m, 1H), 4.51-4.56 (m, 1H), 4.20-4.23 (m, 1H), 3.43-3.46 (m, 1H), 2.38 (s, 3H), 2.06-2.07 (m, 2H), 1.83- 1.87 (m, 4H), 1.67-1.70 (m, 2H) | 16.25 min Method 25 |
| 218 | 0 | 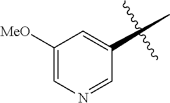 | 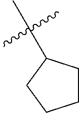 [SMILES: *C1CCCC1] | ¹H NMR (400 MHz, CDCl₃): δ 8.36 (d, J = 8 Hz, 1H), 8.26 (d, J = 4 Hz, 1H), 7.79 (s, 1H), 7.24-7.26 (m, 1H), 5.22 (s, 1H), 5.16 (m, 1H), 4.58-4.64 (m, 1H), 3.89-3.94 (m, 4H), 3.44-3.46 (m, 1H), 2.08- 2.11 (m, 2H), 1.86-1.89 (m, 4H), 1.69-1.72 (m, 2H) | 4.40 min Method 19 |
| 219 | 0 | 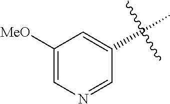 | 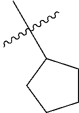 [SMILES: *C1CCCC1] | ¹H NMR (400 MHz, CDCl₃): δ 8.36 (d, J = 8 Hz, 1H), 8.26 (d, J = 4 Hz, 1H), 7.79 (s, 1 H), 7.25-7.27 (m, 1H), 5.26 (s, 1H), 5.16 (m, 1H), 4.58-4.64 (m, 1H), 3.89-3.94 (m, 4H), 3.44-3.48 (m, 1H), 2.08- 2.11 (m, 2H), 1.87-1.96 (m, 4H), 1.69-1.72 (m, 2H) | 5.19 min Method 19 |
| 220 | 0 | 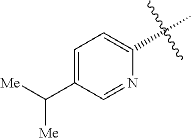 | 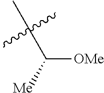 [SMILES: *[C@@H](C)OC] | ¹H NMR (400 MHz, CDCl₃): δ 1.30 (d, J = 6.8 Hz, 6H), 1.67 (d, J = 6.4 Hz, 3H), 2.97-3.01 (m, 1H), 3.32 (s, 3H), 4.27 (dd, J = 5.6 Hz, 11.6 Hz, 1H), 4.56 (dd, J = 9.2 Hz, 12.0 Hz, 1H), 4.90 (q, J = 7.2 Hz, 1H), 5.20 (dd, J = 5.6 Hz, 8.4 Hz, 1H), 5.58 (s, 1H), 7.31 (d, J = 8.0 Hz, 1H), 7.62 (dd, J = 2.0 Hz, 8.0 Hz, 1H), 7.82 (s, 1H), 8.49 (d, J = 2.4 Hz, 1H) | 2.10 min Method 29 |
| 221 | 0 | 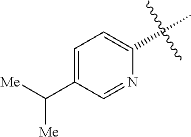 | 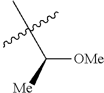 [SMILES: *[C@H](C)OC] | ¹H NMR (400 MHz, CDCl₃): δ 1.30 (d, J = 6.8 Hz, 6H), 1.66 (d, J = 6.4 Hz, 3H), 2.97-3.01 (m, 1H), 3.33 (s, 3H), 4.26 (dd, J = 5.6 Hz, 11.6 Hz, 1H), 4.56 (dd, J = 9.2 Hz, 12.0 Hz, 1H), 4.89 (q, J = 7.2 Hz, 1H), 5.19 (dd, J = 5.6 Hz, 8.4 Hz, 1H), 5.62 (s, 1H), 7.31 (d, J = 8.0 Hz, 1H), 7.62 (dd, J = 2.0 Hz, 8.0 Hz, 1H), 7.81 (s, 1H), 8.49 (d, J = 2.4 Hz, 1H) | 3.20 min Method 29 |
| 222 | 0 | 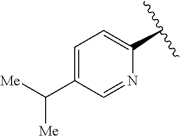 | 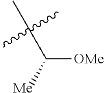 [SMILES: *[C@@H](C)OC] | ¹H NMR (400 MHz, CDCl₃): δ 1.30 (d, J = 6.8 Hz, 6H), 1.66 (d, J = 6.4 Hz, 3H), 2.97-3.01 (m, 1H), 3.33 (s, 3H), 4.26 (dd, J = 5.6 Hz, 11.6 Hz, 1H), 4.56 (dd, J = 9.2 Hz, 12.0 Hz, 1H), 4.89 (q, J = 7.2 Hz, 1H), 5.19 (dd, J = 5.6 Hz, 8.4 Hz, 1H), 5.61 (s, 1H), 7.31 (d, J = 8.0 Hz, 1H), 7.62 (dd. J = 2.0 Hz, 8.0 Hz, 1H), 7.81 (s, 1H), 8.49 (d, J = 2.4 Hz, 1H) | 4.59 min Method 29 |
| 223 | 0 | 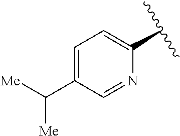 | 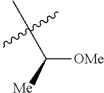 [SMILES: *[C@H](C)OC] | ¹H NMR (400 MHz, CDCl₃): δ 1.30 (d, J = 6.8 Hz, 6H), 1.67 (d, J = 6.4 Hz, 3H), 2.97-3.01 (m, 1H), 3.33 (s, 3H), 4.27 (dd, J = 5.6 Hz, 11.6 Hz, 1H), 4.56 (dd, J = 9.2 Hz, 12.0 Hz, 1H), 4.90 (q, J = 7.2 Hz, 1H), 5.19 (dd. J = 5.6 Hz, 8.4 Hz, 1H), 5.53 (s, 1H), 7.31 (d, J = 8.0 Hz, 1H), 7.62 (dd, J = 2.0 Hz, 8.0 Hz, 1H), 7.82 (s, 1H), 8.49 (d, J = 2.4 Hz, 1H) | 6.12 min Method 29 |
| 224 | 1 | 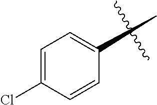 | 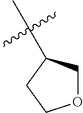 [SMILES: *[C@@H]1CCOC1] | ¹H NMR (400 MHz, CDCl₃): δ 7.80 (s, 1H), 7.42 (d, J = 8.4 Hz, 2H), 7.30 (d, J = 8.8 Hz, 2H), 5.10 (br s, 1H), 4.71-4.67 (m, 1H), 4.30-4.21 (m, 2H), 4.11-4.05 (m, 1H), 4.01-3.97 (m, 2H), 3.89-3.81 (m, 2H), 2.45-2.34 (m, 3H), 2.09- 2.05 (m, 1H) | 4.05 min Method 20 |
| 225 | 1 | 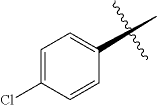 | 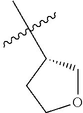 [SMILES: *[C@H]1CCOC1] | ¹H NMR (400 MHz, CDCl₃): δ 7.80 (s, 1H), 7.42 (d, J = 8.4 Hz, 2H), 7.33 (d, J = 8.4 Hz, 2H), 5.12 (br s, 1H), 4.71-4.67 (m, 1H), 4.30-4.20 (m, 2H), 4.11-4.05 (m, 1H), 4.01-3.92 (m, 2H), 3.87-3.82 (m, 2H), 2.50-2.35 (m, 3H), 2.09- 2.05 (m, 1H) | 4.73 min Method 20 |
| 226 | 1 | 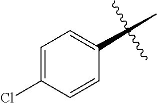 | 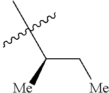 [SMILES: *[C@H](C)CC] | ¹H NMR (400 MHz, CDCl₃): δ 7.82 (s, 1H), 7.43-7.41 (m, 2H), 7.33 (t, J = 14.0 Hz, 2H), 5.04 (s, 1H), 4.69-4.68 (m, 1H), 4.30-4.24 (m, 1H), 3.89-3.82 (m, 1H), 3.24- 3.18 (m, 1H), 2.40-2.36 (m, 1H), 2.11-2.05 (m, 1H), 1.97-1.89 (m, 1H), 1.76-1.42 (m, 1H), 1.35-1.28 (m, 3H), 0.93-0.88 (m, 3H) | 3.28 min Method 3 |
| 227 | 1 | 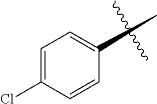 | 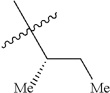 [SMILES: *[C@@H](C)CC] | ¹H NMR (400 MHz, CDCl₃): δ 7.83 (s, 1H), 7.43-7.41 (m, 2H), 7.33 (t, J = 14 Hz, 2 H), 5.05 (s, 1H), 4.70-4.67 (m, 1H), 4.31-4.25 (m, 1H), 3.88-3.81 (m, 1H), 3.25- 3.19 (m, 1H), 2.41-2.36 (m, 1H), 2.09-2.04 (m, 1H), 1.93-1.86 (m, 1H), 1.74-1.64 (m, 1H), 1.37-1.28 (m, 3H), 0.89-0.86 (m, 3H) | 3.81 min Method 3 |

### Example 228

### (2R)-2-(4-Chlorophenyl)-9-(1-methylcyclopropyl)-1,2,3,4-tetrahydro-6H-imidazo[5,1-f]pyrimido[2,1-c][1,2,4]triazin-6-one

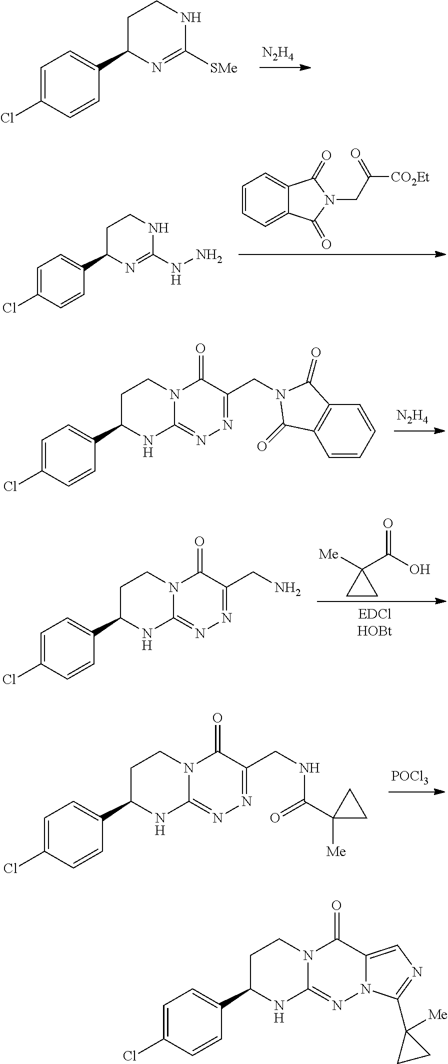

N-{[(8R)-8-(4-Chlorophenyl)-4-oxo-1,6,7,8-tetrahydro-4H-pyrimido[2,1-c][1,2,4]triazin-3-yl]methyl}-1-methylcyclopropanecarboxamide (262 mg, 0.7 mmol), tripotassium phosphate (297 mg, 1.4 mmol) and phosphoryl chloride (10 mL) was flushed with nitrogen (3 times), and then heated to 60° C. for 15 h. Cooled to rt, concentrated to give a residue, adjusted pH=8 with sat. sodium bicarbonate aq. solution, extracted with EtOAc (60 mL×3). The combined organic phase was washed with brine (30 mL), dried with sodium sulfate, filtered and concentrated to give a residue, which was purified on reverse phase chromatography column (A: water (0.01% Ammonia), B: MeCN) to give the titled compound (182 mg, 51.1%) as white solids. ¹H NMR (400 MHz, CDCl₃): δ 7.74 (s, 1H), 7.42 (d, J=8.4 Hz, 2H), 7.34 (d, J=8.8 Hz, 2H), 5.10 (br s, 1H), 4.71-4.66 (m, 1H), 4.28-4.24 (m, 1H), 3.88-3.84 (m, 1H), 2.40-2.34 (m, 1H), 2.10-2.04 (m, 1H), 1.54 (s, 3H), 1.29-1.24 (m, 2H), 0.79-0.76 (m, 2H). MS (ESI): m/e=356 [M+H]⁺.

### (4R)-4-(4-Chlorophenyl)tetrahydro-2(1H)-pyrimidinone hydrazone

To a solution of (4R)-4-(4-chlorophenyl)-2-(methylthio)-1,4,5,6-tetrahydropyrimidine (Reference Example 7, 240 mg, 1 mmol) in EtOH (3 mL), was added hydrazine monohydrate (250 mg, 5 mmol). The mixture was heated to reflux for 1 h, filtered. The filtrate was concentrated and used for next step. MS: m/e=225 [M+H]⁺.

### 2-{[(8R)-8-(4-Chlorophenyl)-4-oxo-1,6,7,8-tetrahydro-4H-pyrimido[2,1-c][1,2,4]triazin-3-yl]methyl}-1H-isoindole-1,3(2H)-dione

To a solution of (4R)-4-(4-chlorophenyl)tetrahydro-2(1H)-pyrimidinone hydrazine (1.12 g, 5 mmol) in EtOH (20 mL) was added ethyl 3-(1,3-dioxo-1,3-dihydro-2H-isoindol-2-yl)-2-oxopropanoate (1.31 g, 5 mmol). The mixture was heated to reflux and stirred for 2 d. Filtered and concentrated to afford the titled compound (2.1 g, 100%) as white solids, which were used directly for the next step. MS: m/e=422 [M+H]⁺.

### (8R)-3-(Aminomethyl)-8-(4-chlorophenyl)-1,6,7,8-tetrahydro-4H-pyrimido[2,1-c][1,2,4]triazin-4-one

To a solution of 2-{[(8R)-8-(4-Chlorophenyl)-4-oxo-1,6,7,8-tetrahydro-4H-pyrimido[2,1-c][1,2,4]triazin-3-yl]methyl}-1H-isoindole-1,3(2H)-dione (3 g, 7.11 mmol) in EtOH (20 mL) was added in hydrazine (1.42 g, 28.44 mmol). The mixture was heated to reflux for 4 h, filtered and concentrated to give the crude product (1.8 g, 86.9%) as yellow solids. MS: m/e=292 [M+H]⁺.

### N-{[(8R)-8-(4-Chlorophenyl)-4-oxo-1,6,7,8-tetrahydro-4H-pyrimido[2,1-c][1,2,4]triazin-3-yl]methyl}-1-methylcyclopropanecarboxamide

1-Methylcyclopropanecarboxylic acid (100 mg, 1.0 mmol) in DMF (6 mL) was added to EDCI (384 mg, 2.0 mmol), HOBt hydrate (270 mg, 2.0 mmol) and DIPEA (387 mg, 3.0 mmol), and stirred at rt for 0.5 h, then (8R)-3-(aminomethyl)-8-(4-chlorophenyl)-1,6,7,8-tetrahydro-4H-pyrimido[2,1-c][1,2,4]triazin-4-one (292 mg, 1.0 mmol) was added to the mixture and stirred at rt for 4 h. Purified on reverse phase chromatography column (A: water (0.01% ammonia), B: MeCN) to give the titled compound (286 mg, 76.7%) as white solids. MS (ESI): m/e=374 [M+H]⁺.

The compounds of Example 229 to 250 were synthesized in a similar manner to Example 228.

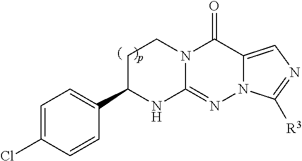

| Ex. | | | |
|---|---|---|---|
| No. | p | R³— | ¹H NMR |
| 229 | 0 | 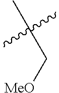 [SMILES: *COC] | ¹H NMR (400 MHz. CDCl₃): δ 7.81 (s, 1H), 7.42-7.40 (m, 2H), 7.34-7.32 (m, 2H), 5.21 (s, 1H), 5.13-5.09 (m, 1H), 4.70 (s, 2H), 4.57 (dd, J1 = 11.6 Hz, J2 = 8.8 Hz, 1H), 3.89 (dd, J1 = 11.6 Hz, J2 = 7.6 Hz, 1H), 3.44 (s, 3H) |
| 230 | 0 | 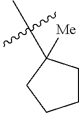 [SMILES: *C1(C)CCCC1] | ¹H NMR (400 MHz, CDCl₃): δ 7.76 (s, 1H), 7.43 (d, J = 8.8 Hz, 2H), 7.37 (d, J = 8.4 Hz, 2H), 5.19 (s, 1H), 5.11 (t, J = 8.2 Hz, 1H), 4.58 (dd, J1 = 8.8 Hz, J2 = 11.6 Hz, 1H), 3.87 (dd, J1 = 8.0 Hz, J2 = 9.0 Hz, 1H), 2.44-2.43 (m, 2H), 1-84-1.72 (m, 6H), 1.47 (s, 3H) |
| 231 | 0 | 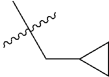 [SMILES: *CC1CC1] | ¹H NMR (400 MHz, CDCl₃): δ 7.80 (s, 1H), 7.44-7.35 (m, 4H), 5.17-5.12 (m, 2H), 4.62-4.57 (m, 1H), 3.92-3.89 (m, 1H), 2.83 (d, J = 8.2 Hz, 2H), 1.27-1.25 (m, 1H), 0.58-0.56 (m, 2H), 0.32-0.31 (m, 2H) |
| 232 | 0 | 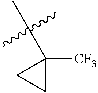 [SMILES: *C1(C(F)(F)F)CC1] | ¹H NMR (400 MHz, CDCl₃): δ 7.84 (s, 1H), 7.45-7.29 (m, 4H), 5.28 (s, 1H), 5.15 (t, J = 8.4 Hz, 1H), 4.60 (dd, J1 = 8.8 Hz, J2 = 11.6 Hz, 1H), 3.90 (dd, J1 = 7.6 Hz, J2 = 11.6 Hz, 1H), 1.56-1.53 (m, 2H), 1.39-1.36 (m, 2H) |
| 233 | 0 | 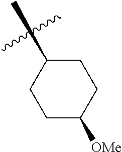 | ¹H NMR (400 MHz, CDCl₃) δ 7.78 (s, 1H), 7.42-7.44 (m, 2H), 7.34-7.37 (m, 2H), 5.20 (s, 1H), 5.12 (t, J = 8.4 Hz, 1H), 4.56 (dd, J1 = 11.2 Hz, J2 = 8.0 Hz, 1H), 3.90 (dd, J1 = 11.2 Hz, J2 = 7.2 Hz, 1H), 3.40 (s, 3H), 3.22-3.29 (m, 1H), 3.03-3.09 (m, 1H), 2.22-2.25 (m, 2H), 2.08-2.13 (m, 2H), 1.70-1.82 (m, 2H), 1.34-1.44 (m, 2H) |
| 234 | 0 | 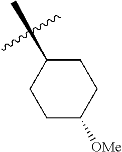 | ¹H NMR (400 MHz, CDCl₃) δ 7.80 (s, 1H), 7.40 (dd, J1 = 30.0 Hz, J2 = 8.4 Hz, 4H), 5.07-5.13 (m, 2H), 4.57 (dd, J1 = 12.0 Hz, J2 = 8.8 Hz, 1H), 3.90 (dd, J1 = 11.2 Hz, J2 = 7.2 Hz, 1H), 3.53 (s, 1H), 3.35 (s, 3H), 3.09-3.14 (m, 1H), 2.06-2.13 (m, 4H), 1.76-1.80 (m, 2H), 1.55-.161 (m, 2H) |
| 235 | 0 | 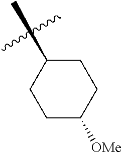 | ¹H NMR (400 MHz, CDCl₃): δ 7.79 (s, 1H), 7.42 (m, 2H), 7.33 (d, J = 8.4 Hz, 2 H), 5.08 (br s, 1H), 4.70-4.66 (m, 1H), 4.28-4.24 (m, 1H), 3.88-3.83 (m, 1H), 3.41(s, 3H), 3.26-3.23 (m, 1H), 3.08-3.02 (m, 1H), 2.45-2.39 (m, 1H), 2.25-2.21 (m, 2H), 2.12-2.05 (m, 3H), 1.80-1.72 (m, 2H), 1.41-1.34 (m, 2H) |
| 236 | 1 | 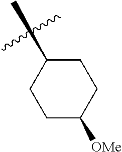 | ¹H NMR (400 MHz, CDCl₃): δ 7.80 (s, 1H), 7.41 (d, J = 8.4 Hz, 2H), 7.33 (d, J = 8.4 Hz, 2H), 5.11 (br s, 1H), 4.70-4.66 (m, 1H), 4.27-4.23 (m, 1H), 3.88-3.82 (m, 1H), 3.54-3.50 (m, 1H), 3.42 (s, 3H), 3.12-3.07 (m, 1H), 2.38-2.33 (m, 1H), 3.13-3.03 (m, 5H), 1.79-1.74 (m, 2H), 1.61-1.52 (m, 2H) |
| 237 | 1 | 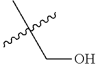 [SMILES: *CO] | ¹H NMR (400 MHz, DMSO-d₆): 8.03 (s, 1H), 7.63 (s, 1H), 7.45- 7.40 (m, 4H), 5.28 (br s, 1H), 4.72-4.68 (m, 1H), 4.62 (s, 2H), 3.94-3.89 (m, 1H), 3.70-3.65 (m, 1H), 2.28-2.23 (m, 1H), 1.99- 1.95 (m, 1H) |
| 238 | 1 | 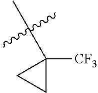 [SMILES: *C1(C(F)(F)F)CC1] | ¹H NMR (400 MHz, CDCl₃): δ 7.83 (s, 1H), 7.43 (d, J = 4.0 Hz, 2H), 7.34 (d, J = 4.0 Hz, 2H), 5.24 (s, 1H), 4.72-4.68 (m, 1H), 4.31- 4.25 (m, 1H), 3.90-3.83 (m, 1H), 2.42-2.37 (m, 1H), 2.12-2.07 (m, 1H), 1.51 (t, J = 4.0 Hz, 2H), 1.36 (s, 2H) |
| 239 | 1 | 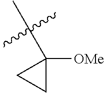 [SMILES: *C1(OC)CC1] | ¹H NMR (400 MHz, CDCl₃): δ 7.80 (s, 1H), 7.43-7.39 (m, 2H), 7.34-7.32 (m, 2H), 5.37 (s, 1H), 4.71-4.68 (m, 1H), 4.29-4.23 (m, 1H), 3.93-3.86 (m, 1H), 3.32 (s, 3H), 2.40-2.36 (m, 1H), 2.13- 2.03 (m, 1H), 1.28-1.24 (m, 2H), 1.23-1.18 (m, 2H) |
| 240 | 1 | 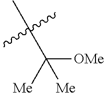 [SMILES: *C(C)(C)OC] | ¹H NMR (400 MHz, CDCl₃): δ 7.80 (s, 1H), 7.41 (d, J = 8.8 Hz, 2H), 7.32 (d, J = 8.4 Hz, 2H), 5.31 (br s, 1H), 4.69-4.65 (m, 1H), 4.31-4.25 (m, 1H), 3.89-3.82 (m, 1H), 3.08 (s, 3H), 2.40-2.34 (m, 1H), 2.10-2.05 (m, 1H), 1,77(s, 3H), 1.76 (s, 3H) |
| 241 | 1 | 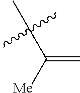 [SMILES: *C(=C)C] | ¹H NMR (400 MHz, CDCl₃): δ 7.89 (s, 1H), 7.43-7.31 (m, 2H), 7.34-7.28 (m, 2H), 6.41 (s, 1H), 5.53 (s, 1H), 5.15 (s, 1H), 4.72- 4.69 (m, 1H), 4.30-4.23 (m, 1H), 3.92-3.85 (m, 1H), 2.41-2.37 (m, 1H), 2.31 (s, 3H), 2.12-2.03 (m, 1H) |
| 242 | 1 | 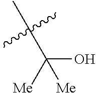 [SMILES: *C(C)(C)O] | ¹H NMR (400 MHz, CDCl₃): δ 7.78 (s, 1H), 7.45-7.42 (m, 2H), 7.33 (d, J = 8.5 Hz, 2H), 5.23 (br s, 1 H), 4.74-4.70 (m, 2H), 4.31- 4.26 (m, 1H), 3.90-3.84 (m, 1H), 3.42-3.38 (m, 1H), 2.11-2.07 (m, 1H), 1.72 (s, 3H), 1.71 (s, 3H) |
| 243 | 1 | 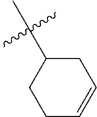 [SMILES: *C1CC=CCC1] | ¹H NMR (400 MHz, CD₃OD): δ 7.94 (d, J = 2.4 Hz, 1H), 7.58 (s, 1H), 7.45-7.40 (m, 4H), 5.75 (s, 2H), 4.69 (br s, 1H), 3.94-3.88 (m, 1H), 3.69-3.65 (m, 1H), 3.29-3.24 (m, 1H), 2.33-2.22 (m, 3H), 2.18-2.10 (m, 2H), 2.00-1.94 (m, 2H), 1.84-1.72 (m, 1H) |
| 244 | 1 | 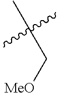 [SMILES: *COC] | ¹H NMR (400 MHz, CDCl₃): δ 7.84 (s, 1H), 7.43-7.40 (m, 2H), 7.33 (d, J = 8.4 Hz, 2H), 5.21 (br s, 1H), 4.75-4.68 (m, 3H), 4.28- 4.22 (m, 1H), 3.91-3.84 (m, 1H), 3.45 (s, 3H), 2.41-2.34 (m, 1H), 2.11-2.02 (m, 1H) |
| 245 | 1 | 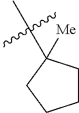 [SMILES: *C1(C)CCCC1] | ¹H NMR (400 MHz, CDCl₃): δ 7.77 (s, 1H), 7.41-7.43 (m, 2H), 7.34 (d, J = 8.4 Hz, 2H), 5.04 (s, 1H), 4.66-4.69 (m, 1H), 4.25-4.28 (m, 1H), 3.84-3.86 (m, lH), 2.36-2.46 (m, 3H), 2.03-2.07 (m, 1H), 1.67-1.81 (m, 6H), 1.46 (s, 3H) |
| 246 | 1 | —CF3 | ¹H NMR (400 MHz, CDCl₃): δ 7.86 (s, 1H), 7.43 (d, J = 4.2 Hz, 2H), 7.33 (d, J = 4.2 Hz, 2H), 5.37 (s, 1H), 4.75-4.72 (m, 1H), 4.32- 4.26 (m, 1H), 3.94-3.88 (m, 1H), 2.44-2.39 (m, 1H), 2.14-2.08 (m, 1H) |
| 247 | 1 | 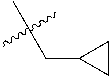 [SMILES: *CC1CC1] | ¹H NMR (400 MHz, CDCl₃): δ 7.82 (s, 1H), 7.43-7.40 (m, 2H), 7.33 (d, J = 8.8 Hz, 2H), 5.06 (br s, 1H), 4.71-4.67 (m, 1H), 4.30- 4.24 (m, 1H), 3.88-3.81 (m, 1H), 2.82 (d, J = 7.2 Hz, 2 H), 2.41- 2.35 (m, 1H), 2.09-2.04 (m, 1H), 1.27-1.23 (m, 1H), 0.58-0.53 (m, 2H), 0.33-0.29 (m, 2H) |
| 248 | 1 | 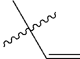 [SMILES: *C=C] | ¹H NMR (400 MHz, CDCl₃): δ 7.87 (s, 1H), 7.42 (d, J = 8.4 Hz, 2H), 7.33 (d, J = 8.8 Hz, 2H), 6.93 (dd, J1 = 17.6 Hz, J2 = 11.2 Hz, 1H), 6.41 (dd, J1 = 18.0 Hz, J2 = 1.6 Hz, 1H), 5.57 (dd, J1 = 11.2 Hz, J2 = 1.6 Hz, 1H), 5.15 (br s, 1H), 4.72-4.68 (m, 1H), 4.30- 4.23 (m, 1H), 3.90-3.83 (m, 1H), 2.42-2.37 (m, 1H), 2.09-2.05 (m, 1H) |
| 249 | 1 | 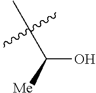 [SMILES: *[C@H](C)O] | ¹H NMR (400 MHz, DMSO-d₆): δ 7.79 (s, 1H), 7.42 (d, J = 8.4 Hz, 2H), 7.33 (d, J = 8.4 Hz, 2H), 5.21 (br s, 2H), 4.74-4.69 (m, 1H), 4.31-4.25 (m, 1H), 3.90-3.83 (m, 1H), 3.54-3.48 (m, 1H), 2.42- 2.37 (m, 1H), 2.10-2.05 (m, 1H), 1.70 (d, J = 6.4 Hz, 3H) |
| 250 | 1 | 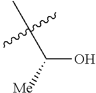 [SMILES: *[C@@H](C)O] | ¹H NMR (400 MHz, DMSO-d₆): δ 7.77 (s, 1H), 7.42 (d, J = 8.4 Hz, 2 H), 7.33 (d, J = 8.4 Hz, 2 H), 5.36 (br s, 1H), 5.24-5.18 (m, 1H), 4.71-4.67 (m, 1H), 4.28-4.22 (m, 1H), 3.90-3.83 (m, 1H), 3.66 (br s, 1H), 2.41-2.37 (m, 1H). 2.09-2.04 (m, 1H), 1.70 (d, J = 6.8 Hz, 3H) |

### Example 251

### (2S)-2-(4-Chlorophenyl)-8-(trifluoromethyl)-2,10-dihydro-3H,5H-diimidazo[2,1-c:5′,1′-f][1,2,4]triazin-5-one

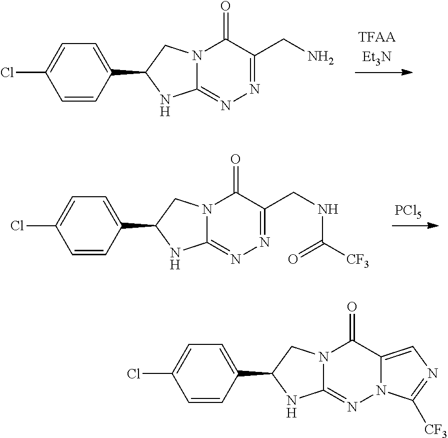 [SMILES: NCc1nnc2n(c1=O)C[C@H](c1ccc(Cl)cc1)N2 ; O=C(NCc1nnc2n(c1=O)C[C@H](c1ccc(Cl)cc1)N2)C(F)(F)F ; "PCl5" ; O=c1c2cnc(C(F)(F)F)n2nc2n1C[C@H](c1ccc(Cl)cc1)N2]

To a solution of N-{[(7S)-7-(4-chlorophenyl)-4-oxo-1,4,6,7-tetrahydroimidazo[2,1-c][1,2,4]triazin-3-yl]methyl}-2,2,2-trifluoroacetamide (50 mg, 0.133 mmol) in tetrahydrothiophene 1,1-dioxide (2 mL) was added pentachlorophosphorane (0.139 g, 0.669 mmol). The reaction mixture was heated to 100° C. and stirred at that temperature for 3 days. The oil was purified by reverse phase HPLC with MeCN (30%) and water (70%) to provide the titled compound as white solids. ¹H NMR (400 MHz, CDCl₃): δ 8.84 (d, J=4.8 Hz, 1H), 7.80 (s, 1H), 7.52-7.46 (m, 4H), 5.20-5.15 (m, 1H), 4.56-4.51 (m, 1H), 3.77-3.3-72 (m, 1 N. MS (ESI): m/e=356 [M+H]⁺.

### N-{[(7S)-7-(4-Chlorophenyl)-4-oxo-1,4,6,7-tetrahydroimidazo[2,1-c][1,2,4]triazin-3-yl]methyl}-2,2,2-trifluoroacetamide

To a solution of (7S)-3-(aminomethyl)-7-(4-chlorophenyl)-6,7-dihydroimidazo[2,1-c][1,2,4]triazin-4(1H)-one (synthesized in the similar manner to Example 228, 400 mg, 1.44 mmol) in DCM (5 mL) was added TEA (0.44 g, 4.32 mmol) and TFAA (0.36 g, 1.73 mmol). The reaction was stirred at rt for 2 h. Concentrated, the resulting oil was purified by reverse phase HPLC with MeCN (25%) and water (75%) to provide the titled compound (0.27 g, 50%) as white solids. MS (ESI): m/e=374 [M+H]⁺.

### Example 252

### (2R)-9-Acetyl-2-(4-chlorophenyl)-1,2,3,4-tetrahydro-6H-imidazo[5,1-f]pyrimido[2,1-c][1,2,4]triazin-6-one

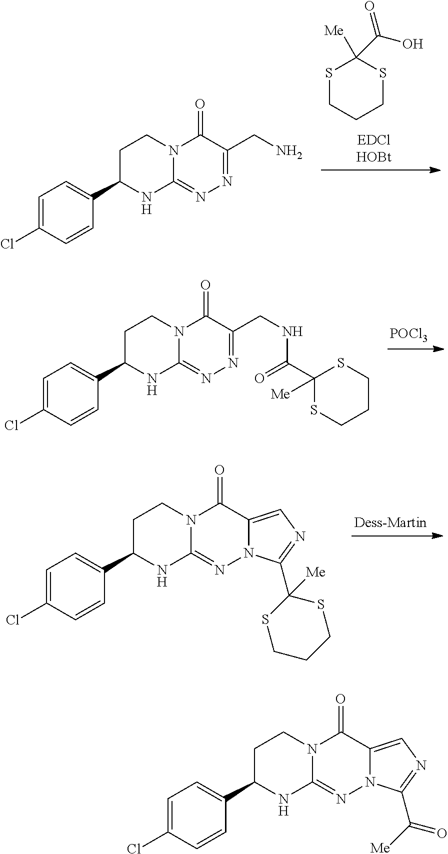 [SMILES: NCc1nnc2n(c1=O)CC[C@H](c1ccc(Cl)cc1)N2 ; CC1(C(=O)O)SCCCS1 ; CC1(C(=O)NCc2nnc3n(c2=O)CC[C@H](c2ccc(Cl)cc2)N3)SCCCS1 ; "POCl3" ; CC1(c2ncc3c(=O)n4c(nn23)N[C@@H](c2ccc(Cl)cc2)CC4)SCCCS1 ; CC(=O)c1ncc2c(=O)n3c(nn12)N[C@@H](c1ccc(Cl)cc1)CC3]

To a solution of (2R)-2-(4-chlorophenyl)-9-(2-methyl-1,3-dithian-2-yl)-1,2,3,4-tetrahydro-6H-imidazo[5,1-f]pyrimido[2,1-c][1,2,4]triazin-6-one (100 mg, 0.23 mmol) in MeCN (1.6 mL), DCM (0.2 mL), and H₂O (0.2 mL) was added Dess-Martin reagent (0.39 g, 0.92 mmol). The mixture was stirred at rt for 4 h, quenched with 50% sodium carbonate aq. solution, extracted with DCM (15 mL×3), dried over sodium sulfate, filtered and concentrated. The crude was purified on flash column to give the titled compound (50 mg, 65%) as yellow solids. ¹H NMR (400 MHz, CDCl₃): δ 7.88 (s, 1H), 7.34 (t, 3=12.0 Hz, 2H), 7.26 (t, J=6.0 Hz, 2H), 6.40 (s, 1H), 4.72-4.70 (m, 1H), 4.29-4.24 (m, 1H), 3.92-3.85 (m, 1H), 2.73 (d, J=6.0 Hz, 3H), 7.41-7.18 (m, 1H), 9.12-9.07 (m, 1H). MS:: m/e==344 [M+H]⁺.

### N-{[(8R)-8-(4-Chlorophenyl)-4-oxo-1,6,7,8-tetrahydro-4H-pyrimido[2,1-c][1,2,4]triazin-3-yl]methyl}-2-methyl-1,3-dithiane-2-carboxamide

To a solution of 2-methyl-1,3-dithiane-2-carboxylic acid (1.32 g, 7.4 mmol) in DMF (5 mL) were added EDCI (2.4 g, 12.33 mmol), HOBt monohydrate (1.67 g, 12.33 mmol) and DIPEA (2.4 g, 18.5 mmol) at rt. After 15 min, (8R)-3-(aminomethyl)-8-(4-chlorophenyl)-1,6,7,8-tetrahydro-4H-pyrimido[2,1-c][1,2,4]triazin-4-one (see Example 228 intermediate, 1.8 g, 6.17 mmol) was added in and the mixture was stirred at rt for 2 h. After quenching with water (1 mL), the mixture was purified on flash column to give the titled compound (1.89 g, 68%) as white solids. MS: m/e=452 [M+H]⁺.

### (2R)-2-(4-Chlorophenyl)-9-(2-methyl-1,3-dithian-2-yl)-1,2,3,4-tetrahydro-6H-imidazo[5,1-f]pyrimido[2,1-c][1,2,4]triazin-6-one

N-{[(8R)-8-(4-Chlorophenyl)-4-oxo-1,6,7,8-tetrahydro-4H-pyrimido[2,1-c][1,2,4]triazin-3-yl]methyl}-2-methyl-1,3-dithiane-2-carboxamide (1.89 g, 4.18 mmol) was dissolved in phosphoryl chloride (20 mL). The mixture was heated to reflux for 2 d, concentrated and acidified to pH 8 with sodium bicarbonate aq. solution, extracted with EtOAc, dried over sodium sulfate, filtered and concentrated. The crude was purified on column chromatography (DCM/MeOH=20/1) to give the titled compound (1.5 g, 83.3%) as white solids. MS: m/e=434 [M+H]⁺.

### Example 253

### 2-(4-Chlorophenyl)-9-(4-piperidinyl)-1,2,3,4-tetrahydro-6H-imidazo[5,1-f]pyrimido[2,1-c][1,2,4]triazin-6-one

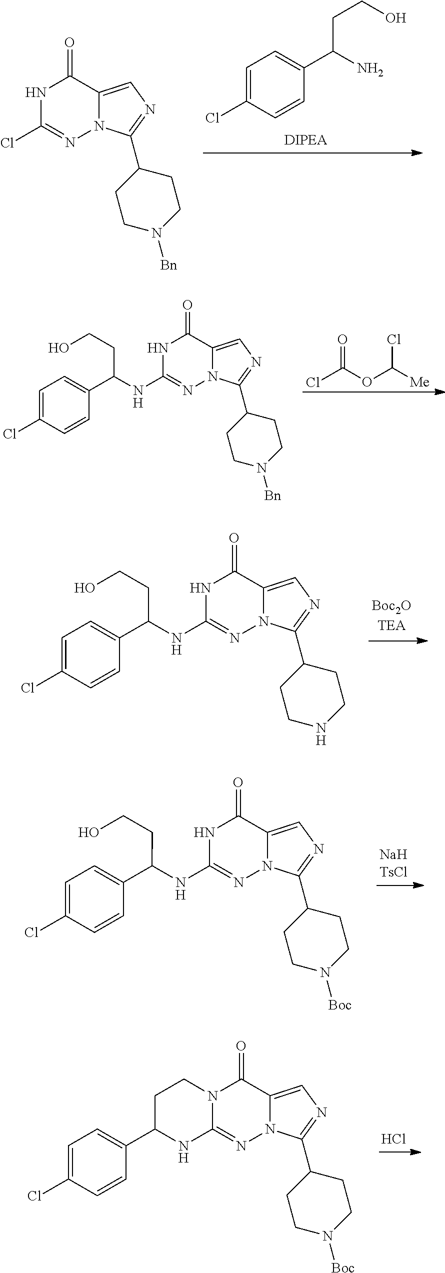

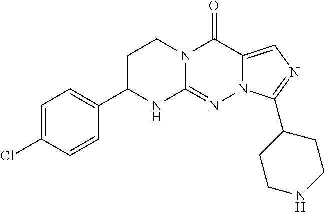 [SMILES: O=c1c2cnc(C3CCNCC3)n2nc2n1CCC(c1ccc(Cl)cc1)N2]

2-Methyl-2-propanyl 4-[2-(4-chlorophenyl)-6-oxo-1,2,3,4-tetrahydro-6H-imidazo[5,1-f]pyrimido[2,1-c][1,2,4]triazin-9-yl]-1-piperidinecarboxylate (20 mg, 0.04 mmol) was dissolved in hydrochloric acid in dioxane (3 mL) and the solution was stirred at rt for 2 h. Concentration gave the titled compound (8 mg, 53%) as white solids. ¹H NMR (400 MHz, CD₃OD): δ 7.65 (s, 1H), 7.43 (s, 4H), 7.01-6.98 (m, 1H), 4.89-4.72 (m, 1H), 4.09-4.06 (m, 1H), 3.94-3.89 (m, 1H), 3.71-3.32 (m, 1H), 3.24-3.20 (m, 2H), 2.85-2.76 (m, 2H), 2.39-2.34 (m, 2H), 2.08-1.95 (m, 3H), 1.94-1.88 (m, 2H). MS (ESI): m/e=0.385.1 [M+1]⁺.

### 7-(1-Benzyl-4-piperidinyl)-2-{[1-(4-chlorophenyl)-3-hydroxypropyl]amino}imidazo[5,1-f][1,2,4]triazin-4(1H)-one

To a mixture of 3-amino-3-(4-chlorophenyl)1-propanol (93 mg, 0.5 mmol), 7-(1-benzyl-4-piperidinyl)-2-chloroimidazo[5,1-f][1,2,4]triazin-4(1H)-one (synthesized in the similar procedure to Reference Example 2, 172 mg, 0.5 mmol) and sodium iodide (75 mg, 0.5 mmol) in 1-butanol (5 mL) was added DIPEA (194 mg, 1.5 mmol). The mixture was flushed with nitrogen (3 times), and then heated at 170° C. under microwave for 15 h. Cooled to r.t., filtered, concentrated to dry to give a residue, which was purified on reverse phase chromatography column (A: water (0.01% Ammonia), B: MeCN) to give the titled compound (212 mg, 43.0%) as white solids. MS (ESI): m/e=493 [M+H]⁺.

### 2-{[1-(4-Chlorophenyl)-3-hydroxypropyl]amino}-7-(4-piperidinyl)imidazo[5,1-f][1,2,4]triazin-4(1H)-one

To the solution of 7-(1-benzyl-4-piperidinyl)-2-{[1-(4-chlorophenyl)-3-hydroxypropyl]amino}imidazo[5,1-f][1,2,4]triazin-4(1H)-one (197 mg, 0.4 mmol) in DME (10 mL) was added 1-chloroethyl carbonochloridate (86 mg, 0.6 mmol). The reaction mixture was heated to reflux overnight. After concentration in vacuo, purified with flash column to afford the titled compound (114 mg, 70.7%). MS (ESI): m/e=0.403 [M+1]⁺.

### 2-Methyl-2-propanyl 4-(2-{[1-(4-chlorophenyl)-3-hydroxypropyl]amino}-4-oxo-1,4-dihydroimidazo[5,1-f][1,2,4]triazin-7-yl)-1-piperidinecarboxylate

To the solution of 2-{[1-(4-chlorophenyl)-3-hydroxypropyl]amino}-7-(4-piperidinyl)imidazo[5,1-f][1,2,4]triazin-4(1H)-one (63 mg, 0.16 mmol) in DCM (10 mL) was added Boc₂O (41 mg, 0.19 mmol) and TEA (58 mg, 0.57 mmol). The reaction mixture was stirred at rt for 3 h. Quenched with MeOH, after concentration in vacuo, purified with flash column to give the titled compound (50 mg, 62.5%) as white solids. MS: m/e=503 [M+H]⁺.

### 2-Methyl-2-propanyl 4-[2-(4-chlorophenyl)-6-oxo-1,2,3,4-tetrahydro-6H-imidazo[5,1-f]pyrimido[2,1-c][1,2,4]triazin-9-yl]-1-piperidinecarboxylate

To the solution of 2-methyl-2-propanyl 4-(2-{[1-(4-chlorophenyl)-3-hydroxypropyl]amino}-4-oxo-1,4-dihydroimidazo[5,1-f][1,2,4]triazin-7-yl)-1-piperidinecarboxylate (50 mg, 0.1 mmol) in THF (5 mL) was added sodium hydride (5.28 mg, 0.22 mmol), followed by p-tosyl chloride (21 mg, 0.12 mmol). The reaction mixture was stirred at rt for 3 h. H₂O (1 mL) was added and the solution was extracted with EtOAc. The combined organic layer was washed with brine and dried in sodium sulfate. After evaporation of the solvent, the residue was purified with column chromatography (DCM:MeOH=100:1) to yield the titled compound (20 mg, 41%) as white solids. MS (ESI): m/e=0.485 [M+1]⁺.

### Example 254

### 1-Isopropyl-6,7,8,10-tetrahydropyrazolo[3,4-d]pyrimido[1,2-a]pyrimidin-4(1H)-one

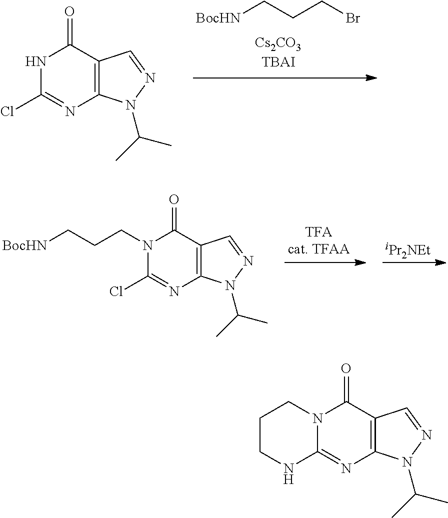 [SMILES: "iPr2NEt" ; CC(C)n1ncc2c(=O)[nH]c(Cl)nc21 ; CC(C)(C)OC(=O)NCCCBr ; CC(C)n1ncc2c(=O)n(CCCNC(=O)OC(C)(C)C)c(Cl)nc21 ; CC(C)n1ncc2c(=O)n3c(nc21)NCCC3]

A mixture of 2-methyl-2-propanyl[3-(6-chloro-1-isopropyl-4-oxo-1,4-dihydro-5H-pyrazolo[3,4-d]pyrimidin-5-yl)propyl]carbamate (910 mg, 2.46 mmol), TFA (1.9 mL, 24.6 mmol) and TFAA (17 μL, 0.123 mmol) in DCM (4.9 mL) was stirred at rt overnight. The reaction was concentrated in vacuo. A mixture of the above crude and DIPEA in THF was stirred at rt for 12 h. The resulting mixture was diluted with water, and extracted with EtOAc. The combined organic extracts were dried over sodium sulfate, filtered, and concentrated in vacuo. The residue was purified by silica gel chromatography (chloroform:MeOH) to give the titled compound (482 mg, 84%). ¹H NMR (CDCl₃) δ: 7.89 (s, 1H), 5.26 (br s, 1H), 4.76-4.69 (m, 1H), 4.05-4.02 (m, 2H), 3.48-3.44 (m, 2H), 2.09-2.03 (m, 2H), 1.45 (d, J=6.7 Hz, 6H). LCMS: m/z=234 [M+H]⁺.

### 2-Methyl-2-propanyl[3-(6-chloro-1-isopropyl-4-oxo-1,4-dihydro-5H-pyrazolo[3,4-d]pyrimidin-5-yl)propyl]carbamate

A mixture of 6-chloro-1-isopropyl-1,5-dihydro-4H-pyrazolo[3,4-d]pyrimidin-4-one (Reference Example 1, 2.1 g, 9.88 mmol), cesium carbonate (4.82 g, 1.8 mmol), tetra-n-butylammonium iodide (365 mg, 0.988 mmol), and tert-butyl n-(3-bromopropyl)carbonate (2.35 g, 9.88 mmol) in DMF (33 mL) was stirred at rt for 2 days. The reaction was quenched with water, and extracted with EtOAC. The combined organic extracts were washed with brine, dried over sodium sulfate, filtered, and concentrated in vacuo. The residue was purified by silica gel chromatography (hexane:EtOAc) to give the titled compound (910 mg, 25%). LCMS: m/z=370 [M+H]⁺.

### Example 255

### (2R)-2-(4-Methoxyphenyl)-8-[1-(trifluoromethyl)cyclopropyl]-2,10-dihydro-3H,5H-diimidazo[2,1-c:5′,1′-f][1,2,4]triazin-5-one

### Example 256

### (2S)-2-(4-Methoxyphenyl)-8-[1-(trifluoromethyl)cyclopropyl]-2,10-dihydro-3H,5H-diimidazo[2,1-c:5′,1′-f][1,2,4]triazin-5-one

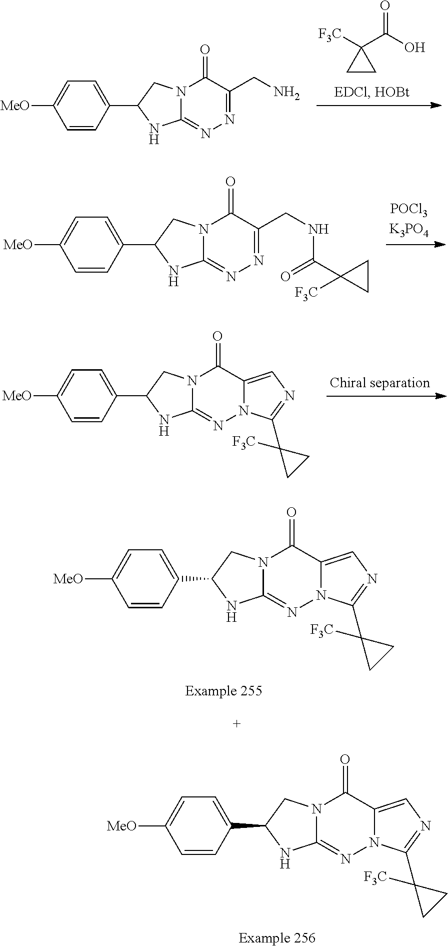

2-(4-Methoxyphenyl)-8-[1-(trifluoromethyl)cyclopropyl]-2,10-dihydro-3H,5H-diimidazo[2,1-c:5′,1′-f][1,2,4]triazin-5-one (470 mg) was separated by Chiral-HPLC to afford the titled compounds (Example 255: 62 mg; Example 256: 34 mg).

Example 255: ¹H NMR (400 MHz, DMSO-d₆) δ 8.54 (s, 1H), 7.66 (s, 1H), 7.37 (d, J=6.8 Hz, 2H), 6.96 (d, J=6.8 Hz, 2H), 5.08 (t, J=6.8 Hz, 1H), 4.47 (dd, J1=8.8 Hz, J2=7.6 Hz, 1H), 3.76 (s, 3H), 3.70 (dd, J1=8.8 Hz, J2=6.0 Hz, 1H), 1.46-1.52 (m, 2H), 1.31-1.38 (m, 2H). Chiral separation: retention time 2.22 min (Method 9)

Example 256: ¹H NMR (400 MHz, DMSO-d₆): δ 8.54 (s, 1H), 7.66 (s, 1H), 7.37 (d, J=6.8 Hz, 2H), 6.96 (d, J=6.8 Hz, 2H), 5.08 (t, J=6.8 Hz, 1H), 4.47 (dd, J1=8.8 Hz, J2=7.2 Hz, 1H), 3.76 (s, 3H), 3.71 (dd, J1=8.8 Hz, J2=6.0 Hz, 1H), 1.49 (br s, 2H), 1.31-1.38 (m, 2H). Chiral separation: retention time 4.00 min (Method 9)

### N-{[7-(4-Methoxyphenyl)-4-oxo-1,4,6,7-tetrahydroimidazo[2,1-c][1,2,4]triazin-3-yl]methyl}-1-(trifluoromethyl)cyclopropanecarboxamide

To the solution of 3-(aminomethyl)-7-(4-methoxyphenyl)-6,7-dihydroimidazo[2,1-c][1,2,4]triazin-4(1H)-one (synthesized in a similar manner to Example 228, 800 mg, 2.93 mmol) in DMF (8 mL) was added 1-(trifluoromethyl)cyclopropanecarboxylic acid (680 mg, 4.40 mmol), EDCI (1.12 g, 5.86 mmol), HOBt monohydrate (790 mg, 5.86 mmol), and DIPEA (1.14 g, 8.79 mmol). The mixture was stirred at rt for 12 h. The solution was evaporated in vacuo purified with prep-HPLC to give the titled compound (840 mg, 70%) as white solids. MS (ESI): m/e=410 [M+1]⁺.

### 2-(4-Methoxyphenyl)-8-[1-(trifluoromethyl)cyclopropyl]-2,10-dihydro-3H,5H-diimidazo[2,1-c:5′,1′-f][1,2,4]triazin-5-one

To a 25 mL flask was added N-{[7-(4-methoxyphenyl)-4-oxo-1,4,6,7-tetrahydroimidazo[2,1-c][1,2,4]triazin-3-yl]methyl}-1-(trifluoromethyl)cyclopropanecarboxamide (1 g, 2.44 mmol), tri potassium phosphate (2.07 g, 9.76 mmol) and phosphoryl chloride (20 mL) and the mixture was stirred at 80° C. for 20 h. After evaporation of solvent, the crude was purified with prep-HPLC to give the titled compound (470 mg, 50%) as white solids. MS (ESI): m/e=392 [M+1]⁺.

The compounds of Example 257 to 260 were synthesized in a similar manner to Example 255 and 256.

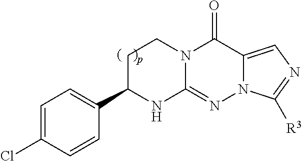

| | | | | Separation: |
|---|---|---|---|---|
| Ex. | | | | retention time |
| No. | p | R³— | ¹H NMR | method |
| 257 | 0 | 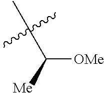 [SMILES: *[C@H](C)OC] | ¹H NMR (400 MHz, CDCl₃): δ 7.83 (s, 1H), 7.43 (d, J = 8.4 Hz, 2H), 7.36 (d, J = 8.4 Hz, 2H), 5.36 (s, 1H), 5.14 (t, J = 8.2 Hz, 1H), 4.88 (dd, J1 = 6.8 Hz , J2 = 6.8 Hz, 1H), 4.62-4.57 (m, 1H), 3.90 (dd, J1 = 7.2 Hz, J2 = 7.2 Hz, 1H), 3.31 (s, 3H), 1.69-1.66 (m, 3H) | 3.28 min Method 1 |
| 258 | 0 | 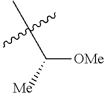 [SMILES: *[C@@H](C)OC] | ¹H NMR (400 MHz, CDCl₃): δ 7.83 (s, 1H), 7.43 (d, J = 8.4 Hz, 2H), 7.36 (d, J = 8.4 Hz, 2H), 5.36 (s, 1H), 5.14 (t, J = 8.2 Hz, 1H), 4.88 (dd, J1 = 6.8 Hz, J2 = 6.8 Hz, 1H), 4.62-4.57 (m, 1H), 3.90 (dd, J1 = 7.2 Hz, J2 = 7.2 Hz, 1H), 3.31 (s, 3H), 1.69-1.66 (m, 3H) | 5.41 min Method 1 |
| 259 | 1 | 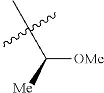 [SMILES: *[C@H](C)OC] | ¹H NMR (400 MHz, DMSO-d₆): δ 7.85 (s, 1H), 7.42 (d, J = 8.4 Hz, 2H), 7.31 (d, J = 8.8 Hz, 2H), 5.16 (br s, 1H), 4.89 (q, J = 6.8 Hz, 1H), 4.71-4.67 (m, 1H), 4.29-4.24 (m, 1H), 3.90-3.83 (m, 1H), 3.32 (s, 3H), 2.41-2.36 (m, 1H), 2.09-2.05 (m, 1H), 1.68 (d, J = 6.4 Hz, 3H) | 4.72 min Method 7 |
| 260 | 1 | 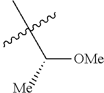 [SMILES: *[C@@H](C)OC] | ¹H NMR (400 MHz, DMSO-d₆): δ 7.85 (s, 1H), 7.42 (d, J = 8.8 Hz, 2H), 7.31 (d, J = 8.4 Hz, 2H), 5.16 (br s, 1H), 4.88 (q, J = 6.8 Hz, 1H), 4.71-4.66 (m, 1H), 4.30-4.24 (m, 1H), 3.91-3.84 (m, 1H), 3.34 (s, 3H), 2.41-2.36 (m, 1H), 2.10-2.06 (m, 1H), 1.67 (d, J = 6.8 Hz, 3H) | 4.96 min Method 7 |

### Example 261

### (2S)-2-(4-Hydroxyphenyl)-8-isopropyl-2,10-dihydro-3H,5H-diimidazo[2,1-c:5′,1′-f][1,2,4]triazin-5-one

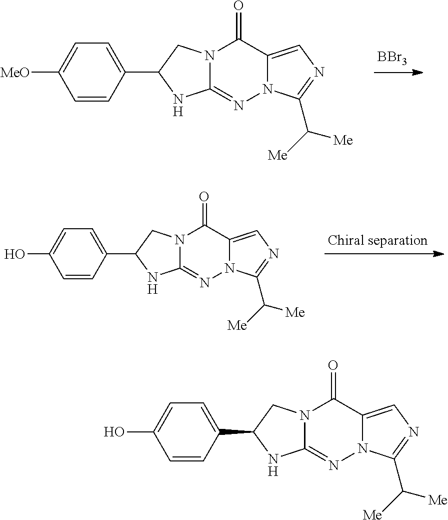 [SMILES: COc1ccc(C2Cn3c(nn4c(C(C)C)ncc4c3=O)N2)cc1 ; "BBr3" ; CC(C)c1ncc2c(=O)n3c(nn12)NC(c1ccc(O)cc1)C3 ; CC(C)c1ncc2c(=O)n3c(nn12)N[C@@H](c1ccc(O)cc1)C3]

2-(4-Hydroxyphenyl)-8-isopropyl-2,10-dihydro-3H,5H-diimidazo[2,1-c:5′,1′-f][1,2,4]triazin-5-one (470 mg) was separated by Chiral-HPLC to afford the titled compound (150 mg, 25%). ¹H NMR (400 MHz, DMSO-d₆): δ 9.51 (s, 1H), 8.24 (s, 1H), 7.55 (s, 1H), 7.24 (d, J=8.0 Hz, 2H), 6.77 (d, J=8.0 Hz, 2H), 4.99 (t, J=8 Hz, 1H), 4.39-4.44 (m, 1H), 3.64-3.68 (m, 1H), 3.29-3.34 (m, 1H), 1.25-1.29 (m, 6H). Chiral separation: retention time 3.63 min (Method 10).

### 2-(4-Hydroxyphenyl)-8-isopropyl-2,10-dihydro-3H,5H-diimidazo[2,1-c:5′,1′-f][1,2,4]triazin-5-one

To solution of 8-isopropyl-2-(4-methoxyphenyl)-2,10-dihydro-3H,5H-diimidazo[2,1-c:5′,1′-f][1,2,4]triazin-5-one (Example 80, 1 g, 3.07 mmol) in DCM (10 mL) added tribromoborane (2.28 g, 9.23 mmol). The mixture was stirred at rt for 2 h. The crude product was purified by silica gel chromatography (eluted with DCM:MeOH-20:1) to give the titled compound 600 mg, 62.7%) as white solids. MS (ESI): m/e=312 [M+H]⁺.

### Example 262

### 2-[4-(Fluoromethoxy)phenyl]-8-isopropyl-2,10-dihydro-3H,5H-diimidazo[2,1-c:5′,1′-f][1,2,4]triazin-5-one

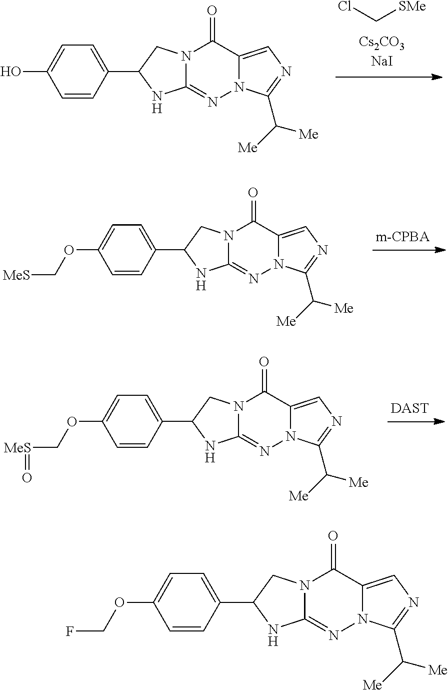

To a solution of 8-isopropyl-2-{4-[(methylsulfinyl)methoxy]phenyl}-2,10-dihydro-3H,5H-diimidazo[2,1-c:5′,1′-f][1,2,4]triazin-5-one (60 mg, 0.1549 mmol) in DCM (2 mL), was added DAST (0.1248 g, 0.7745 mmol). The reaction was stirred at rt for 2 h. Treated with water, the resulting oil was purified by reverse phase HPLC with MeCN (15%) and water (85%) to provide the titled compound (2.35 mg, 4%) as white solids. ¹H NMR (400 MHz, CDCl₃): δ7.78 (s, 1H), 7.37 (d, J=8.8 Hz, 2H), 7.13 (d, J=8.4 Hz, 2H), 5.73 (d, J=54.4 Hz, 2H), 5.09 (t, J=7.6 Hz, 1H), 5.00 (s, 1H), 4.54 (dd, J=8.4, 11.6 Hz, 1H), 3.89 (dd, J=7.2, 11.6 Hz, 1H), 3.40-3.36 (m, 1H), 1.40-1.37 (m, 6H). MS: m/e=344 [M+H]⁺.

### 8-Isopropyl-2-{4-[(methylthio)methoxy]phenyl}-2,10-dihydro-3H,5H-diimidazo[2-c:5′,1′-f][1,2,4]triazin-5-one

To a solution of 2-(4-hydroxyphenyl)-8-isopropyl-2,10-dihydro-3H,5H-diimidazo[2,1-c:5′,1′-f][1,2,4]triazin-5-one (see Example 261, 0.1557 g, 0.5 mmol) in DMF (2 mL), was added cesium carbonate (0.4887 g, 1.5 mmol) and sodium iodide (0.0075 g, 0.05 mmol). To the mixture, was added (chloromethyl)(methyl)sulfane (0.0483 g, 0.5 mmol) at 0° C. The reaction was stirred at 0° C. for 24 h. Filtered and concentrated, the resulting oil was purified by reverse phase HPLC with MeCN (25%) and water (75%) to give the titled compound (60 mg, 32%) as white solids. MS: m/e=372 [M+H]⁺.

### 8-Isopropyl-2-{4-[(methylsulfinyl)methoxy]phenyl}-2,10-dihydro-3H,5H-diimidazo[2,1-c:5′,1′-f][1,2,4]triazin-5-one

To a solution of 8-isopropyl-2-{4-[(methylthio)methoxy]phenyl}-2,10-dihydro-3H,5H-diimidazo[2,1-c:5′,1′-f][1,2,4]triazin-5-one (60 mg, 0.1615 mmol) in DCM (2 mL), was added m-CPBA (0.0307 g, 0.1777 mmol). The reaction was stirred at rt for 2 h. Concentrated, the resulting solid was purified by reverse phase HPLC with MeCN (15%) and water (85%) to provide the titled compound (60 mg, 96%) as white solids. MS: m/e=388 [M+H]⁺.

### Example 263

### (2S)-2-(3-Methylphenyl)-9-(tetrahydro-2H-pyran-4-yl)-1,2,3,4-tetrahydro-6H-imidazo[5,1-f]pyrimido[2,1-c][1,2,4]triazin-6-one

### Example 264

### (2R)-2-(3-Methylphenyl)-9-(tetrahydro-2H-pyran-4-yl)-1,2,3,4-tetrahydro-6H-imidazo[5,1-f]pyrimido[2,1-c][1,2,4]triazin-6-one

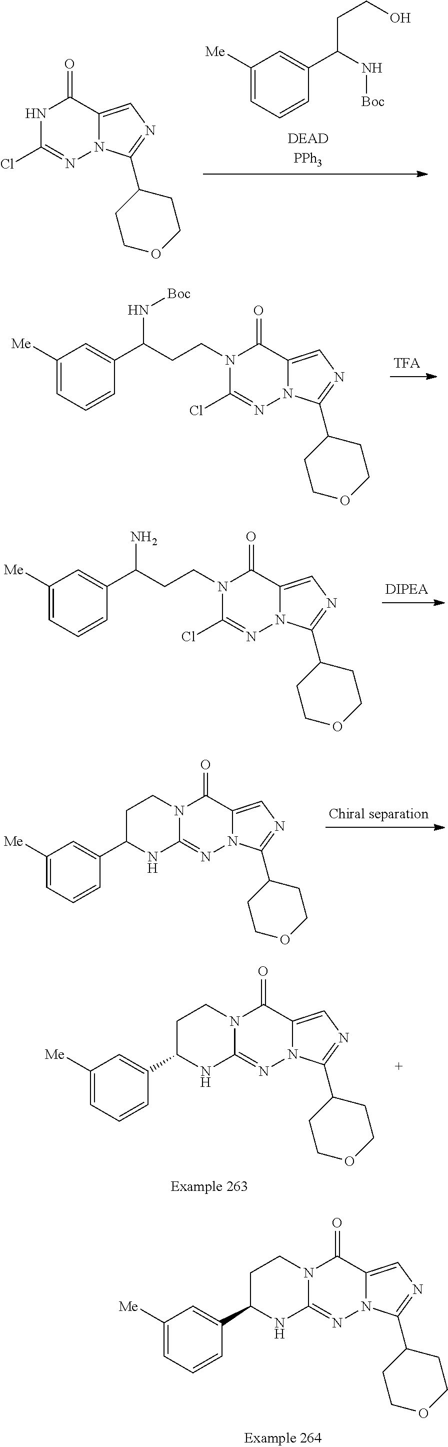

2-(3-Methylphenyl)-9-(tetrahydro-2H-pyran-4-yl)-1,2,3,4-tetrahydro-6H-imidazo[5,1-f]pyrimido[2,1-c][1,2,4]triazin-6-one (450 mg) was separated by chiral-HPLC to give the titled compounds (Example 263: 115 mg; Example 264: 112 mg).

Example 263: ¹H NMR (400 MHz, CDCl₃): δ 7.81 (s, 1H), 7.32-7.31 (m, 1H), 7.21-7.11 (m, 3H), 5.07 (s, 1H), 4.67-4.64 (m, 1H), 4.33-4.30 (m, 1H), 4.13-4.07 (m, 2H), 3.85-3.83 (m, 1H), 3.62-3.55 (m, 2H), 3.33-3.32 (m, 1H), 2.41 (m, 3H), 2.40-2.36 (m, 1H), 2.14-2.06 (m, 3H), 1.95-1.90 (m, 2H). Chiral separation: retention time 2.10 min (Method 10)

Example 264: ¹H NMR (400 MHz, CDCl₃): δ 7.81 (s, 1H), 7.32-7.31 (m, 1H), 7.21-7.11 (m, 3H), 5.07 (s, 1H), 4.67-4.64 (m, 1H), 4.33-4.30 (m, 1H), 4.13-4.07 (m, 2H), 3.85-3.83 (m, 1H), 3.62-3.55 (m, 2H), 3.33-3.32 (m, 1H), 2.41 (m, 3H), 2.40-2.36 (m, 1H), 2.14-2.06 (m, 3H), 1.95-1.90 (m, 2H). Chiral separation: retention time 3.46 min (Method 10)

### 2-Methyl-2-propanyl{3-[2-chloro-4-oxo-7-(tetrahydro-2H-pyran-4-yl)imidazo[5,1-f][1,2,4]triazin-3(4H)-yl]-1-(3-methylphenyl)propyl}carbamate

To a stirred solution of 2-chloro-7-(tetrahydro-2H-pyran-4-yl)imidazo[5,1-f][1,2,4]triazin-4(1H)-one (Reference Example 2, 1.28 g, 5 mmol) in THF (50 ml) was added 2-methyl-2-propanyl[3-hydroxy-1-(3-methylphenyl)propyl]carbamate (synthesized in a similar manner to Reference Example 7 intermediate, 1.33 g, 5 mmol) and triphenylphosphine (2.62 g, 10 mmol) at 0° C. under N₂, after 10 min. DEAD (1.74 g, 10 mmol) was added to the mixture. The mixture was stirred at rt overnight. The crude product was purified by silica gel chromatography (eluted with PE: EtOAc=4:1) to give the titled compound (1.05 g, 42%). MS: m/e=502 [M+H]⁺.

### 3-[3-Amino-3-(3-methylphenyl)propyl]-2-chloro-7-(tetrahydro-2H-pyran-4-yl)imidazo[5,1-f][1,2,4]triazin-4(3H)-one

To a solution of 2-methyl-2-propanyl{3-[2-chloro-4-oxo-7-(tetrahydro-2H-pyran-4-yl)imidazo[5,1-f][1,2,4]triazin-3(4H)-yl]-1-(3-methylphenyl)propyl}carbamate (1 g, 2 mmol) in DCM (10 mL) was added TFA (5 mL) at 0° C. The mixture was stirred at 0° C. for 1 h, quenched with water (50 mL), adjusted pH to 8 with sodium bicarbonate aq. solution, extracted with EtOAc (100 mL×3), dried over sodium sulfate, filtered, concentrated to give the titled crude as pale yellow solids (652 mg, 81.1%). MS: m/e=402 [M+H]⁺.

### 2-(3-Methylphenyl)-9-(tetrahydro-2H-pyran-4-yl)-1,2,3,4-tetrahydro-6H-imidazo[5,1-f]pyrimido[2,1-c][1,2,4]triazin-6-one

To a stirred solution of 3-[3-amino-3-(3-methylphenyl)propyl]-2-chloro-7-(tetrahydro-2H-pyran-4-yl)imidazo[5,1-f][1,2,4]triazin-4(3H)-one (603 mg, 1.5 mmol) in THF (20 mL) was added DIPEA (387 mg, 3 mmol) under N₂ atmosphere at rt, then heated to 45° C. for 2 h, cooled to rt, diluted with water (30 mL), extracted with EtOAc (100 mL×3), dried over sodium sulfate, filtered, concentrated to give a residue, which was purified by CC eluting with 4% MeOH in DCM to give the titled compound (450 mg, 82.1%) as pale yellow solids. MS: m/e=366 [M+H]⁺.

### Example 265

### 7-(4-Chlorophenyl)-4-(tetrahydro-2H-pyran-4-yl)-5,7,8,9-tetrahydro-11H-dipyrimido[1,2-a:5′,4′-d]pyrimidin-11-one

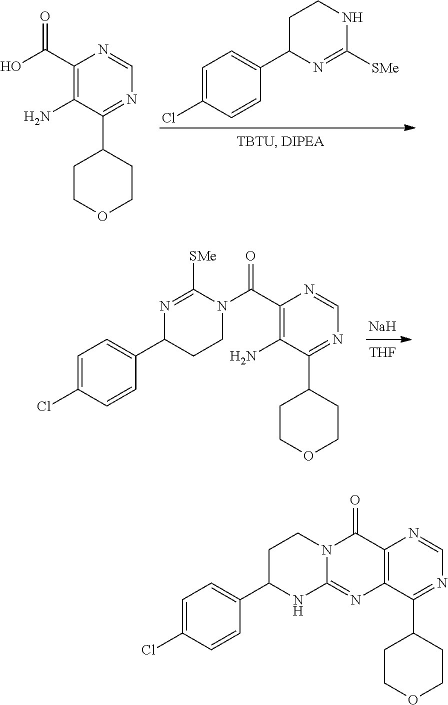

To a solution of [5-Amino-6-(tetrahydro-2H-pyran-4-yl)-4-pyrimidinyl][4-(4-chlorophenyl)-2-(methylthio)-5,6-dihydro-1(4H)-pyrimidinyl]methanone (60 mg, 0,135 mmol) in anhydrous THF (2 mL) was added dropwise sodium hydride (60% in mineral oil, 11 mg, 0.269 mmol) at 10° C. Then the mixture was heated to reflux for 1 h. The mixture was quenched with water (15 mL) and extracted with EtOAc (10 mL×3). The combined organic layers was dried over sodium sulfate and concentrated to dryness. The residue was purified by prep-TLC (EtOAc) to give the titled compound (35 mg, 65%) as white solids. ¹H NMR (400 MHz, CDCl₃) δ: 9.01 (s, 1H), 7.41 (d, J=8.4 Hz, 2H>, 7.32 (d, J=8.4 Hz, 2H), 5.68 (br s, 1H), 4.77-4.74 (m, 1H), 4.40-4.33 (m, 1H), 4.13-4.07 (m, 2H), 4.04-3.96 (m, 1H), 3.84-3.76 (m, 1H), 3.64-3.56 (m, 2H), 2.44-2.38 (m, 1H), 2.15-1.95 (m, 3H), 1.80-1.76 (m, 2H).

### [5-Amino-6-(tetrahydro-2H-pyran-4-yl)-4-pyrimidinyl][4-(4-chlorophenyl)-2-(methylthio)-5,6-dihydro-1(4H)-pyrimidinyl]methanone

To a solution of 5-amino-6-(tetrahydro-2H-pyran-4-yl)-4-pyrimidinecarboxylic acid (Reference Example 5, 93 mg, 0.417 mmol) and (4R)-4-(4-chlorophenyl)-2-(methylthio)-1,4,5,6-tetrahydropyrimidine (Reference Example 7, 100 mg, 0.417 mmol) in anhydrous DMF (2 mL), were added TBTU (201 mg, 0.625 mmol) and DIPEA (1 ml). The mixture was stirred at 10° C. for 16 h. The mixture was purified by prep-HPLC (0.1% NH₃.H₂O as additive) to give the titled compound (70 mg, 38%) as white solids.

### In Vitro HTRF PDE1 Inhibition Assay

An exemplary procedure for the in vitro Homogenous Time Resolved Fluorescence (HTRF) assay, which can be used to determine the inhibitory action of compounds of the invention toward PDE1 or its isoforms, follows.

The HTRF PDE1 assay utilized the HTRF technology, which is based on the competition between unlabeled cyclic nucleotide and cyclic nucleotide labeled with XL665 for the binding to cyclic nucleotide-specific antibody labeled with cryptate. The HTRF signal is thus inversely proportional to the concentration of cyclic nucleotide being measured. Since phosphodiesterases break down cyclic nucleotides the HTRF signal was used to determine PDE activity.

The Cisbio cGMP HTRF assay kit (Cat no: 62GM2PEC) was utilized. Cyclic GMP was diluted to 200 nM in HTRF assay buffer (1 mM CaCl₂, 10 mM MgCl₂, 10 mM Tris-HCl, 0.1% BSA, pH7.4). 10 μl of compound or DMSO was diluted in 200 nM cyclic GMP solution and added to wells of a 96 well white plate to give 100 nM cyclic GMP in 1% DMSO final concentration. PDE (1A3, 1B or 1C) was diluted to 2× working concentration in HTRF assay buffer with 4 μg/ml Calmodulin, and 10 μl was added to initiate the reaction. The plate was then incubated for 45 minutes at 37° C. d2-Labelled cyclic GMP and anti-cGMP cryptate were diluted in 50 mM phosphate buffer, 0.8M KF, 1% Triton X100, 0.2% BSA, pH7.0. Following incubation 10 μl d2-cGMP, then 10 μl anti cGMP cryptate were added to each well and the plate was incubated for 45 minutes at rt. The plate was then read on Perkin Elmer Victor at 2 different FRET readings ex/emm 340 nm/665 nm and 340 nm/615 nm.

Data obtained from the HTRF assay for selected compounds of the invention are listed in Table 2 to 4 below. Compounds having an IC₅₀ of <1 μM, are denoted as +++. Compounds having an IC₅₀ of 1-10 μM, are denoted as ++. Compounds having an IC₅₀ of 10-100 μM, are denoted as +.

TABLE 2
Results of in vitro PDE1B HTRF Assay

| Ex. No. | IC₅₀ (μM) |
|---|---|
| 1 | +++ |
| 2 | +++ |
| 3 | ++ |
| 4 | +++ |
| 5 | + |
| 6 | +++ |
| 7 | +++ |
| 8 | +++ |
| 9 | +++ |
| 10 | +++ |
| 11 | +++ |
| 12 | ++ |
| 13 | +++ |
| 14 | +++ |
| 15 | +++ |
| 16 | +++ |
| 17 | +++ |
| 18 | +++ |
| 19 | +++ |
| 20 | +++ |
| 21 | +++ |
| 22 | +++ |
| 23 | +++ |
| 24 | +++ |
| 25 | ++ |
| 26 | ++ |
| 27 | +++ |
| 28 | ++ |
| 29 | +++ |
| 30 | +++ |
| 31 | +++ |
| 32 | +++ |
| 33 | ++ |
| 34 | +++ |
| 35 | +++ |
| 36 | +++ |
| 37 | +++ |
| 38 | +++ |
| 39 | ++ |
| 40 | +++ |
| 41 | ++ |
| 42 | ++ |
| 43 | +++ |
| 44 | +++ |
| 45 | + |
| 46 | +++ |
| 47 | +++ |
| 48 | +++ |
| 49 | ++ |
| 50 | +++ |
| 51 | +++ |
| 52 | +++ |
| 53 | +++ |
| 54 | +++ |
| 55 | +++ |
| 56 | +++ |
| 57 | +++ |
| 58 | +++ |
| 59 | +++ |
| 60 | +++ |
| 61 | +++ |
| 62 | +++ |
| 63 | +++ |
| 64 | +++ |
| 65 | +++ |
| 66 | +++ |
| 67 | +++ |
| 68 | +++ |
| 69 | +++ |
| 70 | ++ |
| 71 | ++ |
| 72 | ++ |
| 73 | ++ |
| 75 | +++ |
| 76 | +++ |
| 77 | +++ |
| 78 | +++ |
| 79 | +++ |
| 80 | +++ |
| 81 | +++ |
| 82 | +++ |
| 83 | +++ |
| 84 | +++ |
| 85 | +++ |
| 86 | +++ |
| 87 | +++ |
| 88 | +++ |
| 89 | +++ |
| 90 | +++ |
| 91 | +++ |
| 92 | +++ |
| 93 | +++ |
| 94 | +++ |
| 95 | +++ |
| 96 | +++ |
| 97 | +++ |
| 98 | +++ |
| 99 | +++ |
| 100 | +++ |
| 101 | +++ |
| 102 | +++ |
| 103 | +++ |
| 104 | +++ |
| 105 | +++ |
| 106 | +++ |
| 107 | +++ |
| 108 | +++ |
| 109 | +++ |
| 110 | + |
| 111 | + |
| 112 | +++ |
| 113 | ++ |
| 114 | ++ |
| 115 | +++ |
| 116 | ++ |
| 117 | +++ |
| 118 | +++ |
| 119 | +++ |
| 120 | +++ |
| 121 | +++ |
| 122 | +++ |
| 123 | ++ |
| 124 | ++ |
| 125 | +++ |
| 126 | +++ |
| 127 | +++ |
| 128 | +++ |
| 129 | +++ |
| 130 | +++ |
| 131 | +++ |
| 132 | +++ |
| 133 | +++ |
| 134 | +++ |
| 135 | +++ |
| 136 | +++ |
| 137 | +++ |
| 138 | +++ |
| 139 | +++ |
| 140 | +++ |
| 143 | +++ |
| 144 | +++ |
| 145 | ++ |
| 146 | +++ |
| 147 | ++ |
| 148 | +++ |
| 149 | ++ |
| 150 | +++ |
| 151 | +++ |
| 152 | ++ |
| 153 | + |
| 154 | ++ |
| 155 | ++ |
| 156 | + |
| 157 | ++ |
| 158 | ++ |
| 159 | + |
| 160 | +++ |
| 161 | +++ |
| 162 | +++ |
| 163 | +++ |
| 164 | +++ |
| 165 | +++ |
| 166 | +++ |
| 167 | +++ |
| 172 | ++ |
| 173 | +++ |
| 174 | +++ |
| 175 | +++ |
| 176 | +++ |
| 177 | ++ |
| 178 | ++ |
| 179 | ++ |
| 180 | ++ |
| 181 | +++ |
| 183 | +++ |
| 184 | ++ |
| 185 | +++ |
| 186 | +++ |
| 187 | +++ |
| 188 | +++ |
| 189 | + |
| 190 | +++ |
| 191 | +++ |
| 192 | +++ |
| 193 | +++ |
| 194 | ++ |
| 195 | +++ |
| 196 | ++ |
| 197 | ++ |
| 198 | +++ |
| 199 | ++ |
| 200 | +++ |
| 201 | ++ |
| 202 | +++ |
| 203 | +++ |
| 204 | ++ |
| 205 | + |
| 206 | +++ |
| 207 | + |
| 208 | +++ |
| 209 | + |
| 210 | + |
| 211 | ++ |
| 212 | ++ |
| 213 | ++ |
| 214 | ++ |
| 215 | +++ |
| 216 | +++ |
| 217 | ++ |
| 218 | +++ |
| 219 | ++ |
| 220 | +++ |
| 221 | +++ |
| 222 | + |
| 223 | + |
| 224 | +++ |
| 225 | +++ |
| 226 | +++ |
| 227 | +++ |
| 229 | +++ |
| 230 | +++ |
| 231 | +++ |
| 232 | +++ |
| 233 | ++ |
| 234 | ++ |
| 235 | +++ |
| 236 | ++ |
| 237 | ++ |
| 238 | +++ |
| 239 | +++ |
| 240 | +++ |
| 241 | +++ |
| 242 | +++ |
| 243 | +++ |
| 244 | +++ |
| 245 | +++ |
| 246 | ++ |
| 247 | +++ |
| 248 | ++ |
| 249 | ++ |
| 250 | +++ |
| 251 | ++ |
| 252 | ++ |
| 253 | ++ |
| 254 | +++ |
| 255 | +++ |
| 256 | +++ |
| 257 | +++ |
| 258 | +++ |
| 259 | +++ |
| 260 | +++ |
| 261 | +++ |
| 262 | +++ |
| 263 | +++ |
| 264 | +++ |
| 265 | +++ |

TABLE 3
Results of in vitro PDE1A HTRF Assay

| Ex. No. | IC₅₀ (μM) |
|---|---|
| 1 | ++ |
| 4 | ++ |
| 9 | +++ |
| 18 | +++ |
| 27 | +++ |
| 29 | +++ |
| 46 | + |
| 52 | ++ |
| 55 | +++ |
| 57 | +++ |
| 61 | +++ |
| 65 | +++ |
| 69 | +++ |
| 96 | +++ |
| 99 | +++ |
| 100 | +++ |
| 120 | +++ |
| 139 | +++ |
| 144 | +++ |
| 151 | +++ |
| 160 | +++ |
| 165 | +++ |
| 175 | +++ |
| 176 | +++ |
| 198 | +++ |
| 202 | +++ |
| 224 | +++ |
| 225 | +++ |
| 226 | +++ |
| 227 | +++ |
| 238 | +++ |
| 239 | +++ |
| 242 | +++ |
| 259 | +++ |
| 260 | +++ |
| 265 | +++ |

TABLE 4
Results of in vitro PDE1C HTRF Assay

| Ex. No. | IC₅₀ (μM) |
|---|---|
| 1 | ++ |
| 4 | ++ |
| 9 | ++ |
| 18 | ++ |
| 27 | ++ |
| 29 | ++ |
| 46 | ++ |
| 52 | ++ |
| 55 | ++ |
| 57 | +++ |
| 61 | +++ |
| 65 | +++ |
| 69 | +++ |
| 96 | +++ |
| 99 | +++ |
| 100 | +++ |
| 120 | +++ |
| 139 | ++ |
| 144 | +++ |
| 151 | +++ |
| 160 | ++ |
| 165 | ++ |
| 175 | +++ |
| 176 | ++ |
| 198 | +++ |
| 202 | +++ |
| 224 | ++ |
| 225 | +++ |
| 226 | +++ |
| 227 | +++ |
| 238 | ++ |
| 239 | ++ |
| 242 | ++ |
| 259 | +++ |
| 260 | ++ |
| 265 | ++ |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I: 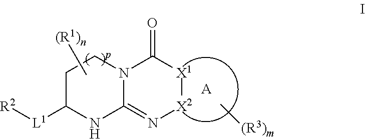
or a pharmaceutically acceptable salt thereof, wherein:
X¹ and X² are each independently C or N;
Ring A is a 5-6 membered heteroaryl ring having 1-4 heteroatoms independently selected from nitrogen, oxygen and sulfur;
L¹ is a covalent bond, or a C₁₋₄ bivalent straight or branched hydrocarbon chain, wherein one or more hydrogen atoms of the chain are optionally and independently replaced by halogen, and wherein one or two methylene units of the chain are optionally and independently replaced by —N(R)—, —N(R)C(O)—, —C(O)N(R)—, —N(R)C(O)N(R)—, —N(R)C(S)N(R)—, —N(R)S(O)₂—, —S(O)₂N(R)—, —C(O)—, —OC(O)—, —C(O)O—, —O—, —S—, —S(O)— or S(O)₂—;
each R¹ is independently halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)S(O)₂R, —S(O)₂N(R)₂, —OC(O)OR, —C(O)OR, —OC(O)R, —C(O)R; —S(O)R, or —S(O)₂R;
R² is halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)SO₂R, —S(O)₂N(R)₂, —C(O)R, —C(O)OR, —OC(O)R, —S(O)R, —S(O)₂R, or Cy;
Cy is a ring, substituted with q instances of R⁴; wherein said ring is a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; or an 8-10 membered bicyclic heteroaromatic ring having 1-5 heteroatoms independently selected from nitrogen, oxygen, and sulfur;
each R is independently
(i) a hydrogen,
(ii) a C₁₋₆ aliphatic (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) an oxo), or
(iii) a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring; a 5-6 membered monocyclic heteroaromatic ring; or an 8-10 membered bicyclic heteroaromatic ring, wherein each of said groups is optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) a cyano;
each R³ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, or —B(OR)₂;
each R⁴ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, or —B(OR)₂; or
two R⁴ are taken together with their intervening atoms to form a 5-6 membered saturated, partially unsaturated, or aromatic ring having 0-3 heteroatoms independently selected from nitrogen, oxygen, and sulfur;
wherein one or more of {two instances of R¹}, {R¹ and an R²}, and {two instances of R³} may be taken together with their intervening atoms to form a ring, substituted with r instances of R⁴; wherein said ring is a 3-8 membered saturated or partially unsaturated monocyclic carbocyclic ring; phenyl; a 4-8 membered saturated or partially unsaturated monocyclic heterocyclic ring having 1-2 heteroatoms independently selected from nitrogen, oxygen, and sulfur; or a 5-6 membered monocyclic heteroaromatic ring having 1-4 heteroatoms independently selected from nitrogen, oxygen, and sulfur;
m is 0-4;
n is 0-3;
p is 0-2;
q is 0-5; and
r is 0-5.

2. The compound of claim 1, wherein the compound is a compound of formula I-a, I-b, or I-c: 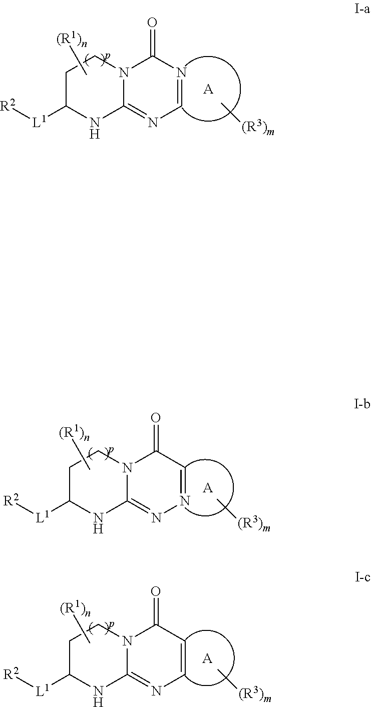
or a pharmaceutically acceptable salt thereof.

3. The compound of claim 1 wherein the compound is a compound of formula II-a, II-b, II-c, II-d, II-e, II-f, II-g, II-h, II-i, II-j or II-k: 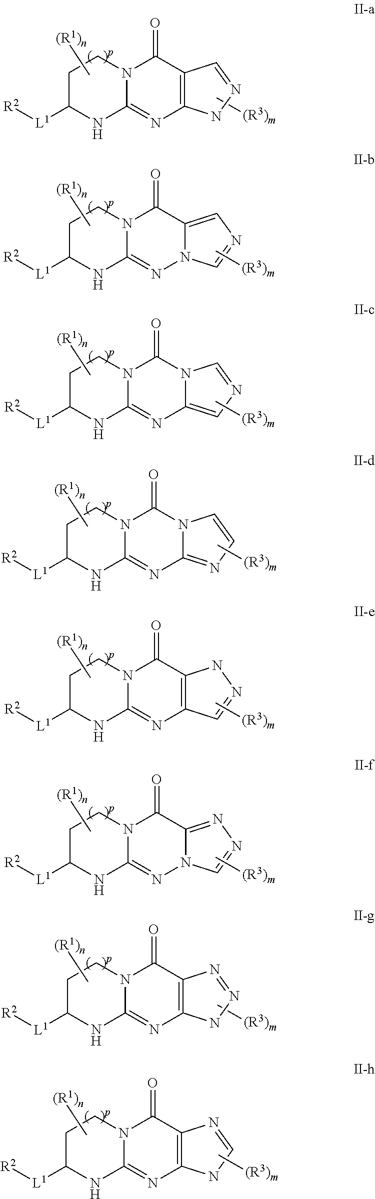 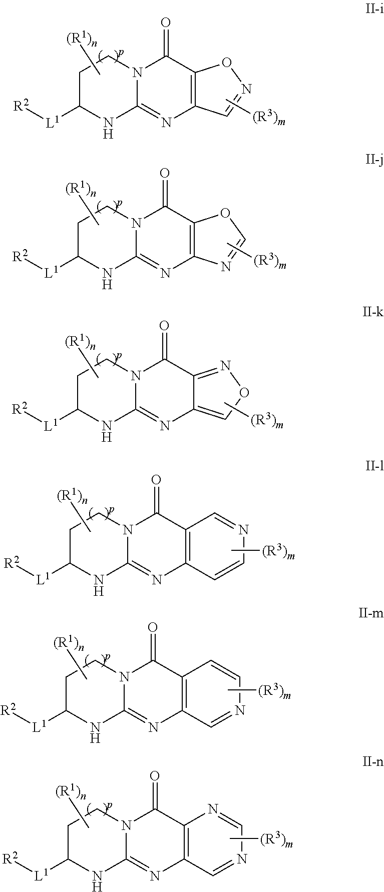
or a pharmaceutically acceptable salt thereof.

4. The compound of claim 3, or a pharmaceutically acceptable salt thereof, wherein the compound is a compound of formula II-a II-b, II-k, II-l, or II-m.

5. The compound of claim 1 or a pharmaceutically acceptable salt thereof wherein R¹ is
(i) a hydrogen, or
(ii) a phenyl (said group being optionally substituted with the same or different 1 to 4 group(s) selected from
(a) a halogen,
(b) a C₁₋₆ alkyl (said group being optionally substituted with the same or different 1 to 3 halogen), and
(c) a C₁₋₆ alkoxy (said group being optionally substituted with the same or different 1 to 3 halogen)).

6. The compound of claim 1, wherein L¹ is a covalent bond.

7. The compound of claim 1, wherein L¹ is methylene.

8. The compound of claim 1, wherein R² is a hydrogen or Cy.

9. The compound of claim 1, wherein R² is a C₃₋₇ cycloaliphatic; a phenyl; a 5-6 membered monocyclic heteroaryl, or a 4-8 membered saturated or partially unsaturated monocyclic heterocyclyl, each of said group is optionally substituted with the same or different 1 to 4 group(s) selected from the group consisting of
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) a cyano.

10. The compound of claim 1, or a pharmaceutically acceptable salt thereof, wherein R³ is
(i) a hydrogen,
(ii) a halogen,
(iii) a C₁₋₆ aliphatic (said group being optionally substituted with the same or different 1 to 4 group(s) selected from
(a) a halogen,
(b) a C₁₋₆ alkoxy (said group being optionally substituted with the same or different 1 to 3 halogen),
(c) a hydroxy, and
(d) an oxo), or
(iv) 4-8 membered saturated or partially unsaturated monocyclic heterocyclyl (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆alkoxy (said group being optionally substituted with the same or different 1 to 3 halogen),
(d) a hydroxy, and
(e) a cyano).

11. A composition comprising the compound according to claim 1 and a pharmaceutically acceptable carrier, adjuvant, or vehicle.

12. A method of inhibiting PDE1 in a patient in need thereof, comprising administering to said patient the composition according to claim 11.

13. A method of inhibiting PDE1 in a biological sample, comprising contacting the biological sample with the compound according to claim 1.

14. A method for treating a neurological or psychiatric disorder in a patient in need thereof, comprising administering to said patient the composition according to claim 11.

15. The method according to claim 14, wherein the neurological or psychiatric disorder is Alzheimer's Disease, Parkinson's Disease, depression, cognitive impairment, stroke, schizophrenia, Down Syndrome, or Fetal Alcohol Syndrome.

16. The method according to claim 14, wherein the neurological or psychiatric disorder involves a deficit in one or more cognitive domains as defined by DSM-5.
